(19) United States
(12) Patent Application Publication
(10) Pub. No.: US 2025/0333405 A1
(43) Pub. Date: Oct. 30, 2025

(54) ANTAGONISTS OF GPR39 PROTEIN

(71) Applicant: VASOCARDEA, INC., Portland, OR (US)

(72) Inventors: AGOSTINO CIANCIULLI, VERONA (IT); FABRIZIO MICHELI, VERONA (IT); LUNI MARGARET LAURA TRIST, VERONA (IT)

(21) Appl. No.: 18/645,159

(22) Filed: Apr. 24, 2024

(51) Int. Cl. C07D 417/14; A61K 31/4025; A61K 31/407; A61K 31/4196; A61K 31/439; A61K 31/444; A61K 31/454; A61K 31/4545; A61K 31/496; A61K 31/506; A61K 31/5377; A61K 31/5386; A61K 31/541; C07D 207/06; C07D 401/10; C07D 401/14; C07D 403/10; C07D 403/14; C07D 409/10; C07D 413/10; C07D 413/14; C07D 417/10; C07D 471/04; C07D 491/107; C07D 498/08; C07F 5/02

(52) U.S. Cl. CPC C07D 417/14; A61K 31/4025; A61K 31/407; A61K 31/4196; A61K 31/439; A61K 31/444; A61K 31/454; A61K 31/4545; A61K 31/496; A61K 31/506; A61K 31/5377; A61K 31/5386; A61K 31/541; C07D 207/06; C07D 401/10; C07D 401/14; C07D 403/10; C07D 403/14; C07D 409/10; C07D 413/10; C07D 413/14; C07D 417/10; C07D 471/04; C07D 491/107; C07D 498/08; C07F 5/025

(57) ABSTRACT

Novel compounds that act as antagonists to human GPR39 protein are disclosed. Pharmaceutical compositions and methods of use for antagonists to human GPR39 protein are disclosed. In particular, methods of using the antagonists in the treatment of diseases or conditions including cardiovascular conditions, endocrine system and hormone disorders, cancer disorders, metabolic diseases, gastrointestinal and liver diseases, hematological disorders, neurological disorders and respiratory diseases are disclosed herein.

### CROSS REFERENCE TO RELATED APPLICATIONS

This application is a continuation of International Patent Application PCT/US2022/047658, filed Oct. 25, 2022, and entitled “ANTAGONISTS OF GPR39 PROTEIN,” which claims priority to U.S. Provisional Patent Application No. 63/272,076, filed on Oct. 26, 2021, and entitled “ANTAGONISTS OF GPR39 PROTEIN,” the complete disclosure of both of which are hereby incorporated by reference in their entirety.

### FIELD OF THE INVENTION

The present disclosure concerns novel compounds that act as antagonists to human GPR39 protein. Additionally, the present disclosure relates to pharmaceutical compositions and methods of using antagonists to human GPR39 protein in the treatment of diseases or conditions including cardiovascular conditions, endocrine system and hormone disorders, cancer disorders, metabolic diseases, gastrointestinal and liver diseases, hematological disorders, neurological disorders and respiratory diseases.

### BACKGROUND OF THE INVENTION

Use of GPR39 antagonists has been described for methods of treating pain sensitivity, including hyperalgesia, and suppressing appetite (U.S. Pat. Publication 2009/0298756-Jin et al.).

The use of GPR39 agonists and/or antagonists in enhancing glucose regulation and treating impaired carbohydrate metabolism, including in disorders such as diabetes and metabolic syndrome are discussed in WO 2007/141322—Moreaux et al.—Janssen Pharmaceutica N.V.

The article Altered Gastrointestinal and Metabolic Function in the GPR39-Obestatin Receptor—Knockout Mouse, Gastroenterology, Moechars et al., October 2006, Vol. 131, Issue 4, pp. 1131-1141, discloses conclusions that GPR39 receptor antagonists may be useful in disorders affecting stomach motility, including such as functional dyspepsia and diabetic gastroparesis and/or colorectal motility such as irritable bowel syndrome, diarrhea, or chronic constipation.

The role of the obestatin/GPR39 system in human gastric adenocarcinomas, Alen et al., Oncotarget 2016, 7:5957-5971 addresses the role of the obestatin/GPR39 system in regulating motility, EMT, and invasion of gastric adenocarcinoma cells.

GPR39 antagonists are also discussed in the article Enhanced ZnR/GPR39 Activity in Breast Cancer, an Alternative Trigger of Signaling Leading to Cell Growth, Ventura-Bixenshpaner et al., Scientific Reports (2018) 8:8119, particularly their use in treating breast cancer, including ER negative breast cancer.

The use of GPR39 antagonists in the treatment of various cancers is discussed in U.S. 2004/0071708 (Claassen et al.).

Inge Depoortere discloses uses of GPR39 antagonists in treating motility disorders, such as functional dyspepsia, hypoparesis, and chronic constipation in her article, GI functions of GPR39: novel biology, Current Opinion in Pharmacology, 2012, 12:647-652.

The article The zinc sensing receptor, ZnR/GPR39, controls proliferation and differentiation of colonocytes and thereby tight junction formation in the colon, Cohen et al., Cell Death and Disease (2014) 5, e1307, discusses potential uses for GPR antagonists in promoting or enhancing colon epithelial function and tight junction barrier integrity, including treating ulcerative colon diseases, such as ulcerative colitis, and diarrheal pathologies.

### BRIEF SUMMARY OF THE INVENTION

Provided is a compound of Formula (I-A), or a pharmaceutically acceptable salt thereof:

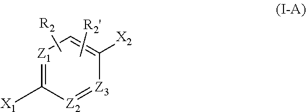

wherein:
X₁ is selected from the group of:

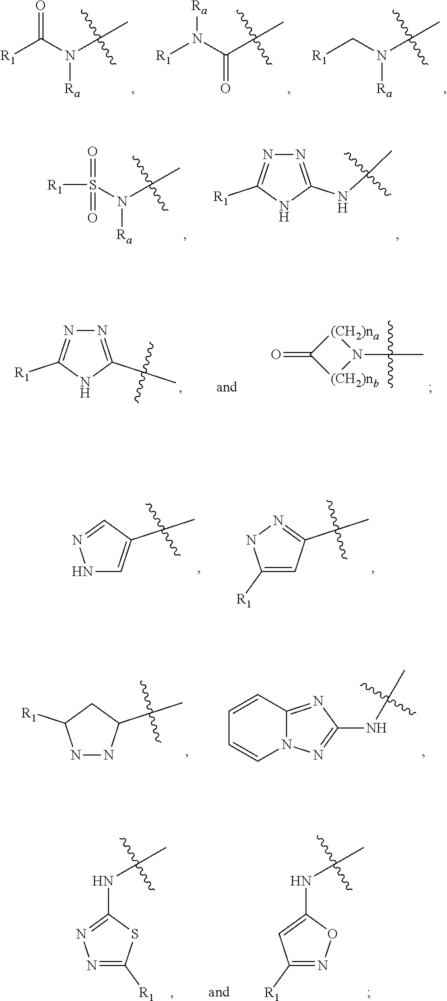

nₐ is an integer selected from the group of 0, 1, and 2;
n_{b} is an integer selected from the group of 0, 1, 2, 3, and 4;
with the proviso that the sum of nₐ+n_{b} is not less than 2 and not greater than 4;
or X₁ and Z₁ together form a fused ring system of the formula (Ia):

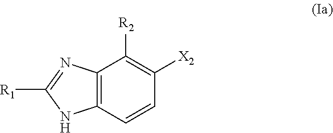

Rₐ is selected from the group of hydrogen and C₁-C₃ alkyl;
X₂ is selected from the group of:

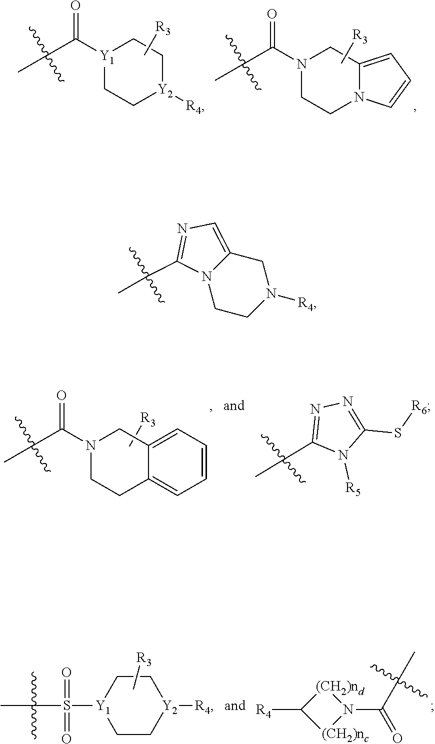

the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} is not less than 2 and not greater than 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of carbocycle and a monocyclic heterocyclic ring or a bicyclic or spirocyclic heterocyclic ring system bound through a carbon atom and containing 3, 4, 5, 6, 7, or 8 ring carbon atoms and 1, 2, 3, or 4 ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl, with the proviso that R₂ is not pyridinyl unless substituted at least once by —(CH₂)ₙ₁—C₃-C₆ cycloalkyl or phenyl;
R₂′ is selected from the group of hydrogen, OH, halogen, C₁-C₆ alkyl, and —CF₃;
R₃ is present one or more times and is independently selected from the group of:
   a) hydrogen, halogen, cyano, or OH;
   b) —CO₂H or —CO₂—(C₁-C₆ alkyl);
   c) C₁-C₆ alkyl or —O—C₁-C₆ alkyl substituted by 0, 1, 2, 3, 4 or 5 substituents selected from the group of halogen, CF₃, and OH;
   d) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
   e) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
   f) two R₃ form a bridge between carbon atoms, wherein the atoms of the bridge comprise 1, 2, or 3 atoms independently selected from the group of carbon, N, O, and S;
R₄ is present one or two times and is independently selected from the group of H, halogen, oxo, C₁-C₆ alkyl, —(CH₂)ₙ₂—C₃-C₆ cycloalkyl, —O—(CH₂)ₙ₂—C₃-C₆ cycloalkyl, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a spirocyclic carbocycle or spirocyclic heterocycle;
wherein the R₄ C₁-C₆ alkyl, —C(═O)—O—C₁-C₆ alkyl, and —O—C₁-C₆ alkyl groups and the rings of the —(CH₂)ₙ₂—C₃-C₆ cycloalkyl and —O—(CH₂)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n2 in each instance is an integer selected from the group of 0, 1, 2, and 3.

Further provided is a compound of Formula (I-B), or a pharmaceutically acceptable salt thereof:

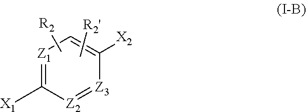

wherein:
X₁ is selected from the group of:

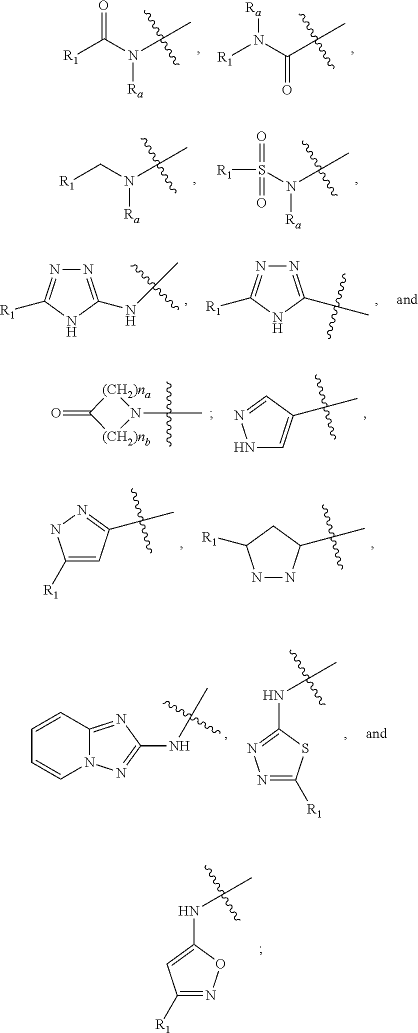

nₐ is an integer selected from the group of 0, 1, and 2;
n_{b} is an integer selected from the group of 0, 1, 2, 3, and 4;
with the proviso that the sum of nₐ+np is not less than 2 and not greater than 4;
or X₁ and Z₁ together form a fused ring system of the formula (Ia):

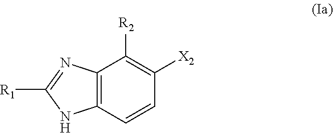

Rₐ is selected from the group of hydrogen and C₁-C₃ alkyl;
X₂ is selected from the group of:

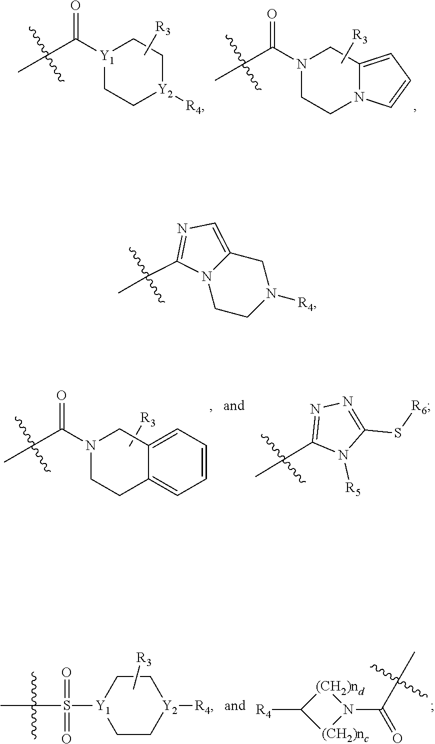

the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} is not less than 2 and not greater than 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl, pyridinyl bound through a carbon atom, and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl, with the proviso that R₂ is not pyridinyl unless substituted at least once by —(CH₂)ₙ₁—C₃-C₆ cycloalkyl or phenyl;
R₂′ is selected from the group of OH, halogen, C₁-C₆ alkyl, and —CF₃;
R₃ is present one or more times and is independently selected from the group of:
   g) hydrogen, halogen, cyano, or OH;
   h) —CO₂H or —CO₂—(C₁-C₆ alkyl);
   i) C₁-C₆ alkyl or —O—C₁-C₆ alkyl substituted by 0, 1, 2, 3, 4 or 5 substituents selected from the group of halogen, CF₃, and OH;
   j) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
   k) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
   l) two R₃ form a bridge between carbon atoms, wherein the atoms of the bridge comprise 1, 2, or 3 atoms independently selected from the group of carbon, N, O, and S;
R₄ is present one or two times and is independently selected from the group of H, halogen, oxo, C₁-C₆ alkyl, —(CH₂)ₙ₂—C₃-C₆ cycloalkyl, —O—(CH₂)ₙ₂—C₃-C₆ cycloalkyl, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a spirocyclic carbocycle or spirocyclic heterocycle;
wherein the R₄ C₁-C₆ alkyl, —C(═O)—O—C₁-C₆ alkyl, and —O—C₁-C₆ alkyl groups and the rings of the —(CH₂)ₙ₂—C₃-C₆ cycloalkyl and —O—(CH₂)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n2 in each instance is an integer selected from the group of 0, 1, 2, and 3.

Also provided is a compound of Formula (I-C), or a pharmaceutically acceptable salt thereof:

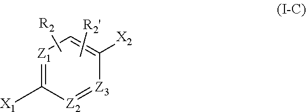

wherein:
X₁ is selected from the group of:

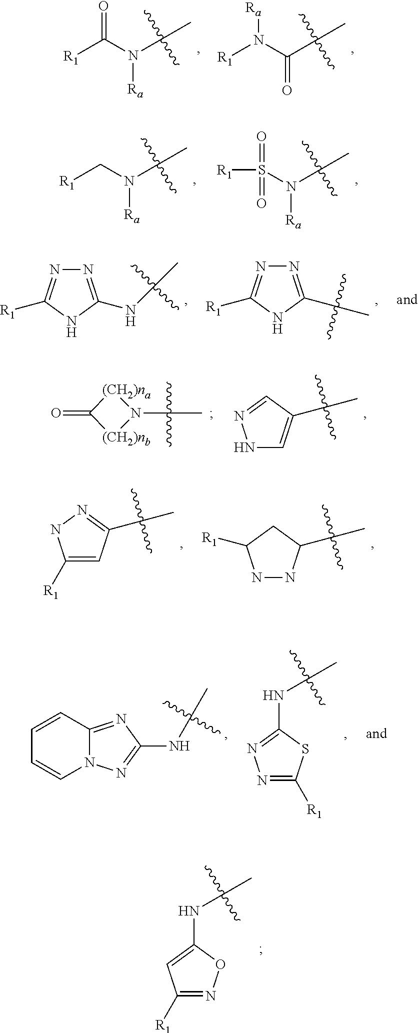

nₐ is an integer selected from the group of 0, 1, and 2;
n_{b} is an integer selected from the group of 0, 1, 2, 3, and 4;
with the proviso that the sum of nₐ+np is not less than 2 and not greater than 4;
or X₁ and Z₁ together form a fused ring system of the formula (Ia):

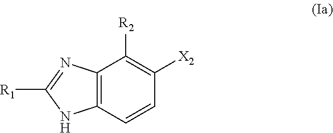

Rₐ is selected from the group of hydrogen and C₁-C₃ alkyl;
X₂ is selected from the group of:

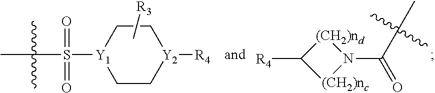

the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} 2, 3, 5, or 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl, pyridinyl bound through a carbon atom, and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl, with the proviso that R₂ is not pyridinyl unless substituted at least once by —(CH₂)ₙ₁—C₃-C₆ cycloalkyl or phenyl;
R₂′ is selected from the group of hydrogen, OH, halogen, C₁-C₆ alkyl, and —CF₃;
R₃ is present one or more times and is independently selected from the group of:
   m) hydrogen, halogen, cyano, or OH;
   n) —CO₂H or —CO₂—(C₁-C₆ alkyl);
   o) C₁-C₆ alkyl or —O—C₁-C₆ alkyl substituted by 0, 1, 2, 3, 4 or 5 substituents selected from the group of halogen, CF₃, and OH;
   p) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
   q) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
   r) two R₃ form a bridge between carbon atoms, wherein the atoms of the bridge comprise 1, 2, or 3 atoms independently selected from the group of carbon, N, O, and S;
R₄ is present one or two times and is independently selected from the group of H, halogen, oxo, C₁-C₆ alkyl, —(CH₂)ₙ₂—C₃-C₆ cycloalkyl, —O—(CH₂)ₙ₂—C₃-C₆ cycloalkyl, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a spirocyclic carbocycle or spirocyclic heterocycle;
wherein the R₄ C₁-C₆ alkyl, —C(═O)—O—C₁-C₆ alkyl, and —O—C₁-C₆ alkyl groups and the rings of the —(CH₂)ₙ₂—C₃-C₆ cycloalkyl and —O—(CH₂)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n2 in each instance is an integer selected from the group of 0, 1, 2, and 3.

Further also provided is a compound of Formula (I-D), or a pharmaceutically acceptable salt thereof:

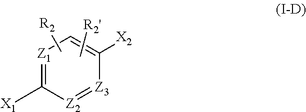

wherein:
X₁ is selected from the group of:

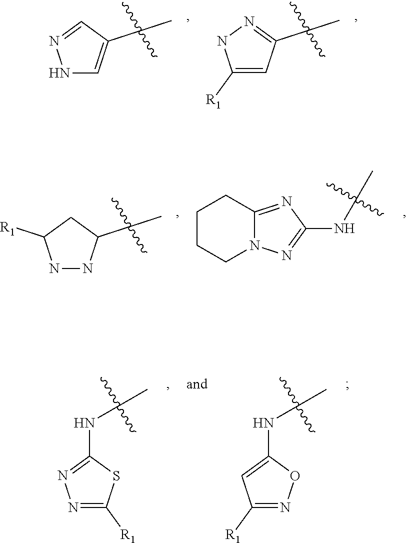

nₐ is an integer selected from the group of 0, 1, and 2;
n_{b} is an integer selected from the group of 0, 1, 2, 3, and 4;
with the proviso that the sum of nₐ+n_{b} is not less than 2 and not greater than 4;
or X₁ and Z₁ together form a fused ring system of the formula (Ia):

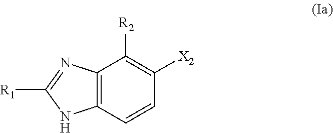

Rₐ is selected from the group of hydrogen and C₁-C₃ alkyl;
X₂ is selected from the group of:

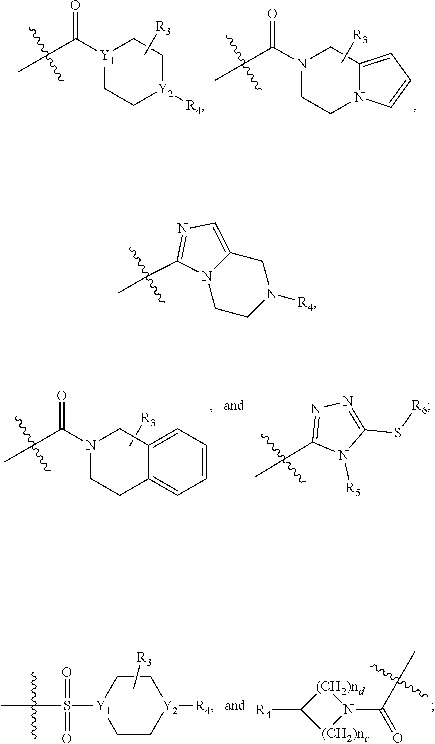

the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} is not less than 2 and not greater than 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl, pyridinyl bound through a carbon atom, and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl, with the proviso that R₂ is not pyridinyl unless substituted at least once by —(CH₂)ₙ₁—C₃-C₆ cycloalkyl or phenyl;
R₂′ is selected from the group of hydrogen, OH, halogen, C₁-C₆ alkyl, and —CF₃;
R₃ is present one or more times and is independently selected from the group of:
   s) hydrogen, halogen, cyano, or OH;
   t) —CO₂H or —CO₂—(C₁-C₆ alkyl);
   u) C₁-C₆ alkyl or —O—C₁-C₆ alkyl substituted by 0, 1, 2, 3, 4 or 5 substituents selected from the group of halogen, CF₃, and OH;
   v) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
   w) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
   x) two R₃ form a bridge between carbon atoms, wherein the atoms of the bridge comprise 1, 2, or 3 atoms independently selected from the group of carbon, N, O, and S;
R₄ is present one or two times and is independently selected from the group of H, halogen, oxo, C₁-C₆ alkyl, —(CH₂)ₙ₂—C₃-C₆ cycloalkyl, —O—(CH₂)ₙ₂—C₃-C₆ cycloalkyl, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a spirocyclic carbocycle or spirocyclic heterocycle;
wherein the R₄ C₁-C₆ alkyl, —C(═O)—O—C₁-C₆ alkyl, and —O—C₁-C₆ alkyl groups and the rings of the —(CH₂)ₙ₂—C₃-C₆ cycloalkyl and —O—(CH₂)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n2 in each instance is an integer selected from the group of 0, 1, 2, and 3.

Further yet provided is a compound of Formula (I-E), or a pharmaceutically acceptable salt thereof:

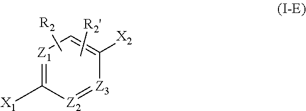

wherein:
X₁ is selected from the group of:

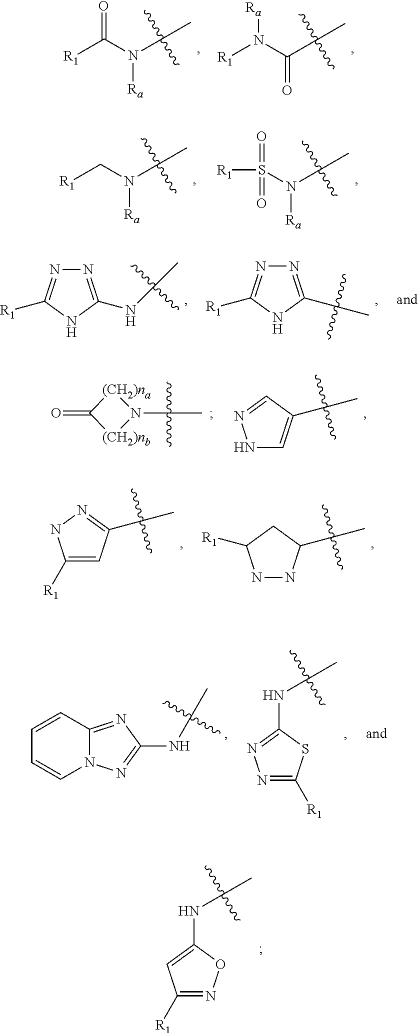

nₐ is an integer selected from the group of 0, 1, and 2;
n_{b} is an integer selected from the group of 0, 1, 2, 3, and 4;
with the proviso that the sum of nₐ+n_{b} is not less than 2 and not greater than 4;
or X₁ and Z₁ together form a fused ring system of the formula (Ia):

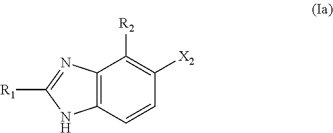

Rₐ is selected from the group of hydrogen and C₁-C₃ alkyl;
X₂ is selected from the group of:

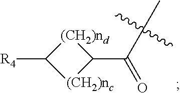

the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} is not less than 2 and not greater than 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl, pyridinyl bound through a carbon atom, and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂-C₁-C₆ alkyl, —C(═O)—NR^{x}R^{y}, —N(R^{x})(S(═O)₂-C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, with the proviso that R₂ is not pyridinyl unless substituted at least once by —(CH₂)ₙ₁—C₃-C₆ cycloalkyl or phenyl;
R₂′ is selected from the group of hydrogen, OH, halogen, C₁-C₆ alkyl, and —CF₃;
R₃ is present one or more times and is independently selected from the group of:
   y) hydrogen, halogen, cyano, or OH;
   z) —CO₂H or —CO₂—(C₁-C₆ alkyl);
   aa) C₁-C₆ alkyl or —O—C₁-C₆ alkyl substituted by 0, 1, 2, 3, 4 or 5 substituents selected from the group of halogen, CF₃, and OH;
   bb) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
   cc) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
   dd) two R₃ form a bridge between carbon atoms, wherein the atoms of the bridge comprise 1, 2, or 3 atoms independently selected from the group of carbon, N, O, and S;
R₄ is present one or two times and is halogen;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n2 in each instance is an integer selected from the group of 0, 1, 2, and 3,
with the proviso that the compound is not N-[4-(4,4-difluoropiperidine-1-carbonyl)-3-pyrrolidin-1-ylphenyl]cyclopropanecarboxamide; (4,4-difluoropiperidin-1-yl)-[4-(5-methyl-4H-1,2,4-triazol-3-yl)-2-[3-(trifluoromethyl) pyrazol-1-yl]phenyl]methanone; [2-(3-cyclopropylpyrazol-1-yl)-4-(5-methyl-4H-1,2,4-triazol-3-yl)phenyl]-(4,4-difluoropiperidin-1-yl)methanone; (4,4-difluoropiperidin-1-yl)-[4-(5-methyl-4H-1,2,4-triazol-3-yl)-2-(3-propan-2-ylpyrazol-1-yl)phenyl]methanone; or N-[4-(4-fluoropiperidine-1-carbonyl)-3-pyrrolidin-1-ylphenyl]cyclopropanecarboxamide.

### DETAILED DESCRIPTION OF THE INVENTION

Within the scope of compounds of Formula (I-A), (I-B), (I-C), (I-D) and (I-E), or pharmaceutically acceptable salts thereof, there are three additional embodiments comprising, respectively, a compound of Formula (I′) or a pharmaceutically acceptable salt thereof, a compound of Formula (I″) or a pharmaceutically acceptable salt thereof, and a compound of Formula (I′″) or a pharmaceutically acceptable salt thereof:

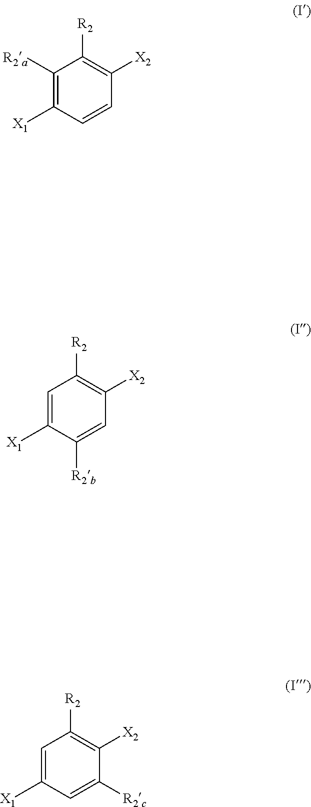

wherein, in each instance, when present, X₁, X₂, R₂, Z₁, Z₂, Z₃, and all other variables and provisos are as defined for Formulas (I-A), (I-B), (I-C), (I-D), or (I-E), above.

It is also understood that there are separate and independent embodiments within the scope of Formula (I) and based upon the definitions of X₁ and X₂, above, comprising, respectively, a compound of Formulas (I-1), (I-2), (I-3), (I-4), (I-5), (I-6), and (I-7), or a pharmaceutically acceptable salt thereof:

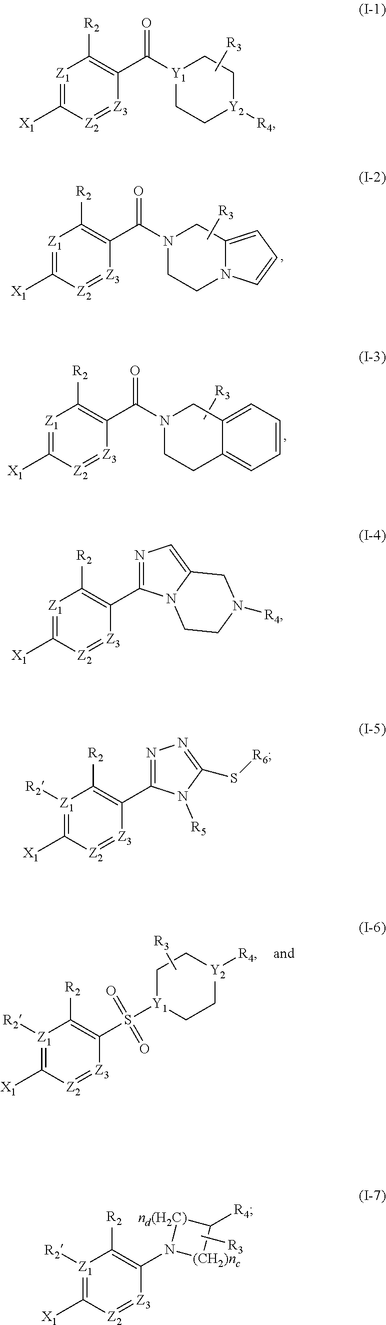

wherein, in each instance in which they are present, all variables, including X₁, Rₐ, R₁, R₂, R³, R₄, Y₁, Y₂, Z₁, Z₂, Z₃, nₐ, n_{b}, n1, n2, and the provisos are as defined above for Formulas (I-A), (I-B), (I-C), (I-D), or (I-E).

It is also understood that there are separate and independent embodiments within the scope of Formulas (I-A), (I-B), (I-C), (I-D), and (I-E), and based upon the definitions of X₁ and X₂, above, comprising, respectively, a compound of Formulas (I-a) through (I-II), below, or a pharmaceutically acceptable salt thereof:

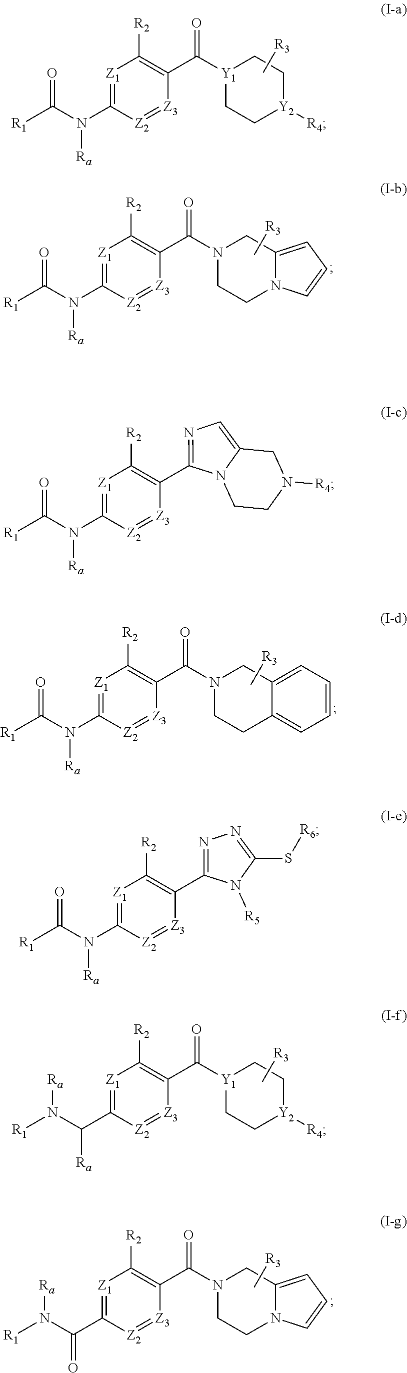

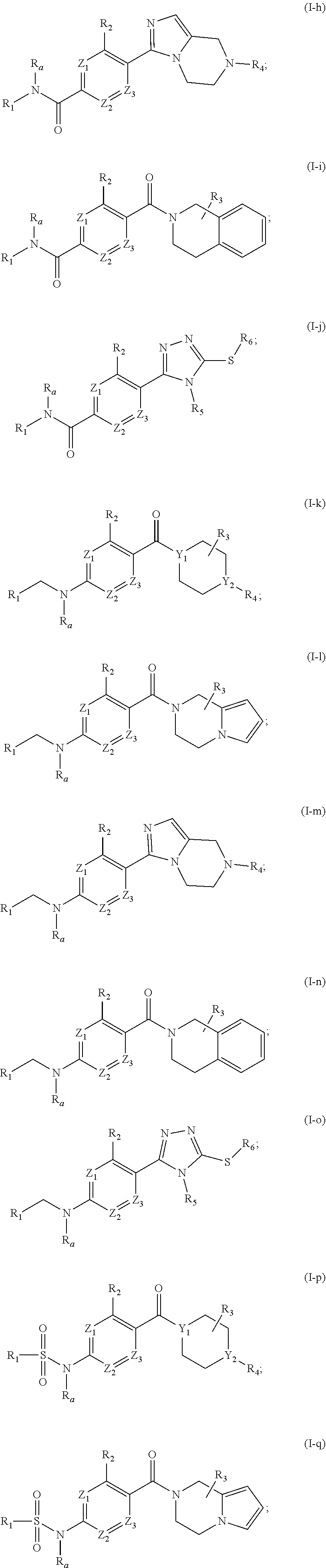

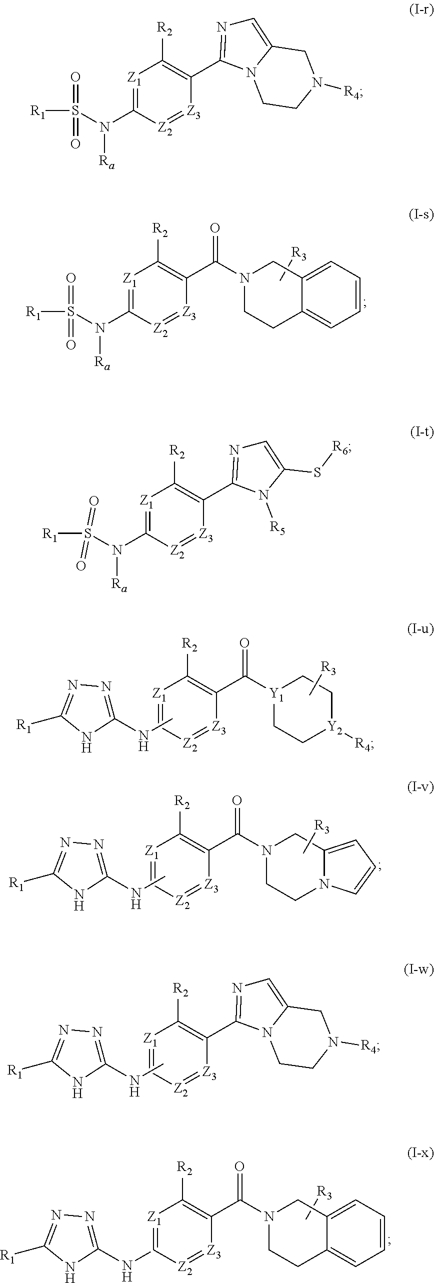

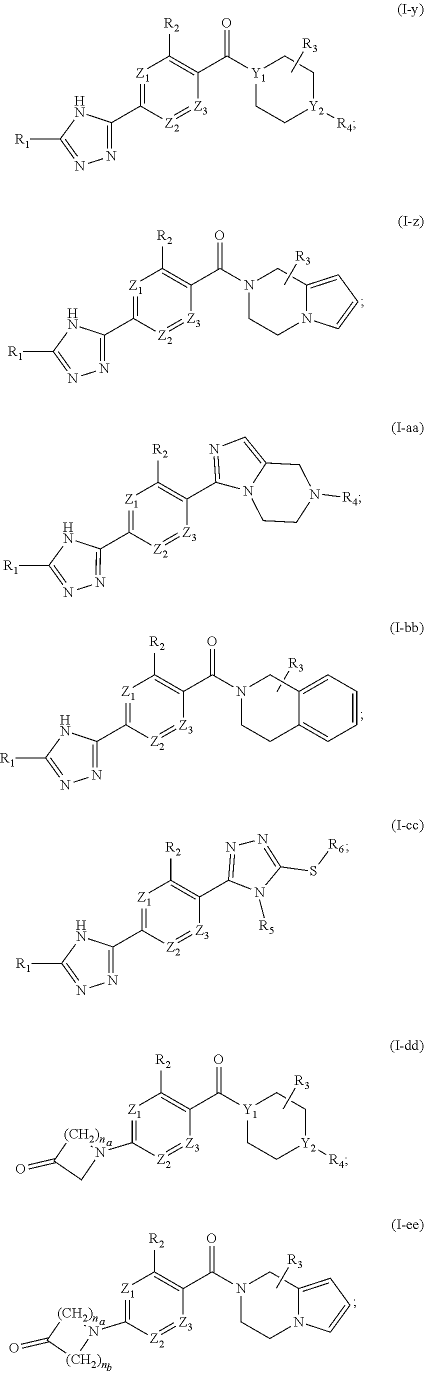

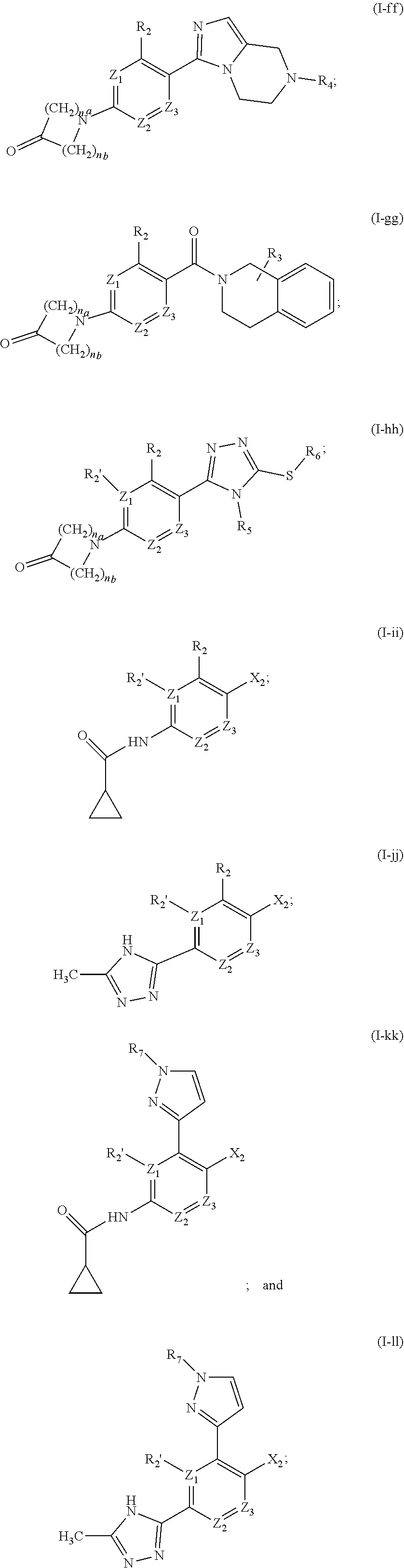

wherein, in each instance in which they are present, all variables, including Rₐ, R₁, R₂, R³, R₄, R₅, R₆, Y₁, Y₂, Z₁, Z₂, Z₃, nₐ, n_{b}, n1, n2, and the provisos are as defined above for Formulas (I-A), (I-B), (I-C), (I-D), or (I-E).

Other separate embodiments provide a compound of each of Formulas (I-A), (I-B), (I-C), (I-D), and (I-E), Formulas (I′), (I″), and (I′″), Formulas (I-1) through (I-7), and Formulas (I-a) through (I-II), above, or a pharmaceutically acceptable salt thereof, wherein in each embodiment Z₁, Z₂, and Z₃ are each C, and Rₐ, R₁, R₂, R³, R₄, R₅, R₆, Y₁, Y₂, nₐ, no, n1, n2, and the provisos are as defined above for the corresponding Formulas (I-A), (I-B), (I-C), (I-D), or (I-E) through (I-II).

Other separate embodiments provide a compound of each of Formulas (I-A), (I-B), (I-C), (I-D), and (I-E), Formulas (I′), (I″), and (I′″), Formulas (I-1) through (I-7), and Formulas (I-a) through (I-II), above, or a pharmaceutically acceptable salt thereof, wherein in each embodiment Z₁, Z₂, and Z₃ are each N, and Rₐ, R₁, R₂, R³, R₄, R₅, R₆, Y₁, Y₂, nₐ, no, n1, n2, and the provisos are as defined above for the corresponding Formulas (I-A), (I-B), (I-C), (I-D), or (I-E) through (I-II).

Additional separate embodiments provide a compound of each of Formulas (I-A), (I-B), (I-C), (I-D), and (I-E), Formulas (I′), (I″), and (I′″), Formulas (I-1) through (I-7), and Formulas (I-a) through (I-II), above, or a pharmaceutically acceptable salt thereof, wherein in each embodiment, Z₁ and Z₂ are each C, Z₃ is N, and Rₐ, R₁, R₂, R³, R₄, R₅, R₆, Y₁, Y₂, nₐ, n_{b}, n1, n2, and the provisos are as defined above for the corresponding Formulas (I-A), (I-B), (I-C), (I-D), or (I-E) through (I—; II).

Additional separate embodiments provide a compound of each of Formulas (I-A), (I-B), (I-C), (I-D), and (I-E), Formulas (I′), (1″), and (I′″), Formulas (I-1) through (I-7), and Formulas (I-a) through (I-II), above, or a pharmaceutically acceptable salt thereof, wherein in each embodiment, Z₁ and Z₃ are each C, Z₂ is N, and Rₐ, R₁, R₂, R³, R₄, R₅, R₆, Y₁, Y₂, nₐ, np, n1, n2, and the provisos are as defined above for the corresponding Formulas (I-A), (I-B), (I-C), (I-D), or (I-E) through (I-II).

Additional separate embodiments provide a compound of each of Formulas (I-A), (I-B), (I-C), (I-D), and (I-E), Formulas (I′), (I″), and (I′″), Formulas (I-1) through (I-7), and Formulas (I-a) through (I-II), above, or a pharmaceutically acceptable salt thereof, wherein in each embodiment, Z₁ and Z₃ are each N, Z₂ is C, and Rₐ, R₁, R₂, R³, R₄, R₅, R₆, Y₁, Y₂, nₐ, n_{b}, n1, n2, and the provisos are as defined above for the corresponding Formulas (I-A), (I-B), (I-C), (I-D), or (I-E) through (I-II).

Additional separate embodiments provide a compound of each of Formulas (I-A), (I-B), (I-C), (I-D), and (I-E), Formulas (I′), (I″), and (I″), Formulas (I-1) through (I-7), and Formulas (I-a) through (I-II), above, or a pharmaceutically acceptable salt thereof, wherein in each embodiment, Z₁ and Z₂ are each N, Z₃ is C, and Rₐ, R₁, R₂, R³, R₄, R₅, R₆, Y₁, Y₂, nₐ, no, n1, n2, and the provisos are as defined above for the corresponding Formulas (I-A), (I-B), (I-C), (I-D), or (I-E) through (I-II).

Additional separate embodiments provide a compound of each of Formulas (I-A), (I-B), (I-C), (I-D), and (I-E), Formulas (I′), (I″), and (I′″), Formulas (I-1) through (I-7), and Formulas (I-a) through (I-II), above, or a pharmaceutically acceptable salt thereof, wherein in each embodiment, Z₂ and Z₃ are each N, Z₁ is C, and Rₐ, R₁, R₂, R³, R₄, R₅, R₆, Y₁, Y₂, nₐ, n_{b}, n1, n2, and the provisos are as defined above for the corresponding Formulas (I-A), (I-B), (I-C), (I-D), or (I-E) through (I-II).

Within each of the embodiments herein of Formula (I-A), Formulas (I′), (I″), and (I′″), Formulas (I-1) through (I-7), and Formulas (I-a) through (I-jj), above, or a pharmaceutically acceptable salt thereof, there is a further embodiment in which R₂ is selected from the group of

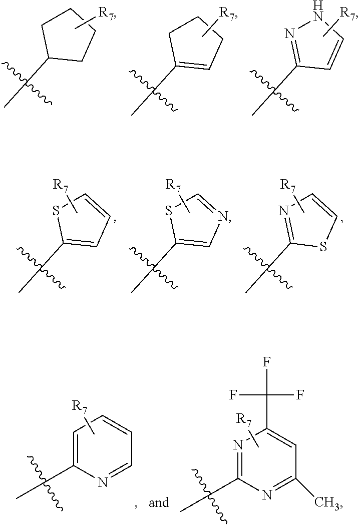

wherein R₇ is present one or two times and is independently selected from the group of hydrogen, —OH; halogen; —C₁-C₆ alkyl; —O—C₁-C₆ alkyl; —(CH₂)ₙ₁—C₃-C₆ cycloalkyl; —(CH₂)ₙ₁—C₃-C₆ heterocycle including at least one heteroatom selected from O, N, and S; cyano; —O—(CH₂)ₙ₂—C₃-C₆ cycloalkyl; —C(═O) OH; —C(═O)—O—C₁-C₆ alkyl; —S(═O)₂-C₁-C₆ alkyl; —C(═O)—NR^{x}R^{y}; —N(R^{x})(S(═O)₂-C₁-C₆ alkyl); and —CF₃, wherein any of the alkyl groups are further substituted with 0, 1, 2, 3, or 4 substituents selected from halogen and —OH, and wherein n1 is an integer selected from the group of 0, 1, 2, and 3.

Within each of the embodiments herein of Formula (I-A), Formulas (I′), (I″), and (I′″), Formulas (I-1) through (I-7), and Formulas (I-a) through (I-jj), above, or a pharmaceutically acceptable salt thereof, there is a further embodiment in which R₂ is selected from the group of

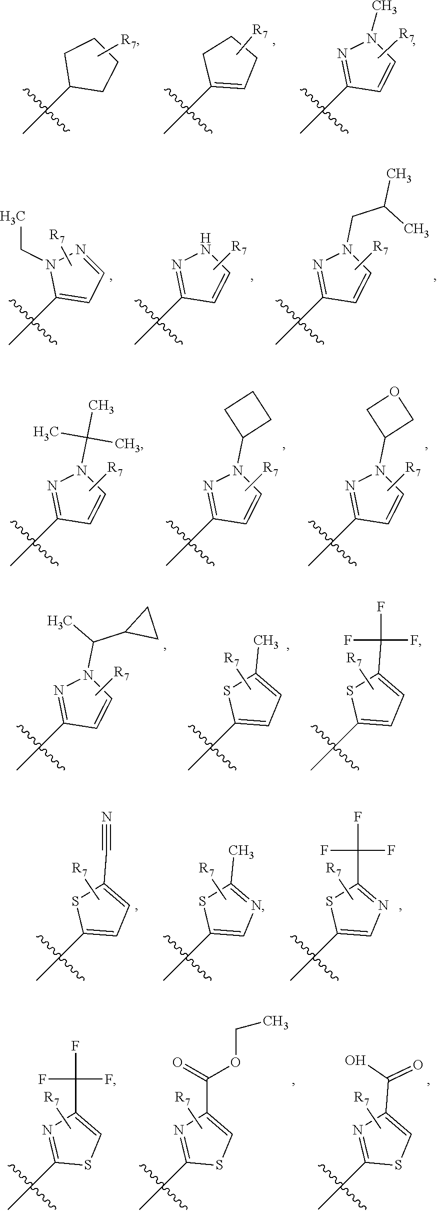

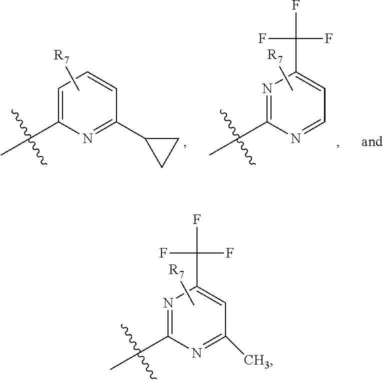

wherein R₇ is present one or two times and is independently selected from the group of hydrogen, —OH; halogen; —C₁-C₆ alkyl; —O—C₁-C₆ alkyl; —(CH₂)ₙ₁—C₃-C₆ cycloalkyl; —(CH₂)ₙ₁—C₃-C₆ heterocycle including at least one heteroatom selected from O, N, and S; cyano; —O—(CH₂)ₙ₂—C₃-C₆ cycloalkyl; —C(═O) OH; —C(═O)—O—C₁-C₆ alkyl; —S(═O)₂-C₁-C₆ alkyl; —C(═O)—NR^{x}R^{y}; —N(R^{x})(S(═O)₂-C₁-C₆ alkyl); and —CF₃, wherein any of the alkyl groups are further substituted with 0, 1, 2, 3, or 4 substituents selected from halogen and —OH, and wherein n1 is an integer selected from the group of 0, 1, 2, and 3.

Within each of the embodiments herein of Formula (I-A), Formulas (I′), (I″), and (I′″), Formulas (I-1) through (I-7), and Formulas (I-a) through (I-jj), above, or a pharmaceutically acceptable salt thereof, there is yet a further embodiment in which R₂ is selected from the group of

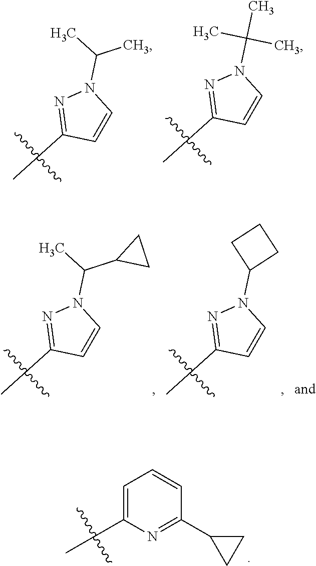

Within each of the embodiments herein of Formulas (I-B), (I-C), (I-D), and (I-E), Formulas (I′), (I″), and (I′″), Formulas (I-1) through (I-7), and Formulas (I-a) through (I-II), above, or a pharmaceutically acceptable salt thereof, there is a further embodiment in which R₂ is selected from the group of

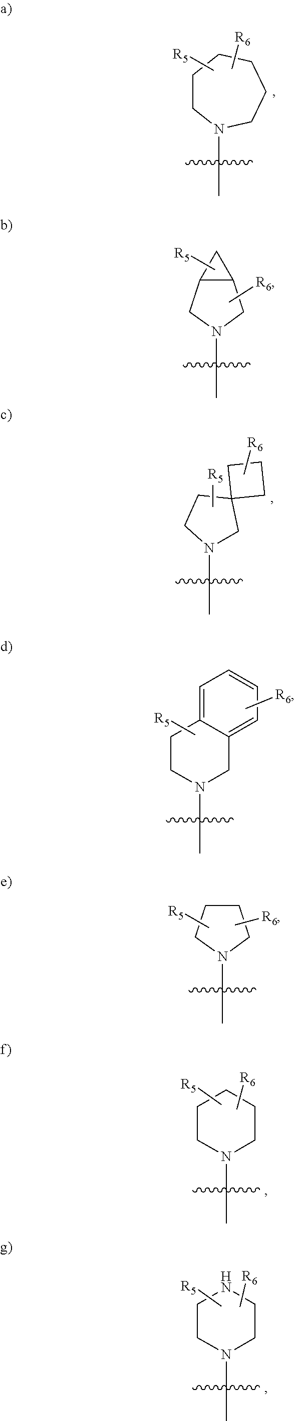

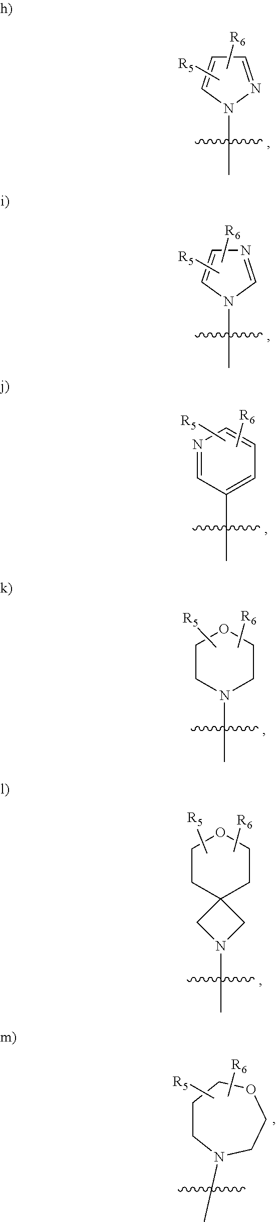

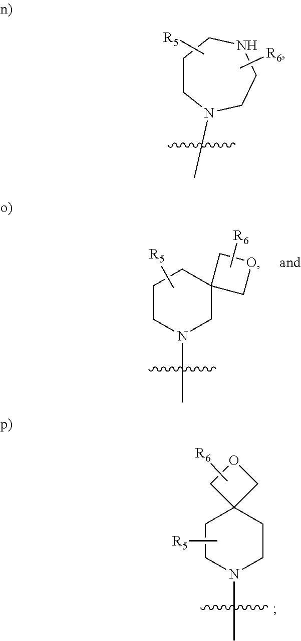

wherein R₇ and R⁸ are in each instance independently selected from hydrogen, halogen, C₁-C₆ alkyl, —O—C₁-C₆ alkyl, —CF₃, and phenyl, with the proviso that only one of R₇ and R₈ may be phenyl. It is understood that in the embodiments herein, in instances wherein the R₂ group is a bicyclic or spirocyclic ring, R₇ and R₈ may be bound to any available ring carbon or nitrogen atom in either of the rings and that R₇ and R⁸ may be bound to ring atoms in the same or different rings.

Within each of the embodiments herein of Formulas (I-B), (I-C), (I-D), and (I-E), Formulas (I′), (I″), and (I′″), Formulas (I-1) through (I-7), and Formulas (I-a) through (I-II), above, or a pharmaceutically acceptable salt thereof, there is yet a further embodiment in which R₂ is selected from the group of:

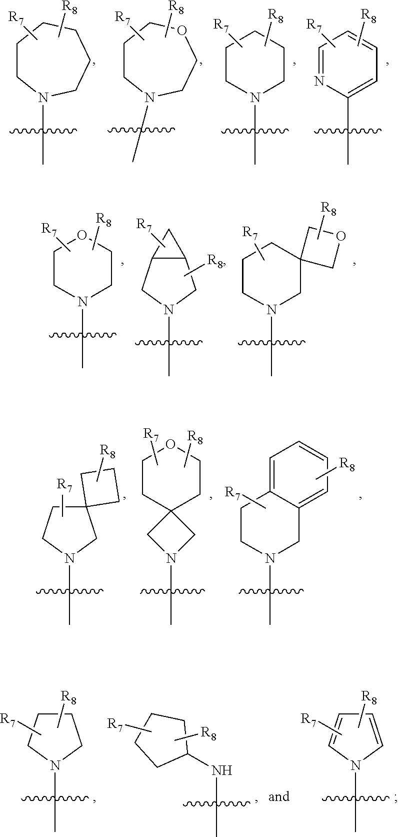

wherein R₇ and R₈ are in each instance independently selected from hydrogen, halogen, C₁-C₆ alkyl, —O—C₁-C₆ alkyl, —CF₃, and phenyl, with the proviso that only one of R₇ and R₈ may be phenyl.

Also provided are separate embodiments comprising, respectfully, compounds for each of the Formulas (I-B), (I-C), (I-D), and (I-E), Formulas (I′), (I″), and (I′″), Formulas (I-1) through (I-7), and Formulas (I-a) through (I-II), or a pharmaceutically acceptable salt thereof, wherein separate embodiments comprise a compound of each formula wherein R₂ is defined above. For instance, provided is a compound of Formula (I-B), (I-C), (I-D), or (I-E), or a pharmaceutically acceptable salt thereof, wherein R₂ is the azepane ring of group a) substituted by variable groups R₇ and R₈. Another embodiment comprises a compound of Formula (I-B), (I-C), (I-D), or (I-E), wherein R₂ is the azabicyclo[3.10.0]hexanyl ring of group b) substituted by variable groups R₇ and R₈. This pattern continues to define the remainder of the embodiments, each with the separate referenced R₂ group from the list above.

Within each of the embodiments above for a compound of each of Formulas (I-B), (I-C), (I-D), and (I-E), Formulas (I′), (I″), and (I″), Formulas (I-1) through (I-7), and Formulas (I-a) through (I-II), or a pharmaceutically acceptable salt thereof, there is also a further embodiment wherein each variable, including Z₁, Z₂, Z₃, Rₐ, R₁, R₃, R₄, R₅, R₆, Y₁, Y₂, nₐ, n_{b}, n1, n2, and the provisos are as defined above for the corresponding Formulas (I-B), (I-C), (I-D), and (I-E), Formulas (I′), (I″), and (I′″), Formulas (I-1) through (I-7), and Formulas (I-a) through (I-II), and R₂ is selected from the group of:

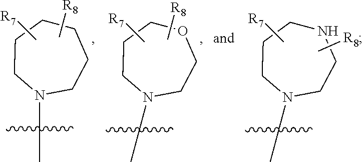

wherein R₇ and R₈ are each independently selected from hydrogen, halogen, C₁-C₆ alkyl, —O—C₁-C₆ alkyl, halogen, and —CF₃.

Within each of the embodiments above for a compound of each of Formulas (I-B), (I-C), (I-D), and (I-E), Formulas (I′), (I″), and (I′″), Formulas (I-1) through (I-7), and Formulas (I-a) through (I-II), or a pharmaceutically acceptable salt thereof, there is also a further embodiment wherein each variable, including Z₁, Z₂, Z₃, Rₐ, R₁, R₃, R₄, R₅, R₆, Y₁, Y₂, nₐ, no, n1, n2, and the provisos are as defined above for the corresponding Formula (I), Formulas (I-1) through (I-7), and Formulas (I-a) through (I-II), and R₂ is selected from the group of:

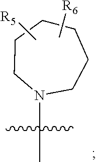

wherein R₇ and R⁸ are each independently selected from hydrogen, halogen, C₁-C₆ alkyl, —O—C₁-C₆ alkyl, and —CF₃.

Within each of the embodiments above for a compound of each of Formulas (I-B), (I-C), (I-D), and (I-E), Formulas (I′), (I″), and (I′″), Formulas (I-1) through (I-7), and Formulas (I-a) through (I-II), or a pharmaceutically acceptable salt thereof, there is also a further embodiment wherein each variable, including Z₁, Z₂, Z₃, Rₐ, R₁, R₃, R₄, R₅, R₆, Y₁, Y₂, nₐ, n_{b}, n1, n2, and the provisos are as defined above for the corresponding Formula (I), Formulas (I-1) through (I-7), and Formulas (I-a) through (I-II), and R₂ is selected from the group of:

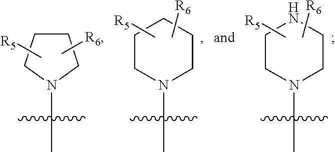

wherein R₇ and R₅ are each independently selected from hydrogen, halogen, C₁-C₆ alkyl, —O—C₁-C₆ alkyl, and —CF₃.

Within each of the embodiments above for a compound of each of Formula (I), Formulas (I-1) through (I-7), and Formulas (I-a) through (I-II), or a pharmaceutically acceptable salt thereof, there is also a further embodiment wherein each variable, including Z₁, Z₂, Z₃, Rₐ, R₁, R₃, R₄, R₅, R₆, Y₁, Y₂, nₐ, n_{b}, n1, n2, and the provisos are as defined above for the corresponding Formula (I), Formulas (I-1) through (I-7), and Formulas (I-a) through (I-II), and R₂ is selected from the group of:

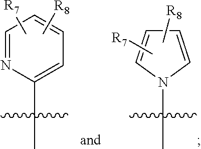

wherein R₇ and R₈ are each independently selected from hydrogen, halogen, C₁-C₆ alkyl, —O—C₁-C₆ alkyl, and —CF₃.

Within each of the embodiments above for a compound of each of Formula (I), Formulas (I-1) through (I-7), and Formulas (I-a) through (I-II), or a pharmaceutically acceptable salt thereof, there is also a further embodiment wherein each variable, including Z₁, Z₂, Z₃, Rₐ, R₁, R₃, R₄, R₅, R₆, Y₁, Y₂, nₐ, n_{b}, n1, n2, and the provisos are as defined above for the corresponding Formula (I), Formulas (I-1) through (I-7), and Formulas (I-a) through (I-II), and R₂ is selected from the group of:

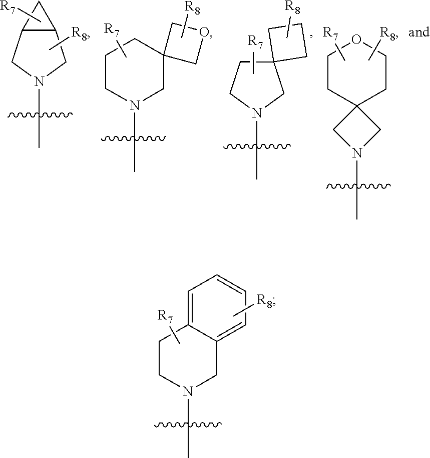

wherein R₇ and R₈ are each independently selected from hydrogen, halogen, C₁-C₆ alkyl, —O—C₁-C₆ alkyl, and —CF₃.

In some embodiments herein, R₇ and R₈ are selected from hydrogen, halogen, —CF₃, C₁-C₄ alkyl, and C₁-C₄ alkoxy. In other embodiments, R₇ and R₈ are selected from hydrogen, halogen, —CF₃, C₁-C₃ alkyl, and C₁-C₃ alkoxy. In still further embodiments, hydrogen, F, Cl, —CF₃, C₁-C₃ alkyl, and C₁-C₃ alkoxy.

Within each of the embodiments for compounds and salts herein comprising a R₃ moiety, there is a further embodiment of the same scope, except R₃ is present one to three times. Within each of the embodiments for compounds and salts herein comprising a R₃ moiety, there is a further embodiment of the same scope, except R₃ is present one to two times. Within each of the embodiments for compounds and salts herein comprising a R₃ moiety, there is a further embodiment of the same scope, except R₃ is present only one time. Within each of the embodiments for compounds and salts herein comprising a R₃ moiety, there is a further embodiment of the same scope, except R₃ is selected from the group of hydrogen, C₁-C₄ alkyl, C₃-C₆ cycloalkyl, —CF₃, —C₁-C₄ alkyl-OH, phenyl, pyrazolyl, and thiophenyl, wherein the phenyl, pyrazolyl, and thiophenyl rings are substituted by 0, 1, 2, or 3 substituents selected from the group of halogen, OH, —CF₃, C₁-C₄ alkyl, and C₁-C₄ alkoxy.

Within each of the embodiments for compounds and salts herein comprising a R₃ moiety, there is a further embodiment of the same scope, except R₃ is selected from the group of phenyl, pyrazolyl, and thiophenyl, substituted by 0, 1, 2, or 3 substituents selected from the group of halogen, OH, —CF₃, C₁-C₄ alkyl, and C₁-C₄ alkoxy.

Also included for each compound of Formulas (I-A), (I-B), (I-C), (I-D), and (I-E) and all other formulas and specifically named compounds herein are the pharmaceutically acceptable salts, pharmaceutically acceptable co-crystals, pharmaceutically acceptable esters, pharmaceutically acceptable solvates, hydrates, isomers (including optical isomers, racemates, or other mixtures thereof), tautomers, isotopes, polymorphs, and pharmaceutically acceptable prodrugs of such compounds.

The compounds of the disclosure may possess an asymmetric center, and can be produced as a racemic mixture or as individual enantiomers. The individual enantiomers may be obtained by asymmetric synthesis or by resolving a racemic or non-racemic mixture of an intermediate at some appropriate stage of the synthesis. The individual enantiomers may also be obtained by resolution of the compound by conventional means, such as crystallization in the presence of a resolving agent, or chromatography, using, for example a chiral high pressure liquid chromatography (HPLC) column. The individual enantiomers as well as racemic and non-racemic mixtures of enantiomers are within the scope of the present disclosure, all of which are intended to be included within the structures depicted in this specification unless otherwise specifically indicated.

### Methods of Use

Hypertension is the most common chronic disease, affecting 1.13 billion people and accounting for 10 million deaths every year worldwide. Despite the availability of antihypertensive drugs that successfully reduced blood pressure (BP) for millions of patients, important unmet needs remain. Only half of people with hypertension have their BP under control, and current antihypertensive medications fail to restore normal BP in a large proportion of patients. Treatment-resistant hypertension (TRH), also known as resistant hypertension, is on the rise due to the rising rates of diabetes and obesity, which contribute up to 75% of the risk for hypertension and to most cases of TRH. Identifying new targets and developing novel strategies for preventing and treating hypertension and its complications remain a high public health and medical priority.

Provided is a method of inhibiting the activity of a GPR39 protein in a subject, the method comprising administering to the subject in need thereof a pharmaceutically effective amount of a compound of Formula (I-A), (I-B), (I-C), (I-D), or (I-E), or a pharmaceutically acceptable salt thereof.

That said, in certain embodiments, the method comprises administering to the subject in need thereof a pharmaceutically effective amount of a compound of Formula (I-A), (I-B), (I-C), (I-D), or (I-E), of a compound of any subformula of Formula (I-A), (I-B), (I-C), (I-D), or (I-E), of any of the specifically named compounds herein, or a pharmaceutically acceptable salt of any of the foregoing.

Provided is a method of treating hypertension in a human in need thereof, the method comprising administering to the human a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing. In one embodiment, the hypertension to be treated is primary hypertension, which may also be referred to as essential hypertension.

In another embodiment, the hypertension to be treated is secondary hypertension, including, but not limited to, hypertension caused by sleep apnea, blocked renal arteries, abnormal levels of hormones, enzymes, growth factors, or other agents controlling systemic or localized blood pressure (including, but not limited to, renin, angiotensin I and II, and aldosterone, angiotensin converting enzyme, catecholamines, thrombin, prostaglandins, natriuretic peptides, vasopressin, adrenomedullin, Substance P, calcitonin gene related peptide, kallikreins, kininogens, kinins, kinin-degrading enzymes, phosducin, adipokines, and leptin), adrenal gland disease, thyroid abnormalities (including hyperthyroidism, hypothyroidism, Cushing's Disease, Pheochromocytoma, and excess growth hormone), and constrictions of the aorta, including coarctation.

In one embodiment, the hypertension to be treated is treatment-resistant or refractory hypertension.

In still another embodiment, the hypertension to be treated is hypertensive urgency.

In another embodiment, the hypertension to be treated is malignant hypertension, which may also be referred to as hypertensive emergency or hypertensive crisis.

In an additional embodiment, the hypertension to be treated is isolated systolic hypertension.

In a further embodiment, the hypertension to be treated is salt-sensitive hypertension.

In another embodiment, the hypertension to be treated is pulmonary hypertension.

Current therapies for stroke involve dissolving or removing the clot responsible for vessel occlusion. Yet, despite opening large vessels, small blood vessels, remain compromised in a significant proportion of patients, ranging from 30-50%, which can lead to ineffective therapy. A compound as described herein can be combined with current stroke therapy to improve outcome for a large segment of patients.

Accordingly, provided is a method of treating stroke in a human in need thereof, the method comprising administering to the human a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

Vascular dementia a small vessel disease in the brain compromises blood flow and oxygen delivery to brain cells, resulting in neuronal dysfunction. Dementia due to small vessel disease is the second leading cause of dementia after Alzheimer's disease, and is the predominant cause in individuals over 70 and in developing countries. Provided is a method of treating of dementia due to small vessel disease in a human in need thereof, the method comprising administering to the human a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

In light of their effects on relaxing and protecting microvessels, the compounds and compositions herein may also be used in methods of treating conditions associated with microvascular dysfunction and microvascular disease, with each method comprising administering to a subject in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing. This includes methods of treating microvascular complications associated with or caused by diabetes. In one embodiment, the diabetes in question is Type I diabetes mellitus. In another embodiment the diabetes in question is Type II diabetes mellitus. This also includes methods of treating microvascular complications associated with or caused by prediabetic conditions, also known as prediabetes.

Also provided is a method of treating heart failure in a human, the method comprising administering to the human in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

Included are methods of treating microvascular angina, cerebral small vessel disease (cSVD), vascular cognitive impairment (VCI), Systemic Microvascular Endothelial Dysfunction (alone or presenting with Infective Endocarditis), and Chronic Kidney Disease.

Peripheral arterial disease, which is mostly small vessel disease and is generally not helped by the surgical and stenting techniques that target large vessels, but do not treat small vessel disease.

Provided herein is a method of treatment of peripheral artery disease in a human, the method comprising administering to a mammal in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

The compounds herein may also be used in the treatment of preeclampsia, the pregnancy complication characterized by high blood pressure and signs of damage to another organ system, most often the liver and kidneys. Provided herein is a method of treatment of preeclampsia in a human, the method comprising administering to a mammal in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

The compounds herein are also useful in the treatment of myocardial infarction, particularly in cases where no-reflow phenomenon is present. A no-reflow phenomenon is present when, despite restoration of the coronary flow of blood to the myocardium, breakdown of or obstruction to coronary microvasculature can markedly reduce blood flow to the infarct zone. Flow to the microcirculation in such instances may occur in one third of myocardial infarction patients.

As such, provided is a method of treatment of myocardial infarction in a human, the method comprising administering to a mammal in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

Also provided is a method of treatment of myocardial infarction with the presence of no-reflow phenomenon in a human, the method comprising administering to a mammal in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

The compounds of the present invention may also be used in situations of chronic coronary artery disease in which the large arteries are bypassed or stented and, yet, about a third of the patients continue to have angina due to microvascular dysfunction.

As such, provided is a method of treatment of chronic coronary artery disease in a human, the method comprising administering to a mammal in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

Also provided is a method of treatment of microvascular dysfunction associated with chronic coronary artery disease in a human, the method comprising administering to a mammal in need thereof a pharmaceutically effective amount a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

The compounds of the present invention may also be used for conditions referred to as Syndrome X, wherein a subject is experiencing a cardiac microvascular dysfunction or constriction causing angina despite normal epicardial coronary artery activity, such as diagnosed by angiography. As such, provided is a method of treatment of Syndrome X in a human, the method comprising administering to a mammal in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

Provided is a method of treating breast cancer in a human in need thereof, the method comprising administering to the human a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing. In some embodiments, the breast cancer treated in this method is ER negative breast cancer.

Also provided is a method of treating gastric adenocarcinomas in a human, the method comprising administering to the human in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

Also provided is a method of promoting or enhancing colon epithelial function and tight junction barrier integrity in a human, the method comprising administering to the human in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

Similarly provided is a method of treating ulcerative colon diseases in a human, the method comprising administering to the human in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing. In one embodiment, the ulcerative colon disease to be treated is ulcerative colitis. In another embodiment, the ulcerative colon disease to be treated is Crohn Disease or Crohn's Disease. In still another embodiment, the ulcerative colon disease to be treated is Irritable Bowel Syndrome (IBS), which may also be referred to as colitis, enteritis, ileitis, or proctitis.

Also provided is a method of treating Inflammatory Bowel Disease (IBD) in a human, the method comprising administering to the human in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

Also provided is a method of treating diarrhea in a human, the method comprising administering to the human in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing.

As microvascular complications can inhibit uniform delivery of anesthetics, in some embodiments the compounds herein may be used in methods of distributing local, regional, or systemic delivery of anesthetics and improve their effects. As such, also provided is a method of enhancing the delivery of anesthetics to a human experiencing microvascular complications, the method comprising administering to the human in need thereof a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing, and a pharmaceutically effective amount of an anesthetic. In some embodiments, the compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt of any of the foregoing, is administered to the human prior to the administration of anesthetic.

### Pharmaceutical Compositions

Also provided herein is a pharmaceutical composition comprising a pharmaceutically effective amount of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt or co-crystal of any of the foregoing, and a pharmaceutically acceptable carrier or excipient. Additional pharmaceutical compositions comprise, respectively, a pharmaceutically effective amount of a compound selected from those of Formulas (I′), (I″), (I′″), (I-1), (I-2), (I-3), (I-4), (I-5), (I-6), and (I-7), and each of the formulas from (I-a)-(I-II), as well as the specifically named compounds herein, and a pharmaceutically acceptable carrier or excipient.

Further provided herein is the use of a compound of Formula (I-A), (I-B), (I-C), (I-D), or (I-E), a compound of any subformula of Formula (I-A), (I-B), (I-C), (I-D), or (I-E), any of the specifically named compounds herein, or a pharmaceutically acceptable salt or co-crystal of any of the foregoing, in the preparation of a medicament. Additional uses include the compounds that comprise, respectively, a pharmaceutically effective amount of a compound selected from those of Formulas (I′), (I″), (I′″), (I-1), (I-2), (I-3), (I-4), (I-5), (I-6), and (I-7), and each of the formulas from (I-a)-(I-II), as well as the specifically named compounds herein, for use in the preparation of a medicament.

The term “carrier” refers to an excipient or vehicle that includes without limitation diluents, disintegrants, precipitation inhibitors, surfactants, glidants, binders, lubricants, and the like with which the compound is administered. Pharmaceutically acceptable carriers are generally described herein and also in “Remington's Pharmaceutical Sciences” by E. W. Martin. Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

Some examples of suitable excipients in an oral formulation include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

As used herein, a “pharmaceutically acceptable excipient” is a pharmaceutically acceptable vehicle that includes, without limitation, any and all carriers, solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt or co-crystal thereof, may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline may also conventionally be used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. In some embodiments, for parenteral administration, sterile injectable solutions are prepared containing a therapeutically effective amount, e.g., 0.1 to 1000 mg, of the compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt or co-crystal thereof. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, the severity of the subject's symptoms, and the like.

Oral administration is another route for administration of the compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt or co-crystal thereof. Administration may be via capsule or enteric coated tablets, or the like. In making the pharmaceutical compositions that include the compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt or co-crystal thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

In some embodiments, for parenteral administration, each dosage unit contains from 0.1 mg to 1 g, 0.1 mg to 700 mg, or 0.1 mg to 100 mg of a compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt or co-crystal thereof. In some embodiments, a therapeutically effective amount or a pharmaceutically effective amount of a compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt thereof, comprises from about 0.1 mg to about 500 mg per dose, given once or twice daily. In some embodiments, the individual dose is selected from 1 mg, 5 mg, 10 mg, 20 mg, 30 mg, 40 mg, 50 mg, 60 mg, 75 mg, 100 mg, 150 mg, 200 mg, 250 mg, 300 mg, 350 mg, 400 mg, and 500 mg per dose.

For any of the dosage units as described herein, it will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, the severity of the subject's symptoms, and the like.

### Articles of Manufacture and Kits

Compositions (including, for example, formulations and unit dosages) comprising the compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt or co-crystal thereof, can be prepared and placed in an appropriate container, and labeled for treatment of an indicated condition. Accordingly, provided is also an article of manufacture, such as a container comprising a unit dosage form of the compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt or co-crystal thereof, and a label containing instructions for use of the compounds. In some embodiments, the article of manufacture is a container comprising a unit dosage form of the compound of Formula (I-A), (I-B), (I-C), (I-D), or (I-E)), a compound of any subformula of Formula (I-A), (I-B), (I-C), (I-D), or (I-E), any of the specifically named compounds herein, or a pharmaceutically acceptable salt or co-crystal thereof, and at least one pharmaceutically acceptable vehicle. The article of manufacture may be a bottle, vial, ampoule, single-use disposable applicator, or the like, containing the pharmaceutical composition provided in the present disclosure. The container may be formed from a variety of materials, such as glass or plastic and in one aspect also contains a label on, or associated with, the container which indicates directions for use in the treatment of cancer or inflammatory conditions. It should be understood that the active ingredient may be packaged in any material capable of improving chemical and physical stability, such as an aluminum foil bag. In some embodiments, diseases or conditions indicated on the label can include, for example, treatment of cancer.

### Definitions

Terms such as “microvascular tone”, “microcirculatory tone”, “microcirculatory vascular tone”, and “arteriolar tone” refer to the degree of constriction experienced by a microcirculatory blood vessel relative to its maximally dilated state. Microcirculation may be anatomically defined as blood vessels having a diameter of between about 250 μm to about 100 μm, particularly including those between about 200 μm to about 150 μm, and includes arterioles, capillaries, and venules (post-capillary venules). Collectively, these vessels may be referred to as “microvessels”, “microcirculatory vessels”, and the like. In embodiments herein, it is a goal to provide compounds, compositions, and methods for relaxing microvessels in a subject, reducing vascular resistance and protecting the microvessels, themselves, and reducing or elimination end-organ damage associated with microvascular resistance, particularly that seen in the eyes, kidneys, and heart, as well as damage to nerve sheaths associated with microvessels. In some embodiments, the microcirculation is defined as blood vessels <200 μm in diameter. In the human body approximately 90% of the volume of blood resides in these vessels that include arterioles, capillary and venules. Arterioles range in diameter of approximately from 50-200 μm. Some venules contain vascular smooth muscle, and some capillaries are surrounded by pericytes, both of which are contractile cells that allow these vessels to constrict and relax, thus allowing more or less blood and oxygen to be delivered to cells, and cell waste removed. The size of these arterioles is tightly regulated to meet the oxygen demand of the cells they supply.

“Coronary microvascular disease” is heart disease that affects the walls and inner lining of tiny blood vessels branching from larger coronary arteries. Coronary microvascular disease may also be referred to as “Cardiac Syndrome X” or “nonobstructive coronary heart disease”. In the heart or elsewhere it may also be referred to as “small artery disease”, “small vessel disease”, or “arteriolosclerosis.”

Such diseases of the small vessels are often seen in primary hypertension, stroke, and hypertensive renal disease. Manifestations as kidney lesions typically involve afferent arterioles and the interlobular artery and may involve intimal thickening, vascular smooth muscle cell proliferation, and extracellular matrix deposition, increasing the media-to-lumen ration. Areas of vascular smooth muscle cells may then be replaced by fibrosis and cell loss.

“Microvascular angina” refers to a form of chest pain due to abnormalities in cardiac microvessels, including, but not limited to, faulty relaxation of or spasms therein.

A “heterocycle” or “heterocyclic group” herein refers to a chemical ring containing carbon atoms and at least one ring heteroatom selected from O, S, and N, including saturated, unsaturated, partially saturated, and aromatic rings.

Examples of 3-membered heterocycles, such as seen in the definitions of R₃ and R₄ herein, include by way of example and not limitation aziridinyl, azirinyl, oxiranyl, and thiiranyl groups.

Examples of 4-membered heterocycles, such as seen in the definitions of R₃ and R₄ herein, include by way of example and not limitation azetidinyl, dihydro-1λ⁴-azetyl, azetyl, 1,3-diazetidinyl, and oxetanyl groups.

Examples of 5-membered heterocycles, such as seen in the definitions of R₃ and R₄ herein, include by way of example and not limitation thiazolyl, tetrahydrothiophenyl, sulfur oxidized tetrahydrothiophenyl, furanyl, thienyl, pyrrolyl, dihydropyrrolyl, pyrazolyl, imidazolyl, tetrazolyl, pyrrolidinyl, 2-pyrrolidonyl, dihydropyrrolidinyl, pyrrolinyl, tetrahydrofuranyl, triazinyl, thienyl, 2H-pyrrolyl, isothiazolyl, isoxazolyl, imidazolidinyl, imidazolinyl, pyrazolidinyl, pyrazolinyl, and oxazolidinyl groups.

Examples of 6-membered heterocycles, such as seen in the definitions of R₃ and R₄ herein, include by way of example and not limitation pyridyl, dihydroypyridyl, tetrahydropyridyl (piperidyl), pyrimidinyl, piperidinyl, thiadiazinyl, thiazinyl, 2H,6H-1,5,2-dithiazinyl, pyranyl, pyrazinyl, pyridazinyl, piperazinyl, thienyl, thiopyran, dithanyl, morpholinyl, thiomorpholinyl, groups

Examples of 7-membered heterocycles herein, include by way of example and not limitation borepinyl, azepanyl, azepinyl, oxepanyl, oxepinyl, theipinyl, thiepanyl, diazepanyl, diazepinyl, and thiazepinyl groups.

Examples of 8-membered heterocycles herein, include by way of example and not limitation azocinyl, azocinyl, oxocanyl, oxocinyl, thiocanyl, and thiocinyl groups.

Examples of 9-membered heterocycles herein, include by way of example and not limitation monocyclic heterocycles such as azonanyl, azoninyl, oxonanyl, oxoninyl, thionanyl, and thioninyl groups and fused heterocycles, such as indolyl, indolinyl, isoindolyl, indolizinyl, indazolyl, azaindolyl, benzimidazolyl, azaindazolyl, pyrazolopyrimidinyl, purinyl, benzofuranyl, isobenzofuranyl, benzothiophenyl, benzo[d]isoxazolyl, benzo[d]isothiazolyl, benzo[d]oxazolyl, benzo[c][1,2,5]thiadiazolyl, benzo[d]thiazolyl, benzisothiazolyl, adeninyl, and guaninyl groups.

Examples of 10-membered heterocycles herein, include by way of example and not limitation decahydroisoquinolinyl, decahydroquinolinyl, tetrahydroquinolinyl, quinolinyl, dihydroquinolinyl, isoquinolinyl, quinoxalinyl, quinolizinyl, phthalazinyl, quinazolinyl, cinnolinyl, chromenyl, isochromenyl, naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, benzo[e][1,2]oxazinyl, benzo[e][1,3]oxazinyl, benzo[b][1,4]oxazinyl, quinolinonyl, thienoppyrimidinyl (including thieno[3,2-d]pyrimidinyl), and isoquinolinonyl groups.

It is understood that the general terms for the heterocycles referenced herein include each isomeric form of the heterocycle, such as the term “dithianyl” including 1,2 dithianyl, 1,3-dithianyl, and 1,4-dithianyl groups, the term “thiadiazinyl” including 1,2,5 thiadiazineyl and 1,3,4-thiadiainyl groups, the term “azaindolyl” including 4-azaindolyl, 5-azaindolyl, 6-azaindolyl, and 7-azaindolyl groups, and “benzothiophenyl” including benzo[b]thiophenyl and benzo[c]thiophenyl groups.

Similarly, general heterocycle names include each variance in one or more points of unsaturation. For instance, the term “dihydropyrrolyl” refers to “2,3-dihydro-1H-pyrrolyl” and “2,5-dihydro-1H-pyrrolyl” groups.

The term “alkyl” refers to a straight or branched hydrocarbon. For example, an alkyl group can have a specified number of chain carbons, such as 1 to 6 carbon atoms (i.e., C₁-C₆ alkyl or C₁-6 alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃) CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃) CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃) CH(CH₃)₂), 3-methyl-1-butyl (—CH₂CH₂CH(CH₃)₂), 2-methyl-1-butyl (—CH₂CH(CH₃) CH₂CH₃), 1-hexyl (—CH₂CH₂CH₂CH₂CH₂CH₃), 2-hexyl (—CH(CH₃) CH₂CH₂CH₂CH₃), 3-hexyl (—CH(CH₂CH₃) (CH₂CH₂CH₃)), 2-methyl-2-pentyl (—C(CH₃)₂CH₂CH₂CH₃), 3-methyl-2-pentyl (—CH(CH₃) CH(CH₃) CH₂CH₃), 4-methyl-2-pentyl (—CH(CH₃) CH₂CH(CH₃)₂), 3-methyl-3-pentyl (—C(CH₃) (CH₂CH₃)₂), 2-methyl-3-pentyl (—CH(CH₂CH₃) CH(CH₃)₂), 2,3-dimethyl-2-butyl (—C(CH₃)₂CH(CH₃)₂), and 3,3-dimethyl-2-butyl (—CH(CH₃) C(CH₃) 3.

The term “alkoxy” refers to a group having the formula “—O-alkyl,” in which an alkyl group, as defined above, is attached to the parent molecule via an oxygen atom. The alkyl portion of an alkoxy group can have a specified number of carbon chain atoms, such as 1 to 6 carbon atoms (i.e., C₁-C₆ alkoxy or C₁-6 alkoxy). Examples of suitable alkoxy groups include, but are not limited to, methoxy (—O—CH₃ or —OMe), ethoxy (—OCH₂CH₃ or —OEt), t-butoxy (—O—C(CH₃)₃ or —OtBu) and the like.

The term “carbocycle” or “carbocyclic group” refers to a chemical ring containing only carbon atoms, including saturated, unsaturated, partially saturated, and aromatic rings.

The term “cycloalkyl” refers to a saturated ring having 3 to 6 carbon atoms as a monocycle, including cyclopropyl, cyclobutyl, cyclopentyl, and cyclohexyl groups.

The term “halogen” refers to an atom selected from the group of elements chlorine, fluorine, bromine, and iodine.

The term “oxo” refers to double-bonded oxygen “═O”.

The terms “therapeutically effective amount” and “pharmaceutically effective amount” refer to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound of Formula (I-A), (I-B), (I-C), (I-D), or (I-E), or a pharmaceutically acceptable salt or co-crystal thereof, is an amount sufficient to antagonize GPR39 expression or activity, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to inhibition of GPR39 activity.

“Treatment” or “treating” is an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

The terms “inhibiting” or “inhibition” indicates a decrease, such as a significant decrease, in the baseline activity of a biological activity or process. “Inhibition of GPR39 activity” refers to a decrease in GPR39 activity as a direct or indirect response to the presence of a compound of Formula (I-A), (I-B), (I-C), (I-D), or (I-E), or a pharmaceutically acceptable salt or co-crystal thereof, relative to the activity of GPR39 in the absence of such compound or a pharmaceutically acceptable salt or co-crystal thereof. The decrease in activity may be due to the direct interaction of the compound with GPR39, or due to the interaction of the compound(s) described herein with one or more other factors that in turn affect GPR39 expression and/or activity. For example, the presence of the compound(s) may decrease GPR39 activity by directly binding to the GPR39, by causing (directly or indirectly) another factor to decrease GPR39 expression or activity, or by (directly or indirectly) decreasing the amount of GPR39 protein present in the cell or organism. In some embodiments, the inhibition of GPR39 activity may be compared in the same subject prior to treatment, or other subjects not receiving the treatment. The term “inhibitor” is understood to refer to a compound or agent that, upon administration to a human in need thereof at a pharmaceutically or therapeutically effective dose, provides the inhibition activity desired.

“Delaying” the development of a disease or condition means to defer, hinder, slow, retard, stabilize, and/or postpone development of the disease or condition. This delay can be of varying lengths of time, depending on the history of the disease or condition, and/or subject being treated. A method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed and/or claimed herein are inclusive of the recited endpoint and independently combinable (for example, the ranges of “from 2 to 10” and “2-10” are inclusive of the endpoints, 2 and 10, and all the intermediate values 3, 4, 5, 6, 7, 8, and 9).

By “significant” is meant any detectable change that is statistically significant in a standard parametric test of statistical significance such as Student's T-test, where p<0.05.

“Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The term “crystal forms” and related terms herein refer to the various crystalline modifications of a given substance, including, but not limited to, polymorphs, solvates, hydrates, co-crystals, and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates, such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding and solvent-drop grinding.

The term “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a compound of Formula (I-A), (I-B), (I-C), (I-D), or (I-E), and a guest (or co-former) molecule or molecules. In particular embodiments the pharmaceutically acceptable co-crystal of the compound of Formula (I-A), (I-B), (I-C), (I-D), or (I-E) with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

As used herein, the terms “isotope” and “isotopic” in reference to a compound as disclosed herein means that one or more atoms of the compound is replaced with an isotope of such one or more atoms. An “isotope” refers to any of two or more forms of a chemical element, having the same number of protons in the nucleus, but having different numbers of neutrons in the nucleus. For example, an isotopic compound includes a compound in which one or more hydrogen atoms (H) has been replaced with one or more deuterium atoms (D). In this example, deuterium is an isotope of hydrogen, and replacing a hydrogen atom with deuterium (at one or more positions) renders the resulting compound an isotopic compound. For example, and in reference to Formula (I-A), (I-B), (I-C), (I-D), or (I-E), replacing the two methyl groups of the isopropyl moiety (—CH(CH₃)₂) with fully deuterated methyl groups (—CH(CD₃)₂) would be an isotopic compound of Formula (I-A), (I-B), (I-C), (I-D), or (I-E). In addition to replacing hydrogen with deuterium, other stable (non-radioactive) isotope substitutions include replacing carbon 12 with carbon 13, while unstable (radioactive) isotopes include replacing hydrogen with tritium, replacing carbon 12 with carbon 14, replacing iodine 127 with iodine 123 or iodine 125, and the like. Accordingly, all reference herein to isotopic compounds of Formula (I-A), (I-B), (I-C), (I-D), or (I-E), as well as all reference to the various embodiments thereof, refers to a compound having one or more isotopic substitutions, including (but not limited to) substitutions of one or more hydrogen atoms with one or more deuterium atoms and any occurrence(s) in the compound. To this end, the isotopic compounds disclosed herein provide improved advantages relative to their non-isotopic forms. To this end, isotopic modification provides a means of improving existing drugs and/or as a tool in the design of new drugs. For example, isotopic drug design has proven successful in the context of the deuterium (D) kinetic isotope effect. Due to the twofold higher mass of D compared with H, the C-D bond is much more resistant toward oxidative processes (such as its ability to be catalyzed by CYP450 or by other enzymes involved in metabolism), while retaining very similar steric properties. Therefore, H-D isosteric replacement usually retains the pharmacodynamics of the compound, while improving its pharmacokinetics with a repercussion on half-life and/or of area under the curve values and, ultimately, on dose and/or dosing regimen. For example, drug exposure may be enhanced with isotopic modification, and/or a decrease of clearance. Such benefits are provided to the compounds disclosed hereby by way their isotopic derivation.

Terms such as “subject” and “patient” refer to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a compound of Formula (I-A), (I-B), (I-C), (I-D), or (I-E), or a pharmaceutically acceptable salt or co-crystal thereof, as described herein. This includes a subject who may be determined to be at risk of or susceptible to such diseases or conditions, such that treatment would prevent the disease or condition from developing.

The terms “prediabetes” or “prediabetic condition” refers to a condition in which a subject's blood sugar levels are not high enough to be considered diabetic but may be a precursor to Type 2 diabetes. A prediabetic condition may be defined in subjects having a fasting blood glucose level of 100 mg/dl or more, but less than 126 mg/dl (the level which is diagnostic for diabetes). Hemoglobin A1c (HbA1c) levels are another laboratory test for diabetes. HbA1c levels of 6.5% or greater are characteristic of diabetes, while levels from 5.7% to 6.4% suggest prediabetes.

### EXAMPLES

### Measurement of Intracellular Calcium Response in PC3 Cells, Expressing the Endogenous Human GPR39 Receptor

Human prostate adenocarcinoma (PC3) cells endogenously expressing human GPR39 receptor were seeded into black walls clear-base 384-well plates at a density of 7,500 cells per well in DMEM (Low Glucose) supplemented with 10% heat-inactivated foetal bovine serum and 1% of Pen/Strep and grown overnight at 37° C., 5% CO2. After washing with assay buffer (20 mM HEPES, 137 mM NaCl, 5.4 mM KCl, 10 mM glucose, 0.8 mM MgSO4, 1.3 mM CaCl2), 0.3 mM Na2HPO4, 0.4 mM KH2PO4 4.2 mM NaHCO₃, pH 7.4), cells were incubated with Calcium-6 probe (Molecular Devices) at 37° C. for 2 h and then equilibrated at RT for 30 minutes on metal block. The fluorescence was measured in real time by Fluorometric Imaging Plate Reader (FLIPR TETRA, Molecular Devices) (excitation wavelength at 470-495 nm, emission wavelength at 515-575 nm).
Compounds of invention were serially diluted in neat DMSO at 1000 fold concentrations and plated into 384-well plates and then 200-fold diluted in assay buffer containing 150 μM ZnCl2 (30 μM final concentration) and 1.1% DMSO (0.32% final concentration).
A dual addition FLIPR protocol was used which included a first addition of compounds 200-fold diluted in Assay Buffer containing 150 μM ZnCl2 and 1.1% DMSO and then after 10 minutes a second addition of a submaximal concentration of the hGPR39 receptor agonist, C3 (Tocris, TC-G 1008) at EC90 (concentration producing 90% of the maximal response) value of 1.5 μM.
Fluorescence was monitored throughout the run assay. The peak of Ca2+ stimulation (baseline subtracted) was expressed as percentage of the maximal inhibition of the EC90 C₃ response and was plotted versus the concentration of test compound. Curve fitting and IC50 estimations are carried out using a four-parameter logistic model using XLfit Software.
The potency of compounds was reported as functional pKi (−Log10Ki) obtained using the modified Cheng-Prusoff relationship (Cheng, Y., Prusoff, W. H. 1973.)
fKi=IC50/(1+[L]/EC50) where IC50 is the concentration of antagonist required for 50% inhibition of the maximum response, [L] is the concentration of the agonist used (EC90) and EC50 is the concentration of agonist need to induced 50% of the maximum response (obtained in each experimental plate).
fpKi data is shown in Table 1 below. An fpKi less than 5.2 is reported as “+/−”. An fpKi ranging from 5.2 to 5.9 is reported as “+”. An fpKi from 6.0 to 6.5 is reported as “++”. An fpKi from 6.6 to 7.2 is reported as “+++”. An fpKi equal to or greater than 7.3 is reported as “++++”.

TABLE 1

| Example No. | fpKi activity |
|---|---|
| 1 | + |
| 2 | + |
| 3 | + |
| 4 | + |
| 5 | +/− |
| 6 | ++ |
| 7 | + |
| 8 | + |
| 9 | ++ |
| 18 | + |
| 19 | +/− |
| 20 | + |
| 21 | +/− |
| 22 | +++ |
| 23 | ++++ |
| 24 | ++++ |
| 25 | ++ |
| 26 | + |
| 27 | + |
| 28 | ++++ |
| 29 | ++ |
| 30 | ++ |
| 31 | +/− |
| 32 | + |
| 33 | +/− |
| 34 | +++ |
| 37 | ++++ |
| 38 | ++++ |
| 39 | ++++ |
| 40 | +++ |
| 41 | ++++ |
| 42 | ++++ |
| 43 | ++++ |
| 44 | + |
| 45 | ++++ |
| 46 | +++ |
| 47 | ++++ |
| 48 | ++++ |
| 49 | ++ |
| 50 | ++++ |
| 51 | ++++ |
| 52 | ++++ |
| 53 | ++++ |
| 54 | ++++ |
| 55 | ++++ |
| 56 | ++++ |
| 57a | ++++ |
| 57b | ++++ |
| 58 | ++++ |
| 59 | ++++ |
| 60 | ++++ |
| 61 | ++++ |
| 62 | ++++ |
| 63 | ++++ |
| 64 | ++++ |
| 65 | ++++ |
| 66 | ++++ |
| 67 | ++++ |
| 68 | ++++ |
| 69 | ++++ |
| 70 | ++++ |
| 71 | ++++ |
| 72 | ++++ |
| 73 | ++++ |
| 74 | ++++ |
| 75 | ++++ |
| 76 | ++++ |
| 77 | ++++ |
| 78 | ++++ |
| 79 | ++++ |
| 80 | ++++ |
| 81 | +++ |
| 82 | + |
| 84 | ++ |
| 85 | + |
| 86 | ++++ |
| 87 | +++ |
| 88 | ++ |
| 89 | +++ |
| 90 | +/− |
| 91 | ++ |
| 92 | +/− |
| 93 | +/− |
| 94 | ++ |
| 95 | +/− |
| 96 | + |
| 97 | ++ |
| 98 | ++ |
| 99 | +/− |
| 100 | + + |
| 101 | +/− |
| 102 | + |
| 103 | + |
| 104 | + |
| 105 | +/− |
| 106 | ++ |
| 107 | +++ |
| 109 | +++ |
| 110 | ++++ |
| 111 | +++ |
| 112 | +++ |
| 113 | +/− |
| 114 | +/− |
| 115 | + |
| 116 | +++ |
| 117 | + |
| 118 | ++++ |
| 119 | ++++ |
| 120 | ++ |
| 121 | ++++ |
| 122 | ++++ |
| 123 | +/− |
| 124 | +/− |
| 110a | ++++ |
| 110b | + |
| 125a | ++ |
| 125b | ++++ |
| 125c | +++ |
| 125d | ++++ |

### Synthesis

The compounds described herein may be prepared by methods known in the art and are exemplified by the following non-limiting descriptions.

Unless otherwise stated, all reactions are typically performed under inert atmosphere (for example under Nitrogen). The following abbreviations are used in the text:
EtOAc, AcOEt, EA=ethyl acetate
Et₂O=diethyl ether
CH₃CN=acetonitrile
MeOH=methanol
THF=tetrahydrofuran
r.t. (RT)=room temperature
DMSO=dimethyl sulfoxide
DMF=N,N′-dimethylformamide
DCM=dichloromethane
EtOH=ethanol
RP=reverse phase
FA=formic acid
Cy, cHex=cyclohexane
TEA=triethylamine
DIPEA=N, N-Diisopropylethylamine
Boc₂O=Di-tert-butyl dicarbonate
TFA=trifluoroacetic acid
HATU=1-[Bis(dimethylamino)methylene]-1H-1,2,3-triazolo[4,5-b]pyridinium 3-oxid hexafluorophosphate
AcOH=acetic acid
SCX Cartridge=Strong Cation Exchange Cartridge
FC=Flash chromatography
s.s.=saturated solution
ON=overnight

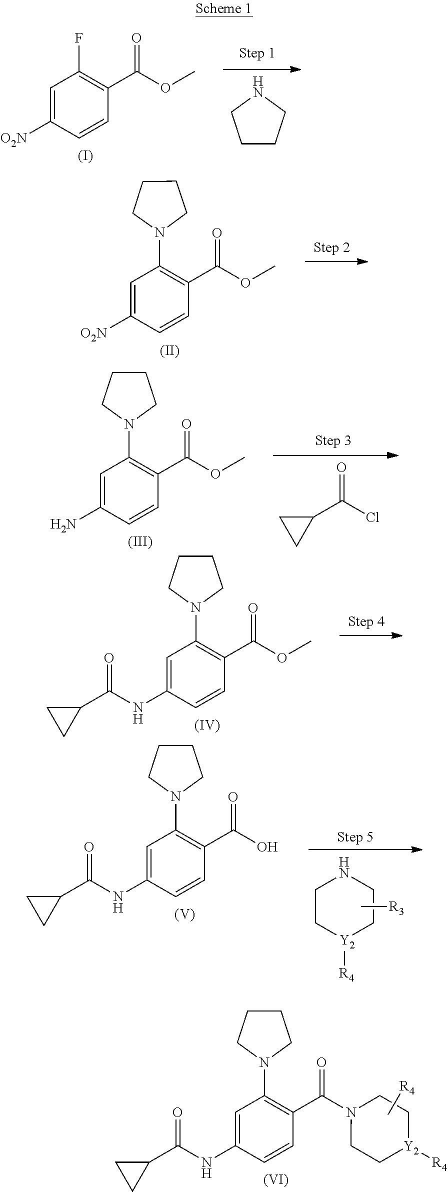

### Step 1

Compound of formula II may be obtained from compound of formula I (commercially available from Enamine) by SNAr substitution with pyrrolidine in a suitable aprotic solvent, e.g. CH₃CN, typically at 80° C. The reaction takes about 1 hour to complete.

### Step 2

Compound of formula III may be obtained by nitro reduction of compound of formula II under catalytic hydrogenation conditions, using for example Pd/C in a suitable solvent such as acetic acid, typically at room temperature. The reaction takes about 4 hours to complete

### Step 3

Compound of formula IV may be obtained by coupling reaction between compound of formula III and a proper commercially available acyl chloride, e.g. cyclopropanecarbonyl chloride, in presence of an organic base, e.g. TEA. The reaction is carried out in a suitable solvent such as DCM, typically at room temperature. The reaction takes about 12 hours to complete.

### Step 4

Compound of formula V may be obtained from compound of formula IV by hydrolysis, e.g. using LiOH, in a suitable mixture of solvents such as MeOH/THF/H₂O, typically at 50° C. The reaction takes about 36 hours to complete.

### Step 5

Compound of formula VI may be obtained from compound of formula V by coupling with the proper amine in the presence of coupling agent, e.g. HATU, and an organic base, e.g. DIPEA. The reaction is carried out in a suitable solvent such as DMF, typically at room temperature. The reaction takes from about 3 hours to about 12 hours to complete.

### Scheme 1, Cpd II Preparation: methyl 4-nitro-2-pyrrolidin-1-ylbenzoate

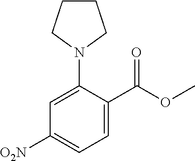 [SMILES: COC(=O)c1ccc([N+](=O)[O-])cc1N1CCCC1]

To a solution of Methyl 2-Fluoro-4-nitrobenzoato (5.0 g, 25.11 mmol) in MeCN (50 mL), pyrrolidine (5.36 g, 75.32 mmol) was added and the reaction was stirred at 80° C. for 1 h. After this time the reaction was cooled down to RT and then concentrated under vacuum. The residue was taken up with water and filtered. The solid was dried under vacuum affording the product of formula methyl 4-nitro-2-pyrrolidin-1-ylbenzoate which was used in the next step without further purification.

### Yield: 6 g

1H NMR (400 MHZ, DMSO-d6) δ 7.64 (d, 1H), 7.49 (d, 1H), 7.44 (dd, 1H), 3.86 (s, 3H), 3.26-3.20 (m, 4H), 1.95-1.90 (m, 4H); LC-MS: 251.05 (MH+).

### Scheme 1, Cpd Ill Preparation: methyl 4-amino-2-pyrrolidin-1-ylbenzoate

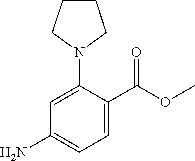 [SMILES: COC(=O)c1ccc(N)cc1N1CCCC1]

To a solution of methyl 4-nitro-2-pyrrolidin-1-ylbenzoate (4.75 g, 18.98 mmol), in Acetic acid (190 mL) Pd—C 10% (2.02 g, 1.9 mmol) was added. The reaction was stirred for 4 hours under H₂ atmosphere at room temperature. After this time the mixture was filtered through a celite pad, washed with MeOH and the solvent was evaporated under vacuum. The residue was taken up with a s. s. of NaHCO₃ and extracted with AcOEt 3 times. The combined organic fractions were washed with brine, dried over Na₂SO₄, filtered and concentrated under vacuum affording the product of formula methyl 4-amino-2-pyrrolidin-1-ylbenzoate, which was used in the next step without further purification.

### Yield: 3.95 g

1H NMR (400 MHZ, DMSO-d6) δ 7.29 (d, 1H), 6.02-5.88 (m, 2H), 5.47 (s, 2H), 3.67 (s, 3H), 3.16-2.99 (m, 4H), 2.00-1.71 (m, 4H); LC-MS: m/z 221.39 (MH+).

### Scheme 1, Cpd IV Preparation: methyl 4-(cyclopropanecarbonylamino)-2-pyrrolidin-1-ylbenzoate

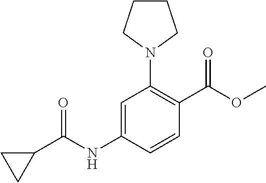 [SMILES: COC(=O)c1ccc(NC(=O)C2CC2)cc1N1CCCC1]

A solution of methyl 4-amino-2-pyrrolidin-1-ylbenzoate (5.75 g, 26.1 mmol) and N,N-Diisopropylethylamine (9.09 mL, 52.21 mmol) in dry DCM (145.57 mL) was cooled to 0° C. and stirred for 10 min. After this time cyclopropanecarbonyl chloride (3.08 mL, 33.94 mmol) was added dropwise and the reaction mixture was allowed to reach room temperature and stirred overnight. The day after the reaction was diluted with DCM and washed with a saturated solution of NaHCO₃. The organic phase was washed with brine, dried over a phase separator and the solvent was evaporated under reduced pressure. The residue was purified by FC on silica gel (eluting from cHex/AcOEt 9:1 to cHex/AcOEt 9:1) to afford the product of formula methyl 4-(cyclopropanecarbonylamino)-2-pyrrolidin-1-ylbenzoate.

### Yield: 5.6 g

1H NMR (400 MHZ, DMSO-d6) δ 10.22 (s, 1H), 7.41 (d, 1H), 7.22 (d, 1H), 6.92 (dd, 1H), 3.75 (s, 3H), 3.11 (q, 4H), 1.92-1.83 (m, 4H), 1.83-1.75 (m, 1H), 0.83-0.76 (m, 4H); LC-MS: m/z 289.12 (MH+).

### Scheme 1, Cpd V Preparation: 4-(cyclopropanecarbonylamino)-2-pyrrolidin-1-ylbenzoic acid

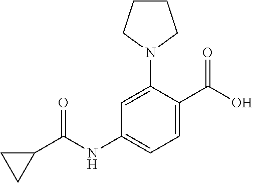 [SMILES: O=C(O)c1ccc(NC(=O)C2CC2)cc1N1CCCC1]

To a solution of methyl 4-(cyclopropanecarbonylamino)-2-pyrrolidin-1-ylbenzoate (5.0 g, 17.34 mmol) in THF (150 mL)/MeOH (20 mL), a 6N solution of NaOH (5.78 mL, 34.68 mmol) was added. The reaction was stirred at 50° C. overnight.
The day after further sodium hydroxide solution 6 N (5.78 mL, 34.68 mmol) was added followed by 10 mL of MeOH and the reaction was stirred at 50° C. for 1 day. After this time the reaction was cooled down to RT and concentrated under vacuum. The aqueous solution was washed with DCM (3 times), acidified with HCl 12 N until pH=1 and extracted with AcOEt 5 times. The combined organic fractions were washed with Brine, dried over Na₂SO₄, filtered and concentrated under vacuum. The residue was taken up with Et₂O and filtered, the solid was washed with Et₂O and dried under vacuum affording the product of formula 4-(cyclopropanecarbonylamino)-2-pyrrolidin-1-ylbenzoic acid, which was used in the next step without further purification.

### Yield: 2.5 g

1H NMR (400 MHZ, DMSO-d6) δ 13.10 (s, 1H), 10.25 (s, 1H), 7.52 (d, 1H), 7.34 (d, 1H), 6.99 (dd, 1H), 3.21-3.04 (m, 4H), 2.01-1.78 (m, 4H), 1.79 (tt, 1H), 0.86-0.73 (m, 4H); LC-MS: m/z 275.08 (MH+).

### Preparation: N-[4-(2-phenylpyrrolidine-1-carbonyl)-3-pyrrolidin-1-ylphenyl]cyclopropanecarboxamide

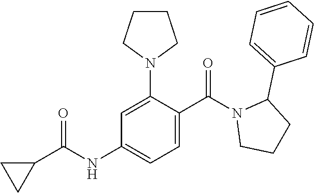 [SMILES: O=C(Nc1ccc(C(=O)N2CCCC2c2ccccc2)c(N2CCCC2)c1)C1CC1]

### Example 1

To a solution of 4-(cyclopropanecarbonylamino)-2-pyrrolidin-1-yl benzoic acid (46.58 mg, 0.170 mmol) and (HATU) [dimethylamino (3-triazolo[4,5-b]pyridinyloxy)methylidene]-dimethylammonium hexafluorophosphate (96.85 mg, 0.250 mmol) and N,N-Diisopropylethylamine (0.09 mL, 0.510 mmol) in DMF (2 mL) a solution 2-phenylpyrrolidine (25.0 mg, 0.170 mmol) in DMF (1 mL) was added. The reaction mixture was stirred at RT/ON. After completion of the reaction the mixture was diluted with a s. s. of NaHCO₃ and extracted with AcOEt 3 times. The combined organic fractions were washed with brine, dried over Na₂SO₄, filtered and concentrated under vacuum. The residue was purified by FC on NH column (eluting from cHex/AcOEt 8:2 to cHex/AcOEt 2:8) affording the product of formula N-[4-(2-phenylpyrrolidine-1-carbonyl)-3-pyrrolidin-1-ylphenyl]cyclopropanecarboxamide (see Example 1).

### Yield: 34.2 mg

¹H NMR (400 MHZ, DMSO-d₆) δ 10.14-9.87 (m, 1H), 7.38-7.27 (m, 3H), 7.26-7.12 (m, 2H), 7.09-6.92 (m, 3H), 5.17-4.78 (m, 1H), 3.61 (d, 2H), 3.08 (d, 4H), 2.39-2.17 (m, 1H), 2.04-1.62 (m, 8H), 0.84-0.62 (m, 4H); LC-MS: m/z 404.3 (MH+).

### Preparation: N-[4-(3-phenylpyrrolidine-1-carbonyl)-3-pyrrolidin-1-ylphenyl]cyclopropanecarboxamide

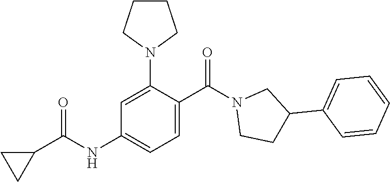 [SMILES: O=C(Nc1ccc(C(=O)N2CCC(c3ccccc3)C2)c(N2CCCC2)c1)C1CC1]

### Example 2

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein 3-phenylpyrrolidine was used instead of 2-phenylpyrrolidine. The title compound was obtained in a yield of 38% (26 mg).

1H NMR (400 MHZ, DMSO-d6) δ 10.08 (d, 1H), 7.39-7.18 (m, 5H), 7.10-6.97 (m, 2H), 6.96-6.85 (m, 1H), 3.99-3.65 (m, 2H), 3.38 (d, 3H), 3.26-2.99 (m, 4H), 2.25 (d, 1H), 2.08-1.70 (m, 6H), 0.77 (dtd, 4H); LC-MS: m/z 404.3 (MH+).

### Preparation: N-[4-(2-oxa-7-azaspiro[3.4]octane-7-carbonyl)-3-pyrrolidin-1-ylphenyl]cyclopropanecarboxamide

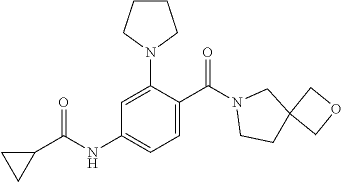 [SMILES: O=C(Nc1ccc(C(=O)N2CCC3(COC3)C2)c(N2CCCC2)c1)C1CC1]

### Example 3

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein 2-oxa-6-azaspiro[3.4]octane was used instead of 2-phenylpyrrolidine. The title compound was obtained in a yield of 61% (40 mg).

1H NMR (400 MHZ, DMSO-d6) δ 10.08 (s, 1H), 7.10-7.02 (m, 1H), 6.99-6.86 (m, 2H), 4.57 (d, 1H), 4.49-4.38 (m, 3H), 3.65 (s, 1H), 3.41 (s, 2H), 3.11 (d, 5H), 2.12 (dt, 2H), 1.89-1.81 (m, 4H), 1.77 (qd, 1H), 0.77 (dt, 4H); LC-MS: m/z 370.2 (MH+).

### Preparation: N-[4-[(trans)-2,6-dimethylmorpholine-4-carbonyl]-3-pyrrolidin-1-ylphenyl]cyclopropanecarboxamide

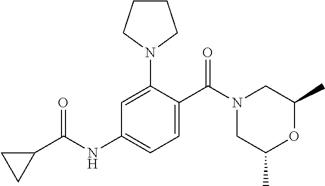 [SMILES: C[C@@H]1CN(C(=O)c2ccc(NC(=O)C3CC3)cc2N2CCCC2)C[C@@H](C)O1]

### Example 4

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein trans-2,6-dimethylmorpholine was used instead of 2-phenylpyrrolidine. The title compound was obtained in a yield of 50% (33 mg) as a racemic mixture. 1H NMR (400 MHZ, DMSO-d6) δ ppm 10.09 (s, 1H), 7.06 (s, 1H), 6.99-6.88 (m, 2H), 4.11-3.67 (m, 3H), 3.41-2.72 (m, 7H), 1.95-1.80 (m, 4H), 1.80-1.70 (m, 1H), 1.32-1.09 (m, 3H), 0.99 (dd, 3H), 0.82-0.69 (m, 4H); LC-MS: m/z 372.23 (MH+).

### Preparation: N-[4-(2,6-dimethyl-1,1-dioxo-1,4-thiazinane-4-carbonyl)-3-pyrrolidin-1-ylphenyl]cyclopropanecarboxamide

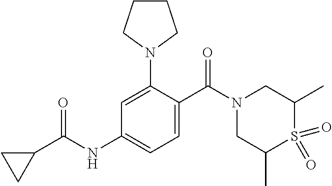 [SMILES: CC1CN(C(=O)c2ccc(NC(=O)C3CC3)cc2N2CCCC2)CC(C)S1(=O)=O]

### Example 5

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein 2,6-dimethyl-1,4-thiazinane 1,1-dioxide hydrochloride was used instead of 2-phenylpyrrolidine. The title compound was obtained in a yield of 44% (34 mg).

1H NMR (400 MHZ, DMSO-d6) δ 10.13 (d, 1H), 7.28-6.72 (m, 3H), 4.79 (d, 1H), 3.83 (dd, 1H), 3.48-3.38 (m, 1H), 3.25-2.76 (m, 7H), 1.97-1.65 (m, 5H), 1.25 (t, 3H), 1.06 (dd, 3H), 0.81-0.74 (m, 4H); LC-MS: m/z 420.2 (MH+).

### Preparation: N-[4-(8-oxa-3-azabicyclo[3.2.1]octane-3-carbonyl)-3-pyrrolidin-1-ylphenyl]cyclopropanecarboxamide

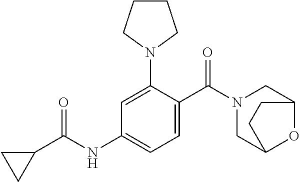 [SMILES: O=C(Nc1ccc(C(=O)N2CC3CCC(C2)O3)c(N2CCCC2)c1)C1CC1]

### Example 6

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein 8-oxa-3-azabicyclo[3.2.1]octane hydrochloride was used instead of 2-phenylpyrrolidine. The title compound was obtained in a yield of 44% (34 mg).

1H NMR (400 MHZ, DMSO-d6) δ 10.09 (s, 1H), 7.06 (dd, 1H), 7.00-6.82 (m, 2H), 4.47-4.00 (m, 3H), 3.24-2.82 (m, 7H), 1.98-1.44 (m, 9H), 0.77 (dt, 4H); LC-MS: m/z 370.2 (MH+).

### Preparation: N-[4-(3-oxa-8-azabicyclo[3.2.1]octane-8-carbonyl)-3-pyrrolidin-1-ylphenyl]cyclopropanecarboxamide

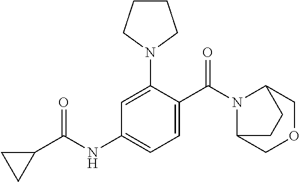 [SMILES: O=C(Nc1ccc(C(=O)N2C3CCC2COC3)c(N2CCCC2)c1)C1CC1]

### Example 7

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein 3-oxa-8-azabicyclo[3.2.1]octane hydrochloride was used instead of 2-phenylpyrrolidine. The title compound was obtained in a yield of 13% (9 mg).

1H NMR (400 MHZ, DMSO-d6) δ 10.13 (s, 1H), 7.33-6.83 (m, 3H), 4.53 (s, 1H), 3.84-3.41 (m, 5H), 3.23-2.94 (m, 4H), 1.97-1.70 (m, 9H), 0.82-0.67 (m, 4H); LC-MS: m/z 370.2 (MH+).

### Preparation: N-[4-(6-oxa-3-azabicyclo[3.1.1]heptane-3-carbonyl)-3-pyrrolidin-1-ylphenyl]cyclopropanecarboxamide

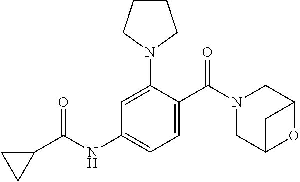 [SMILES: O=C(Nc1ccc(C(=O)N2CC3CC(C2)O3)c(N2CCCC2)c1)C1CC1]

### Example 8

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein 6-oxa-3-azabicyclo[3.1.1]heptane hydrochloride was used instead of 2-phenylpyrrolidine. The title compound was obtained in a yield of 75% (49 mg).

1H NMR (400 MHZ, DMF-d7) δ 10.07 (s, 1H), 7.07 (d, 1H), 7.01-6.85 (m, 2H), 4.53 (d, 2H), 3.84 (d, 1H), 3.61-3.44 (m, 2H), 3.26 (s, 2H), 3.24-2.93 (m, 5H), 1.94-1.56 (m, 5H), 0.77 (dd, 4H); LC-MS: m/z 356.2 (MH+)

### Preparation: N-[4-(3-azabicyclo[3.1.1]heptane-3-carbonyl)-3-pyrrolidin-1-ylphenyl]cyclopropanecarboxamide

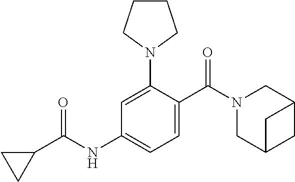 [SMILES: O=C(Nc1ccc(C(=O)N2CC3CC(C3)C2)c(N2CCCC2)c1)C1CC1]

### Example 9

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein 3-oxa-8-azabicyclo[3.2.1]octane hydrochloride was used instead of 2-phenylpyrrolidine. The title compound was obtained in a yield of 78% (50 mg).

1H NMR (400 MHZ, DMSO-d6) δ 10.06 (s, 1H), 7.05 (s, 1H), 6.93 (s, 2H), 3.73-3.34 (m, 4H), 3.29-3.01 (m, 6H), 2.39-2.32 (m, 1H), 2.17-2.04 (m, 2H), 1.97-1.68 (m, 4H), 1.33-1.13 (m, 2H), 0.78 (dd, 4H); LC-MS: m/z 354.2 (MH+)

### Preparation: tert-butyl N-[1-[4-(cyclopropanecarbonylamino)-2-pyrrolidin-1-ylbenzoyl]piperidin-4-yl]-N-methylcarbamate

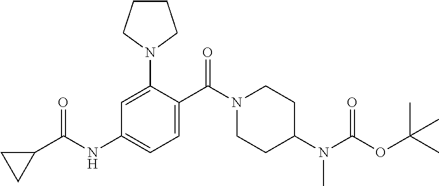 [SMILES: CN(C(=O)OC(C)(C)C)C1CCN(C(=O)c2ccc(NC(=O)C3CC3)cc2N2CCCC2)CC1]

### Example 10

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein 4-N—BOC-4-methyl-aminopiperidine was used instead of 2-phenylpyrrolidine. The title compound was obtained in a quantitative yield (140 mg).

### LC-MS: m/z 471.49 (MH+)

### Preparation: tert-butyl N-[1-[4-(cyclopropanecarbonylamino)-2-pyrrolidin-1-ylbenzoyl]piperidin-4-yl]carbamate

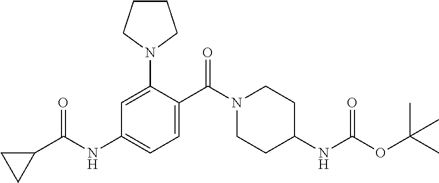 [SMILES: CC(C)(C)OC(=O)NC1CCN(C(=O)c2ccc(NC(=O)C3CC3)cc2N2CCCC2)CC1]

### Example 11

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein N-(4-piperidinyl) carbamic acid tert-butyl ester was used instead of 2-phenylpyrrolidine. The title compound was obtained in a quantitative yield (140 mg).

### LC-MS: m/z 457.48 (MH+)

### Preparation: tert-butyl 4-[4-(cyclopropanecarbonylamino)-2-pyrrolidin-1-ylbenzoyl]-3-[2-(trifluoromethyl)phenyl]piperazine-1-carboxylate

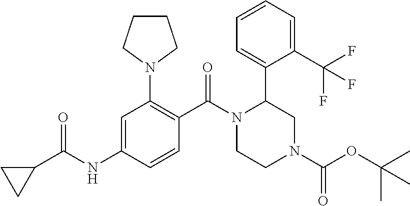 [SMILES: CC(C)(C)OC(=O)N1CCN(C(=O)c2ccc(NC(=O)C3CC3)cc2N2CCCC2)C(c2ccccc2C(F)(F)F)C1]

### Example 12

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein tert-butyl 3-[2-(trifluoromethyl)phenyl]piperazine-1-carboxylate was used instead of 2-phenylpyrrolidine. The title compound was obtained as a racemic mixture in a yield of 19% (24 mg).

### LC-MS: m/z 587.30 (MH+).

### Preparation: tert-butyl 3-(3-cyanophenyl)-4-[4-(cyclopropanecarbonylamino)-2-pyrrolidin-1-ylbenzoyl]piperazine-1-carboxylate

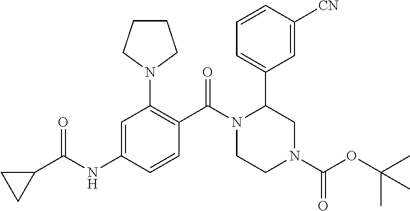 [SMILES: CC(C)(C)OC(=O)N1CCN(C(=O)c2ccc(NC(=O)C3CC3)cc2N2CCCC2)C(c2cccc(C#N)c2)C1]

### Example 13

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein tert-butyl 3-(3-cyanophenyl) piperazine-1-carboxylate was used instead of 2-phenylpyrrolidine. The title compound was obtained as a racemic mixture in a yield of 47% (55 mg).

### LC-MS: m/z 544.27 (MH+).

### Preparation: tert-butyl 4-[4-(cyclopropanecarbonylamino)-2-pyrrolidin-1-ylbenzoyl]-3-(4,5-dimethyl-1,3-thiazol-2-yl) piperazine-1-carboxylate

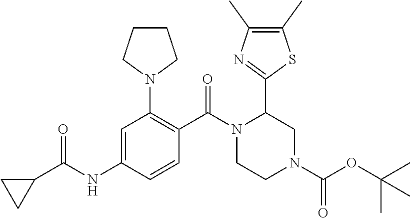 [SMILES: Cc1nc(C2CN(C(=O)OC(C)(C)C)CCN2C(=O)c2ccc(NC(=O)C3CC3)cc2N2CCCC2)sc1C]

### Example 14

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein tert-butyl 3-(4,5-dimethyl-1,3-thiazol-2-yl) piperazine-1-carboxylate was used instead of 2-phenylpyrrolidine. The title compound was obtained as a racemic mixture in a yield of 60% (73 mg).

### LC-MS: m/z 554.25 (MH+)

### Preparation: tert-butyl 4-[4-(cyclopropanecarbonylamino)-2-pyrrolidin-1-ylbenzoyl]-3-(4-methylphenyl) piperazine-1-carboxylate

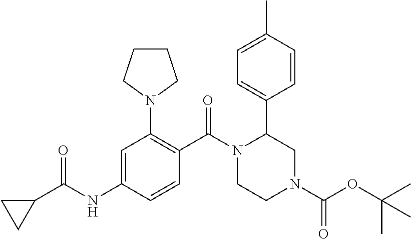 [SMILES: Cc1ccc(C2CN(C(=O)OC(C)(C)C)CCN2C(=O)c2ccc(NC(=O)C3CC3)cc2N2CCCC2)cc1]

### Example 15

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein tert-butyl 3-(4-methylphenyl) piperazine-1-carboxylate was used instead of 2-phenylpyrrolidine. The title compound was obtained as a racemic mixture in a yield of 70% (96 mg).

### Preparation: tert-butyl 3-(3-chloro-5-methylphenyl)-4-[4-(cyclopropanecarbonylamino)-2-pyrrolidin-1-ylbenzoyl]piperazine-1-carboxylate

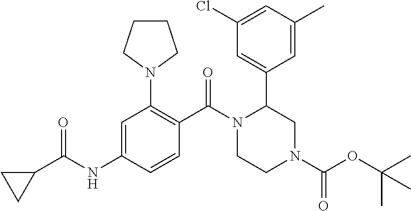 [SMILES: Cc1cc(Cl)cc(C2CN(C(=O)OC(C)(C)C)CCN2C(=O)c2ccc(NC(=O)C3CC3)cc2N2CCCC2)c1]

### Example 16

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein tert-butyl 3-(3-chloro-5-methylphenyl) piperazine-1-carboxylate was used instead of 2-phenylpyrrolidine. The title compound was obtained as a racemic mixture in a yield of 90% (130 mg).

### LC-MS: m/z 567.29 (MH+).

### Preparation: tert-butyl 4-[4-(cyclopropanecarbonylamino)-2-pyrrolidin-1-ylbenzoyl]-3-(3-methylphenyl) piperazine-1-carboxylate

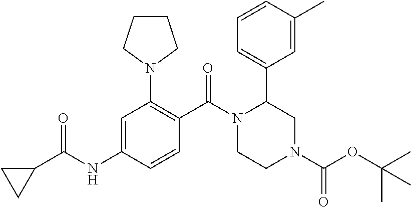 [SMILES: Cc1cccc(C2CN(C(=O)OC(C)(C)C)CCN2C(=O)c2ccc(NC(=O)C3CC3)cc2N2CCCC2)c1]

### Example 17

The synthesis of the title compound was effected analogously to the synthesis of the compound of Example 1 N-[4-(2-phenylpyrrolidine-1-carbonyl)-3-pyrrolidin-1-ylphenyl]cyclopropanecarboxamide wherein tert-butyl 3-(3-methylphenyl) piperazine-1-carboxylate was used instead of 2-phenylpyrrolidine. The title compound was obtained as a racemic mixture in a quantitative yield (150 mg).

### LC-MS: m/z 533.33 (MH+).

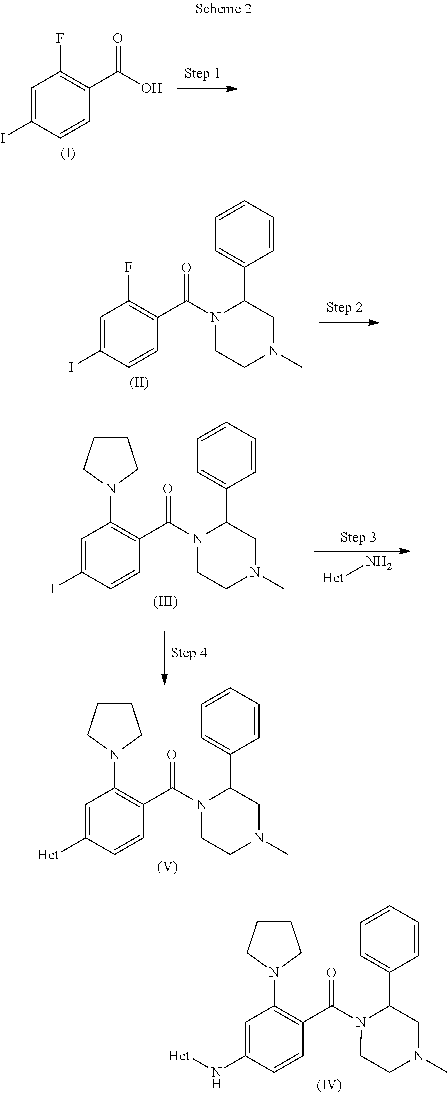

### Step 1

Compound of formula II may be obtained via acyl chloride formation of compound of formula I (commercially available from Enamine) by treatment with a suitable chlorinating agent, such as SOCl₂, typically at 90° C., followed by coupling with 1-Methyl-3-phenylpiperazine (commercially available from Enamine) in the presence of a suitable organic base, e.g. DIPEA. The reaction is carried out in a suitable solvent such as DCM, typically at room temperature. The reaction takes about 12 hours to complete.

### Step 2

Compound of formula II may be obtained from compound of formula II by SNAr substitution with pyrrolidine in a suitable aprotic solvent, e.g. DMSO, typically at 120° C. The reaction takes about 36 hours to complete.

### Step 3

Compound of formula IV may be obtained from compound of formula III by Buchwald reaction with the proper heteroaryl aniline in the presence of a suitable palladium precatalyst, e.g. tBuBrettPhos Pd G3, and a suitable base, such as K₃PO₄. The reaction is carried out in a suitable solvent, e.g. 1-butanol, typically at 120° C. The reaction takes from about 12 hours to about 24 hours to complete.

### Step 4

Compound of formula V may be obtained via Suzuki coupling between compound of formula III and the appropriate commercially available boronic acid in the presence of a suitable catalyst, e.g. palladium tetrakis triphenylphosphine, suitable inorganic base Na₂CO₃, in a suitable solvent, 1,2-dimethoxyethane, typically at 95° C. The reaction takes about 2 days to complete.

### Scheme 2, Cpd=Preparation: (2-fluoro-4-iodophenyl)-(4-methyl-2-phenylpiperazin-1-yl)methanone

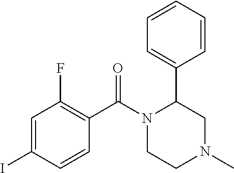 [SMILES: CN1CCN(C(=O)c2ccc(I)cc2F)C(c2ccccc2)C1]

A mixture 2-fluoro-4-iodobenzoic acid (2.0 g, 7.52 mmol) and thionyl dichloride (10.97 mL, 150.37 mmol) was heated to reflux for 2.5 hours. After this time the reaction was cooled down to RT and then concentrated under vacuum. The mixture was stripped three times with cyclohexane in order to remove the excess of thionyl dichloride. The residue was suspended in dry DCM (40 mL) and triethylamine (3.14 mL, 22.56 mmol) was added followed by 1-methyl-3-phenylpiperazine (1.33 g, 7.52 mmol). The reaction mixture was stirred overnight at RT. The day after, the mixture was diluted with DCM and washed with a s.s NaHCO₃. The combined organic phases were dried over a phase separator and concentrated under vacuum affording the product of formula (2-fluoro-4-iodophenyl)-(4-methyl-2-phenylpiperazin-1-yl)methanone as racemic mixture. The product was used in the next step without further purification.

### Yield: 3.2 g

¹H NMR (400 MHZ, DMSO-d₆) δ 7.75 (dd, 2H), 7.47 (d, 2H), 7.42-7.20 (m, 4H), 4.74-3.64 (m, 1H), 3.55-3.34 (m, 1H), 3.25-2.57 (m, 2H), 2.36-2.24 (m, 1H), 2.18 (d, 3H), 2.07-1.83 (m, 1H), 0.97 (t, 1H); LC-MS: m/z 425.05 (MH+).

### Scheme 2, Cpd III Preparation: (4-iodo-2-pyrrolidin-1-ylphenyl)-(4-methyl-2-phenylpiperazin-1-yl)methanone

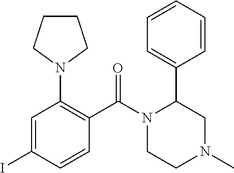 [SMILES: CN1CCN(C(=O)c2ccc(I)cc2N2CCCC2)C(c2ccccc2)C1]

To a solution of (2-fluoro-4-iodophenyl)-(4-methyl-2-phenylpiperazin-1-yl)methanone (2.79 g, 6.56 mmol) in DMSO (50 mL) pyrrolidine (1.64 mL, 19.69 mmol) was added and the reaction mixture was stirred ON at 120° C. After 16 h further pyrrolidine (0.55 mL, 6.56 mmol) was added and the reaction was stirred for 1 day. The day after the reaction was cooled down to RT and diluted with water and the aqueous phase was extracted with AcOEt three times. The combined organic phases were washed with brine, dried over Na₂SO₄, filtered and concentrated under vacuum. The residue was purified by FC on reverse phase using acid conditions (eluting from 5:95 of CH₃CN/H₂O+0.1% of HCOOH to 95:5 of CH₃CN/H₂O+0.1% of HCOOH). Pure fractions were collected and concentrated under vacuum. The residue was taken up with a s.s. of NaHCO₃ and extracted 3 times with AcOEt. The combined organic fractions were washed with brine, dried over Na₂SO₄, filtered and concentrated under vacuum affording the product of formula (4-iodo-2-pyrrolidin-1-ylphenyl)-(4-methyl-2-phenylpiperazin-1-yl)methanone.

### Yield: 1.0 g

1H NMR (400 MHZ, DMSO) δ 7.73 (d, 1H), 7.53-7.14 (m, 4H), 7.10-6.76 (m, 2H), 6.68 (d, 1H), 5.84-5.57 (m, 1H), 4.77-4.29 (m, 1H), 3.14-2.58 (m, 7H), 2.43-1.99 (m, 7H), 1.75-1.49 (m, 2H); LC-MS: m/z 476.1 (MH+).

### Preparation: [4-[(3-methyl-1,2-oxazol-5-yl)amino]-2-pyrrolidin-1-ylphenyl]-(4-methyl-2-phenylpiperazin-1-yl)methanone

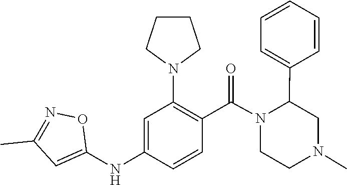

### Example 18

A mixture of (4-iodo-2-pyrrolidin-1-ylphenyl)-(4-methyl-2-phenylpiperazin-1-yl)methanone (150.0 mg, 0.320 mmol), 3-methyl-5-isoxazolamine (37.15 mg, 0.380 mmol), tripotassium phosphate (66.98 mg, 0.320 mmol) and tBuBrettPhos Pd G3 (26.96 mg, 0.030 mmol) in 1-Butanol (1.2 mL) was degassed by N₂-vacuum cycles. The reaction was stirred at 120° C. for 24 hours. After this time the reaction was cooled down to RT, diluted with MeOH and purified by SCX first washing with MeOH and then eluting with NH₃ 1N in MeOH. Basic fractions were collected and concentrated under vacuum. The residue was purified by FC on reverse phase using acid conditions (eluting from 5:95 of CH₃CN/H₂O+0.1% of HCOOH to 95:5 of CH₃CN/H₂O+0.1% of HCOOH). Pure fractions were collected and concentrated under vacuum. The residue was taken up with a s.s. of NaHCO₃ and extracted 3 times with AcOEt. The combined organic fractions were washed with brine, dried over Na₂SO₄, filtered and concentrated under vacuum affording the product of formula [4-[(3-methyl-1,2-oxazol-5-yl)amino]-2-pyrrolidin-1-ylphenyl]-(4-methyl-2-phenylpiperazin-1-yl)methanone as a racemic mixture.

### Yield: 16 mg

¹H NMR (400 MHZ, DMSO-d₆) δ 9.75 (d, 1H), 7.83-7.14 (m, 6H), 6.97 (ddd, 1H), 6.78-6.66 (m, 1H), 6.61-6.35 (m, 1H), 3.55-3.30 (m, 2H), 3.10-2.55 (m, 7H), 2.37-2.10 (m, 7H), 1.94 (s, 4H), 1.67 (d, 1H); LC-MS: m/z 446.48 (MH+).

### Preparation: (4-methyl-2-phenylpiperazin-1-yl)-[2-pyrrolidin-1-yl-4-([1,2,4]triazolo[1,5-a]pyridin-2-ylamino)phenyl]methanone

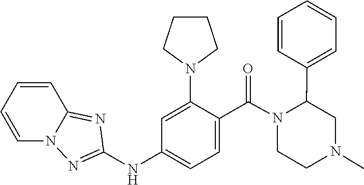 [SMILES: CN1CCN(C(=O)c2ccc(Nc3nc4ccccn4n3)cc2N2CCCC2)C(c2ccccc2)C1]

### Example 19

The synthesis of the title compound was effected analogously to the synthesis of the compound of Example 18 [4-[(3-methyl-1,2-oxazol-5-yl)amino]-2-pyrrolidin-1-ylphenyl]-(4-methyl-2-phenylpiperazin-1-yl)methanone wherein [1,2,4]triazolo[1,5-a]pyridin-2-amine was used instead of 3-methyl-5-isoxazolamine. The title compound was obtained in a yield of 16% (25 mg).

1H NMR (500 MHZ, DMSO-d6) δ ppm 9.52 (br d, 1H), 8.74 (d, 1H), 7.84-6.50 (m, 11H), 5.93-4.61 (m, 1H), 4.54-3.36 (m, 2H), 3.30-3.17 (m, 2H), 3.06 (br s, 4H), 2.44-2.09 (m, 4H), 2.09-1.51 (m, 5H); LC-MS: m/z 482.24 (MH+).

### Preparation: (4-methyl-2-phenylpiperazin-1-yl)-[4-[(5-methyl-1,3,4-thiadiazol-2-yl)amino]-2-pyrrolidin-1-ylphenyl]methanone

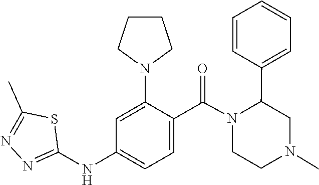 [SMILES: Cc1nnc(Nc2ccc(C(=O)N3CCN(C)CC3c3ccccc3)c(N3CCCC3)c2)s1]

### Example 20

The synthesis of the title compound was effected analogously to the synthesis of the compound of Example 18 [4-[(3-methyl-1,2-oxazol-5-yl)amino]-2-pyrrolidin-1-ylphenyl]-(4-methyl-2-phenylpiperazin-1-yl)methanone wherein 5-methyl-1,3,4-thiadiazol-2-amine was used instead of 3-methyl-5-isoxazolamine. The title compound was obtained in a yield of 39% (56 mg).

1H NMR (400 MHZ, DMSO-d6) δ 10.18 (d, 1H), 7.76-7.68 (m, 1H), 7.54-7.17 (m, 5H), 7.15-6.99 (m, 1H), 6.94-6.81 (m, 1H), 5.90-4.27 (m, 1H), 3.43 (d, 1H), 3.12-2.53 (m, 8H), 2.37 (dt, 1H), 2.22 (d, 4H), 2.07-1.84 (m, 4H), 1.81-1.55 (m, 2H); LC-MS: m/z 463.2 (MH+).

### Preparation: (4-methyl-2-phenylpiperazin-1-yl)-[4-(1H-pyrazol-4-yl)-2-pyrrolidin-1-ylphenyl]methanone

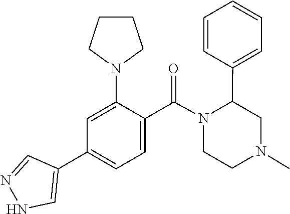 [SMILES: CN1CCN(C(=O)c2ccc(-c3cn[nH]c3)cc2N2CCCC2)C(c2ccccc2)C1]

### Example 21

A mixture of (4-bromo-2-pyrrolidin-1-ylphenyl)-(4-methyl-2-phenylpiperazin-1-yl)methanone (50.0 mg, 0.120 mmol), 1H-pyrazole-4-boronic acid (14.37 mg, 0.130 mmol) and a 2M aqueous solution of Na₂CO₃ (0.18 mL, 0.350 mmol) in 1,2-dimethoxyethane (1.052 mL) was degassed by 5 cycles of nitrogen/vacuum. Then palladium tetrakis triphenylphosphine (6.74 mg, 0.010 mmol) was added and the vessel was sealed. The reaction mixture was degassed again by 5 cycles of nitrogen/vacuum and then was heated to 95° C. for 2 days. After this time the mixture was directly purified by FC on RP using acid conditions (eluting from 5:95 of CH₃CN/H₂O+0.1% of HCOOH to 50:50 of CH₃CN/H₂O+0.1% of HCOOH) and then by SCX first washing with MeOH and then eluting with NH₃ 1N in MeOH. Basic fractions were collected and concentrated under vacuum affording the product of formula (4-methyl-2-phenylpiperazin-1-yl)-[4-(1H-pyrazol-4-yl)-2-pyrrolidin-1-ylphenyl]methanone.

### Yield: 14 mg

¹H NMR (400 MHZ, DMSO-d₆) δ 12.91 (bs, 1H), 8.29-8.15 (m, 1H), 7.98-7.85 (m, 1H), 7.74 (d, 1H), 7.60-7.15 (m, 4H), 7.08-6.80 (m, 3H), 5.98-4.37 (m, 1H), 3.52-3.39 (m, 2H), 3.16-2.61 (m, 5H), 2.44-2.13 (m, 5H), 2.05-1.90 (m, 3H), 1.83-1.63 (m, 2H); LC-MS: m/z 416.18 (MH+).

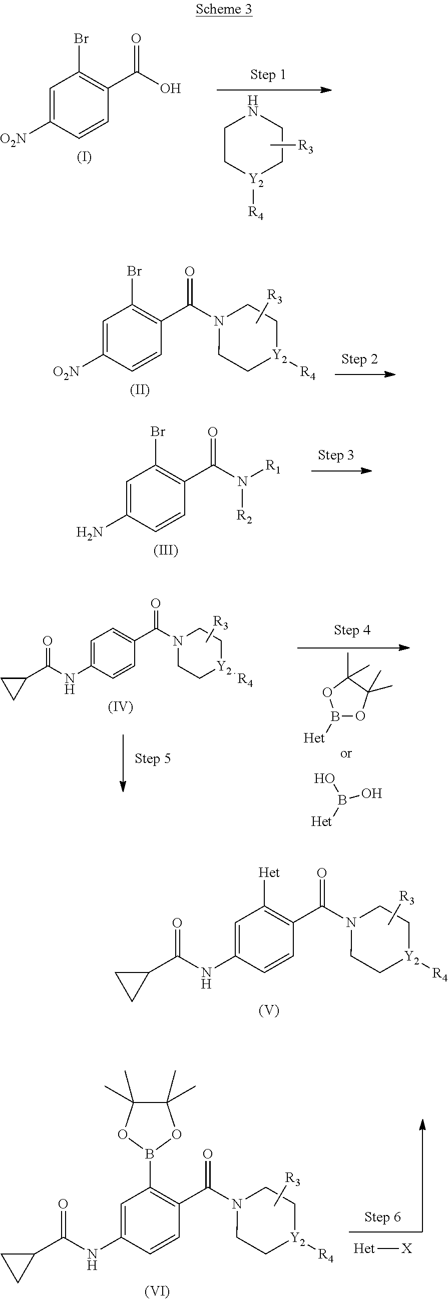

### Step 1

Compound of formula II may be obtained from compound of formula I (commercially available from Enamine) by coupling with a proper amine in the presence of coupling agent, e.g. HATU, and an organic base, e.g. DIPEA. The reaction is carried out in a suitable solvent such as DMF, typically at room temperature. The reaction takes from about 3 hours to about 12 hours to complete. Alternatively compound of formula II may be obtained via acyl chloride formation of compound of formula I (commercially available from Enamine) by treatment with a suitable chlorinating agent, such as SOCl₂, typically at 90° C., followed by coupling with the proper amine in the presence of a suitable organic base, e.g. TEA. The reaction is carried out in a suitable solvent such as DCM, typically at room temperature. The reaction takes from about 1 hour to about 12 hours to complete.

### Step 2

Compound of formula III may be obtained by nitro reduction of compound of formula II using Zn dust in a suitable solvent, e.g. AcOH, typically at room temperature. The reaction takes from about 3 hours to about 12 hours to complete. Alternatively, the compound of formula III may be obtained by reduction of compound of formula II using Tin (III) chloride dehydrate in a suitable solvent, e.g. EtOH, typically at 80° C. The reaction takes about 12 hours to complete.

### Step 3

Compound of formula IV may be obtained by coupling reaction between compound of formula III and a proper commercially available acyl chloride, e.g. Cyclopropanecarbonyl chloride, in presence of an organic base, e.g. TEA. The reaction is carried out in a suitable solvent such as DCM, typically at room temperature. The reaction takes about 3 hours to complete.

### Step 4

Compound of formula V may be obtained via Suzuki coupling between compound of formula IV and the desired heteroaryl boronic ester or heteroaryl boronic acid, in the presence of a suitable precatalyst, e.g. XPhos Pd G2, a suitable base, e.g. K₃PO₄, in a suitable mixture of solvents, e.g. 1,2-dimethoxyethane/water, typically at 100° C. The reaction takes about from 3 hours to 12 hours to complete.

### Step 5

Compound of formula VI may be obtained via Miyaura coupling between compound of formula IV, the desired Aryl Bromide, and Bis(pinacolato)diboron, in presence of a suitable catalyst, such as PdCl₂(dppf)·CH₂Cl₂, a suitable base, e.g. AcOK, in a suitable solvent, e.g. 1,4 dioxane, typically at 100° C. The reaction takes about 12 hours to complete.

### Step 6

Compound of formula V may be obtained via Suzuki coupling between compound of formula VI and commercially available heteroaryl halide in the presence of a suitable catalyst, e.g. palladium tetrakis triphenylphosphine, suitable inorganic base Na₂CO₃, in a suitable solvent, 1,2-dimethoxyethane, typically at 95° C. The reaction takes about 12 hours to complete.

### Scheme 3, Cpd (II)*a Preparation: (2-bromo-4-nitrophenyl)-(4,4-difluoropiperidin-1-yl)methanone

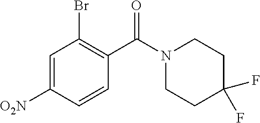 [SMILES: O=C(c1ccc([N+](=O)[O-])cc1Br)N1CCC(F)(F)CC1]

To a solution of [dimethylamino (3-triazolo[4,5-b]pyridinyloxy)methylidene]-dimethylammonium hexafluorophosphate, HATU, (2.32 g, 6.1 mmol), 2-bromo-4-notrobenzoic acid (1.0 g, 4.06 mmol) and N,N-Diisopropylethylamine (3.15 g, 24.39 mmol) in DMF (25 mL), 4,4-difluoropiperidine hydrochloride (768.7 mg, 4.88 mmol) was added. The reaction was stirred at RT for 3 h. After this time a saturated aqueous solution of NaHCO₃ was added to the reaction mixture and the aqueous phase was extracted with EtOAc (×3). The organic portions were collected, washed with brine, residual water was removed by adding Na₂SO₄, the solvent was filtered and evaporated under reduced pressure. The residue was purified by FC on silica gel (eluting from 100% of cHex to AcOEt/cHex 25:75) affording the product of formula (2-bromo-4-nitrophenyl)-(4,4-difluoropiperidin-1-yl)methanone.

### Yield: 1.2 g

1H NMR (400 MHZ, DMSO) δ 8.51 (d, 1H), 8.32 (dd, 1H), 7.77 (d, 1H), 3.89 (dt, 1H), 3.68 (ddd, 1H), 3.25 (t, 2H), 2.32-1.88 (m, 4H); LC-MS: m/z 349.0, 351.0 (MH+)

### Scheme 3, Cpd (II)*b Preparation: (2-bromo-4-nitrophenyl)-(4-propylpiperazin-1-yl)methanone

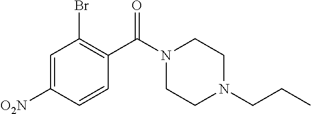 [SMILES: CCCN1CCN(C(=O)c2ccc([N+](=O)[O-])cc2Br)CC1]

A mixture of 2-bromo-4-nitrobenzoic acid (1.5 g, 6.1 mmol) and thionyl dichloride (8.9 mL, 121.95 mmol) was heated to 90° C. and stirred for 2 hours. The reaction was cooled down to RT and then concentrated under vacuum. The mixture was stripped three times with cyclohexane in order to remove thionyl dichloride excess. The residue was taken up with DCM (10 mL) and added to a solution of triethylamine (5.96 mL, 42.68 mmol) and 1-propylpiperazine dihydrobromide (1.77 g, 6.1 mmol) in DCM (75 mL). The reaction mixture was stirred for 1 h at RT, then it was diluted with DCM, washed with a s.s. of NaHCO₃, brine, dried over Na₂SO₄, filtered and concentrated under vacuum affording the product of formula (2-bromo-4-nitrophenyl)-(4-propylpiperazin-1-yl)methanone. The product was used in the next step without further purification.

### Yield: 2.2 g

1H NMR (400 MHZ, DMSO) δ 8.49 (d, 1H), 8.29 (dd, 1H), 7.65 (d, 1H), 3.65 (qdd, 2H), 3.18-3.05 (m, 2H), 2.49-2.38 (m, 2H), 2.37-2.31 (m, 2H), 2.30-2.24 (m, 2H), 1.44 (h, 2H), 0.86 (t, 3H); LC-MS: m/z 256.05, 358.07 (MH+).

### Scheme 3, Cpd (II)*c Preparation: (2-bromo-4-nitrophenyl)-(1,1-dioxo-1,4-thiazinan-4-yl)methanone

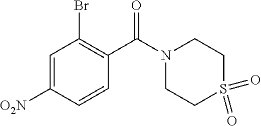 [SMILES: O=C(c1ccc([N+](=O)[O-])cc1Br)N1CCS(=O)(=O)CC1]

The synthesis of the title compound was effected analogously to the synthesis of the compound (2-bromo-4-nitrophenyl)-(4-propylpiperazin-1-yl)methanone wherein 1,4-thiazinane 1,1-dioxide hydrochloride was used instead of 1-propylpiperazine dihydrobromide. The title compound was obtained in a yield of 74% (3.3 g).

1H NMR (400 MHZ, DMSO) δ 8.52 (d, 1H), 8.36 (dd, 1H), 7.89 (d, 1H), 4.50-4.36 (m, 1H), 3.74 (ddd, 1H), 3.60-3.51 (m, 2H), 3.42 (d, 1H), 3.28-3.23 (m, 1H), 3.12-3.05 (m, 2H); LC-MS: m/z 362.87, 364.94 (MH+).

### Scheme 3, Cpd (II)*d Preparation: (2-bromo-4-nitrophenyl)-(4-methyl-2-phenylpiperazin-1-yl)methanone

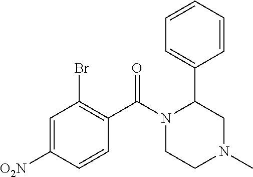 [SMILES: CN1CCN(C(=O)c2ccc([N+](=O)[O-])cc2Br)C(c2ccccc2)C1]

The synthesis of the title compound was effected analogously to the synthesis of the compound (2-bromo-4-nitrophenyl)-(4-propylpiperazin-1-yl)methanone wherein 1-methyl-3-phenylpiperazine was used instead of 1-propylpiperazine dihydrobromide. The title compound was obtained in a yield of 100% (1.8 g).

1H NMR (400 MHZ, DMSO) δ 8.50 (td, 1H), 8.37-8.09 (m, 1H), 7.89-7.17 (m, 6H), 5.95-5.71 (m, 1H), 4.47 (d, 1H), 3.50 (dd, 1H), 3.17-2.57 (m, 2H), 2.40 (td, 1H), 2.30-1.90 (m, 4H); LC-MS: 404.01, 405.96 (MH+).

### Scheme 3, Cpd (III)*a Preparation: (4-amino-2-bromophenyl)-(4,4-difluoropiperidin-1-yl)methanone

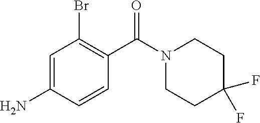 [SMILES: Nc1ccc(C(=O)N2CCC(F)(F)CC2)c(Br)c1]

To a solution of (2-bromo-4-nitrophenyl)-(4,4-difluoropiperidin-1-yl)methanone (1.17 g, 3.35 mmol) in AcOH (28 mL) zinc (2.19 g, 33.51 mmol) was added and the reaction mixture was stirred at RT for 24 h. The reaction was filtered and concentrated under vacuum. The residue was taken up with a s. s of NaHCO₃ and extracted with AcOEt (3 times). The combined organic fractions were washed with brine, dried over Na₂SO₄, filtered and solvent was evaporated. The crude was purified by FC on silica gel (eluting from cHex/EtOAc 80:20 to cHex/EtOAc 4:6) affording the product of formula (4-amino-2-bromophenyl)-(4,4-difluoropiperidin-1-yl)methanone.

### Yield: 650 mg

¹H NMR (400 MHZ, DMSO-d₆) δ 7.00 (d, 1H), 6.80 (d, 1H), 6.57 (dd, 1H), 5.62 (s, 2H), 3.86-3.73 (m, 2H), 3.67-3.55 (m, 2H), 2.13-1.91 (m, 4H); LC-MS: m/z 319.25, 321.25 (MH+).

### Scheme 3, Cpd (III)*b Preparation: (4-amino-2-bromophenyl)-(1,1-dioxo-1,4-thiazinan-4-yl)methanone

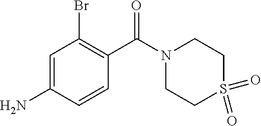 [SMILES: Nc1ccc(C(=O)N2CCS(=O)(=O)CC2)c(Br)c1]

A mixture of (2-bromo-4-nitrophenyl)-(1,1-dioxo-1,4-thiazinan-4-yl)methanone (1.2 g, 3.3 mmol) and Tin (III) chloride dihydrate (2.26 g, 9.91 mmol) in Ethanol (24 mL) was heated to reflux for 8 h. After this time the mixture was cooled down to RT, filtered and concentrated under vacuum. The residue was purified by FC on silica gel (eluting from 100% of DCM to DCM/MeOH 95:5 as eluent) to obtain the product of formula (4-amino-2-bromophenyl)-(1,1-dioxo-1,4-thiazinan-4-yl)methanone.

### Yield: 1.0 g

1H NMR (400 MHZ, DMSO-d6) δ 7.10 (d, 1H), 6.80 (d, 1H), 6.57 (dd, 1H), 5.75 (s, 2H), 7H), 4.27 (s, 1H), 3.40 (d, 7H); LC-MS: m/z 332.93, 334.9 (MH+).

### Scheme 3, Cpd (III)*c Preparation: (4-amino-2-bromophenyl)-(4-propylpiperazin-1-yl)methanone

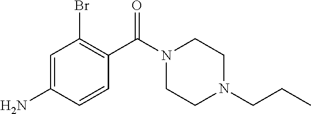 [SMILES: CCCN1CCN(C(=O)c2ccc(N)cc2Br)CC1]

The synthesis of the title compound was effected analogously to the synthesis of the compound (4-amino-2-bromophenyl)-(4,4-difluoropiperidin-1-yl)methanone wherein (2-bromo-4-nitrophenyl)-(4-propylpiperazin-1-yl)methanone was used instead of (2-bromo-4-nitrophenyl)-(4,4-difluoropiperidin-1-yl)methanone. The title compound was obtained in a yield of 88% (1.7 g). 1H NMR (400 MHZ, DMSO) δ 6.91 (d, 1H), 6.79 (d, 1H), 6.56 (dd, 1H), 5.57 (s, 2H), 3.57 (s, 2H), 3.15 (s, 2H), 2.35 (d, 3H), 2.29-2.14 (m, 3H), 1.43 (h, 2H), 0.85 (t, 3H); LC-MS: m/z 326.04, 327.99 (MH+).

### Scheme 3, Cpd (III)*d Preparation: (4-amino-2-bromophenyl)-(4-methyl-2-phenylpiperazin-1-yl)methanone

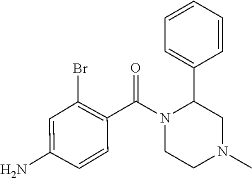 [SMILES: CN1CCN(C(=O)c2ccc(N)cc2Br)C(c2ccccc2)C1]

The synthesis of the title compound was effected analogously to the synthesis of the compound (4-amino-2-bromophenyl)-(4,4-difluoropiperidin-1-yl)methanone wherein (2-bromo-4-nitrophenyl)-(4-methyl-2-phenylpiperazin-1-yl)methanone was used instead of (2-bromo-4-nitrophenyl)-(4,4-difluoropiperidin-1-yl)methanone. The title compound was obtained in a yield 91% (850 mg). 1H NMR (400 MHZ, DMSO) δ 7.57 (d, 1H), 7.30 (d, 5H), 6.80 (td, 2H), 5.75 (s, 1H), 5.60 (s, 2H), 4.95-4.27 (m, 1H), 3.45 (d, 1H), 3.23-2.87 (m, 2H), 2.77-2.57 (m, 1H), 2.38-2.13 (m, 3H), 1.89 (s, 1H); LC-MS: m/z 374.09, 376.08 (MH+).

### Scheme 3, Cpd (IV)*a Preparation: N-[3-bromo-4-(4,4-difluoropiperidine-1-carbonyl)phenyl]cyclopropanecarboxamide

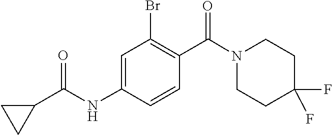 [SMILES: O=C(Nc1ccc(C(=O)N2CCC(F)(F)CC2)c(Br)c1)C1CC1]

To a cooled mixture of N,N-Diisopropylethylamine (0.69 mL, 4.09 mmol) and (4-amino-2-bromophenyl)-(4,4-difluoropiperidin-1-yl)methanone (652.0 mg, 2.04 mmol) in DCM (20 mL), at 0° C., cyclopropanecarbonyl chloride (0.22 mL, 2.45 mmol) was added dropwise. The mixture was stirred for 1 h at RT. After this time, the mixture was diluted with DCM, washed with a s.s. of NaHCO₃ and dried over a phase separator. The solvent was evaporated and crude was purified by FC on silica gel (eluting from 100% of DCM to DCM/MeOH 95:5) affording the product of formula N-[3-bromo-4-(4,4-difluoropiperidine-1-carbonyl)phenyl]cyclopropanecarboxamide.

### Yield: 830 mg

1H NMR (400 MHz, DMSO) δ 10.47 (s, 1H), 8.03 (d, 1H), 7.56 (dd, 1H), 7.33 (d, 1H), 3.82 (s, 1H), 3.66 (d, 1H), 3.24 (t, 2H), 2.17-1.85 (m, 4H), 1.82-1.71 (m, 1H), 0.87-0.80 (m, 4H); LC-MS: m/z 387.16, 389.16 (MH+).

### Scheme 3, Cpd (IV)*b Preparation: N-[3-bromo-4-(4-propylpiperazine-1-carbonyl)phenyl]cyclopropanecarboxamide

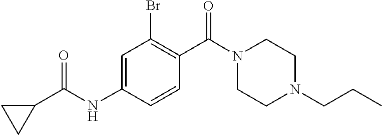 [SMILES: CCCN1CCN(C(=O)c2ccc(NC(=O)C3CC3)cc2Br)CC1]

The synthesis of the title compound was effected analogously to the synthesis of the compound N-[3-bromo-4-(4,4-difluoropiperidine-1-carbonyl)phenyl]cyclopropanecarboxamide wherein (4-amino-2-bromophenyl)-(4-propylpiperazin-1-yl)methanone was used instead of (4-amino-2-bromophenyl)-(4,4-difluoropiperidin-1-yl)methanone. The title compound was obtained in a yield 96% (2.0 g).

1H NMR (400 MHZ, DMSO) δ 10.45 (s, 1H), 8.02 (d, 1H), 7.55 (dd1H), 7.24 (d, 1H), 3.61 (d, 2H), 3.12 (td, 2H), 2.46-2.09 (m, 6H), 1.77 (p, 1H), 1.42 (dt, 2H), 0.92-0.78 (m, 7H); LC-MS: m/z 394.10, 396.04 (MH+).

### Scheme 3, Cpd (IV)*c Preparation: N-[3-bromo-4-(1,1-dioxo-1,4-thiazinane-4-carbonyl)phenyl]cyclopropanecarboxamide

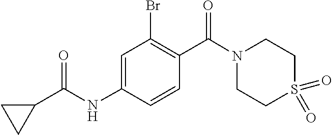 [SMILES: O=C(Nc1ccc(C(=O)N2CCS(=O)(=O)CC2)c(Br)c1)C1CC1]

The synthesis of the title compound was effected analogously to the synthesis of the compound N-[3-bromo-4-(4,4-difluoropiperidine-1-carbonyl)phenyl]cyclopropanecarboxamide wherein (4-amino-2-bromophenyl)-(1,1-dioxo-1,4-thiazinan-4-yl)methanone was used instead of (4-amino-2-bromophenyl)-(4,4-difluoropiperidin-1-yl)methanone. The title compound was obtained in a yield 43% (1.6 g).

1H NMR (400 MHZ, DMSO) δ 10.49 (s, 1H), 8.04 (d, 1H), 7.59 (dd, 1H), 7.46 (d, 1H), 4.35 (s, 1H), 3.75 (s, 1H), 3.56 (s, 3H), 3.23 (s, 2H), 3.06 (s, 1H), 1.78 (p, 1H), 0.84 (d, 4H); LC-MS: m/z 401.27, 403.27 (MH+).

### Scheme 3, Cpd (IV)*d Preparation: N-[3-bromo-4-(4-methyl-2-phenylpiperazine-1-carbonyl)phenyl]cyclopropanecarboxamide

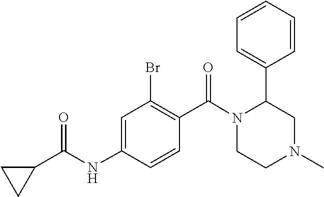 [SMILES: CN1CCN(C(=O)c2ccc(NC(=O)C3CC3)cc2Br)C(c2ccccc2)C1]

The synthesis of the title compound was effected analogously to the synthesis of the compound N-[3-bromo-4-(4,4-difluoropiperidine-1-carbonyl)phenyl]cyclopropanecarboxamide wherein (4-amino-2-bromophenyl)-(4-methyl-2-phenylpiperazin-1-yl)methanone was used instead of (4-amino-2-bromophenyl)-(4,4-difluoropiperidin-1-yl)methanone. The title compound was obtained in a yield of 69% (3.9 g).

¹H NMR (400 MHZ, DMSO-d₆) δ 10.44 (d, 1H), 8.14-7.94 (m, 1H), 7.58 (d, 2H), 7.31 (dt, 5H), 5.84-5.52 (m, 1H), 3.47 (d, 1H), 3.17-2.57 (m, 2H), 2.46-2.12 (m, 4H), 2.11-1.60 (m, 2H), 1.08 (dt, 1H), 0.82 (d, 4H); LC-MS: m/z 442.12, 444.14 (MH+).

### Preparation: N-[4-(4,4-difluoropiperidine-1-carbonyl)-3-(1-methylpyrazol-3-yl)phenyl]cyclopropanecarboxamide

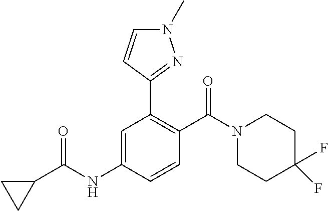 [SMILES: Cn1ccc(-c2cc(NC(=O)C3CC3)ccc2C(=O)N2CCC(F)(F)CC2)n1]

### Example 22

A mixture of 1-methyl-1H-pyrazole-3-boronic acid pinacol ester (83.83 mg, 0.400 mmol), N-[3-bromo-4-(4,4-difluoropiperidine-1-carbonyl)phenyl]cyclopropanecarboxamide (120.0 mg, 0.310 mmol), X-Phos aminobiphenyl palladium chloride precatalyst (24.35 mg, 0.030 mmol) and tripotassium phosphate (197.35 mg, 0.930 mmol) in Water (0.3 mL)/1,2-dimethoxyethane (3 mL was degassed under nitrogen flux for 5 min. and then heated to 100° C. overnight. The day after the reaction was cooled down to room temperature, diluted with water and extracted with EtOAc (3×).

The organic layers were collected together and washed with brine, dried over Na₂SO₄, filtered and the solvent removed under vacuum. The residue was purified by FC on silica gel (eluting from DCM/EtOAc 90:10 to DCM:AcOEt 20:80) and again by FC on reverse phase using acid conditions (eluting from 5:95 of CH₃CN/H₂O+0.1% of HCOOH to 50:50 of CH₃CN/H₂O+0.1% of HCOOH). Pure fractions were collected and concentrated under vacuum. The residue was taken up with a s.s. of NaHCO₃ and extracted 3 times with AcOEt. The combined organic fractions were washed with brine, dried over Na₂SO₄, filtered and concentrated under vacuum affording the product of formula N-[4-(4,4-difluoropiperidine-1-carbonyl)-3-(1-methylpyrazol-3-yl)phenyl]cyclopropanecarboxamide.

### Yield: 22 mg

1H NMR (400 MHZ, DMSO) δ 10.36 (s, 1H), 7.95 (d, 1H), 7.73 (d, 1H), 7.63 (dd, 1H), 7.20 (d, 1H), 6.35 (d, 1H), 3.85 (s, 4H), 3.51 (ddd, 1H), 3.13 (tt, 2H), 2.13-1.89 (m, 2H), 1.79 (tt, 2H), 1.60-1.47 (m, 1H), 0.85-0.75 (m, 4H); LC-MS: m/z 389.2 (MH+)

### Preparation: N-[4-(4,4-difluoropiperidine-1-carbonyl)-3-(1-propan-2-ylpyrazol-3-yl)phenyl]cyclopropanecarboxamide

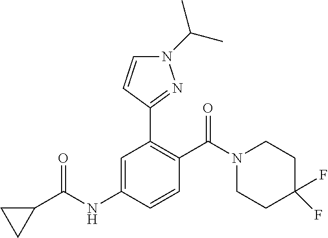 [SMILES: CC(C)n1ccc(-c2cc(NC(=O)C3CC3)ccc2C(=O)N2CCC(F)(F)CC2)n1]

### Example 23

The synthesis of the title compound was effected analogously to the synthesis of the compound of Example 22 N-[4-(4,4-difluoropiperidine-1-carbonyl)-3-(1-methylpyrazol-3-yl)phenyl]cyclopropanecarboxamide wherein 1-isopropyl-3-(4,4,5,5-tetramethyl-1,3,2-dioxaborolan-2-yl)-1H-pyrazole was used instead of 1-methyl-1H-pyrazole-3-boronic acid pinacol ester. The title compound was obtained in a yield of 42% (54 mg). 1H NMR (400 MHZ, DMSO) δ 10.36 (s, 1H), 7.91 (d, 1H), 7.80 (d, 1H), 7.65 (dd, 1H), 7.21 (d, 1H), 6.34 (d, 1H), 4.51 (hept, 1H), 3.93-3.82 (m, 1H), 3.51 (ddd, 1H), 3.20-3.02 (m, 2H), 2.14-1.66 (m, 4H), 1.42 (dd, 6H), 1.36-1.21 (m, 1H), 0.85-0.75 (m, 4H); LC-MS: m/z 417.3 (MH+).

### Preparation: N-[3-(1-propan-2-ylpyrazol-3-yl)-4-(4-propylpiperazine-1-carbonyl)phenyl]cyclopropanecarboxamide

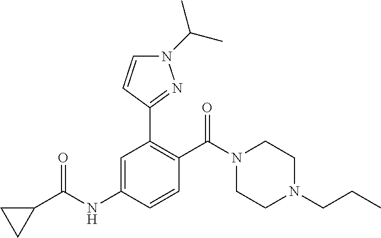 [SMILES: CCCN1CCN(C(=O)c2ccc(NC(=O)C3CC3)cc2-c2ccn(C(C)C)n2)CC1]

### Example 24

The synthesis of the title compound was effected analogously to the synthesis of the compound of Example 22 N-[4-(4,4-difluoropiperidine-1-carbonyl)-3-(1-methylpyrazol-3-yl)phenyl]cyclopropanecarboxamide wherein N-[3-bromo-4-(4-propylpiperazine-1-carbonyl)phenyl]cyclopropanecarboxamide and 1-isopropyl-3-(4,4,5,5-tetramethyl-1,3,2-dioxaborolan-2-yl)-1H-pyrazole were used instead of N-[3-bromo-4-(4,4-difluoropiperidine-1-carbonyl)phenyl]cyclopropanecarboxamide and 1-methyl-1H-pyrazole-3-boronic acid pinacol ester respectively. The title compound was obtained in a yield of 38% (61 mg).

1H NMR (400 MHZ, DMSO) δ 10.34 (s, 1H), 7.91 (d, 1H), 7.78 (d, 1H), 7.64 (dd, 1H), 7.13 (d, 1H), 6.30 (d, 1H), 4.52 (hept, 1H), 3.62-3.43 (m, 2H), 3.06-2.96 (m, 1H), 2.90-2.78 (m, 1H), 2.30 (dt, 2H), 2.20-2.03 (m, 3H), 1.88-1.66 (m, 2H), 1.44 (dd, 6H), 1.36 (h, 2H), 0.84-0.74 (m, 7H); LC-MS: m/z 424.3 (MH+).

### Preparation: N-[4-(1,1-dioxo-1,4-thiazinane-4-carbonyl)-3-(5-methylthiophen-2-yl)phenyl]cyclopropanecarboxamide

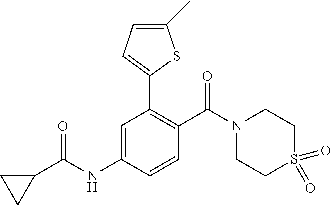 [SMILES: Cc1ccc(-c2cc(NC(=O)C3CC3)ccc2C(=O)N2CCS(=O)(=O)CC2)s1]

### Example 25

4,4,5,5-tetramethyl-2-(5-methyl-2-thiophenyl)-1,3,2-dioxaborolane (72.61 mg, 0.320 mmol), N-[3-bromo-4-(1,1-dioxo-1,4-thiazinane-4-carbonyl)phenyl]cyclopropanecarboxamide (100.0 mg, 0.250 mmol), X-Phos aminobiphenyl palladium chloride precatalyst (19.58 mg, 0.020 mmol) and tripotassium phosphate (158.69 mg, 0.750 mmol) were suspended in a mixture of Water (0.3 mL) and 1,2-dimethoxyethane (3.3 mL). The resulting suspension was degassed under nitrogen flux for 5 min and then heated to 90° C. overnight. The day after the reaction mixture was cooled down to room temperature, diluted with water and extracted with EtOAc (3×). The organic layers were collected together and washed with brine, dried over Na₂SO₄, filtered and the solvent removed under vacuum. The crude was purified by FC on reverse phase using acid conditions (eluting from 5:95 of CH₃CN/H₂O+0.1% of HCOOH to 60:40 of CH₃CN/H₂O+0.1% of HCOOH) affording the product of formula N-[4-(1,1-dioxo-1,4-thiazinane-4-carbonyl)-3-(5-methylthiophen-2-yl)phenyl]cyclopropanecarboxamide.

### Yield: 23 mg

1H NMR (400 MHZ, DMSO) δ 10.42 (s, 1H), 7.83 (d, 1H), 7.59 (dd, 1H), 7.35 (d, 1H), 6.94 (d, 1H), 6.84 (dt, 1H), 4.06-3.98 (m, 1H), 3.97-3.90 (m, 1H), 3.53-3.44 (m, 1H), 3.33 (d, 1H), 3.10-2.97 (m, 2H), 2.50 (d, 1H), 2.47 (d, 3H), 2.28 (d, 1H), 1.84-1.73 (m, 1H), 0.85-0.78 (m, 4H); LC-MS: m/z 419.1 (MH+).

### Preparation: N-[3-(5-cyanothiophen-2-yl)-4-(1,1-dioxo-1,4-thiazinane-4-carbonyl)phenyl]cyclopropanecarboxamide

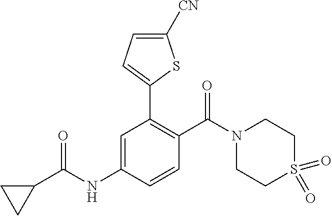 [SMILES: N#Cc1ccc(-c2cc(NC(=O)C3CC3)ccc2C(=O)N2CCS(=O)(=O)CC2)s1]

### Example 26

The synthesis of the title compound was effected analogously to the synthesis of the compound of Example 25 N-[4-(1,1-dioxo-1,4-thiazinane-4-carbonyl)-3-(5-methylthiophen-2-yl)phenyl]cyclopropanecarboxamide wherein (5-cyanothiophen-2-yl) boronic acid was used instead of 4,4,5,5-tetramethyl-2-(5-methyl-2-thiophenyl)-1,3,2-dioxaborolane. The title compound was obtained in a yield of 11% (11 mg).

1H NMR (400 MHZ, DMSO-d6) δ 10.53 (s, 1H), 7.95 (dd, 2H), 7.72 (dd, 1H), 7.53 (d, 1H), 7.26 (d, 1H), 4.27 (s, 1H), 3.55 (dd, 3H), 3.12 (s, 3H), 2.67 (p, 1H), 1.79 (p, 1H), 0.84 (d, 4H); LC-MS: m/z 430.2 (MH+).

### Preparation: N-[4-(1,1-dioxo-1,4-thiazinane-4-carbonyl)-3-[2-(trifluoromethyl)-1,3-thiazol-5-yl]phenyl]cyclopropanecarboxamide

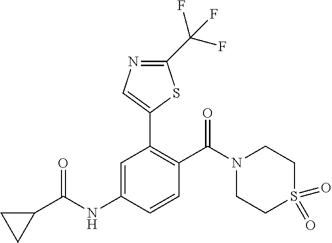 [SMILES: O=C(Nc1ccc(C(=O)N2CCS(=O)(=O)CC2)c(-c2cnc(C(F)(F)F)s2)c1)C1CC1]

### Example 27

The synthesis of the title compound was effected analogously to the synthesis of the compound of Example 25 N-[4-(1,1-dioxo-1,4-thiazinane-4-carbonyl)-3-(5-methylthiophen-2-yl)phenyl]cyclopropanecarboxamide wherein 5-(tetramethyl-1,3,2-dioxaborolan-2-yl)-2-(trifluoromethyl)-1,3-thiazole was used instead of 4,4,5,5-tetramethyl-2-(5-methyl-2-thiophenyl)-1,3,2-dioxaborolane. The title compound was obtained in a yield of 55% (62 mg).

1H NMR (400 MHZ, DMSO-d6) δ10.56 (s, 1H), 8.13 (q, 1H), 7.97 (d, 1H), 7.74 (dd, 1H), 7.61 (d, 1H), 4.37 (s, 1H), 3.57 (s, 3H), 3.30 (s, 4H), 1.80 (p, 1H), 0.88-0.81 (m, 4H); LC-MS: m/z 474.1 (MH+).

### Preparation: N-[4-(1,1-dioxo-1,4-thiazinane-4-carbonyl)-3-(1-propan-2-ylpyrazol-3-yl)phenyl]cyclopropanecarboxamide

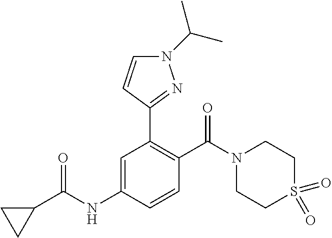 [SMILES: CC(C)n1ccc(-c2cc(NC(=O)C3CC3)ccc2C(=O)N2CCS(=O)(=O)CC2)n1]

### Example 28

The synthesis of the title compound was effected analogously to the synthesis of the compound of Example 25 N-[4-(1,1-dioxo-1,4-thiazinane-4-carbonyl)-3-(5-methylthiophen-2-yl)phenyl]cyclopropanecarboxamide wherein 1-propan-2-yl-3-(4,4,5,5-tetramethyl-1,3,2-dioxaborolan-2-yl)pyrazole was used instead of 4,4,5,5-tetramethyl-2-(5-methyl-2-thiophenyl)-1,3,2-dioxaborolane. The title compound was obtained in a yield of 37% (38 mg).

¹H NMR (400 MHZ, DMSO-d₆) δ 10.38 (s, 1H), 7.91 (d, 1H), 7.84 (d, 1H), 7.66 (dd, 1H), 7.28 (d, 1H), 6.38 (d, 1H), 4.52 (p, 1H), 4.09 (s, 1H), 3.97-3.86 (m, 1H), 3.56-3.36 (m, 2H), 3.10-3.00 (m, 2H), 2.58-2.53 (m, 2H), 1.80 (p, 1H), 1.44 (dd, 6H), 0.85-0.68 (m, 4H); LC-MS: m/z 431.2 (MH+).

### Preparation: N-[4-(1,1-dioxo-1,4-thiazinane-4-carbonyl)-3-[5-(trifluoromethyl)thiophen-2-yl]phenyl]cyclopropanecarboxamide

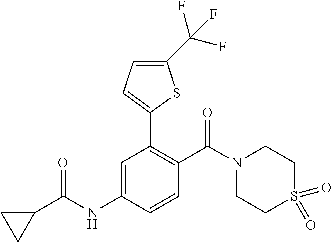 [SMILES: O=C(Nc1ccc(C(=O)N2CCS(=O)(=O)CC2)c(-c2ccc(C(F)(F)F)s2)c1)C1CC1]

### Example 29

The synthesis of the title compound was effected analogously to the synthesis of the compound of Example 25 N-[4-(1,1-dioxo-1,4-thiazinane-4-carbonyl)-3-(5-methylthiophen-2-yl)phenyl]cyclopropanecarboxamide wherein [5-(trifluoromethyl)thiophen-2-yl]boronic acid was used instead of 4,4,5,5-tetramethyl-2-(5-methyl-2-thiophenyl)-1,3,2-dioxaborolane. The title compound was obtained in a yield of 11% (12 mg).

1H NMR (400 MHZ, DMSO-d6) δ 10.54 (s, 1H), 7.96 (d, 1H), 7.76-7.68 (m, 2H), 7.51 (d, 1H), 7.22 (dq, 1H), 4.21 (s, 1H), 3.74 (s, 1H), 3.56 (s, 1H), 3.32 (s, 3H), 3.17 (d, 1H), 3.04 (s, 1H), 1.79 (p, 1H), 0.87-0.80 (m, 4H); LC-MS: m/z 473.1 (MH+).

### Preparation: N-[4-(4-methyl-2-phenylpiperazine-1-carbonyl)-3-(1-propan-2-ylpyrazol-3-yl)phenyl]cyclopropanecarboxamide

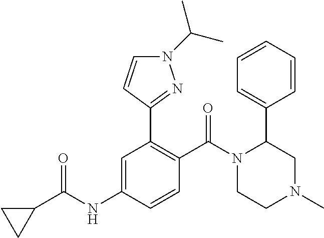 [SMILES: CC(C)n1ccc(-c2cc(NC(=O)C3CC3)ccc2C(=O)N2CCN(C)CC2c2ccccc2)n1]

### Example 30

A mixture of X-Phos aminobiphenyl palladium chloride precatalyst (10.94 mg, 0.010 mmol), tripotassium phosphate (88.68 mg, 0.420 mmol), 1-propan-2-yl-3-(4,4,5,5-tetramethyl-1,3,2-dioxaborolan-2-yl)pyrazole (32.88 mg, 0.140 mmol) and N-[3-bromo-4-(4-methyl-2-phenylpiperazine-1-carbonyl)phenyl]cyclopropanecarboxamide (80.0 mg, 0.140 mmol) in Water (0.195 mL)/1,2-dimethoxyethane (1.945 mL) was degassed for 5 min and then stirred at 100° C. ON. The day after the mixture was diluted with water and extracted with EtOAc ×3. The combined organic phases were washed with brine, dried over Na₂SO₄, filtered and concentrated under vacuum. The residue was purified by FC on reverse phase using basic conditions (eluting from 5:95 of CH₃CN/H₂O+0.1% of NH₄OH to 40:60 of CH₃CN/H₂O+0.1% of NH₄OH) and again by FC on NH column (eluting from 100% of DCM to DCM/EtOAc 60:40) affording the product of formula N-[4-(4-methyl-2-phenylpiperazine-1-carbonyl)-3-(1-propan-2-ylpyrazol-3-yl)phenyl]cyclopropanecarboxamide as a racemic mixture.

### Yield: 24 mg

1H NMR (400 MHZ, DMSO-d6) δ ppm 10.42-10.28 (m, 1H), 8.00-6.93 (m, 10H), 6.47-5.66 (m, 3H), 4.70-4.30 (m, 3H), 3.41-3.33 (m, 1H), 3.10-2.55 (m, 4H), 2.41-1.72 (m, 2H), 1.56-1.00 (m, 5H), 0.86-0.75 (m, 4H); LC-MS: m/z 472.28 (MH+).

### Preparation: N-[4-(4-methyl-2-phenylpiperazine-1-carbonyl)-3-[2-(trifluoromethyl)-1,3-thiazol-5-yl]phenyl]cyclopropanecarboxamide

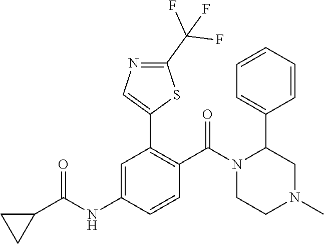 [SMILES: CN1CCN(C(=O)c2ccc(NC(=O)C3CC3)cc2-c2cnc(C(F)(F)F)s2)C(c2ccccc2)C1]

### Example 31

The synthesis of the title compound was effected analogously to the synthesis of the compound of Example 30 N-[4-(4-methyl-2-phenylpiperazine-1-carbonyl)-3-(1-propan-2-ylpyrazol-3-yl)phenyl]cyclopropanecarboxamide wherein 5-(tetramethyl-1,3,2-dioxaborolan-2-yl)-2-(trifluoromethyl)-1,3-thiazole was used instead of 1-propan-2-yl-3-(4,4,5,5-tetramethyl-1,3,2-dioxaborolan-2-yl)pyrazole. The title compound was obtained in a yield of 14% (10 mg) as a racemic mixture.

1H NMR (400 MHZ, DMSO-d6) δ ppm 10.64-10.49 (m, 1H), 8.38-7.12 (m, 8H), 5.80-5.64 (m, 1H), 4.43-4.32 (m, 1H), 3.35-3.28 (m, 2H), 2.44-1.70 (m, 8H), 0.88-0.72 (m, 4H); LC-MS: m/z 515.17 (MH+).

### Preparation: N-[4-(4-methyl-2-phenylpiperazine-1-carbonyl)-3-(2-methyl-1,3-thiazol-5-yl)phenyl]cyclopropanecarboxamide

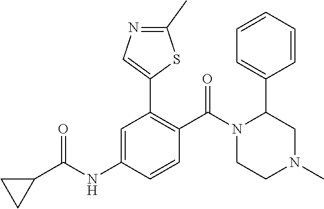 [SMILES: Cc1ncc(-c2cc(NC(=O)C3CC3)ccc2C(=O)N2CCN(C)CC2c2ccccc2)s1]

### Example 32

The synthesis of the title compound was effected analogously to the synthesis of the compound of Example 30 N-[4-(4-methyl-2-phenylpiperazine-1-carbonyl)-3-(1-propan-2-ylpyrazol-3-yl)phenyl]cyclopropanecarboxamide wherein 2-methyl-5-(4,4,5,5-tetramethyl-1,3,2-dioxaborolan-2-yl)-1,3-thiazole was used instead of 1-propan-2-yl-3-(4,4,5,5-tetramethyl-1,3,2-dioxaborolan-2-yl) pyrazole. The title compound was obtained in a yield of 47% (19 mg) as a racemic mixture.

¹H NMR (400 MHZ, DMSO-d₆) δ 10.54-10.34 (m, 1H), 7.94-7.60 (m, 2H), 7.55-7.16 (m, 6H), 5.73-5.64 (m, 1H), 4.60-4.28 (m, 2H), 3.12-2.53 (m, 5H), 2.02 (s, 6H), 1.17-0.90 (m, 1H), 0.89-0.74 (m, 4H); LC-MS: m/z 461.25 (MH+).

### Preparation: N-[4-(4-methyl-2-phenylpiperazine-1-carbonyl)-3-[5-(trifluoromethyl)thiophen-2-yl]phenyl]cyclopropanecarboxamide

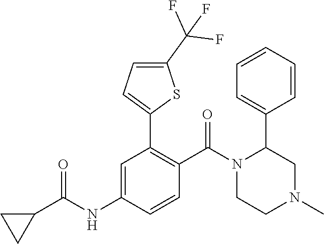 [SMILES: CN1CCN(C(=O)c2ccc(NC(=O)C3CC3)cc2-c2ccc(C(F)(F)F)s2)C(c2ccccc2)C1]

### Example 33

The synthesis of the title compound was effected analogously to the synthesis of the compound of Example 30 N-[4-(4-methyl-2-phenylpiperazine-1-carbonyl)-3-(1-propan-2-ylpyrazol-3-yl)phenyl]cyclopropanecarboxamide wherein [5-(trifluoromethyl)thiophen-2-yl]boronic acid was used instead of 1-propan-2-yl-3-(4,4,5,5-tetramethyl-1,3,2-dioxaborolan-2-yl)pyrazole. The title compound was obtained in a yield of 20% (11 mg) as a racemic mixture.

1H NMR (400 MHZ, DMSO-d6) δ ppm 10.56-10.41 (m, 1H), 8.04-7.15 (m, 10H), 5.78-5.66 (m, 1H), 4.55-4.28 (m, 1H), 3.39-3.25 (m, 1H), 3.13-2.54 (m, 3H), 2.41-2.27 (m, 1H), 2.23-2.12 (m, 2H), 2.05-1.73 (m, 1H), 1.57-1.45 (m, 1H), 0.83 (br d, 4H); LC-MS: m/z 514.29 (MH+).

### Preparation: N-[4-(4-methyl-2-phenylpiperazine-1-carbonyl)-3-(5-methylthiophen-2-yl)phenyl]cyclopropanecarboxamide

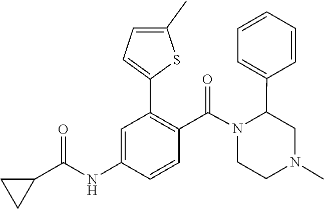 [SMILES: Cc1ccc(-c2cc(NC(=O)C3CC3)ccc2C(=O)N2CCN(C)CC2c2ccccc2)s1]

### Example 34

The synthesis of the title compound was effected analogously to the synthesis of the compound of Example 3.1 N-[4-(4-methyl-2-phenylpiperazine-1-carbonyl)-3-(1-propan-2-ylpyrazol-3-yl)phenyl]cyclopropanecarboxamide wherein 4,4,5,5-tetramethyl-2-(5-methyl-2-thiophenyl)-1,3,2-dioxaborolane was used instead of 1-propan-2-yl-3-(4,4,5,5-tetramethyl-1,3,2-dioxaborolan-2-yl) pyrazole. The title compound was obtained in a yield of 61% (38 mg) as a racemic mixture.

¹H NMR (400 MHZ, DMSO-d₆) δ 10.48-10.26 (m, 1H), 7.90-7.56 (m, 2H), 7.50-7.33 (m, 2H), 7.25 (dtd, 4H), 7.09-6.55 (m, 2H), 5.86-4.17 (m, 1H), 3.38-3.29 (m, 2H), 3.08-2.56 (m, 3H), 2.45-2.11 (m, 3H), 2.05-1.52 (m, 4H), 1.38-0.74 (m, 5H); LC-MS: m/z 460.18 (MH+).

### Preparation: N-[4-(1,1-dioxo-1,4-thiazinane-4-carbonyl)-3-(4,4,5,5-tetramethyl-1,3,2-dioxaborolan-2-yl)phenyl]cyclopropanecarboxamide

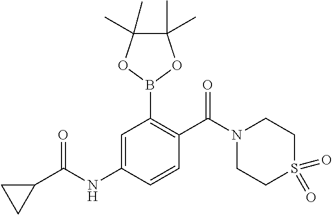 [SMILES: CC1(C)OB(c2cc(NC(=O)C3CC3)ccc2C(=O)N2CCS(=O)(=O)CC2)OC1(C)C]

### Example 35

A mixture of N-[3-bromo-4-(1,1-dioxo-1,4-thiazinane-4-carbonyl)phenyl]cyclopropanecarboxamide (100.0 mg, 0.240 mmol), 4,4,5,5-tetramethyl-2-(4,4,5,5-tetramethyl-1,3,2-dioxaborolan-2-yl)-1,3,2-dioxaborolane (74.42 mg, 0.290 mmol), potassium acetate (72.64 mg, 0.730 mmol) and [1,1′-Bis(diphenylphosphino)ferrocene]dichloropalladium (II), complex with dichloromethane (4.0 mg, 0 mmol) in 1,4-Dioxane (2.442 mL) was degassed using Shlenk line technique and stirred at 100° C. overnight. The day after the reaction was cooled down to RT and filtered through a pad of celite washing with EtOAc. The solution was concentrated under vacuum affording the product of formula N-[4-(1,1-dioxo-1,4-thiazinane-4-carbonyl)-3-(4,4,5,5-tetramethyl-1,3,2-dioxaborolan-2-yl)phenyl]cyclopropanecarboxamide, which was used in the next step without further purification.

### Yield: 100 mg

1H NMR (400 MHZ, DMSO) δ 10.37 (d, 1H), 7.81 (dd, 1H), 7.70-7.60 (m, 1H), 7.44 (d, 1H), 3.57 (s, 8H), 1.84-1.77 (m, 1H), 1.27 (s, 6H), 1.07 (s, 6H), 0.90-0.76 (m, 4H); LC-MS: m/z 449.21 (MH+).

### Preparation: N-[4-(2-oxa-7-azaspiro[3.5]nonane-7-carbonyl)-3-(4,4,5,5-tetramethyl-1,3,2-dioxaborolan-2-yl)phenyl]cyclopropanecarboxamide

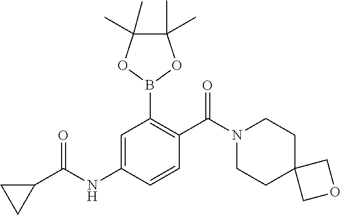 [SMILES: CC1(C)OB(c2cc(NC(=O)C3CC3)ccc2C(=O)N2CCC3(CC2)COC3)OC1(C)C]

### Example 36

The synthesis of the title compound was effected analogously to the synthesis of the compound of Example 3.N N-[4-(1,1-dioxo-1,4-thiazinane-4-carbonyl)-3-(4,4,5,5-tetramethyl-1,3,2-dioxaborolan-2-yl)phenyl]cyclopropanecarboxamide wherein N-[3-bromo-4-(2-oxa-7-azaspiro[3.5]nonane-7-carbonyl)phenyl]cyclopropanecarboxamide was used instead of N-[3-bromo-4-(1,1-dioxo-1,4-thiazinane-4-carbonyl)phenyl]cyclopropanecarboxamide. The title compound was obtained in a quantitative yield (80 mg).

### LC-MS: m/z 441.25 (MH+)

### Preparation: N-[4-(1,1-dioxo-1,4-thiazinane-4-carbonyl)-3-[6-(trifluoromethyl)pyridin-2-yl]phenyl]cyclopropanecarboxamide

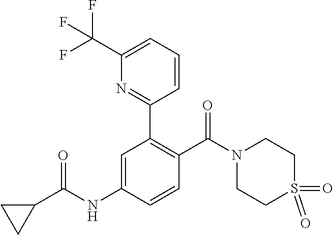 [SMILES: O=C(Nc1ccc(C(=O)N2CCS(=O)(=O)CC2)c(-c2cccc(C(F)(F)F)n2)c1)C1CC1]

### Example 37

Palladium tetrakis triphenylphosphine (14.43 mg, 0.010 mmol) and N-[4-(1,1-dioxo-1,4-thiazinane-4-carbonyl)-3-(4,4,5,5-tetramethyl-1,3,2-dioxaborolan-2-yl)phenyl]cyclopropanecarboxamide (112.0 mg, 0.250 mmol) were suspended in 1,2-dimethoxyethane (2.5 mL) and then a 2 M aqueous solution of sodium carbonate (0.37 mL, 0.750 mmol) was added. The mixture was degassed by 5 cycles of nitrogen/vacuum and 2-bromo-6-(trifluoromethyl)pyridine (62.1 mg, 0.270 mmol) was added. The reaction mixture was degassed again by 5 cycles of nitrogen/vacuum and heated to 90° C. overnight. The day after the reaction mixture was cooled down to room temperature and diluted with water and extracted with EtOAc (3×). The organic layers were collected together and washed with brine, dried over Na₂SO₄, filtered and the solvent removed under vacuum. The mixture was purified by FC on RP using acid conditions (eluting from 5:95 of CH₃CN/H₂O+0.1% of HCOOH to 35:65 of CH₃CN/H₂O+0.1% of HCOOH) affording the product of formula N-[4-(1,1-dioxo-1,4-thiazinane-4-carbonyl)-3-[6-(trifluoromethyl)pyridin-2-yl]phenyl]cyclopropanecarboxamide.

### Yield: 22 mg

1H NMR (400 MHZ, DMSO) δ 10.52 (s, 1H), 8.21 (t, 1H), 8.04 (d, 1H), 7.95 (d, 1H), 7.89 (dd, 1H), 7.75 (dd, 1H), 7.55 (d, 1H), 4.37 (s, 1H), 3.30 (s, 7H), 1.81 (p, 1H), 0.84 (d, 4H); LC-MS: m/z 468.17 (MH+).

### Preparation: N-[4-(2-oxa-7-azaspiro[3.5]nonane-7-carbonyl)-3-[6-(trifluoromethyl)pyridin-2-yl]phenyl]cyclopropanecarboxamide

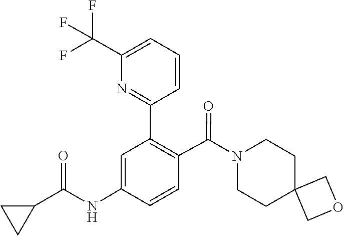 [SMILES: O=C(Nc1ccc(C(=O)N2CCC3(CC2)COC3)c(-c2cccc(C(F)(F)F)n2)c1)C1CC1]

### Example 38

The synthesis of the title compound was effected analogously to the synthesis of the compound of Example 37 N-[4-(1,1-dioxo-1,4-thiazinane-4-carbonyl)-3-[6-(trifluoromethyl)pyridin-2-yl]phenyl]cyclopropanecarboxamide wherein N-[4-(2-oxa-7-azaspiro[3.5]nonane-7-carbonyl)-3-(4,4,5,5-tetramethyl-1,3,2-dioxaborolan-2-yl)phenyl]cyclopropanecarboxamide was used instead of N-[4-(1,1-dioxo-1,4-thiazinane-4-carbonyl)-3-(4,4,5,5-tetramethyl-1,3,2-dioxaborolan-2-yl)phenyl]cyclopropanecarboxamide. The title compound was obtained in a yield of 23% (20 mg).

1H NMR (400 MHZ, DMSO) δ 10.49 (s, 1H), 8.17 (t, 1H), 7.98 (d, 1H), 7.86 (dd, 2H), 7.74 (dd, 1H), 7.30 (d, 1H), 4.44-4.07 (m, 4H), 3.30 (s, 4H), 1.81 (p, 1H), 1.76-1.37 (m, 4H), 0.88-0.79 (m, 4H); LC-MS: m/z 460.20 (MH+).

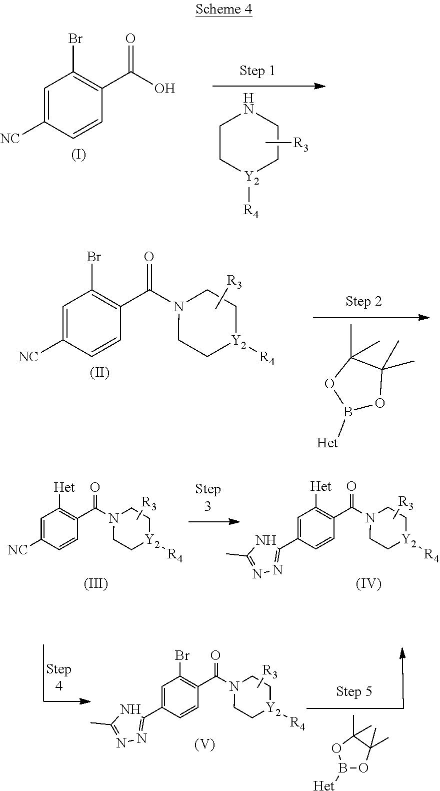

### Step 1

Compound of formula II may be obtained from compound of formula I (commercially available from Astatech) by coupling with a proper amine in the presence of coupling agent, e.g. HATU, and an organic base, e.g. DIPEA. The reaction is carried out in a suitable solvent such as DMF, typically at room temperature. The reaction takes from about 3 hours to about 12 hours to complete.

### Step 2

Compound of formula III may be obtained via Suzuki coupling between compound of formula II and the desired heteroaryl boronic ester, in the presence of a suitable precatalyst, e.g. XPhos Pd G2, a suitable base, e.g. K₃PO₄, in a suitable mixture of solvents, e.g. 1,2-dimethoxyethane/water, typically at 100° C. The reaction takes about from 3 hours to about 12 hours to complete.

### Step 3

Compound of formula IV may be obtained by nitrile cyclization of compound of formula III with ethanimidamide hydrochloride in presence of a suitable catalyst, e.g. CuBr, a suitable base, e.g. Cs₂CO₃, in a suitable solvent, such as DMSO, typically at 120° C. The reaction takes about from 3 hours to about 12 hours to complete.

### Step 4

Compound of formula V may be obtained by nitrile cyclization of compound of formula III with ethanimidamide hydrochloride in presence of a suitable catalyst, e.g. CuBr, a suitable base, e.g. Cs₂CO₃, in a suitable solvent, such as DMSO, typically at 120° C. The reaction takes about from 3 hours to about 12 hours to complete.

### Step 5

Compound of formula IV may be obtained via Suzuki coupling between compound of formula V and the desired heteroaryl boronic ester, in the presence of a suitable precatalyst, e.g. XPhos Pd G2, a suitable base, e.g. K₃PO₄, in a suitable mixture of solvents, e.g. 1,2-dimethoxyethane/water, typically at 100° C. The reaction takes about from 3 hours to about 12 hours to complete.

### Scheme 4, Cpd Preparation: (II)*a 3-bromo-4-(4-methoxy-4-methyl-piperidine-1-carbonyl)benzonitrile

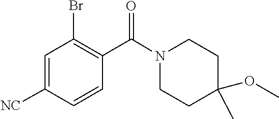 [SMILES: COC1(C)CCN(C(=O)c2ccc(C#N)cc2Br)CC1]

To a solution of HATU, (0.63 g, 1.66 mmol), 2-bromo-4-cyanobenzoic acid (250.0 mg, 1.11 mmol) and N,N-Diisopropylethylamine (1.13 mL, 6.64 mmol) in DMF (7.374 mL), 4-methoxy-4-methyl-piperidine hydrochloride (219.87 mg, 1.33 mmol) was added. The reaction was stirred at RT for 2.5 h. After this time a saturated solution of NaHCO₃ was added to the reaction mixture and the aqueous phase was extracted with EtOAc (×3). The organic portions were collected, washed with brine, residual water was removed by adding Na₂SO₄ and the solvent was evaporated under reduced pressure. The residue was purified by FC on silica gel (eluting from cHex/AcOEt 95:5 to cHex/AcOEt 50:50) affording the product of formula 3-bromo-4-(4-methoxy-4-methyl-piperidine-1-carbonyl)benzonitrile.

### Yield: 170 mg

1H NMR (400 MHZ, DMSO) δ 8.27 (t, 1H), 7.94 (ddd, 1H), 7.62-7.50 (m, 1H), 4.13 (ddd, 1H), 3.22-3.02 (m, 5H), 3.00-2.89 (m, 1H), 1.86-1.72 (m, 1H), 1.68-1.57 (m, 1H), 1.57-1.33 (m, 2H), 1.12 (d, 3H); LC-MS: m/z 337.25, 339.25 (MH+).

### Scheme 4, Cpd (II)*b Preparation: 3-bromo-4-(4-tert-butylpiperidine-1-carbonyl)benzonitrile

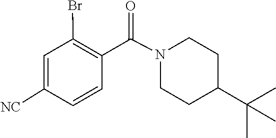 [SMILES: CC(C)(C)C1CCN(C(=O)c2ccc(C#N)cc2Br)CC1]

The synthesis of the title compound was effected analogously to the synthesis of the compound 3-bromo-4-(4-methoxy-4-methyl-piperidine-1-carbonyl)benzonitrile wherein 4-tert-butylpiperidine was used instead of 4-methoxy-4-methyl-piperidine hydrochloride. The title compound was obtained in a yield of 90% (346 mg).

¹H NMR (400 MHZ, DMSO-d₆) δ 8.30-8.25 (m, 1H), 7.98-7.87 (m, 1H), 7.66-7.47 (m, 1H), 4.59 (t, 1H), 3.23-3.11 (m, 1H), 3.02-2.91 (m, 1H), 2.76-2.63 (m, 1H), 1.90-1.50 (m, 2H), 1.34-1.03 (m, 3H), 0.89-0.81 (m, 9H); LC-MS: m/z 349.17, 351.13 (MH+).

### Scheme 4, Cpd (II)*c Preparation: 3-bromo-4-(4,4-difluoropiperidine-1-carbonyl)benzonitrile

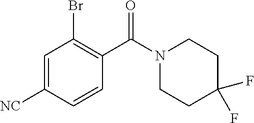 [SMILES: N#Cc1ccc(C(=O)N2CCC(F)(F)CC2)c(Br)c1]

The synthesis of the title compound was effected analogously to the synthesis of the compound 3-bromo-4-(4-methoxy-4-methyl-piperidine-1-carbonyl)benzonitrile wherein 4,4-difluoropiperidine hydrochloride was used instead of 4-methoxy-4-methyl-piperidine hydrochloride. The title compound was obtained in a yield of 77% (500 mg).

¹H NMR (400 MHZ, DMSO-d₆) δ 8.31 (d, 1H), 7.98 (dd, 1H), 7.66 (d, 1H), 3.95-3.83 (m, 1H), 3.78-3.61 (m, 1H), 3.23 (t2H), 2.23-1.87 (m, 4H); LC-MS: m/z 329.25, 331.25 (MH+).

### Scheme 4, Cpd (II)*d Preparation: 3-bromo-4-(1-oxa-7-azaspiro[3.5]nonane-7-carbonyl)benzonitrile

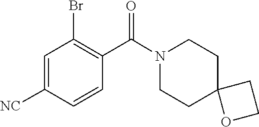 [SMILES: N#Cc1ccc(C(=O)N2CCC3(CCO3)CC2)c(Br)c1]

The synthesis of the title compound was effected analogously to the synthesis of the compound 3-bromo-4-(4-methoxy-4-methyl-piperidine-1-carbonyl)benzonitrile wherein 1-oxa-7-azaspiro[3.5]nonane was used instead of 4-methoxy-4-methyl-piperidine hydrochloride. The title compound was obtained in a yield of 74% (487 mg).

1H NMR (400 MHz, DMSO-d6) δ ppm 8.27 (d, 1H), 7.93 (dt, 1H), 7.56 (dd, 1H), 4.46-4.33 (m, 2H), 3.73-3.51 (m, 2H), 3.27 (s, 1H), 2.99 (s, 2H), 2.37 (br d, 2H), 1.96-1.70 (m, 3H); LC-MS: m/z 335.09, 337.06 (MH+).

### Scheme 4, Cpd (II)*e Preparation: 3-bromo-4-(4-ethoxypiperidine-1-carbonyl)benzonitrile

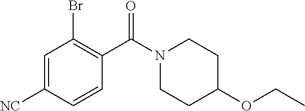 [SMILES: CCOC1CCN(C(=O)c2ccc(C#N)cc2Br)CC1]

The synthesis of the title compound was effected analogously to the synthesis of the compound 3-bromo-4-(4-methoxy-4-methyl-piperidine-1-carbonyl)benzonitrile wherein 4-ethoxypiperidine hydrochloride was used instead of 4-methoxy-4-methyl-piperidine hydrochloride. The title compound was obtained in a yield of 56% (210 mg).

1H NMR (400 MHZ, DMSO) δ 8.28 (t, 1H), 7.94 (dd, 1H), 7.56 (dd, 1H), 3.97 (td, 1H), 3.54 (tt, 1H), 3.46 (qd, 2H), 3.35-3.31 (m, 1H), 3.19 (ddt, 1H), 2.99 (tdd, 1H), 1.89 (s, 1H), 1.81-1.72 (m, 1H), 1.58-1.31 (m, 2H), 1.10 (t, 3H); LC-MS: m/z 337.35, 339.35 (MH+).

### Scheme 4, Cpd (II)*f Preparation: 3-bromo-4-(6,6-difluoro-2-azaspiro[3.3]heptane-2-carbonyl)benzonitrile

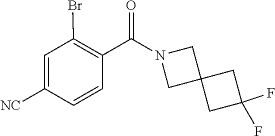 [SMILES: N#Cc1ccc(C(=O)N2CC3(C2)CC(F)(F)C3)c(Br)c1]

The synthesis of the title compound was effected analogously to the synthesis of the compound 3-bromo-4-(4-methoxy-4-methyl-piperidine-1-carbonyl)benzonitrile wherein 6,6-difluoro-2-azaspiro[3.3]heptane hydrochloride was used instead of 4-methoxy-4-methyl-piperidine hydrochloride. The title compound was obtained in a yield of 50% (70 mg).

¹H NMR (400 MHZ, DMSO-d₆) δ 8.30 (d, 1H), 7.96 (dd, 1H), 7.61 (d, 1H), 4.19 (s, 2H), 3.97 (s, 2H), 2.96-2.78 (m, 4H); LC-MS: m/z 341.25, 343.25 (MH+)

### Scheme 4, Cpd Preparation: 3-bromo-4-[4-(trifluoromethoxy) piperidine-1-carbonyl]benzonitrile

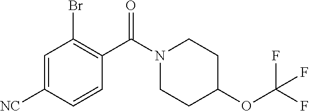 [SMILES: N#Cc1ccc(C(=O)N2CCC(OC(F)(F)F)CC2)c(Br)c1]

The synthesis of the title compound was effected analogously to the synthesis of the compound 3-bromo-4-(4-methoxy-4-methyl-piperidine-1-carbonyl)benzonitrile wherein 4-(trifluoromethoxy) piperidine hydrochloride was used instead of 4-methoxy-4-methyl-piperidine hydrochloride. The title compound was obtained in a yield of 62% (260 mg).

1H NMR (400 MHZ, DMSO) δ 8.29 (dd, 1H), 7.96 (ddd, 1H), 7.72-7.49 (m, 1H), 4.74 (dt, 1H), 4.02 (td, 1H), 3.42 (dddd, 1H), 3.30-3.06 (m, 2H), 2.09-1.97 (m, 1H), 1.90 (s, 1H), 1.84-1.56 (m, 2H); LC-MS: m/z 377.26, 379.26 (MH+).

### Scheme 4, Cpd (II)*h Preparation: 3-bromo-4-[4-(trifluoromethyl) piperidine-1-carbonyl]benzonitrile

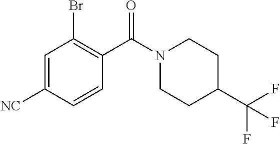 [SMILES: N#Cc1ccc(C(=O)N2CCC(C(F)(F)F)CC2)c(Br)c1]

The synthesis of the title compound was effected analogously to the synthesis of the compound 3-bromo-4-(4-methoxy-4-methyl-piperidine-1-carbonyl)benzonitrile wherein 4-(trifluoromethoxy) piperidine hydrochloride was used instead of 4-methoxy-4-methyl-piperidine hydrochloride. The title compound was obtained in a yield of 78% (560 mg).

1H NMR (400 MHZ, DMSO) δ 8.29 (dd, 1H), 7.95 (ddd, 1H), 7.71-7.50 (m, 1H), 4.60 (t, 1H), 3.26 (d, 1H), 3.09 (dtd, 1H), 2.83 (tdd1H), 2.66 (d, 1H), 1.94 (t, 1H), 1.82-1.65 (m, 1H), 1.55-1.29 (m, 2H); LC-MS: m/z 361.10, 363.09 (MH+).

### Scheme 4, Cpd (II)*i Preparation: 3-bromo-4-(4-fluoro-4-methylpiperidine-1-carbonyl)benzonitrile

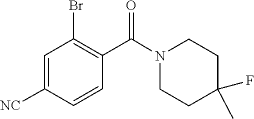 [SMILES: CC1(F)CCN(C(=O)c2ccc(C#N)cc2Br)CC1]

The synthesis of the title compound was effected analogously to the synthesis of the compound 3-bromo-4-(4-methoxy-4-methyl-piperidine-1-carbonyl)benzonitrile wherein 4-fluoro-4-methylpiperidine hydrochloride was used instead of 4-methoxy-4-methyl-piperidine hydrochloride. The title compound was obtained in a yield of 52% (186 mg).

1H NMR (400 MHZ, DMSO) δ 8.29 (d, 1H), 7.95 (ddd, 1H), 7.65-7.53 (m, 1H), 4.26 (d, 1H), 3.25-2.97 (m, 3H), 1.94-1.79 (m, 1H), 1.78-1.54 (m, 3H), 1.42-1.28 (m, 3H); LC-MS: m/z 325.25, 327.25 (MH+).

### Scheme 4, Cpd (II)*j Preparation: 3-bromo-4-(4-methoxypiperidine-1-carbonyl)benzonitrile

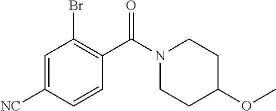 [SMILES: COC1CCN(C(=O)c2ccc(C#N)cc2Br)CC1]

The synthesis of the title compound was effected analogously to the synthesis of the compound 3-bromo-4-(4-methoxy-4-methyl-piperidine-1-carbonyl)benzonitrile wherein 4-methoxypiperidine was used instead of 4-methoxy-4-methyl-piperidine hydrochloride. The title compound was obtained in a yield of 56% (200 mg).

1H NMR (400 MHZ, DMSO) δ 8.28 (d, 1H), 7.94 (dt, 1H), 7.56 (dd, 1H), 3.97-3.87 (m, 1H), 3.49-3.33 (m, 2H), 3.25 (s, 3H), 3.18 (qd, 1H), 3.06-2.92 (m, 1H), 1.95-1.84 (m, 1H), 1.76 (s, 1H), 1.59-1.34 (m, 2H); LC-MS: 323.25, 325.25 (MH+).

### Scheme 4, Cpd (II)*k Preparation 3-bromo-4-(4-methylpiperidine-1-carbonyl)benzonitrile

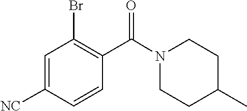 [SMILES: CC1CCN(C(=O)c2ccc(C#N)cc2Br)CC1]

The synthesis of the title compound was effected analogously to the synthesis of the compound 3-bromo-4-(4-methoxy-4-methyl-piperidine-1-carbonyl)benzonitrile wherein 4-methylpiperidine was used instead of 4-methoxy-4-methyl-piperidine hydrochloride. The title compound was obtained in a yield of 78% (265 mg).

1H NMR (400 MHZ, DMSO) δ 8.27 (dd, 1H), 7.93 (ddd, 1H), 7.60-7.46 (m, 1H), 4.45 (t, 1H), 3.14 (d, 1H), 3.07-2.91 (m, 1H), 2.78 (tt, 1H), 1.76-1.45 (m, 3H), 1.26-0.97 (m, 2H), 0.91 (d, 3H); LC-MS: m/z 307.10, 309.09 (MH+).

### Scheme 4, Cpd (II)*l Preparation: 4-(6-azaspiro[2.5]octane-6-carbonyl)-3-bromobenzonitrile

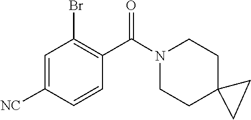 [SMILES: N#Cc1ccc(C(=O)N2CCC3(CC2)CC3)c(Br)c1]

The synthesis of the title compound was effected analogously to the synthesis of the compound 3-bromo-4-(4-methoxy-4-methyl-piperidine-1-carbonyl)benzonitrile wherein 6-azaspiro[2.5]octane hydrochloride was used instead of 4-methoxy-4-methyl-piperidine hydrochloride. The title compound was obtained in a yield of 78% (550 mg).

¹H NMR (400 MHZ, DMSO-d₆) δ 8.28 (d, 1H), 7.94 (dd, 1H), 7.57 (d, 1H), 3.67 (t, 2H), 3.16-3.05 (m, 2H), 1.56-1.20 (m, 4H), 0.51-0.24 (m, 4H); LC-MS: m/z 319.14, 321.10 (MH+).

### Scheme 4, Cpd (III)*a Preparation: 3-(1-isopropylpyrazol-3-yl)-4-(4-methoxy-4-methyl-piperidine-1-carbonyl)benzonitrile

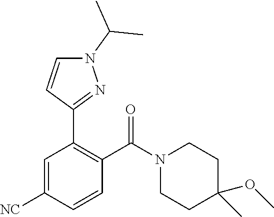 [SMILES: COC1(C)CCN(C(=O)c2ccc(C#N)cc2-c2ccn(C(C)C)n2)CC1]

A mixture of 1-(propan-2-yl)-3-(tetramethyl-1,3,2-dioxaborolan-2-yl)-1H-pyrazole (138.64 mg, 0.590 mmol), 3-bromo-4-(4-methoxy-4-methyl-piperidine-1-carbonyl)benzonitrile (180.0 mg, 0.530 mmol), X-phos amonobiphenyl palladium chloride precatalyst (41.95 mg, 0.050 mmol), tripotassium phosphate (339.92 mg, 1.6 mmol) in Water (0.755 mL)/1,2-dimethoxyethane (7.554 mL) was degassed under nitrogen flux for 5 min and then heated to 90° C. for 1 h. After this time the reaction mixture was cooled down to room temperature, diluted with water and extracted with AcOEt (3×). The organic layers were collected together and washed with brine, dried over Na₂SO₄, filtered and the solvent removed under vacuum. The residue was purified by FC on silica gel (eluting form 100% of cHex to AcOEt/cHex 30:70) affording the product of formula 3-(1-isopropylpyrazol-3-yl)-4-(4-methoxy-4-methyl-piperidine-1-carbonyl)benzonitrile.

### Yield: 120 mg

1H NMR (400 MHZ, DMSO) δ 8.23-8.05 (m, 1H), 7.89-7.76 (m, 2H), 7.44 (dd, 1H), 6.69-6.50 (m, 1H), 4.52 (dp, 1H), 4.19-4.08 (m, 1H), 3.09-2.79 (m, 6H), 1.75 (t, 1H), 1.52-1.26 (m, 9H), 1.10-0.95 (m, 3H); LC-MS: m/z 367.5 (MH+).

### Scheme 4, Cpd (III)*b Preparation: 4-(4-tert-butylpiperidine-1-carbonyl)-3-(1-isopropylpyrazol-3-yl)benzonitrile

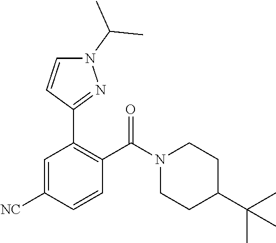 [SMILES: CC(C)n1ccc(-c2cc(C#N)ccc2C(=O)N2CCC(C(C)(C)C)CC2)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-(4-tert-butylpiperidine-1-carbonyl)benzonitrile was used instead of 3-bromo-4-(4-methoxy-4-methyl-piperidine-1-carbonyl)benzonitrile. The title compound was obtained in a yield of 91% (100 mg).

¹H NMR (400 MHZ, DMSO-d₆) δ 8.24-8.06 (m, 1H), 7.87-7.76 (m, 2H), 7.49-7.37 (m, 1H), 6.64-6.47 (m, 1H), 4.71-4.40 (m, 2H), 3.28-3.01 (m, 2H), 2.91-2.53 (m, 2H), 1.83-1.63 (m, 1H), 1.57-1.38 (m, 7H), 1.32-1.13 (m, 2H), 0.91-0.60 (m, 9H); LC-MS: m/z 379.36 (MH+).

### Scheme 4, Cpd (III)*c Preparation: 3-(1-isopropylpyrazol-3-yl)-4-(1-oxa-7-azaspiro[3.5]nonane-7-carbonyl)benzonitrile

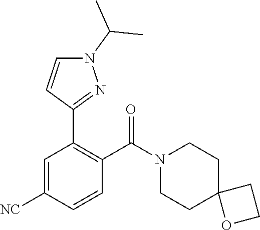 [SMILES: CC(C)n1ccc(-c2cc(C#N)ccc2C(=O)N2CCC3(CCO3)CC2)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-(1-oxa-7-azaspiro[3.5]nonane-7-carbonyl)benzonitrile was used instead of 3-bromo-4-(4-methoxy-4-methyl-piperidine-1-carbonyl)benzonitrile. The title compound was obtained in a yield of 60% (65 mg).

¹H NMR (400 MHZ, DMSO-d₆) δ 8.16 (dd, 1H), 7.86-7.80 (m, 2H), 7.45 (dd, 1H), 6.59 (dd, 1H), 4.57-4.28 (m, 2H), 3.71-3.50 (m, 2H), 3.15-2.79 (m, 2H), 2.43-2.18 (m, 2H), 1.90-1.73 (m, 2H), 1.63-1.55 (m, 1H), 1.49-1.36 (m, 7H), 1.32-1.21 (m, 1H); LC-MS: m/z 365.24 (MH+).

### Scheme 4, Cpd (III)*d Preparation: 4-(4-ethoxypiperidine-1-carbonyl)-3-(1-isopropylpyrazol-3-yl)benzonitrile

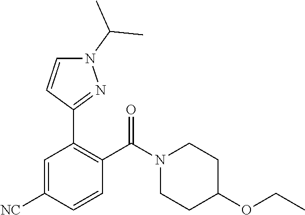 [SMILES: CCOC1CCN(C(=O)c2ccc(C#N)cc2-c2ccn(C(C)C)n2)CC1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-(4-ethoxypiperidine-1-carbonyl)benzonitrile was used instead of 3-bromo-4-(4-methoxy-4-methyl-piperidine-1-carbonyl)benzonitrile. The title compound was obtained in a yield of 79% (180 mg).

1H NMR (400 MHZ, DMSO) δ 8.16 (dd, 1H), 7.88-7.77 (m, 2H), 7.44 (t, 1H), 6.58 (t, 1H), 4.53 (dp, 1H), 4.00-3.84 (m, 1H), 3.44 (qd, 2H), 3.40-2.99 (m, 3H), 2.94-2.73 (m, 1H), 1.88-1.75 (m, 1H), 1.59-1.49 (m, 2H), 1.43 (td, 6H), 1.34-1.18 (m, 1H), 1.07 (dt, 3H); LC-MS: m/z 367.3 (MH+).

### Scheme 4, Cpd (III)*e Preparation: 4-(6,6-difluoro-2-azaspiro[3.3]heptane-2-carbonyl)-3-(1-isopropylpyrazol-3-yl)benzonitrile

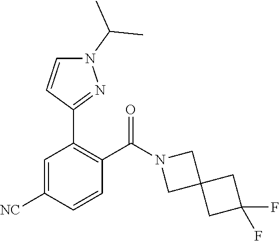 [SMILES: CC(C)n1ccc(-c2cc(C#N)ccc2C(=O)N2CC3(C2)CC(F)(F)C3)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-(6,6-difluoro-2-azaspiro[3.3]heptane-2-carbonyl)benzonitrile was used instead of 3-bromo-4-(4-methoxy-4-methyl-piperidine-1-carbonyl)benzonitrile. The title compound was obtained in a yield of 60% (52 mg).

### LC-MS: m/z 371.46 (MH+)

### Scheme 4, Cpd (III)*f Preparation: 4-(4,4-difluoropiperidine-1-carbonyl)-3-(1-propan-2-ylpyrazol-3-yl)benzonitrile

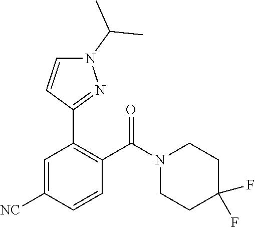 [SMILES: CC(C)n1ccc(-c2cc(C#N)ccc2C(=O)N2CCC(F)(F)CC2)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-(4,4-difluoropiperidine-1-carbonyl)benzonitrile was used instead of 3-bromo-4-(4-methoxy-4-methyl-piperidine-1-carbonyl)benzonitrile. The title compound was obtained in a yield of 100% (90 mg).

¹H NMR (400 MHZ, DMSO-d₆) δ 8.22-8.17 (m, 1H), 7.89-7.82 (m, 2H), 7.57-7.47 (m, 1H), 6.65 (d, 1H), 4.51 (p, 1H), 3.80-3.66 (m, 2H), 3.25-2.99 (m, 2H), 2.16-1.77 (m, 2H), 1.54 (dq, 1H), 1.43 (dd, 7H); LC-MS: m/z 359.2 (MH+).

### Scheme 4, Cpd (III)*g reparation: 4-(4-fluoro-4-methylpiperidine-1-carbonyl)-3-(1-propan-2-ylpyrazol-3-yl)benzonitrile

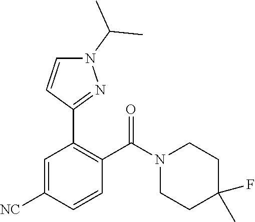 [SMILES: CC(C)n1ccc(-c2cc(C#N)ccc2C(=O)N2CCC(C)(F)CC2)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-(4-fluoro-4-methylpiperidine-1-carbonyl)benzonitrile was used instead of 3-bromo-4-(4-methoxy-4-methyl-piperidine-1-carbonyl)benzonitrile. The title compound was obtained in a yield of 100% (204 mg).

1H NMR (400 MHZ, DMSO) δ 8.22-8.08 (m, 1H), 7.89-7.78 (m, 2H), 7.47 (dd, 1H), 6.67-6.51 (m, 1H), 4.52 (h, 1H), 4.33-4.17 (m, 1H), 3.13-2.88 (m, 3H), 1.84-1.12 (m, 13H); LC-MS: m/z 355.2 (MH+).

### Scheme 4, Cpd (III)*h Preparation: 3-(1-propan-2-ylpyrazol-3-yl)-4-[4-(trifluoromethyl) piperidine-1-carbonyl]benzonitrile

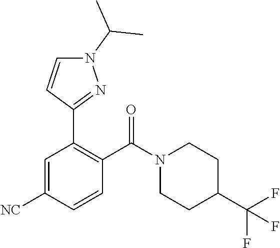 [SMILES: CC(C)n1ccc(-c2cc(C#N)ccc2C(=O)N2CCC(C(F)(F)F)CC2)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-[4-(trifluoromethyl) piperidine-1-carbonyl]benzonitrile was used instead of 3-bromo-4-(4-methoxy-4-methyl-piperidine-1-carbonyl)benzonitrile. The title compound was obtained in a yield of 100% (220 mg).

1H NMR (400 MHZ, DMSO) δ 8.25-8.06 (m, 1H), 7.88-7.77 (m, 2H), 7.56-7.40 (m, 1H), 6.67-6.47 (m, 1H), 4.62 (s, 1H), 4.57-4.46 (m, 1H), 3.26-2.62 (m, 4H), 1.43 (ddd, 6H), 1.96-1.12 (m, 4H); LC-MS: m/z 391.3 (MH+).

### Scheme 4, Cpd (III)*i Preparation: 4-(6-azaspiro[2.5]octane-6-carbonyl)-3-(1-propan-2-ylpyrazol-3-yl)benzonitrile

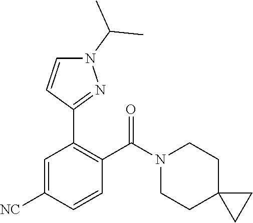 [SMILES: CC(C)n1ccc(-c2cc(C#N)ccc2C(=O)N2CCC3(CC2)CC3)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 4-(6-azaspiro[2.5]octane-6-carbonyl)-3-bromobenzonitrile was used instead of 3-bromo-4-(4-methoxy-4-methyl-piperidine-1-carbonyl)benzonitrile. The title compound was obtained in a yield of 80% (130 mg).

¹H NMR (400 MHZ, DMSO-d₆) δ 8.16 (d, 1H), 7.85 (d, 1H), 7.81 (dd, 1H), 7.45 (d, 1H), 6.60 (d, 1H), 4.60-4.45 (m, 1H), 3.76-3.49 (m, 2H), 3.15-2.85 (m, 2H), 1.50-1.40 (m, 7H), 1.37-1.30 (m, 1H), 1.22-1.12 (m, 1H), 0.93-0.85 (m, 1H), 0.40-0.13 (m, 4H); LC-MS: 349.2 (MH+).

### Scheme 4, Cpd (III)*j Preparation: 4-(4,4-difluoropiperidine-1-carbonyl)-3-(2-ethylpyrazol-3-yl)benzonitrile

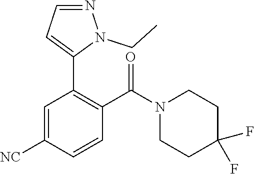 [SMILES: CCn1nccc1-c1cc(C#N)ccc1C(=O)N1CCC(F)(F)CC1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-(4,4-difluoropiperidine-1-carbonyl)benzonitrile and 1-ethyl-5-(4,4,5,5-tetramethyl-1,3,2-dioxaborolan-2-yl)pyrazole were used instead of 3-bromo-4-(4-methoxy-4-methyl-piperidine-1-carbonyl)benzonitrile and 1-(propan-2-yl)-3-(tetramethyl-1,3,2-dioxaborolan-2-yl)-1H-pyrazole respectively. The title compound was obtained in a yield of 30% (76 mg).

1H NMR (400 MHZ, DMSO-d6) δ ppm 8.06-7.99 (m, 2H), 7.77-7.70 (m, 1H), 7.52 (d, 1H), 6.26 (d, 1H), 4.10-3.93 (m, 2H), 3.71-3.42 (m, 2H), 3.26-2.95 (m, 2H), 2.12-1.64 (m, 4H), 1.30 (t, 3H); LC-MS: m/z 345.24 (MH+).

### Scheme 4, Cpd (III)*k Preparation: 3-[1-(2-methylpropyl) pyrazol-3-yl]-4-[4-(trifluoromethyl) piperidine-1-carbonyl]benzonitrile

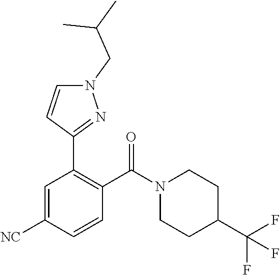 [SMILES: CC(C)Cn1ccc(-c2cc(C#N)ccc2C(=O)N2CCC(C(F)(F)F)CC2)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-[4-(trifluoromethyl) piperidine-1-carbonyl]benzonitrile and 1-(2-methylpropyl)-3-(4,4,5,5-tetramethyl-1,3,2-dioxaborolan-2-yl)pyrazole (see Scheme 12, Cpd (III)*e) were used instead of 3-bromo-4-(4-methoxy-4-methyl-piperidine-1-carbonyl)benzonitrile and 1-(propan-2-yl)-3-(tetramethyl-1,3,2-dioxaborolan-2-yl)-1H-pyrazole respectively. The title compound was obtained in a yield of 35% (12 mg).

1H NMR (400 MHZ, DMSO) δ 8.26-8.11 (m, 1H), 7.87-7.76 (m, 2H), 7.59-7.40 (m, 1H), 6.62 (dd, 1H), 4.73-4.54 (m, 1H), 3.93 (dd, 2H), 3.27-2.53 (m, 4H), 2.11 (dq, 1H), 1.97-1.80 (m, 1H), 1.70-0.89 (m, 3H), 0.90-0.76 (m, 6H); LC-MS: m/z 405.2 (MH+).

### Scheme 4, Cpd (III)*| Preparation: 4-(4,4-difluoropiperidine-1-carbonyl)-3-(1-methylpyrazol-3-yl)benzonitrile

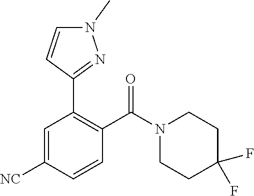 [SMILES: Cn1ccc(-c2cc(C#N)ccc2C(=O)N2CCC(F)(F)CC2)n1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-(4,4-difluoropiperidine-1-carbonyl)benzonitrile and 1-methyl-1H-pyrazole-3-boronic acid pinacol ester were used instead of 3-bromo-4-(4-methoxy-4-methyl-piperidine-1-carbonyl)benzonitrile and 1-(propan-2-yl)-3-(tetramethyl-1,3,2-dioxaborolan-2-yl)-1H-pyrazole respectively. The title compound was obtained in a yield of 56% (56 mg).

¹H NMR (400 MHZ, DMSO-d₆) δ 8.20 (d, 1H), 7.85 (dd, 1H), 7.80 (d, 1H), 7.51 (d, 1H), 6.67 (d, 1H), 3.97-3.84 (m, 4H), 3.59 (ddd, 1H), 3.24-3.05 (m, 2H), 2.21-1.97 (m, 2H), 1.94-1.79 (m, 1H), 1.78-1.59 (m, 1H); LC-MS: m/z 331.12 (MH+).

### Scheme 4, Cpd (III)*m Preparation: 3-(1-cyclobutylpyrazol-3-yl)-4-[4-(trifluoromethyl) piperidine-1-carbonyl]benzonitrile

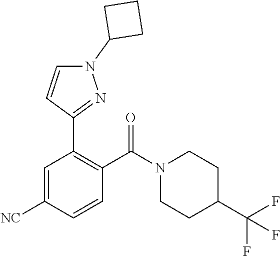 [SMILES: N#Cc1ccc(C(=O)N2CCC(C(F)(F)F)CC2)c(-c2ccn(C3CCC3)n2)c1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 3-bromo-4-[4-(trifluoromethyl) piperidine-1-carbonyl]benzonitrile and 1-cyclobutyl-3-(4,4,5,5-tetramethyl-1,3,2-dioxaborolan-2-yl)pyrazole (see Scheme 12, Cpd (III)*a) were used instead of 3-bromo-4-(4-methoxy-4-methyl-piperidine-1-carbonyl)benzonitrile and 1-(propan-2-yl)-3-(tetramethyl-1,3,2-dioxaborolan-2-yl)-1H-pyrazole respectively. The title compound was obtained in a yield of 79% (165 mg).

1H NMR (400 MHZ, DMSO) δ 8.17 (dd, 1H), 7.92 (q, 1H), 7.84 (ddd, 1H), 7.49 (dd, 1H), 6.60 (dd, 1H), 4.84 (p, 1H), 4.62 (d, 1H), 3.21-3.11 (m, 1H), 3.03-2.69 (m, 2H), 2.57 (d, 1H), 2.44-2.30 (m, 3H), 1.97-1.65 (m, 4H), 1.56-1.12 (m, 3H); LC-MS: m/z 403.2 (MH+).

### Scheme 4, Cpd (III)*n Preparation: 4-(6-azaspiro[2.5]octane-6-carbonyl)-3-(1-propan-2-ylpyrazol-4-yl)benzonitrile

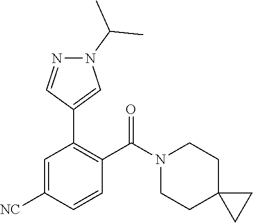 [SMILES: CC(C)n1cc(-c2cc(C#N)ccc2C(=O)N2CCC3(CC2)CC3)cn1]

The synthesis of the title compound was effected analogously to the synthesis of the compound 3-(1-isopropylpyrazol-3-yl)-4-(4-methoxy-4-methyl-piperidine-1-carbonyl)benzonitrile wherein 4-(6-azaspiro[2.5]octane-6-carbonyl)-3-bromobenzonitrile and 1-Isopropyl-4-(4,4,5,5-tetramethyl-1,3,2-dioxaborolan-2-yl)-1H-pyrazole were used instead of 3-bromo-4-(4-methoxy-4-methyl-piperidine-1-carbonyl)benzonitrile and 1-(propan-2-yl)-3-(tetramethyl-1,3,2-dioxaborolan-2-yl)-1H-pyrazole respectively. The title compound was obtained in a yield of 78% (85 mg).

1H NMR (400 MHZ, DMSO) δ 8.08 (d, 1H), 8.03 (d1H), 7.74 (dd, 1H), 7.72 (d, 1H), 7.45 (d, 1H), 4.57 (p, 1H), 3.95-3.82 (m, 1H), 3.43 (ddd, 1H), 3.13-2.93 (m, 1H), 2.93-2.83 (m, 1H), 1.44 (dd, 6H), 1.41-1.33 (m, 1H), 1.27 (s, 1H), 0.99-0.90 (m, 1H), 0.71 (td, 1H), 0.37-0.14 (m, 4H); LC-MS: m/z 349.18 (MH+).

### Scheme 4, Cpd (IV)*a Preparation: [2-(1-isopropylpyrazol-3-yl)-4-(5-methyl-4H-1,2,4-triazol-3-yl)phenyl]-(4-methoxy-4-methyl-1-piperidyl)methanone

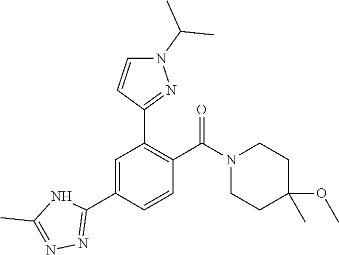

### Example 39

A mixture of 3-(1-isopropylpyrazol-3-yl)-4-(4-methoxy-4-methyl-piperidine-1-carbonyl)benzonitrile (120.0 mg, 0.330 mmol), dicesium carbonate (320.08 mg, 0.980 mmol), ethanimidamide hydrochloride (46.44 mg, 0.490 mmol) and CuBr (2.35 mg, 0.020 mmol) in DMSO (3.713 mL) was stirred at 120° C. for 8 h. After this time the reaction was cooled down to RT and H₂O was added, the mixture was then extracted with AcOEt 3 times. The aqueous phase was acidified until pH=5 with HCl 1M and extracted with AcOEt 3 times. The combined organic fractions were washed with brine, dried over Na₂SO₄, filtered, concentrated under vacuum and purified by FC on NH column (from 100% of DCM to DCM/MeOH 95:5) to give the product of formula [2-(1-isopropylpyrazol-3-yl)-4-(5-methyl-4H-1,2,4-triazol-3-yl)phenyl]-(4-methoxy-4-methyl-1-piperidyl)methanone.

### Yield 63 mg

1H NMR (400 MHZ, DMSO) δ 13.78 (s, 1H), 8.33 (dd, 1H), 7.93 (ddd, 1H), 7.82 (dd, 1H), 7.30 (dd, 1H), 6.43 (dd, 1H), 4.55 (dp, 1H), 4.28-3.96 (m, 1H), 3.15-2.86 (m, 6H), 2.41 (s, 3H), 1.72 (t, 1H), 1.53-1.20 (m, 9H), 1.14-0.87 (m, 3H); LC-MS: m/z 423.5 (MH+).

### Preparation: (4-tert-butyl-1-piperidyl)-[2-(1-isopropylpyrazol-3-yl)-4-(5-methyl-4H-1,2,4-triazol-3-yl)phenyl]methanone

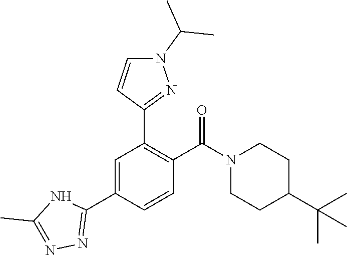 [SMILES: Cc1nnc(-c2ccc(C(=O)N3CCC(C(C)(C)C)CC3)c(-c3ccn(C(C)C)n3)c2)[nH]1]

### Example 40

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(4-tert-butylpiperidine-1-carbonyl)-3-(1-isopropylpyrazol-3-yl)benzonitrile was used instead of 3-(1-isopropylpyrazol-3-yl)-4-(4-methoxy-4-methyl-piperidine-1-carbonyl)benzonitrile. The title compound was obtained in a yield of 20% (22 mg).

1H NMR (400 MHZ, DMSO-d6) δ ppm 8.37 (s, 1H), 7.98-7.90 (m, 1H), 7.79 (s, 1H), 7.36-7.23 (m, 1H), 6.44-6.36 (m, 1H), 4.74-4.45 (m, 2H), 3.28-3.18 (m, 2H), 2.85-2.55 (m, 2H), 2.41 (s, 3H), 1.83-1.41 (m, 7H), 1.29-0.93 (m, 3H), 0.88-0.66 (m, 9H), 0.29-0.15 (m, 1H); 435.38 (MH+).

### Preparation: [2-(1-isopropylpyrazol-3-yl)-4-(5-methyl-4H-1,2,4-triazol-3-yl)phenyl]-(1-oxa-7-azaspiro[3.5]nonan-7-yl)methanone

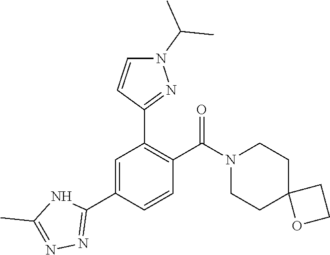 [SMILES: Cc1nnc(-c2ccc(C(=O)N3CCC4(CCO4)CC3)c(-c3ccn(C(C)C)n3)c2)[nH]1]

### Example 41

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 3-(1-isopropylpyrazol-3-yl)-4-(1-oxa-7-azaspiro[3.5]nonane-7-carbonyl)benzonitrile was used instead of 3-(1-isopropylpyrazol-3-yl)-4-(4-methoxy-4-methyl-piperidine-1-carbonyl)benzonitrile. The title compound was obtained in a yield of 50% (37 mg).

1H NMR (400 MHZ, DMSO-d6) δ ppm 8.35-8.28 (m, 1H), 7.98-7.91 (m, 1H), 7.84-7.76 (m, 1H), 7.32-7.26 (m, 1H), 6.46-6.39 (m, 1H), 4.60-4.48 (m, 1H), 4.43-4.28 (m, 2H), 3.75-3.42 (m, 3H), 3.13-2.84 (m, 2H), 2.45-2.16 (m, 4H), 1.88-1.67 (m, 2H), 1.61-1.52 (m, 1H), 1.49-1.39 (m, 6H), 1.30-1.06 (m, 2H); LC-MS: m/z 421.48 (MH+).

### Preparation: (4-ethoxy-1-piperidyl)-[2-(1-isopropylpyrazol-3-yl)-4-(5-methyl-4H-1,2,4-triazol-3-yl)phenyl]methanone

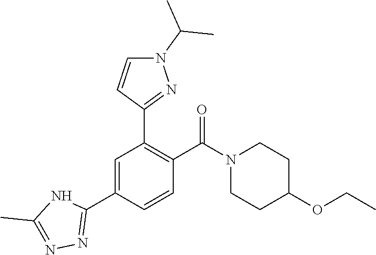 [SMILES: CCOC1CCN(C(=O)c2ccc(-c3nnc(C)[nH]3)cc2-c2ccn(C(C)C)n2)CC1]

### Example 42

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(4-ethoxypiperidine-1-carbonyl)-3-(1-isopropylpyrazol-3-yl)benzonitrile was used instead of 3-(1-isopropylpyrazol-3-yl)-4-(4-methoxy-4-methyl-piperidine-1-carbonyl)benzonitrile. The title compound was obtained in a yield of 38% (80 mg). 1H NMR (400 MHZ, DMSO) δ 13.79 (s, 1H), 8.33 (dd, 1H), 7.93 (dt, 1H), 7.81 (dd, 1H), 7.30 (dd, 1H), 6.42 (t, 1H), 4.53 (dp, 1H), 4.05-3.91 (m, 1H), 3.48-3.36 (m, 2H), 3.28-3.05 (m, 3H), 2.95-2.74 (m, 1H), 2.41 (s, 3H), 1.79 (d, 1H), 1.55-1.32 (m, 8H), 1.29-1.18 (m, 1H), 1.06 (dt, 3H); LC-MS: m/z 423.3 (MH+).

### Preparation: (4-fluoro-4-methylpiperidin-1-yl)-[4-(5-methyl-4H-1,2,4-triazol-3-yl)-2-(1-propan-2-ylpyrazol-3-yl)phenyl]methanone

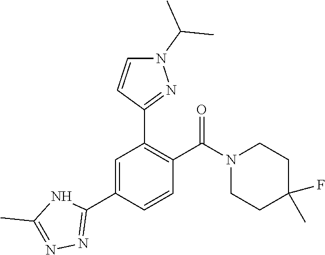 [SMILES: Cc1nnc(-c2ccc(C(=O)N3CCC(C)(F)CC3)c(-c3ccn(C(C)C)n3)c2)[nH]1]

### Example 43

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(4-fluoro-4-methylpiperidine-1-carbonyl)-3-(1-propan-2-ylpyrazol-3-yl)benzonitrile was used instead of 3-(1-isopropylpyrazol-3-yl)-4-(4-methoxy-4-methyl-piperidine-1-carbonyl)benzonitrile. The title compound was obtained in a yield of 75% (32 mg). 1H NMR (400 MHZ, DMSO) δ 13.80 (s, 1H), 8.33 (dd, 1H), 7.94 (td, 1H), 7.82 (dd, 1H), 7.33 (t, 1H), 6.43 (dd, 1H), 4.55 (dp, 1H), 4.40-4.13 (m, 1H), 3.19-2.86 (m, 3H), 2.41 (s, 3H), 1.86-1.40 (m, 10H), 1.37-1.13 (m, 3H); LC-MS: m/z 411.6 (MH+).

### Preparation: (4,4-difluoropiperidin-1-yl)-[2-(2-ethylpyrazol-3-yl)-4-(5-methyl-4H-1,2,4-triazol-3-yl)phenyl]methanone

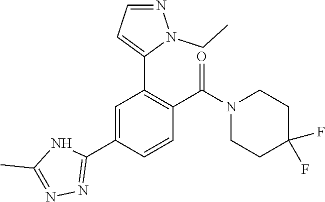 [SMILES: CCn1nccc1-c1cc(-c2nnc(C)[nH]2)ccc1C(=O)N1CCC(F)(F)CC1]

### Example 44

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(4,4-difluoropiperidine-1-carbonyl)-3-(2-ethylpyrazol-3-yl)benzonitrile was used instead of 3-(1-isopropylpyrazol-3-yl)-4-(4-methoxy-4-methyl-piperidine-1-carbonyl)benzonitrile. The title compound was obtained in a yield of 60% (53 mg).

1H NMR (400 MHZ, DMSO-d6) δ ppm 8.15-8.08 (m, 1H), 7.96 (d, 1H), 7.52 (d, 2H), 6.26 (d, 1H), 4.01 (d, 2H), 3.68-3.49 (m, 2H), 3.25-3.01 (m, 3H), 2.41 (s, 3H), 2.10-1.64 (m, 4H), 1.32 (t, 3H); LC-MS: m/z 401.23 (MH+).

### Preparation: [2-[1-(2-methylpropyl) pyrazol-3-yl]-4-(5-methyl-4H-1,2,4-triazol-3-yl)phenyl]-[4-(trifluoromethyl)piperidin-1-yl]methanone

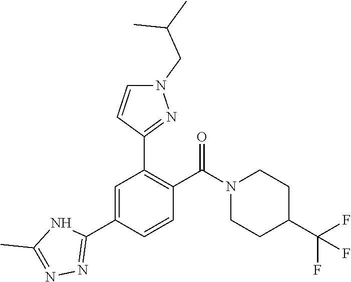 [SMILES: Cc1nnc(-c2ccc(C(=O)N3CCC(C(F)(F)F)CC3)c(-c3ccn(CC(C)C)n3)c2)[nH]1]

### Example 45

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 3-[1-(2-methylpropyl) pyrazol-3-yl]-4-[4-(trifluoromethyl) piperidine-1-carbonyl]benzonitrile was used instead of 3-(1-isopropylpyrazol-3-yl)-4-(4-methoxy-4-methyl-piperidine-1-carbonyl)benzonitrile. The title compound was obtained in a yield of 10% (4 mg).

1H NMR (400 MHZ, DMSO) δ 13.80 (s, 1H), 8.36 (dd, 1H), 7.95 (ddd, 1H), 7.77 (dd, 1H), 7.39-7.25 (m, 1H), 6.52-6.37 (m, 1H), 4.75-4.58 (m, 1H), 3.99-3.91 (m, 2H), 3.02-2.55 (m, 3H), 2.41 (s, 3H), 2.21-2.06 (m, 1H), 1.97-1.79 (m, 1H), 1.73-0.89 (m, 4H), 0.88 (s, 6H); LC-MS: m/z 461.2 (MH+).

### Preparation: (4,4-difluoropiperidin-1-yl)-[2-(1-methylpyrazol-3-yl)-4-(5-methyl-4H-1,2,4-triazol-3-yl)phenyl]methanone

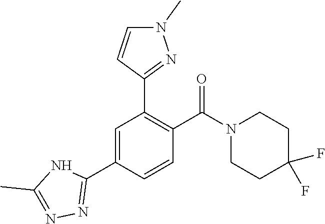 [SMILES: Cc1nnc(-c2ccc(C(=O)N3CCC(F)(F)CC3)c(-c3ccn(C)n3)c2)[nH]1]

### Example 46

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(4,4-difluoropiperidine-1-carbonyl)-3-(1-methylpyrazol-3-yl)benzonitrile was used instead of 3-(1-isopropylpyrazol-3-yl)-4-(4-methoxy-4-methyl-piperidine-1-carbonyl)benzonitrile. The title compound was obtained in a yield of 36% (23 mg).

1H NMR (400 MHZ, DMSO-d6) δ ppm 13.93-13.69 (m, 1H), 8.34 (d, 1H), 7.95 (dd, 1H), 7.77 (d, 1H), 7.37 (d, 1H), 6.49 (d, 1H), 3.88 (s, 4H), 3.64-3.49 (m, 1H), 3.25-3.09 (m, 2H), 2.41 (s, 3H), 2.17-1.77 (m, 3H), 1.69-1.49 (m, 1H); LC-MS: m/z 387.19 (MH+).

### Preparation: [2-(1-cyclobutylpyrazol-3-yl)-4-(5-methyl-4H-1,2,4-triazol-3-yl)phenyl]-[4-(trifluoromethyl)piperidin-1-yl]methanone

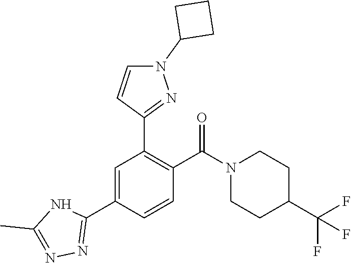 [SMILES: Cc1nnc(-c2ccc(C(=O)N3CCC(C(F)(F)F)CC3)c(-c3ccn(C4CCC4)n3)c2)[nH]1]

### Example 47

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 3-(1-cyclobutylpyrazol-3-yl)-4-[4-(trifluoromethyl) piperidine-1-carbonyl]benzonitrile was used instead of 3-(1-isopropylpyrazol-3-yl)-4-(4-methoxy-4-methyl-piperidine-1-carbonyl)benzonitrile. The title compound was obtained in a yield of 36% (67 mg).

1H NMR (400 MHZ, DMSO) δ 13.80 (s, 1H), 8.33 (dd, 1H), 7.96 (ddd, 1H), 7.86 (dd, 1H), 7.35 (dd, 1H), 6.46 (t, 1H), 4.87 (p, 1H), 4.66 (d, 1H), 3.40 (d, 1H), 3.21 (d, 1H), 3.03-2.70 (m, 2H), 2.56 (d, 1H), 2.47-2.31 (m, 6H), 2.03-1.63 (m, 3H), 1.56-1.18 (m, 2H), 0.40 (qd, 1H); LC-MS: m/z 459.48 (MH+).

### Preparation: [4-(5-methyl-4H-1,2,4-triazol-3-yl)-2-(1-propan-2-ylpyrazol-3-yl)phenyl]-[4-(trifluoromethyl)piperidin-1-yl]methanone

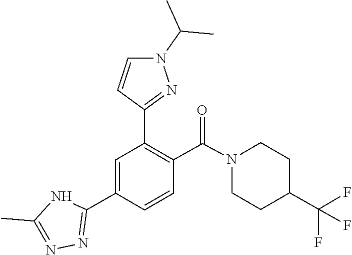 [SMILES: Cc1nnc(-c2ccc(C(=O)N3CCC(C(F)(F)F)CC3)c(-c3ccn(C(C)C)n3)c2)[nH]1]

### Example 48

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 3-(1-propan-2-ylpyrazol-3-yl)-4-[4-(trifluoromethyl) piperidine-1-carbonyl]benzonitrile was used instead of 3-(1-isopropylpyrazol-3-yl)-4-(4-methoxy-4-methyl-piperidine-1-carbonyl)benzonitrile. The title compound was obtained in a yield of 27% (67 mg).

1H NMR (400 MHZ, DMSO) δ 13.80 (s, 1H), 8.43-8.23 (m, 1H), 7.95 (ddd, 1H), 7.81 (dd, 1H), 7.39-7.27 (m, 1H), 6.42 (d, 1H), 4.66 (d, 1H), 4.54 (pd, 1H), 3.47-2.45 (m, 4H), 2.41 (s, 3H), 1.46 (s, 2H), 1.44 (dd, 6H), 1.38-0.29 (m, 2H); LC-MS: m/z 447.2 (MH+).

### Preparation: 6-azaspiro[2.5]octan-6-yl-[4-(5-methyl-4H-1,2,4-triazol-3-yl)-2-(1-propan-2-ylpyrazol-4-yl)phenyl]methanone

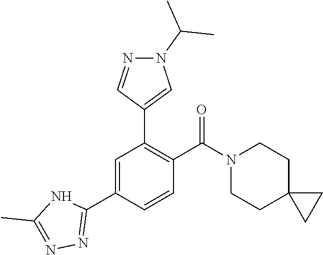 [SMILES: Cc1nnc(-c2ccc(C(=O)N3CCC4(CC3)CC4)c(-c3cnn(C(C)C)c3)c2)[nH]1]

### Example 49

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(6-azaspiro[2.5]octane-6-carbonyl)-3-(1-propan-2-ylpyrazol-4-yl)benzonitrile was used instead of 3-(1-isopropylpyrazol-3-yl)-4-(4-methoxy-4-methyl-piperidine-1-carbonyl)benzonitrile. The title compound was obtained in a yield of 19% (18 mg).

1H NMR (400 MHZ, DMSO) δ 13.77 (s, 1H), 8.09 (d, 1H), 7.97 (s, 1H), 7.88 (dd, 1H), 7.64 (s, 1H), 7.32 (d, 1H), 4.58 (hept, 1H), 3.96-3.80 (m, 1H), 3.41 (ddd, 1H), 3.09-2.88 (m, 2H), 2.41 (s, 3H), 1.45 (dd, 6H), 1.38 (ddd, 1H), 1.23 (dt, 1H), 0.92 (dt, 1H), 0.71 (ddd, 1H), 0.38-0.13 (m, 4H); LC-MS: m/z 405.57 (MH+).

### Preparation: 6-azaspiro[2.5]octan-6-yl-[4-(5-methyl-4H-1,2,4-triazol-3-yl)-2-(1-propan-2-ylpyrazol-3-yl)phenyl]methanone

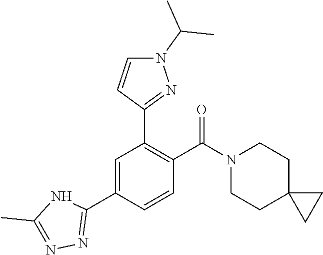 [SMILES: Cc1nnc(-c2ccc(C(=O)N3CCC4(CC3)CC4)c(-c3ccn(C(C)C)n3)c2)[nH]1]

### Example 50

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(6-azaspiro[2.5]octane-6-carbonyl)-3-(1-propan-2-ylpyrazol-3-yl)benzonitrile was used instead of 3-(1-isopropylpyrazol-3-yl)-4-(4-methoxy-4-methyl-piperidine-1-carbonyl)benzonitrile. The title compound was obtained in a yield of 39% (31 mg).

1H NMR (400 MHZ, DMSO-d6) δ 13.80 (s, 1H), 8.35 (d, 1H), 7.94 (dd, 1H), 7.84 (d, 1H), 7.32 (d, 1H), 6.46 (d, 1H), 4.57 (p, 1H), 3.77-3.54 (m, 2H), 3.04 (dddd, 2H), 2.42 (s, 3H), 1.47 (dd, 6H), 1.35 (q, 2H), 1.16-1.06 (m, 1H), 0.85 (ddd, 1H), 0.44-0.05 (m, 4H); LC-MS: m/z 405.24 (MH+).

### Preparation: (4,4-difluoropiperidin-1-yl)-[4-(5-methyl-4H-1,2,4-triazol-3-yl)-2-(1-propan-2-ylpyrazol-3-yl)phenyl]methanone

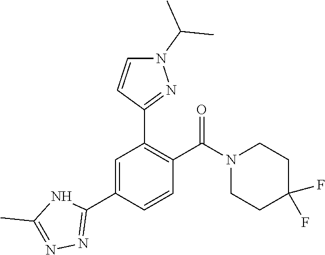 [SMILES: Cc1nnc(-c2ccc(C(=O)N3CCC(F)(F)CC3)c(-c3ccn(C(C)C)n3)c2)[nH]1]

### Example 51

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(4,4-difluoropiperidine-1-carbonyl)-3-(1-propan-2-ylpyrazol-3-yl)benzonitrile was used instead of 3-(1-isopropylpyrazol-3-yl)-4-(4-methoxy-4-methyl-piperidine-1-carbonyl)benzonitrile. The title compound was obtained in a yield of 48% (50 mg).

1H NMR (400 MHZ, DMSO-d6) δ ppm 14.61-12.76 (m, 1H), 8.31 (d, 1H), 7.96 (dd, 1H), 7.84 (d, 1H), 7.38 (d, 1H), 6.48 (d, 1H), 4.54 (spt, 1H), 3.97-3.78 (m, 1H), 3.64-3.52 (m, 1H), 3.23-3.02 (m, 2H), 2.41 (s, 3H), 2.16-1.70 (m, 3H), 1.44 (dd, 6H), 1.54-1.19 (m, 1H); LC-MS: m/z 415.3 (MH+).

### Preparation: (6,6-difluoro-2-azaspiro[3.3]heptan-2-yl)-[2-(1-isopropylpyrazol-3-yl)-4-(5-methyl-4H-1,2,4-triazol-3-yl)phenyl]methanone

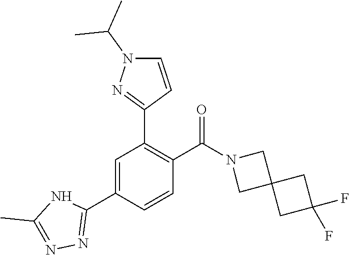 [SMILES: Cc1nnc(-c2ccc(C(=O)N3CC4(C3)CC(F)(F)C4)c(-c3ccn(C(C)C)n3)c2)[nH]1]

### Example 52

The synthesis of the title compound was effected analogously to the synthesis of the compound of Example 39 [2-(1-isopropylpyrazol-3-yl)-4-(5-methyl-4H-1,2,4-triazol-3-yl)phenyl]-(4-methoxy-4-methyl-1-piperidyl)methanone wherein 4-(6,6-difluoro-2-azaspiro[3.3]heptane-2-carbonyl)-3-(1-isopropylpyrazol-3-yl)benzonitrile was used instead of 3-(1-isopropylpyrazol-3-yl)-4-(4-methoxy-4-methyl-piperidine-1-carbonyl)benzonitrile. The title compound was obtained in a yield of 35% (21 mg).

1H NMR (400 MHZ, DMSO-d6) δ 13.73 (s, 1H), 8.30 (d, 1H), 7.93 (d, 1H), 7.84 (d, 1H), 7.33 (d, 1H), 6.48 (d, 1H), 4.56 (p, 1H), 4.06 (s, 2H), 3.69 (s, 2H), 2.89-2.56 (m, 4H), 2.42 (s, 3H), 1.47 (d, 6H); LC-MS: m/z 427.30

### Scheme 4, Cpd (V)*a Preparation: [2-bromo-4-(5-methyl-4H-1,2,4-triazol-3-yl)phenyl]-[4-(trifluoromethoxy)piperidin-1-yl]methanone

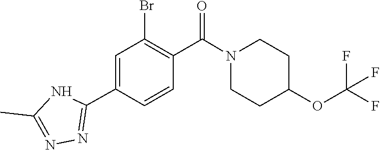 [SMILES: Cc1nnc(-c2ccc(C(=O)N3CCC(OC(F)(F)F)CC3)c(Br)c2)[nH]1]

A mixture of 3-bromo-4-[4-(trifluoromethoxy) piperidine-1-carbonyl]benzonitrile (268.0 mg, 0.710 mmol), dicesium carbonate (694.56 mg, 2.13 mmol), ethanimidamide hydrochloride (100.77 mg, 1.07 mmol) and CuBr (5.1 mg, 0.040 mmol) in DMSO (3.5 mL) was stirred at 120° C. for 7 h. After this time the reaction was cooled down, and directly purified by FC on reverse phase using acid conditions (eluting from 5:95 of CH₃CN/H₂O+0.1% of HCOOH to 50:50 of CH₃CN/H₂O+0.1% of HCOOH) affording the product of formula [2-bromo-4-(5-methyl-4H-1,2,4-triazol-3-yl)phenyl]-[4-(trifluoromethoxy)piperidin-1-yl]methanone.

### Yield: 34 mg

1H NMR (400 MHZ, DMSO) δ 13.88 (s, 1H), 8.18 (d, 1H), 8.05-7.98 (m, 1H), 7.54-7.39 (m, 1H), 4.74 (dt, 1H), 4.08-3.98 (m, 1H), 3.50-3.37 (m, 2H), 3.30-3.11 (m, 1H), 2.41 (s, 3H), 2.14-1.59 (m, 4H); LC-MS: m/z 433.07, 435.05 (MH+).

### Scheme 4, Cpd (V)*b Preparation: [2-bromo-4-(5-methyl-4H-1,2,4-triazol-3-yl)phenyl]-(4-methylpiperidin-1-yl)methanone

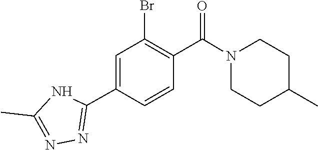

The synthesis of the title compound was effected analogously to the synthesis of the compound [2-bromo-4-(5-methyl-4H-1,2,4-triazol-3-yl)phenyl]-[4-(trifluoromethoxy)piperidin-1-yl]methanone wherein 3-bromo-4-(4-methylpiperidine-1-carbonyl)benzonitrile was used instead of 3-bromo-4-[4-(trifluoromethoxy) piperidine-1-carbonyl]benzonitrile. The title compound was obtained in a yield of 61% (65 mg).

1H NMR (400 MHZ, DMSO) δ 13.82 (s, 1H), 8.17 (d, 1H), 8.00 (ddd, 1H), 7.35 (d, 1H), 4.46 (s, 1H), 3.23 (s, 1H), 3.08-2.92 (m, 1H), 2.77 (dt, 1H), 2.41 (s, 3H), 1.77-1.43 (m, 3H), 1.23-0.98 (m, 2H), 0.92 (d, 3H); LC-MS: m/z 363.10, 365.12 (MH+).

### Scheme 4, Cpd (V)*c Preparation: [2-bromo-4-(5-methyl-4H-1,2,4-triazol-3-yl)phenyl]-(4-methoxypiperidin-1-yl)methanone

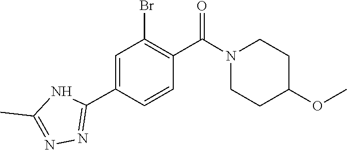

The synthesis of the title compound was effected analogously to the synthesis of the compound [2-bromo-4-(5-methyl-4H-1,2,4-triazol-3-yl)phenyl]-[4-(trifluoromethoxy)piperidin-1-yl]methanone wherein 3-bromo-4-(4-methoxypiperidine-1-carbonyl)benzonitrile was used instead of 3-bromo-4-[4-(trifluoromethoxy) piperidine-1-carbonyl]benzonitrile. The title compound was obtained in a yield of 52% (120 mg).

1H NMR (400 MHZ, DMSO) δ 13.86 (s, 1H), 8.17 (d, 1H), 8.00 (dd, 1H), 7.41 (t, 1H), 3.93 (d, 1H), 3.44 (d, 1H), 3.39-3.32 (m, 2H), 3.25 (s, 3H), 3.10-2.96 (m, 1H), 2.41 (s, 3H), 1.96-1.86 (m, 1H), 1.79 (s, 1H), 1.56-1.32 (m, 2H); LC-MS: m/z 379.36, 381.36 (MH+).

### Scheme 4, Cpd (V)*d Preparation: [2-bromo-4-(5-methyl-4H-1,2,4-triazol-3-yl)phenyl]-[4-(trifluoromethyl)piperidin-1-yl]methanone

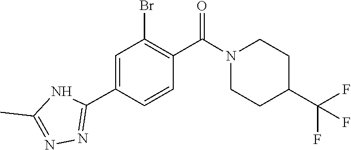 [SMILES: Cc1nnc(-c2ccc(C(=O)N3CCC(C(F)(F)F)CC3)c(Br)c2)[nH]1]

The synthesis of the title compound was effected analogously to the synthesis of the compound [2-bromo-4-(5-methyl-4H-1,2,4-triazol-3-yl)phenyl]-[4-(trifluoromethoxy)piperidin-1-yl]methanone wherein 3-bromo-4-[4-(trifluoromethyl) piperidine-1-carbonyl]benzonitrile was used instead of 3-bromo-4-[4-(trifluoromethoxy) piperidine-1-carbonyl]benzonitrile. The title compound was obtained in a yield of 54% (252 mg).

1H NMR (400 MHZ, DMSO) δ 13.87 (s, 1H), 8.18 (dd, 1H), 8.01 (dt, 1H), 7.51 (dd, 1H), 4.62 (s, 1H), 3.36 (d, 1H), 3.19-3.03 (m, 1H), 2.83 (t, 1H), 2.71-2.61 (m, 1H), 2.41 (s, 3H), 1.94 (s, 1H), 1.84-1.71 (m, 1H), 1.57-1.27 (m, 2H); LC-MS: m/z 417.05, 419.07 (MH+).

### Scheme 4, Cpd (V)*e Preparation: [2-bromo-4-(5-methyl-4H-1,2,4-triazol-3-yl)phenyl]-(4,4-difluoropiperidin-1-yl)methanone

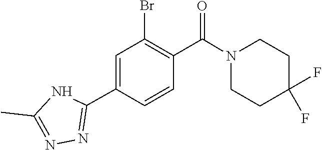 [SMILES: Cc1nnc(-c2ccc(C(=O)N3CCC(F)(F)CC3)c(Br)c2)[nH]1]

The synthesis of the title compound was effected analogously to the synthesis of the compound [2-bromo-4-(5-methyl-4H-1,2,4-triazol-3-yl)phenyl]-[4-(trifluoromethoxy)piperidin-1-yl]methanone wherein 3-bromo-4-[4-(trifluoromethyl) piperidine-1-carbonyl]benzonitrile was used instead of 3-bromo-4-[4-(trifluoromethoxy) piperidine-1-carbonyl]benzonitrile. The title compound was obtained in a yield of 53% (268 mg).

¹H NMR (400 MHZ, DMSO-d₆) δ 13.88 (s, 1H), 8.20 (d, 1H), 8.03 (dd, 1H), 7.52 (d, 1H), 3.92-3.81 (m, 1H), 3.74-3.63 (m, 1H), 3.30-3.14 (m, 2H), 2.42 (s, 3H), 2.17-1.92 (m, 4H); LC-MS: m/z 385.05, 387.06 (MH+).

### Preparation: [2-(1-tert-butylpyrazol-3-yl)-4-(5-methyl-4H-1,2,4-triazol-3-yl)phenyl]-[4-(trifluoromethyl)-1-piperidyl]methanone

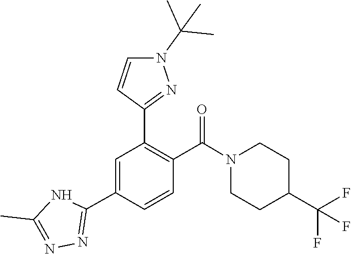 [SMILES: Cc1nnc(-c2ccc(C(=O)N3CCC(C(F)(F)F)CC3)c(-c3ccn(C(C)(C)C)n3)c2)[nH]1]

### Example 53

A mixture of tripotassium phosphate (154.67 mg, 0.730 mmol), X-Phos amonobiphenyl palladium chloride precatalyst (15.07 mg, 0.020 mmol), 1-tert-butyl-3-(4,4,5,5-tetramethyl-1,3,2-dioxaborolan-2-yl)pyrazole (95 mg, 0.380 mmol) and [2-bromo-4-(5-methyl-4H-1,2,4-triazol-3-yl)phenyl]-[4-(trifluoromethyl)piperidin-1-yl]methanone (80.0 mg, 0.190 mmol) in 1,2-dimethoxyethane (2.128 mL)/Water (0.142 mL) was degassed with N₂ for 5 min and then by N₂/vacuum cycles, 3 times. The reaction was stirred ON at 90° C. The reaction mixture was cooled down to room temperature and diluted with water and extracted with EtOAc (3×). The organic layers were collected together and washed with brine, dried over Na₂SO₄, filtered and the solvent removed under vacuum. The mixture was purified by FC on NH column (eluting from 100% of DCM to DCM/MeOH to 95:5) and again by FC on reverse phase using acid conditions (eluting from 5:95 of CH₃CN/H₂O+0.1% of HCOOH to 60:40 of CH₃CN/H₂O+0.1% of HCOOH) affording the product of formula [2-(1-tert-butylpyrazol-3-yl)-4-(5-methyl-4H-1,2,4-triazol-3-yl)phenyl]-[4-(trifluoromethyl)-1-piperidyl]methanone.

### Yield: 35 mg

1H NMR (400 MHZ, DMSO) δ 13.80 (s, 1H), 8.30 (dd, 1H), 7.95 (ddd, 1H), 7.86 (t, 1H), 7.33 (dd, 1H), 6.60-6.26 (m, 1H), 4.65 (d, 1H), 3.45-3.16 (m, 1H), 2.93-2.67 (m, 2H), 2.49 (d, 1H), 2.41 (s, 3H), 1.97-1.63 (m, 1H), 1.55 (d, 9H), 1.47-0.09 (m, 3H); LC-MS: m/z 461.3 (MH+).

### Preparation: [2-(1-tert-butylpyrazol-3-yl)-4-(5-methyl-4H-1,2,4-triazol-3-yl)phenyl]-(4,4-difluoropiperidin-1-yl)methanone

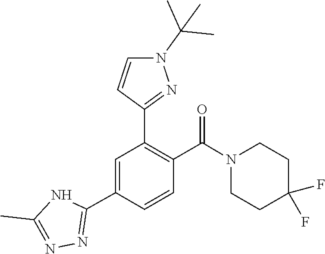 [SMILES: Cc1nnc(-c2ccc(C(=O)N3CCC(F)(F)CC3)c(-c3ccn(C(C)(C)C)n3)c2)[nH]1]

### Example 54

The synthesis of the title compound was effected analogously to the synthesis of the compound of Example 53 [2-(1-tert-butylpyrazol-3-yl)-4-(5-methyl-4H-1,2,4-triazol-3-yl)phenyl]-[4-(trifluoromethyl)-1-piperidyl]methanone wherein [2-bromo-4-(5-methyl-4H-1,2,4-triazol-3-yl)phenyl]-(4,4-difluoropiperidin-1-yl)methanone was used instead of [2-bromo-4-(5-methyl-4H-1,2,4-triazol-3-yl)phenyl]-[4-(trifluoromethyl)piperidin-1-yl]methanone. The title compound was obtained in a yield of 61% (68 mg).

1H NMR (400 MHZ, DMSO-d6) δ ppm 8.29 (d, 1H) 7.92-7.98 (m, 1H) 7.89 (d, 1H) 7.34-7.43 (m, 1H) 6.44-6.50 (m, 1H) 3.67-3.77 (m, 2H) 3.00-3.25 (m, 2H) 2.41 (s, 3H) 1.75-2.15 (m, 3H) 1.55 (s, 9H) 1.25-1.37 (m, 2H); LC-MS: m/z 429.29 (MH+).

### Preparation: [2-(1-cyclobutylpyrazol-3-yl)-4-(5-methyl-4H-1,2,4-triazol-3-yl)phenyl]-(4,4-difluoropiperidin-1-yl)methanone

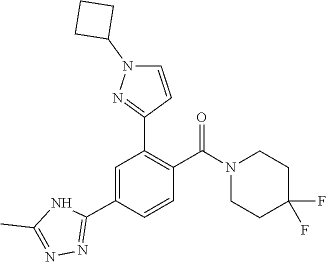 [SMILES: Cc1nnc(-c2ccc(C(=O)N3CCC(F)(F)CC3)c(-c3ccn(C4CCC4)n3)c2)[nH]1]

### Example 55

The synthesis of the title compound was effected analogously to the synthesis of the compound of Example 53 [2-(1-tert-butylpyrazol-3-yl)-4-(5-methyl-4H-1,2,4-triazol-3-yl)phenyl]-[4-(trifluoromethyl)-1-piperidyl]methanone wherein [2-bromo-4-(5-methyl-4H-1,2,4-triazol-3-yl)phenyl]-(4,4-difluoropiperidin-1-yl)methanone and 1-cyclobutyl-3-(4,4,5,5-tetramethyl-1,3,2-dioxaborolan-2-yl)pyrazole (see Scheme 12, Cpd (III)*a) were used instead of [2-bromo-4-(5-methyl-4H-1,2,4-triazol-3-yl)phenyl]-[4-(trifluoromethyl)piperidin-1-yl]methanone and 1-tert-butyl-3-(4,4,5,5-tetramethyl-1,3,2-dioxaborolan-2-yl)pyrazole respectively. The title compound was obtained in a yield of 37% (29 mg).

1H NMR (400 MHZ, DMSO-d6) δ ppm 13.91-13.70 (m, 1H), 8.31 (d, 1H), 7.99-7.94 (m, 1H), 7.88 (d, 1H), 7.39 (d, 1H), 6.51 (d, 1H), 4.87 (p, 1H), 3.82-3.68 (m, 2H), 3.26-3.09 (m, 2H), 2.48-2.35 (m, 6H), 2.15-1.75 (m, 6H), 1.56-1.38 (m, 1H); LC-MS: m/z 427.27 (MH+).

### Preparation: 1-[4-(5-methyl-4H-1,2,4-triazol-3-yl)-2-[1-(propan-2-yl)-1H-pyrazol-3-yl]benzoyl]-4-(trifluoromethoxy) piperidine

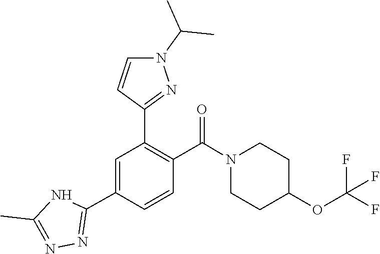 [SMILES: Cc1nnc(-c2ccc(C(=O)N3CCC(OC(F)(F)F)CC3)c(-c3ccn(C(C)C)n3)c2)[nH]1]

### Example 56

The synthesis of the title compound was effected analogously to the synthesis of the compound of Example 53 [2-(1-tert-butylpyrazol-3-yl)-4-(5-methyl-4H-1,2,4-triazol-3-yl)phenyl]-[4-(trifluoromethyl)-1-piperidyl]methanone wherein [2-bromo-4-(5-methyl-4H-1,2,4-triazol-3-yl)phenyl]-[4-(trifluoromethoxy)piperidin-1-yl]methanone and 1-(propan-2-yl)-3-(tetramethyl-1,3,2-dioxaborolan-2-yl)-1H-pyrazole were used instead of [2-bromo-4-(5-methyl-4H-1,2,4-triazol-3-yl)phenyl]-[4-(trifluoromethyl)piperidin-1-yl]methanone and 1-tert-butyl-3-(4,4,5,5-tetramethyl-1,3,2-dioxaborolan-2-yl)pyrazole respectively. The title compound was obtained in a yield of 29% (10 mg).

1H NMR (400 MHZ, DMSO) δ 13.78 (s, 1H), 8.33 (dd, 1H), 7.95 (ddd, 1H), 7.88-7.78 (m, 1H), 7.35 (dd, 1H), 6.45 (dd, 1H), 4.69-4.49 (m, 2H), 4.15-4.00 (m, 1H), 3.26-2.90 (m, 3H), 2.42 (s, 3H), 1.96 (d, 1H), 1.80-0.82 (m, 9H); LC-MS: m/z 463.5 (MH+).

### Preparation: [2-[1-(1-cyclopropylethyl) pyrazol-3-yl]-4-(5-methyl-4H-1,2,4-triazol-3-yl)phenyl]-(4,4-difluoro-1-piperidyl)methanone

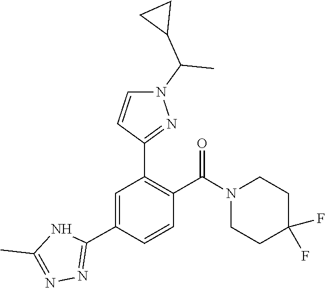 [SMILES: Cc1nnc(-c2ccc(C(=O)N3CCC(F)(F)CC3)c(-c3ccn(C(C)C4CC4)n3)c2)[nH]1]

### Example 57

The synthesis of the title compound was effected analogously to the synthesis of the compound of Example 53 [2-(1-tert-butylpyrazol-3-yl)-4-(5-methyl-4H-1,2,4-triazol-3-yl)phenyl]-[4-(trifluoromethyl)-1-piperidyl]methanone wherein [2-bromo-4-(5-methyl-4H-1,2,4-triazol-3-yl)phenyl]-(4,4-difluoropiperidin-1-yl)methanone and 1-(1-cyclopropylethyl)-3-(4,4,5,5-tetramethyl-1,3,2-dioxaborolan-2-yl)pyrazole were used instead of [2-bromo-4-(5-methyl-4H-1,2,4-triazol-3-yl)phenyl]-[4-(trifluoromethyl)piperidin-1-yl]methanone and 1-tert-butyl-3-(4,4,5,5-tetramethyl-1,3,2-dioxaborolan-2-yl)pyrazole respectively. The title compound was obtained in a yield of 30% (34 mg) as a racemic mixture.

¹H NMR (400 MHZ, DMSO-d₆) δ 8.32 (t, 1H), 8.00-7.93 (m, 1H), 7.87 (t, 1H), 7.39 (d, 1H), 6.48 (dd, 1H), 3.93-3.79 (m, 1H), 3.75-3.64 (m, 1H), 3.64-3.48 (m, 1H), 3.22-3.04 (m, 2H), 2.42 (s, 3H), 2.14-1.97 (m, 1H), 1.97-1.72 (m, 1H), 1.58-1.47 (m, 3H), 1.44-1.15 (m, 1H), 0.71-0.56 (m, 1H), 0.46-0.37 (m, 1H), 0.34 (t, 2H); LC-MS: m/z 441.25 (MH+).

The racemic mixture (Example 57) was then separated into single enantiomers (Examples 57a and 57b) by chiral semi-preparative HPLC

| | | |
|---|---|---|
| Preparative chiral | Column | Chiralpak IC (25 x 2.0 |
| chromatography | | cm), 5 μ |
| protocol: | Mobile phase | n-Hexane/(Ethanol + |
| | 0.1% IPA) | 82/18% v/v |
| | Flow rate (ml/min) | 17 ml/min |
| | DAD detection | 220 nm |
| | Loop | 1000 μL |
| | Total amount | 33 mg |
| | Injection | 9.4 mg (each |
| | | injection) |

| | |
|---|---|
| 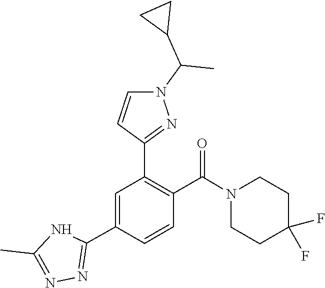 [SMILES: Cc1nnc(-c2ccc(C(=O)N3CCC(F)(F)CC3)c(-c3ccn(C(C)C4CC4)n3)c2)[nH]1] | Enantiomer 1 Rt 19.97 min 100% ee LC-MS: m/z 441.34 (MH+) [2-[1-(1R or 1S-cyclopropylethyl)pyrazol-3-yl]-4-(5- methyl-4H-1,2,4-triazol-3-yl)phenyl]-(4,4-difluoro-1- piperidyl)methanone Example 57a |
| 1R or 1S enantiomer | |
| 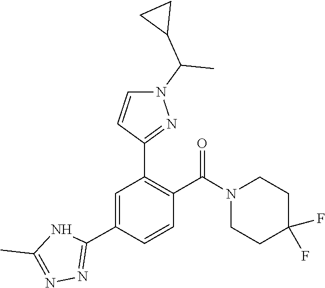 [SMILES: Cc1nnc(-c2ccc(C(=O)N3CCC(F)(F)CC3)c(-c3ccn(C(C)C4CC4)n3)c2)[nH]1] | Enantiomer 2 Rt 21.66 min 90% ee LC-MS: m/z 441.34 (MH+) [2-[1-(1R or 1S-cyclopropylethyl)pyrazol-3-yl]-4-(5- methyl-4H-1,2,4-triazol-3-yl)phenyl]-(4,4-difluoro-1- piperidyl)methanone Example 57b |
| 1R or 1S enantiomer | |

### Preparation: (4-methoxypiperidin-1-yl)-[4-(5-methyl-4H-1,2,4-triazol-3-yl)-2-(1-propan-2-ylpyrazol-3-yl)phenyl]methanone

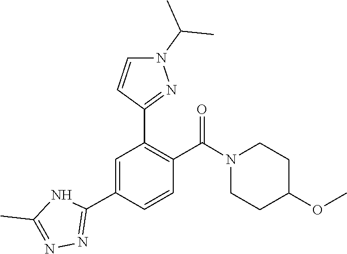

### Example 58

The synthesis of the title compound was effected analogously to the synthesis of the compound of Example 53 [2-(1-tert-butylpyrazol-3-yl)-4-(5-methyl-4H-1,2,4-triazol-3-yl)phenyl]-[4-(trifluoromethyl)-1-piperidyl]methanone wherein [2-bromo-4-(5-methyl-4H-1,2,4-triazol-3-yl)phenyl]-(4-methoxypiperidin-1-yl)methanone and 1-(propan-2-yl)-3-(tetramethyl-1,3,2-dioxaborolan-2-yl)-1H-pyrazole were used instead of [2-bromo-4-(5-methyl-4H-1,2,4-triazol-3-yl)phenyl]-[4-(trifluoromethyl)piperidin-1-yl]methanone and 1-tert-butyl-3-(4,4,5,5-tetramethyl-1,3,2-dioxaborolan-2-yl)pyrazole respectively. The title compound was obtained in a yield of 41% (55 mg).

1H NMR (400 MHZ, DMSO) δ 13.72 (s, 1H), 8.34 (dd, 1H), 7.93 (dt, 1H), 7.81 (dd, 1H), 7.30 (s, 1H), 6.42 (t, 1H), 4.54 (pd, 1H), 4.05-3.84 (m, 1H), 3.30 (s, 2H), 3.24-3.05 (m, 4H), 2.96-2.76 (m, 1H), 2.41 (s, 3H), 1.81 (s, 1H), 1.58-0.68 (m, 9H); LC-MS: m/z 409.2 (MH+).

### Preparation: (4,4-difluoro-1-piperidyl)-[2-(1-ethylpyrazol-3-yl)-4-(5-methyl-4H-1,2,4-triazol-3-yl)phenyl]methanone

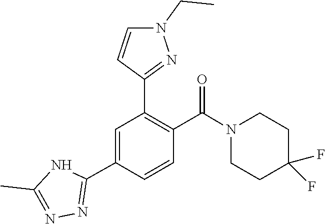 [SMILES: CCn1ccc(-c2cc(-c3nnc(C)[nH]3)ccc2C(=O)N2CCC(F)(F)CC2)n1]

### Example 59

The synthesis of the title compound was effected analogously to the synthesis of the compound of Example 53 [2-(1-tert-butylpyrazol-3-yl)-4-(5-methyl-4H-1,2,4-triazol-3-yl)phenyl]-[4-(trifluoromethyl)-1-piperidyl]methanone wherein 1-ethyl-3-(4,4,5,5-tetramethyl-1,3,2-dioxaborolan-2-yl)pyrazole and [2-bromo-4-(5-methyl-4H-1,2,4-triazol-3-yl)phenyl]-(4,4-difluoropiperidin-1-yl)methanone were used instead of 1-tert-butyl-3-(4,4,5,5-tetramethyl-1,3,2-dioxaborolan-2-yl)pyrazole (383.71 mg, 0.380 mmol) and [2-bromo-4-(5-methyl-4H-1,2,4-triazol-3-yl)phenyl]-[4-(trifluoromethyl)piperidin-1-yl]methanone respectively. The title compound was obtained in a yield of 24% (12 mg).

1H NMR (400 MHZ, DMSO-d6) δ ppm 8.33 (d, 1H), 7.96 (dd, 1H), 7.82 (d, 1H), 7.37 (d, 1H), 6.48 (d, 1H), 4.17 (q, 2H), 3.90 (br d, 1H), 3.56 (m, 1H), 3.24-3.08 (m, 2H), 2.41 (s, 3H), 2.17-1.75 (m, 4H), 1.39 (t, 4H); LC-MS: m/z 401.31 (MH+).

### Preparation: (4,4-difluoro-1-piperidyl)-[4-(5-methyl-4H-1,2,4-triazol-3-yl)-2-[1-(oxetan-3-yl) pyrazol-3-yl]phenyl]methanone

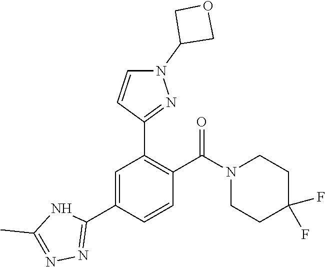 [SMILES: Cc1nnc(-c2ccc(C(=O)N3CCC(F)(F)CC3)c(-c3ccn(C4COC4)n3)c2)[nH]1]

### Example 60

The synthesis of the title compound was effected analogously to the synthesis of the compound of Example 53 [2-(1-tert-butylpyrazol-3-yl)-4-(5-methyl-4H-1,2,4-triazol-3-yl)phenyl]-[4-(trifluoromethyl)-1-piperidyl]methanone wherein 1-(oxetan-3-yl)-3-(4,4,5,5-tetramethyl-1,3,2-dioxaborolan-2-yl)pyrazole and [2-bromo-4-(5-methyl-4H-1,2,4-triazol-3-yl)phenyl]-(4,4-difluoropiperidin-1-yl)methanone were used instead of 1-tert-butyl-3-(4,4,5,5-tetramethyl-1,3,2-dioxaborolan-2-yl)pyrazole and [2-bromo-4-(5-methyl-4H-1,2,4-triazol-3-yl)phenyl]-[4-(trifluoromethyl)piperidin-1-yl]methanone respectively. The title compound was obtained in a yield of 55% (42 mg).

1H NMR (400 MHZ, DMSO-d6) δ ppm 8.36 (d, 1H), 8.02-7.91 (m, 2H), 7.42 (d, 1H), 6.60 (d, 1H), 5.68-5.58 (m, 1H), 5.00-4.84 (m, 4H), 3.86-3.65 (m, 2H), 3.23-3.11 (m, 2H), 2.45-2.35 (m, 3H), 2.17-1.83 (m, 4H), 1.67-1.50 (m, 1H); LC-MS: m/z 429.38 (MH+).

### Preparation: (4-methylpiperidin-1-yl)-[4-(5-methyl-4H-1,2,4-triazol-3-yl)-2-(1-propan-2-ylpyrazol-3-yl)phenyl]methanone

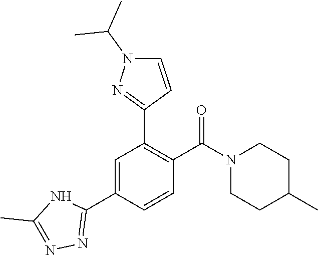

### Example 61

The synthesis of the title compound was effected analogously to the synthesis of the compound of Example 53 [2-(1-tert-butylpyrazol-3-yl)-4-(5-methyl-4H-1,2,4-triazol-3-yl)phenyl]-[4-(trifluoromethyl)-1-piperidyl]methanone wherein 1-(propan-2-yl)-3-(tetramethyl-1,3,2-dioxaborolan-2-yl)-1H-pyrazole and [2-bromo-4-(5-methyl-4H-1,2,4-triazol-3-yl)phenyl]-(4-methylpiperidin-1-yl)methanone were used instead of 1-tert-butyl-3-(4,4,5,5-tetramethyl-1,3,2-dioxaborolan-2-yl)pyrazole and [2-bromo-4-(5-methyl-4H-1,2,4-triazol-3-yl)phenyl]-[4-(trifluoromethyl)piperidin-1-yl]methanone respectively. The title compound was obtained in a yield of 60% (22 mg).

1H NMR (400 MHZ, DMSO) δ 13.79 (s, 1H), 8.42-8.28 (m, 1H), 7.93 (ddd, 1H), 7.81 (dd, 1H), 7.28 (dd, 1H), 6.42 (td, 1H), 4.63-4.45 (m, 2H), 3.26-3.08 (m, 1H), 2.89-2.57 (m, 2H), 2.41 (s, 3H), 1.76-1.32 (m, 9H), 1.24-0.94 (m, 2H), 0.92-0.70 (m, 3H); LC-MS: m/z 393.57 (MH+).

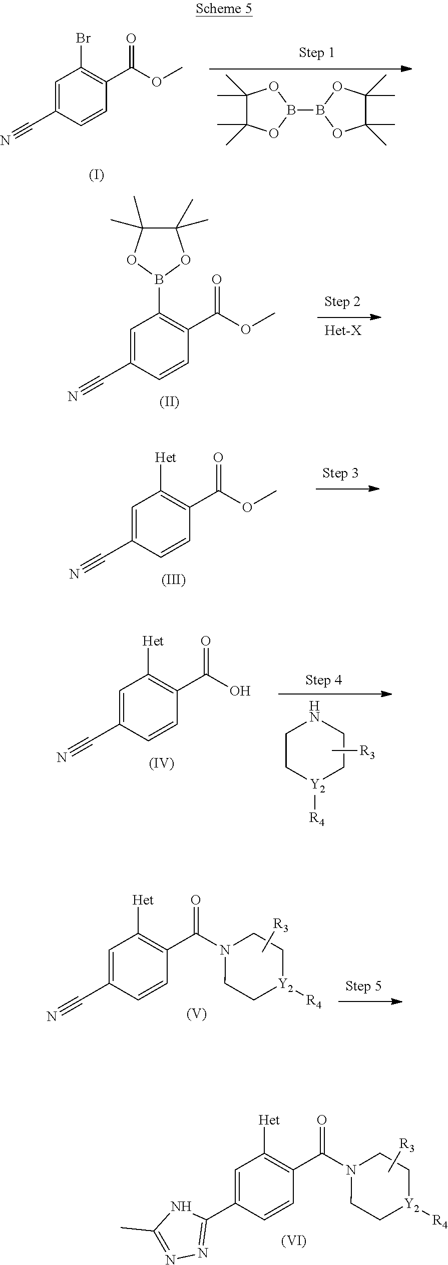

### Step 1

Compound of formula II may be obtained via Miyaura Borylation between compound of formula I (commercially available from Astatech) and B₂Pin₂, in the presence of a suitable transition metal catalyst, e.g. Pd(dppf)Cl₂, a suitable base, e.g. potassium acetate, in a suitable solvents, e.g. 1,4-Dioxane, typically at 100° C. The reaction takes from about 3 hours to about 12 hours to complete.

### Step 2

Compound of formula III may be obtained via Suzuki coupling between compound of formula II and the desired heteroaryl halide, in presence of a suitable precatalyst, e.g. XPhos Pd G2, a suitable base, e.g. K₃PO₄, in a suitable mixture of solvents, e.g. 1,2-dimethoxyethane/water, typically at 100° C. The reaction takes from about hours to about 12 hours to complete.

### Step 3

Compound of formula IV may be obtained by hydrolysis of compound of formula III with LiOH in presence of a suitable mixture of solvents, such as MeOH/THF/water, typically at room temperature. The reaction takes about 12 hours to complete.

### Step 4

Compound of formula V may be obtained from compound of formula IV by coupling with a proper amine in the presence of coupling agent, e.g. HATU, and an organic base, e.g. DIPEA. The reaction is carried out in a suitable solvent such as DMF, typically at room temperature. The reaction takes from about 3 hours to about 12 hours to complete.

### Step 5

Compound of formula VI may be obtained by nitrile cyclization of compound of formula V with ethanimidamide hydrochloride in presence of a suitable catalyst, e.g. CuBr, a suitable base, e.g. Cs₂CO₃, in a suitable solvent, such as DMSO, typically at 120° C. The reaction takes from about 3 hours to about 12 hours to complete.

### Scheme 5, Cpd (II) Preparation: methyl 4-cyano-2-(4,4,5,5-tetramethyl-1,3,2-dioxaborolan-2-yl)benzoate

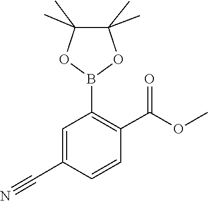 [SMILES: COC(=O)c1ccc(C#N)cc1B1OC(C)(C)C(C)(C)O1]

A suspension of [1,1′-Bis(diphenylphosphino) ferrocene]dichloropalladium (II), complex with dichloromethane (0.07 g, 0.080 mmol), potassium acetate (1.24 g, 12.5 mmol), 4,4,5,5-tetramethyl-2-(4,4,5,5-tetramethyl-1,3,2-dioxaborolan-2-yl)-1,3,2-dioxaborolane (1.27 g, 5 mmol) and methyl 2-bromo-4-cyanobenzoate (1.0 g, 4.17 mmol) in 1,4-Dioxane (30.41 mL) was degassed under nitrogen flux for 10 min and heated to 100° C. overnight. The day after the reaction mixture was cooled down to room temperature and diluted with water and extracted with EtOAc (3×). The organic layers were collected together and washed with brine, dried over Na₂SO₄, filtered and the solvent removed under vacuum affording the product of formula methyl 4-cyano-2-(4,4,5,5-tetramethyl-1,3,2-dioxaborolan-2-yl)benzoate, which was in the next step without further purifications.

### Yield: 1.19 g.

### LC-MS: m/z 288.14 (MH+).

### Scheme 5, Cpd (III)*a Preparation: methyl 4-cyano-2-[4-(trifluoromethyl) thiazol-2-yl]benzoate

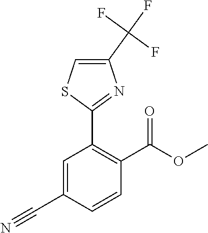 [SMILES: COC(=O)c1ccc(C#N)cc1-c1nc(C(F)(F)F)cs1]

A mixture of 2-bromo-4-(trifluoromethyl) thiazole (169.7 mg, 0.730 mmol), methyl 4-cyano-2-(4,4,5,5-tetramethyl-1,3,2-dioxaborolan-2-yl)benzoate (210.0 mg, 0.730 mmol), X-Phos Pd G2 (0.06 g, 0.070 mmol) and tripotassium phosphate (0.47 g, 2.19 mmol) in Water (0.336 mL) and 1,2-dimethoxyethane (3.356 mL) was degassed under nitrogen flux for 10 min. and heated to 100° C. for 2 h. The reaction mixture was cooled down to room temperature, diluted with water and extracted with EtOAc (3×). The organic layers were collected together and washed with brine, dried over Na₂SO₄, filtered and the solvent removed under vacuum. Crude was purified by FC on silica gel (eluting from 100% of cHex to cHex/EtOAc 90:10) affording the product of formula methyl 4-cyano-2-[4-(trifluoromethyl) thiazol-2-yl]benzoate.

### Yield: 52 mg

¹H NMR (400 MHZ, DMSO-d₆) δ ppm 8.70 (d, 1H), 8.43 (d, 1H), 8.15 (dd, 1H), 7.91 (d, 1H), 3.72 (s, 3H); LC-MS: m/z 313.09 (MH+).

### Scheme 5, Cpd (III)*b Preparation: methyl 4-cyano-2-(6-cyclopropyl-2-pyridyl)benzoate

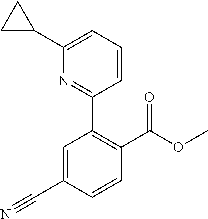 [SMILES: COC(=O)c1ccc(C#N)cc1-c1cccc(C2CC2)n1]

The synthesis of the title compound was effected analogously to the synthesis of the compound methyl 4-cyano-2-[4-(trifluoromethyl) thiazol-2-yl]benzoate wherein 2-bromo-6-cyclopropylpyridine was used instead of 2-bromo-4-(trifluoromethyl) thiazole. The title compound was obtained in a yield of 45% (303.7 mg).

¹H NMR (400 MHZ, DMSO-d₆) δ ppm 8.20 (d, 1H), 7.98 (dd, 1H), 7.81-7.76 (m, 2H), 7.59 (dd, 1H), 7.34 (dd, 1H), 3.66 (s, 3H), 2.17-2.09 (m, 1H), 0.98-0.83 (m, 4H); LC-MS: m/z 279.22 (MH+).

### Scheme 5, Cpd (III)*c Preparation: methyl 2-(1-tert-butylpyrazol-3-yl)-4-cyano-benzoate

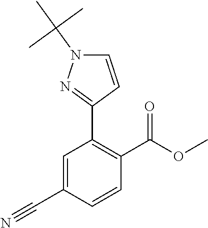 [SMILES: COC(=O)c1ccc(C#N)cc1-c1ccn(C(C)(C)C)n1]

The synthesis of the title compound was effected analogously to the synthesis of the compound methyl 4-cyano-2-[4-(trifluoromethyl) thiazol-2-yl]benzoate wherein 1-tert-butyl-3-iodopyrazole was used instead of 2-bromo-4-(trifluoromethyl) thiazole. The title compound was obtained in a yield of 12% (60 mg).

¹H NMR (400 MHZ, DMSO-d₆) δ ppm 8.20 (d, 1H), 7.90 (d, 1H), 7.84 (dd, 1H), 7.65 (d, 1H), 6.73 (d, 1H), 3.77 (s, 3H) 1.53 (s, 9H); LC-MS: m/z 284.20 (MH+).

### Scheme 5, Cpd (IV)*a Preparation: 4-cyano-2-[4-(trifluoromethyl) thiazol-2-yl]benzoic acid

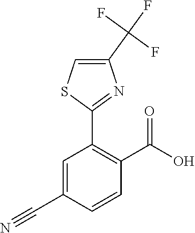 [SMILES: N#Cc1ccc(C(=O)O)c(-c2nc(C(F)(F)F)cs2)c1]

To a solution of methyl 4-cyano-2-[4-(trifluoromethyl) thiazol-2-yl]benzoate (53.0 mg, 0.170 mmol) in THF (1.178 mL) in Methanol (0.294 mL), a solution of lithium hydroxide (8.13 mg, 0.255 mmol) in Water (0.294 mL) was added. The reaction was allowed to stir overnight at room temperature. After this time the solution was evaporated under reduced pressure and then the residue was taken up with water. Aqueous phase was washed with DCM, acidified until pH=4 with a 1N solution of HCl and extracted with EtOAc (×6). The combine organic fractions were dried over Na₂SO₄, filtered and concentrated under vacuum affording the product of formula 4-cyano-2-[4-(trifluoromethyl) thiazol-2-yl]benzoic acid.

### Yield: 44.7 mg.

1H NMR (400 MHZ, DMSO-d6) δ ppm 13.71 (br s, 1H), 8.68 (s, 1H), 8.30 (d, 1H), 8.12 (dd, 1H), 7.93 (d, 1H); LC-MS: m/z 299.07 (MH+).

### Scheme 5, Cpd (IV)*b Preparation: 4-cyano-2-(6-cyclopropyl-2-pyridyl)benzoic acid

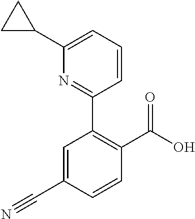 [SMILES: N#Cc1ccc(C(=O)O)c(-c2cccc(C3CC3)n2)c1]

The synthesis of the title compound was effected analogously to the synthesis of the compound 4-cyano-2-[4-(trifluoromethyl) thiazol-2-yl]benzoic acid wherein methyl 4-cyano-2-(6-cyclopropyl-2-pyridyl)benzoate was used instead of methyl 4-cyano-2-[4-(trifluoromethyl) thiazol-2-yl]benzoate. The title compound was obtained in a yield of 93% (269.5 mg).

1H NMR (400 MHZ, DMSO-d6) δ ppm 13.04 (br s, 1H), 8.13 (d, 1H), 7.94 (dd, 1H), 7.78-7.71 (m, 2H), 7.55 (d, 1H), 7.34 (d, 1H), 2.16-2.06 (m, 1H), 1.05-0.87 (m, 4H); LC-MS: m/z 265.22 (MH+).

### Scheme 5, Cpd (IV)*c Preparation: 2-(1-tert-butylpyrazol-3-yl)-4-cyano-benzoic acid

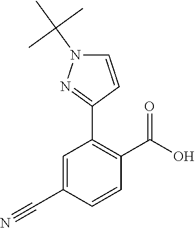 [SMILES: CC(C)(C)n1ccc(-c2cc(C#N)ccc2C(=O)O)n1]

The synthesis of the title compound was effected analogously to the synthesis of the compound 4-cyano-2-[4-(trifluoromethyl) thiazol-2-yl]benzoic acid wherein methyl 2-(1-tert-butylpyrazol-3-yl)-4-cyano-benzoate was used instead of methyl 4-cyano-2-[4-(trifluoromethyl) thiazol-2-yl]benzoate. The title compound was obtained in a yield of 100% (58 mg).

1H NMR (400 MHZ, DMSO-d6) δ ppm 13.19 (br s, 1H), 8.15 (d, 1H), 7.88 (d, 1H), 7.81 (dd, 1H), 7.62 (d, 1H), 6.65 (d, 1H), 1.55 (s, 9H); LC-MS: m/z 270.22 (MH+).

### Scheme 5, Cpd (V)*a Preparation: 4-(4-methylpiperidine-1-carbonyl)-3-[4-(trifluoromethyl) thiazol-2-yl]benzonitrile

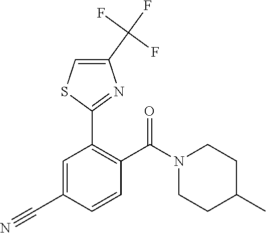 [SMILES: CC1CCN(C(=O)c2ccc(C#N)cc2-c2nc(C(F)(F)F)cs2)CC1]

To a stirred solution of 4-cyano-2-[4-(trifluoromethyl) thiazol-2-yl]benzoic acid (44.7 mg, 0.150 mmol) in DMF (1.041 mL), N,N-Diisopropylethylamine (0.08 mL, 0.450 mmol) and HATU (85.48 mg, 0.220 mmol) were added. The reaction was stirred for 15 minutes at room temperature. Then 4-methylpiperidine (14.86 mg, 0.150 mmol) was added and the reaction mixture was stirred at room temperature overnight. The day after a saturated solution of NaHCO₃ was added to the reaction mixture and the aqueous phase was extracted with EtOAc (×3). The organic portions were collected, washed with brine, residual water was removed by adding Na₂SO₄ and the solvent was evaporated under reduced pressure. The residue was purified by FC on silica gel (eluting from 100% of cHex to cHex/EtOAc 70:30) affording the product of formula 4-(4-methylpiperidine-1-carbonyl)-3-[4-(trifluoromethyl) thiazol-2-yl]benzonitrile.

### Yield: 46.8 mg.

1H NMR (400 MHZ, DMSO-d6) δ ppm 8.71-8.66 (m, 1H), 8.45-8.34 (m, 1H), 8.07 (dd, 1H), 7.62 (dd, 1H), 4.46-4.35 (m, 1H), 3.27 (s, 2H), 3.06-2.97 (m, 1H), 2.81-2.58 (m, 8H); LC-MS: m/z 380.20 (MH+).

### Scheme 5, Cpd_(V)*b Preparation: 3-(6-cyclopropyl-2-pyridyl)-4-(4-methylpiperidine-1-carbonyl)benzonitrile

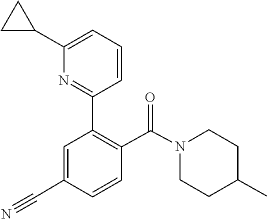 [SMILES: CC1CCN(C(=O)c2ccc(C#N)cc2-c2cccc(C3CC3)n2)CC1]

The synthesis of the title compound was effected analogously to the synthesis of the compound 4-(4-methylpiperidine-1-carbonyl)-3-[4-(trifluoromethyl) thiazol-2-yl]benzonitrile wherein 4-cyano-2-(6-cyclopropyl-2-pyridyl)benzoic acid was used instead of 4-cyano-2-[4-(trifluoromethyl) thiazol-2-yl]benzoic acid. The title compound was obtained in a yield of 85% (55.3 mg).

1H NMR (400 MHZ, DMSO-d6) δ ppm 8.42-8.25 (m, 1H), 8.14-8.06 (m, 1H), 7.92 (q, 1H), 7.75-7.60 (m, 2H), 7.49 (dd, 1H), 4.61-4.50 (m, 1H), 2.96-2.71 (m, 2H), 2.37-2.23 (m, 1H), 1.88-1.49 (m, 3H), 1.43-1.04 (m, 6H), 0.97-0.79 (m, 3H), 0.18-0.09 (m, 1H); LC-MS: m/z 346.25 (MH+).

### Scheme 5, Cpd (V)*c Preparation: 3-(1-tert-butylpyrazol-3-yl)-4-(4-methylpiperidine-1-carbonyl)benzonitrile

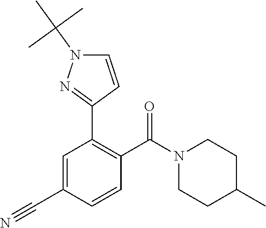 [SMILES: CC1CCN(C(=O)c2ccc(C#N)cc2-c2ccn(C(C)(C)C)n2)CC1]

The synthesis of the title compound was effected analogously to the synthesis of the compound 4-(4-methylpiperidine-1-carbonyl)-3-[4-(trifluoromethyl) thiazol-2-yl]benzonitrile wherein 2-(1-tert-butylpyrazol-3-yl)-4-cyano-benzoic acid was used instead of 4-cyano-2-[4-(trifluoromethyl) thiazol-2-yl]benzoic acid. The title compound was obtained in a yield of 84% (60 mg).

1H NMR (400 MHZ, DMSO-d6) δ ppm 8.15 (dd, 1H), 7.90 (dd, 1H), 7.81 (ddd, 1H), 7.43 (dd, 1H), 6.56 (dd, 1H), 4.55-4.41 (m, 1H), 3.99-2.23 (m, 1H), 2.89-2.64 (m, 2H), 1.54 (d, 10H), 1.29-0.93 (m, 3H), 0.92-0.71 (m, 3H), 0.13 (qd, 1H); LC-MS: m/z 351.35 (MH+).

### Scheme 5, Cpd (V)*d Preparation: 3-(6-cyclopropyl-2-pyridyl)-4-(4-fluoro-4-methyl-piperidine-1-carbonyl)benzonitrile

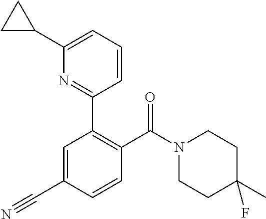 [SMILES: CC1(F)CCN(C(=O)c2ccc(C#N)cc2-c2cccc(C3CC3)n2)CC1]

The synthesis of the title compound was effected analogously to the synthesis of the compound 4-(4-methylpiperidine-1-carbonyl)-3-[4-(trifluoromethyl) thiazol-2-yl]benzonitrile wherein 4-fluoro-4-methyl-piperidine hydrochloride was used instead of 4-methylpiperidine and 4-cyano-2-(6-cyclopropyl-2-pyridyl)benzoic acid was used instead of 4-cyano-2-[4-(trifluoromethyl) thiazol-2-yl]benzoic acid. The title compound was obtained in a yield of 29% (13 mg) LC-MS: m/z 364.35 (MH+).

### Scheme 5, Cpd (V)*e Preparation: 3-(1-tert-butylpyrazol-3-yl)-4-(4-methoxy-4-methyl-piperidine-1-carbonyl)benzonitrile

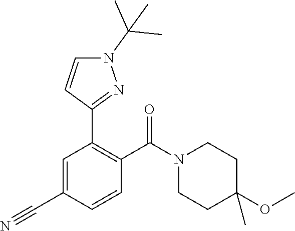 [SMILES: COC1(C)CCN(C(=O)c2ccc(C#N)cc2-c2ccn(C(C)(C)C)n2)CC1]

The synthesis of the title compound was effected analogously to the synthesis of the compound 4-(4-methylpiperidine-1-carbonyl)-3-[4-(trifluoromethyl) thiazol-2-yl]benzonitrile wherein 4-methoxy-4-methyl-piperidine hydrochloride was used instead of 4-methylpiperidine and 2-(1-tert-butylpyrazol-3-yl)-4-cyano-benzoic acid was used instead of 4-cyano-2-[4-(trifluoromethyl) thiazol-2-yl]benzoic acid. The title compound was obtained in a yield of 81% (41 mg).

1H NMR (400 MHZ, DMSO-d6) δ ppm 8.15 (dd, 1H), 7.90 (dd, 1H), 7.84-7.78 (m, 1), 7.45 (dd, 1H), 6.58 (dd, 1H), 4.19-4.05 (m, 1H), 3.14-2.77 (m, 6H), 1.84-1.66 (m, 1H), 1.54 (d, 11H), 1.11-0.89 (m, 3H), 0.57-0.43 (m, 1H); LC-MS: m/z 381.36 (MH+).

### Scheme 5, Cpd (V)*f Preparation: 4-(4-methoxy-4-methyl-piperidine-1-carbonyl)-3-[4-(trifluoromethyl) thiazol-2-yl]benzonitrile

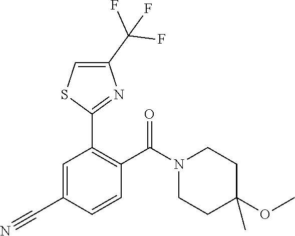 [SMILES: COC1(C)CCN(C(=O)c2ccc(C#N)cc2-c2nc(C(F)(F)F)cs2)CC1]

The synthesis of the title compound was effected analogously to the synthesis of the compound 4-(4-methylpiperidine-1-carbonyl)-3-[4-(trifluoromethyl) thiazol-2-yl]benzonitrile wherein 4-methoxy-4-methyl-piperidine hydrochloride was used instead of 4-methylpiperidine. The title compound was obtained in a yield of 65% (88.8 mg).

1H NMR (400 MHZ, DMSO-d6) δ ppm 8.71-8.65 (m, 1H), 8.45-8.35 (m, 1H), 8.12-8.03 (m, 1H), 7.69-7.57 (m, 1H), 4.19-4.01 (m, 1H), 3.29 (s, 3H), 3.26-2.95 (m, 3H), 1.81-1.70 (m, 1H), 1.60-1.20 (m, 3H), 1.04 (s, 3H); LC-MS: m/z 410.27 (MH+).

### Scheme 5, Cpd (VI)*a Preparation: (4-methyl-1-piperidyl)-[4-(5-methyl-4H-1,2,4-triazol-3-yl)-2-[4-(trifluoromethyl) thiazol-2-yl]phenyl]methanone

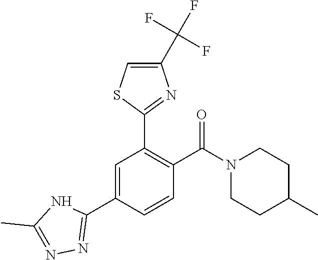

### Example 62

A mixture of dicesium carbonate (120.57 mg, 0.370 mmol), ethanimidamide hydrochloride (17.49 mg, 0.190 mmol), CuBr (0.88 mg, 0.010 mmol) and 4-(4-methylpiperidine-1-carbonyl)-3-[4-(trifluoromethyl) thiazol-2-yl]benzonitrile (46.8 mg, 0.120 mmol) in DMSO (1.199 mL) was stirred at 120° C. for 4 h. After this time the reaction was cooled down to room temperature and H₂O was added. The mixture was then extracted with AcOEt 3 times. Aqueous phase was diluted with a saturated solution of potassium sodium tartrate and extracted with EtOAc and THE ×5. The combined organic fractions were dried over Na₂SO₄, filtered and concentrated under vacuum. The residue was purified by FC on NH column (eluenting from 100% of DCM to DCM/MeOH 95:5) affording the product of formula (4-methyl-1-piperidyl)-[4-(5-methyl-4H-1,2,4-triazol-3-yl)-2-[4-(trifluoromethyl) thiazol-2-yl]phenyl].

### Yield: 26.6 mg.

1H NMR (400 MHZ, DMSO-d6) δ ppm 8.69-8.61 (m, 1H), 8.54-8.38 (m, 1H), 8.20-8.13 (m, 1H), 7.56-7.40 (m, 1H), 4.56-4.41 (m, 1H), 3.09-2.94 (m, 1H), 3.12-2.62 (m, 3H), 2.46 (s, 2H), 1.84-0.71 (m, 9H); LC-MS: m/z 436.28 (MH+).

### Preparation: [2-(6-cyclopropyl-2-pyridyl)-4-(5-methyl-4H-1,2,4-triazol-3-yl)phenyl]-(4-methyl-1-piperidyl)methanone

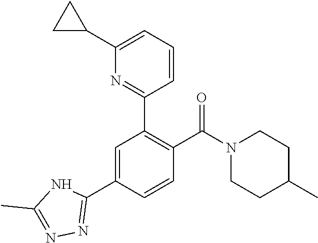

### Example 63

The synthesis of the title compound was effected analogously to the synthesis of the compound of Example 62 (4-methyl-1-piperidyl)-[4-(5-methyl-4H-1,2,4-triazol-3-yl)-2-[4-(trifluoromethyl) thiazol-2-yl]phenyl]methanone wherein 3-(6-cyclopropyl-2-pyridyl)-4-(4-methylpiperidine-1-carbonyl)benzonitrile was used instead of 4-(4-methylpiperidine-1-carbonyl)-3-[4-(trifluoromethyl) thiazol-2-yl]benzonitrile. The title compound was obtained in a yield of 65% (42 mg).

1H NMR (400 MHZ, DMSO-d6) δ ppm 8.23 (br d, 1H), 8.03 (d, 1H), 7.74 (td, 1H), 7.47-7.25 (m, 3H), 4.47-4.35 (m, 1H), 2.84-2.53 (m, 3H), 2.44-2.36 (m, 3H), 2.20-2.06 (m, 1H), 1.74-0.62 (m, 12H), 0.32-0.25 (m, 1H); LC-MS: m/z 402.36 (MH+).

### Preparation: [2-(1-tert-butylpyrazol-3-yl)-4-(5-methyl-4H-1,2,4-triazol-3-yl)phenyl]-(4-methyl-1-piperidyl)methanone

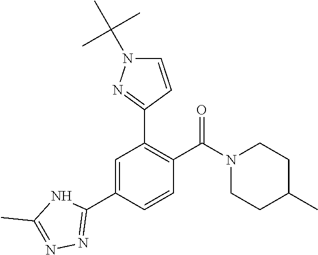

### Example 64

The synthesis of the title compound was effected analogously to the synthesis of the compound of Example 62 (4-methyl-1-piperidyl)-[4-(5-methyl-4H-1,2,4-triazol-3-yl)-2-[4-(trifluoromethyl) thiazol-2-yl]phenyl]methanone wherein 3-(1-tert-butylpyrazol-3-yl)-4-(4-methylpiperidine-1-carbonyl)benzonitrile was used instead of 4-(4-methylpiperidine-1-carbonyl)-3-[4-(trifluoromethyl) thiazol-2-yl]benzonitrile. The title compound was obtained in a yield of 68% (47.3 mg).

¹H NMR (400 MHZ, DMSO-d₆) δ ppm 8.30 (dd, 1H), 7.98-7.79 (m, 2H), 7.28 (t, 1H), 6.42 (dd, 1H), 4.49 (br d, 1H), 2.85-2.57 (m, 2H), 2.40 (s, 3H), 1.55 (d, 11H), 1.30-0.67 (m, 7H), 0.11-0.09 (m, 1H); LC-MS: m/z 407.47 (MH+).

### Preparation: [2-(6-cyclopropyl-2-pyridyl)-4-(5-methyl-4H-1,2,4-triazol-3-yl)phenyl]-(4-fluoro-4-methyl-1-piperidyl)methanone

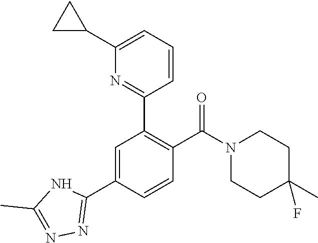

### Example 65

The synthesis of the title compound was effected analogously to the synthesis of the compound of Example 62 (4-methyl-1-piperidyl)-[4-(5-methyl-4H-1,2,4-triazol-3-yl)-2-[4-(trifluoromethyl) thiazol-2-yl]phenyl]methanone wherein 3-(6-cyclopropyl-2-pyridyl)-4-(4-fluoro-4-methyl-piperidine-1-carbonyl)benzonitrile was used instead of 4-(4-methylpiperidine-1-carbonyl)-3-[4-(trifluoromethyl) thiazol-2-yl]benzonitrile. The title compound was obtained in a yield of 53% (8 mg).

¹H NMR (400 MHZ, DMSO-d₆) δ ppm 8.27-8.21 (m, 1H), 8.07-8.00 (m, 1H), 7.79-7.71 (m, 1H), 7.49-7.40 (m, 2H), 7.33-7.26 (m, 1H), 4.29-4.13 (m, 1H), 3.08-2.87 (m, 3H), 2.41 (s, 3H), 2.18-2.06 (m, 1H), 1.88-0.71 (m, 11H), 0.39-0.16 (m, 1H); LC-MS: m/z 420.37 (MH+).

### Preparation: [2-(1-tert-butylpyrazol-3-yl)-4-(5-methyl-4H-1,2,4-triazol-3-yl)phenyl]-(4-methoxy-4-methyl-1-piperidyl)methanone

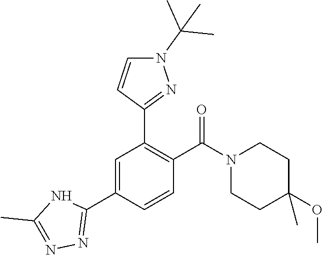

### Example 66

The synthesis of the title compound was effected analogously to the synthesis of the compound of Example 62 (4-methyl-1-piperidyl)-[4-(5-methyl-4H-1,2,4-triazol-3-yl)-2-[4-(trifluoromethyl) thiazol-2-yl]phenyl]methanone wherein 3-(1-tert-butylpyrazol-3-yl)-4-(4-methoxy-4-methyl-piperidine-1-carbonyl)benzonitrile was used instead of 4-(4-methylpiperidine-1-carbonyl)-3-[4-(trifluoromethyl) thiazol-2-yl]benzonitrile. The title compound was obtained in a yield of 40% (19 mg).

1H NMR (400 MHZ, DMSO-d6) δ ppm 8.31 (dd, 1H), 7.98-7.85 (m, 2H), 7.30 (dd, 1H), 6.44 (dd, 1H), 4.22-4.07 (m, 1H), 3.05 (d, 6H), 2.41 (s, 3H), 1.80-1.65 (m, 1H), 1.57 (d, 9H), 1.51-1.22 (m, 3H), 1.12-0.89 (m, 3H), 0.45-0.28 (m, 1H); LC-MS: m/z 437.48 (MH+).

### Preparation: (4-methoxy-4-methyl-1-piperidyl)-[4-(5-methyl-4H-1,2,4-triazol-3-yl)-2-[4-(trifluoromethyl) thiazol-2-yl]phenyl]methanone

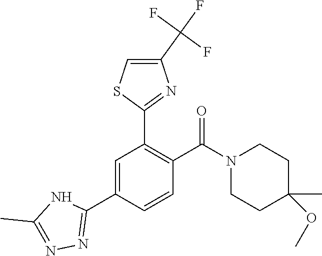

### Example 67

The synthesis of the title compound was effected analogously to the synthesis of the compound of Example 62 (4-methyl-1-piperidyl)-[4-(5-methyl-4H-1,2,4-triazol-3-yl)-2-[4-(trifluoromethyl) thiazol-2-yl]phenyl]methanone wherein 4-(4-methoxy-4-methyl-piperidine-1-carbonyl)-3-[4-(trifluoromethyl) thiazol-2-yl]benzonitrile was used instead of 4-(4-methylpiperidine-1-carbonyl)-3-[4-(trifluoromethyl) thiazol-2-yl]benzonitrile. The title compound was obtained in a yield of 25% (27.3 mg).

1H NMR (400 MHZ, DMSO-d6) δ ppm 8.62 (s, 1H), 8.52-8.35 (m, 1H), 8.15 (dd, 1H), 7.53-7.42 (m, 1H), 4.22-3.98 (m, 1H), 3.23-2.95 (m, 6H), 2.42 (s, 3H), 1.83-1.69 (m, 1H), 1.58-1.36 (m, 3H), 1.25-0.99 (m, 4H); LC-MS: m/z 466.29 (MH+).

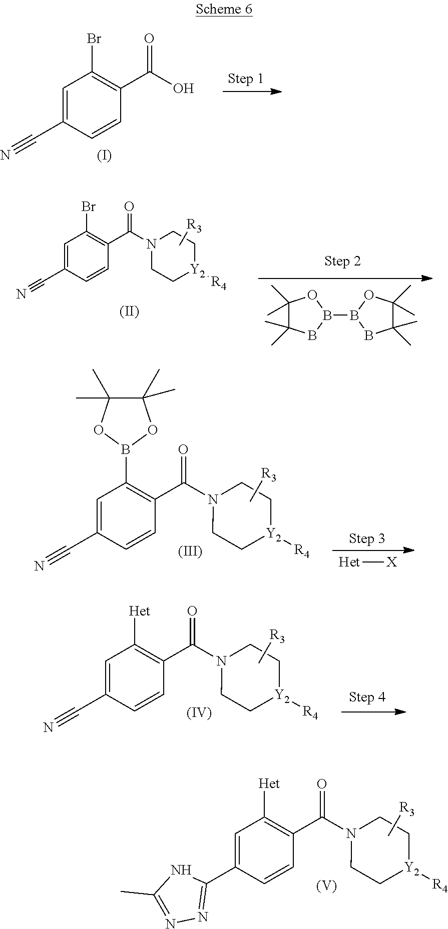

### Step 1

Compound of formula II may be obtained from compound of formula I (commercially available from Astatech) by coupling with a proper amine in presence of coupling agent, e.g. HATU, and a suitable organic base, e.g. DIPEA. The reaction is carried out in a suitable solvent such as DMF, typically at room temperature. The reaction takes from about 3 hours to about 12 hours to complete.

### Step 2

Compound of formula III may be obtained via Miyaura Borylation between compound of formula II and B₂Pin₂, in the presence of a suitable transition metal catalyst, e.g. Pd(dppf)Cl₂, a suitable base, e.g. potassium acetate, in a suitable solvents, e.g. 1,4-Dioxane, typically at 95° C. The reaction takes about 12 hours to complete.

### Step 3

Compound of formula IV may be obtained via Suzuki coupling between compound of formula III and commercially available heteroaryl halide in the presence of a suitable catalyst, e.g. palladium tetrakis triphenylphosphine, suitable inorganic base, e.g. Na₂CO₃, in a suitable mixture of solvents, such as 1,2-dimethoxyethane/water, typically at 95° C. The reaction takes from about 3 hours to about 12 hours to complete.

### Step 4

Compound of formula V may be obtained by nitrile cyclization of compound of formula IV with ethanimidamide hydrochloride in presence of a suitable catalyst, e.g. CuBr, a suitable base, e.g. Cs₂CO₃, in a suitable solvent, such as DMSO, typically at 120° C. The reaction takes from about 3 hours to about 12 hours to complete.

### Scheme 6, Cpd (II)*a Preparation: 3-bromo-4-[4-(trifluoromethyl) piperidine-1-carbonyl]benzonitrile

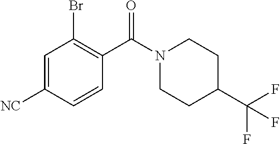 [SMILES: N#Cc1ccc(C(=O)N2CCC(C(F)(F)F)CC2)c(Br)c1]

To a solution of HATU (1.14 g, 2.99 mmol), 2-bromo-4-cyanobenzoic acid (450.0 mg, 1.99 mmol) and 4-(trifluoromethyl) piperidine hydrochloride (415.24 mg, 2.19 mmol) in DMF (9 mL) N,N-Diisopropylethylamine (2.03 mL, 11.95 mmol) was added. The reaction was stirred at RT for 5 h. After this time the reaction was diluted with H₂O and then extracted with AcOEt 3 times. The combined organic fractions were washed with brine, dried over Na₂SO₄, filtered and concentrated under vacuum. The residue was purified by FC on silica gel (eluting from 100% of cHex to AcOEt/cHex 60:40) affording the product of formula 3-bromo-4-[4-(trifluoromethyl) piperidine-1-carbonyl]benzonitrile.

### Yield: 560 mg

1H NMR (400 MHz, DMSO) δ 8.29 (dd, 1H), 7.95 (ddd, 1H), 7.71-7.50 (m, 1H), 4.60 (t, 1H), 3.26 (d, 1H), 3.09 (dtd, 1H), 2.83 (tdd, 1H), 2.66 (d, 1H), 1.94 (t, 1H), 1.82-1.65 (m, 1H), 1.55-1.29 (m, 2H); LC-MS: m/z 361.07, 363.10 (MH+).

### Scheme 6, Cpd (III)*a Preparation: 4-(4,4-difluoropiperidine-1-carbonyl)-3-(4,4,5,5-tetramethyl-1,3,2-dioxaborolan-2-yl)benzonitrile

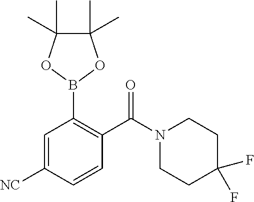 [SMILES: CC1(C)OB(c2cc(C#N)ccc2C(=O)N2CCC(F)(F)CC2)OC1(C)C]

3-bromo-4-(4,4-difluoropiperidine-1-carbonyl)benzonitrile (950.0 mg, 2.8 mmol), 4,4,5,5-tetramethyl-2-(4,4,5,5-tetramethyl-1,3,2-dioxaborolan-2-yl)-1,3,2-dioxaborolane (2.13 g, 8.4 mmol), potassium acetate (832.78 mmol) and mg, 8.4 [1,1′-Bis(diphenylphosphino)ferrocene]dichloropalladium (II), complex with dichloromethane (68.76 mg, 0.080 mmol) were placed in a vial and suspended in 1,4-Dioxane (25 mL). The mixture was degassed through N₂-vacuum cycles (3 times). The mixture was stirred under N₂ atmosphere at 90° C. overnight. After this time the reaction was cooled down, filtered through a pad of celite and then washed with EtOAc. The organic phase was washed with Brine, dried over Na₂SO₄ filtered and concentrated under vacuum affording the product of formula 4-(4,4-difluoropiperidine-1-carbonyl)-3-(4,4,5,5-tetramethyl-1,3,2-dioxaborolan-2-yl)benzonitrile which was used in the next step without further purification.

### Yield: 1.0 g

### LC-MS: m/z 377.26 (MH+).

### Scheme 6, Cpd (III)*b Preparation: 3-(4,4,5,5-tetramethyl-1,3,2-dioxaborolan-2-yl)-4-[4-(trifluoromethyl) piperidine-1-carbonyl]benzonitrile

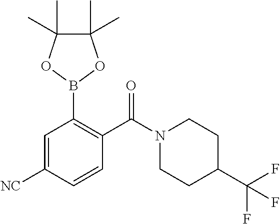 [SMILES: CC1(C)OB(c2cc(C#N)ccc2C(=O)N2CCC(C(F)(F)F)CC2)OC1(C)C]

3-bromo-4-[4-(trifluoromethyl) piperidine-1-carbonyl]benzonitrile (750.0 mg, 2.08 mmol), 4,4,5,5-tetramethyl-2-(4,4,5,5-tetramethyl-1,3,2-dioxaborolan-2-yl)-1,3,2-dioxaborolane (1.58 g, 6.23 mmol), potassium acetate (617.7 mg, 6.23 mmol) and [1,1′-Bis(diphenylphosphino)ferrocene]dichloropalladium (II), complex with dichloromethane (34.0 mg, 0.040 mmol) were suspended in 1,4-Dioxane (20 mL) and the mixture was degassed through N₂-vacuum cycles (3 times). The mixture was stirred under N₂ atmosphere at 95° C. overnight. After this time, complete consumption of starting material was observed so the mixture was cooled down to RT and filtered through a pad of celite washing with EtOAc. The organic phase was washed with Brine, dried over Na₂SO₄ filtered and concentrated under vacuum affording the product of formula 3-(4,4,5,5-tetramethyl-1,3,2-dioxaborolan-2-yl)-4-[4-(trifluoromethyl) piperidine-1-carbonyl]benzonitrile. The product was used in the next step without further purification.

### Yield: 800 mg

### LC-MS: m/z 409.16 (MH+).

### Scheme 6, Cpd (IV)*a Preparation: 4-(4,4-difluoropiperidine-1-carbonyl)-3-(6-isopropyl-2-pyridyl)benzonitrile

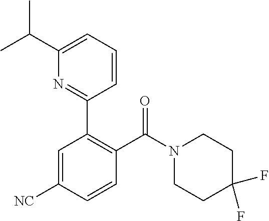 [SMILES: CC(C)c1cccc(-c2cc(C#N)ccc2C(=O)N2CCC(F)(F)CC2)n1]

Palladium tetrakis triphenylphosphine (80.17 mg, 0.070 mmol) and 2-bromo-6-isopropyl-pyridine (277.62 mg, 1.39 mmol) were suspended in 1,2-dimethoxyethane (13.88 mL) and then a 2M aqueous solution of Na₂CO₃ (2.08 mL, 4.16 mmol) was added. The mixture was degassed by 5 cycles of nitrogen/vacuum and 4-(4,4-difluoropiperidine-1-carbonyl)-3-(4,4,5,5-tetramethyl-1,3,2-dioxaborolan-2-yl)benzonitrile (0.5 g, 1.39 mmol) was added. The reaction mixture was degassed again by 5 cycles of nitrogen/vacuum and then was heated to 95° C. for 8 h. The reaction mixture was cooled down to room temperature and diluted with water and extracted with EtOAc (3×). The organic layers were collected together and washed with brine, dried over Na₂SO₄, filtered and the solvent removed under vacuum. The mixture was purified by FC on RP using acid conditions (eluting from 5:95 of CH₃CN/H₂O+0.1% of HCOOH to 60:460 of CH₃CN/H₂O+0.1% of HCOOH) affording the product of formula 4-(4,4-difluoropiperidine-1-carbonyl)-3-(6-isopropyl-2-pyridyl)benzonitrile.

### Yield: 160 mg

¹H NMR (400 MHZ, DMSO-d₆) δ 8.25 (d, 1H), 7.98 (dd, 1H), 7.89-7.85 (m, 1H), 7.72-7.62 (m, 2H), 7.33 (dd, 1H), 4.06-3.97 (m, 1H), 3.40-3.24 (m, 2H), 3.14-3.05 (m, 1H), 2.99 (p, 1H), 2.23-1.84 (m, 3H), 1.71-1.51 (m, 1H), 1.29-1.20 (m, 6H); LC-MS: m/z 370.31 (MH+).

### Scheme 6, Cpd (IV)*b Preparation: 4-(4,4-difluoropiperidine-1-carbonyl)-3-[6-(trifluoromethyl)pyridin-2-yl]benzonitrile

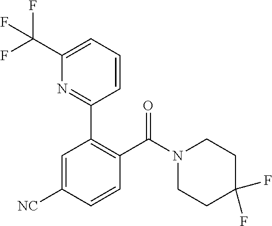 [SMILES: N#Cc1ccc(C(=O)N2CCC(F)(F)CC2)c(-c2cccc(C(F)(F)F)n2)c1]

The synthesis of the title compound was effected analogously to the synthesis of the compound 4-(4,4-difluoropiperidine-1-carbonyl)-3-(6-isopropyl-2-pyridyl)benzonitrile wherein 2-bromo-6-(trifluoromethyl)pyridine was used instead of 2-bromo-6-isopropyl-pyridine. The title compound was obtained in a yield of 40% (130 mg).

1H NMR (400 MHZ, DMSO) δ 8.37 (d, 1H), 8.26 (t, 1H), 8.20 (d, 1H), 8.06 (dd, 1H), 7.95 (dd, 1H), 7.75 (d, 1H), 4.04-3.93 (m, 1H), 3.50-3.39 (m, 1H), 2.54-2.50 (m, 2H), 2.23-1.97 (m, 3H), 1.93-1.83 (m, 1H); LC-MS: m/z 396.26 (MH+).

### Scheme 6, Cpd (IV)*c Preparation: 3-[4-methyl-6-(trifluoromethyl)pyrimidin-2-yl]-4-[4-(trifluoromethyl) piperidine-1-carbonyl]benzonitrile

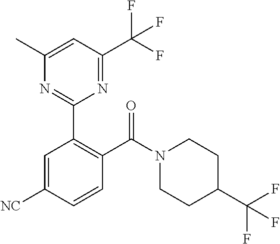 [SMILES: Cc1cc(C(F)(F)F)nc(-c2cc(C#N)ccc2C(=O)N2CCC(C(F)(F)F)CC2)n1]

Palladium tetrakis triphenylphosphine (42.46 mg, 0.040 mmol) and 3-(4,4,5,5-tetramethyl-1,3,2-dioxaborolan-2-yl)-4-[4-(trifluoromethyl) piperidine-1-carbonyl]benzonitrile (0.33 g, 0.730 mmol) were suspended in 1,2-dimethoxyethane (7.349 mL) and then a 2M aqueous solution of Na₂CO₃ (1.1 mL, 2.2 mmol) was added. The reaction was degassed by 5 cycles of nitrogen/vacuum and 2-chloro-4-methyl-6-(trifluoromethyl)pyrimidine (158.9 mg, 0.810 mmol) was added. The reaction mixture was degassed again by 5 cycles of nitrogen/vacuum and then was heated to 95° C. for 8 h. After this time the reaction mixture was cooled down to room temperature, diluted with water and extracted with EtOAc (3×). The organic layers were collected together and washed with brine, dried over Na₂SO₄, filtered and the solvent removed under vacuum. The residue was purified by FC on silica gel (eluting form AcOEt/cHex 5:95 to AcOEt/cHex 60:40) and again by FC on reverse phase using acid conditions (eluting from 5:95 of CH₃CN/H₂O+0.1% of HCOOH to 40:60 of CH₃CN/H₂O+0.1% of HCOOH) affording the product of formula 3-[4-methyl-6-(trifluoromethyl)pyrimidin-2-yl]-4-[4-(trifluoromethyl) piperidine-1-carbonyl]benzonitrile.

### Yield: 34 mg

1H NMR (400 MHZ, DMSO) δ 8.60-8.42 (m, 1H), 8.10 (dd, 1H), 7.99 (s, 1H), 7.73-7.59 (m, 1H), 4.54 (d, 1H), 3.30 (s, 3H), 2.68 (s, 4H), 1.97 (d, 1H), 1.73 (d, 1H), 1.47 (d, 2H); LC-MS: m/z 443.4 (MH+).

### Scheme 6, Cpd (IV)*d Preparation: 3-(4-cyclopropylthiazol-2-yl)-4-[4-(trifluoromethyl) piperidine-1-carbonyl]benzonitrile

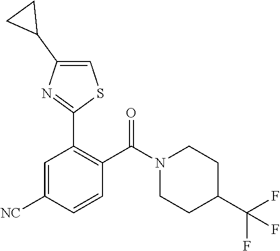 [SMILES: N#Cc1ccc(C(=O)N2CCC(C(F)(F)F)CC2)c(-c2nc(C3CC3)cs2)c1]

The synthesis of the title compound was effected analogously to the synthesis of the compound 3-[4-methyl-6-(trifluoromethyl)pyrimidin-2-yl]-4-[4-(trifluoromethyl) piperidine-1-carbonyl]benzonitrile wherein 2-bromo-4-cyclopropylthiazole was used instead of 2-chloro-4-methyl-6-(trifluoromethyl)pyrimidine. The title compound was obtained in a yield of 11% (33 mg).

1H NMR (400 MHZ, DMSO) δ 8.33-8.22 (m, 1H), 7.98 (td, 1H), 7.65-7.53 (m, 1H), 7.47 (d, 1H), 4.58 (s, 1H), 3.28 (s, 1H), 3.06-2.65 (m, 3H), 2.14-2.06 (m, 1H), 1.99-1.10 (m, 4H), 0.98-0.70 (m, 4H); LC-MS: m/z 406.47 (MH+).

### Scheme 6, Cpd (IV)*e Preparation: 3-(6-propan-2-ylpyridin-2-yl)-4-[4-(trifluoromethyl) piperidine-1-carbonyl]benzonitrile

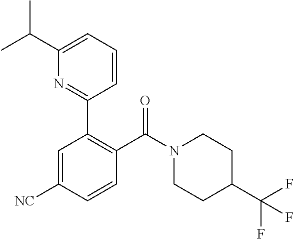 [SMILES: CC(C)c1cccc(-c2cc(C#N)ccc2C(=O)N2CCC(C(F)(F)F)CC2)n1]

The synthesis of the title compound was effected analogously to the synthesis of the compound 3-[4-methyl-6-(trifluoromethyl)pyrimidin-2-yl]-4-[4-(trifluoromethyl) piperidine-1-carbonyl]benzonitrile wherein 2-bromo-6-propan-2-ylpyridine was used instead of 2-chloro-4-methyl-6-(trifluoromethyl)pyrimidine. The title compound was obtained in a yield of 37% (47 mg).

1H NMR (400 MHZ, DMSO) δ 8.27-8.11 (m, 1H), 7.96 (ddd, 1H), 7.84 (td, 1H), 7.64 (dd, 1H), 7.59-7.49 (m, 1H), 7.37-7.26 (m, 1H), 4.52 (d, 1H), 3.30 (s, 4H), 2.50 (p, 1H), 1.94-0.02 (m, 10H); LC-MS: m/z 402.28 (MH+).

### Scheme 6, Cpd (IV)*f Preparation: 3-(6-cyclopropylpyridin-2-yl)-4-[4-(trifluoromethyl) piperidine-1-carbonyl]benzonitrile

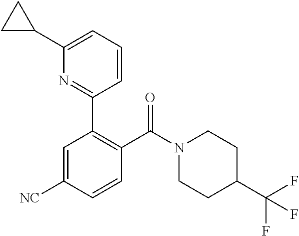 [SMILES: N#Cc1ccc(C(=O)N2CCC(C(F)(F)F)CC2)c(-c2cccc(C3CC3)n2)c1]

The synthesis of the title compound was effected analogously to the synthesis of the compound 3-[4-methyl-6-(trifluoromethyl)pyrimidin-2-yl]-4-[4-(trifluoromethyl) piperidine-1-carbonyl]benzonitrile wherein 2-bromo-6-cyclopropylpyridine was used instead of 2-chloro-4-methyl-6-(trifluoromethyl)pyrimidine. The title compound was obtained in a yield of 34% (90 mg).

¹H NMR (400 MHZ, DMSO-d₆) δ 8.25-8.09 (m, 1H), 8.01-7.86 (m, 1H), 7.82-7.72 (m, 1H), 7.64-7.46 (m, 2H), 7.35-7.25 (m, 1H), 4.54 (d, 1H), 3.12-2.59 (m, 3H), 2.11 (tq, 1H), 1.97-1.63 (m, 2H), 1.54-1.20 (m, 3H), 0.99-0.62 (m, 4H); LC-MS: m/z 400.47 (MH+).

### Scheme 6, Cpd (IV)*g Preparation: 4-[4-(trifluoromethyl) piperidine-1-carbonyl]-3-[4-(trifluoromethyl)-1,3-thiazol-2-yl]benzonitrile

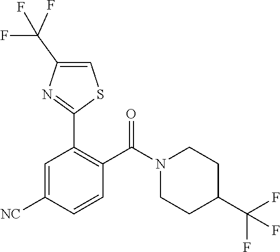 [SMILES: N#Cc1ccc(C(=O)N2CCC(C(F)(F)F)CC2)c(-c2nc(C(F)(F)F)cs2)c1]

The synthesis of the title compound was effected analogously to the synthesis of the compound 3-[4-methyl-6-(trifluoromethyl)pyrimidin-2-yl]-4-[4-(trifluoromethyl) piperidine-1-carbonyl]benzonitrile wherein 2-bromo-4-(trifluoromethyl) thiazole was used instead of 2-chloro-4-methyl-6-(trifluoromethyl)pyrimidine. The title compound was obtained in a yield of 23% (67 mg).

### LC-MS: m/z 434.38 (MH+).

### Scheme 6, Cpd (IV)*h Preparation: 4-[4-(trifluoromethyl) piperidine-1-carbonyl]-3-[6-(trifluoromethyl)pyridin-2-yl]benzonitrile

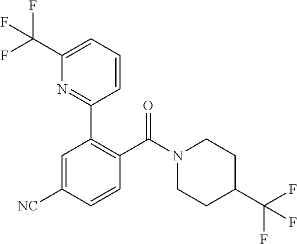 [SMILES: N#Cc1ccc(C(=O)N2CCC(C(F)(F)F)CC2)c(-c2cccc(C(F)(F)F)n2)c1]

The synthesis of the title compound was effected analogously to the synthesis of the compound 3-[4-methyl-6-(trifluoromethyl)pyrimidin-2-yl]-4-[4-(trifluoromethyl) piperidine-1-carbonyl]benzonitrile wherein 2-bromo-6-(trifluoromethyl)pyridine was used instead of 2-chloro-4-methyl-6-(trifluoromethyl)pyrimidine. The title compound was obtained in a yield of 17% (77 mg).

### LC-MS: m/z 428.10 (MH+).

### Scheme 6, Cpd (V)*a Preparation: [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone

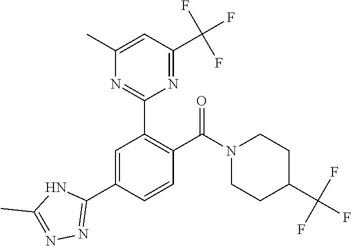 [SMILES: Cc1cc(C(F)(F)F)nc(-c2cc(-c3nnc(C)[nH]3)ccc2C(=O)N2CCC(C(F)(F)F)CC2)n1]

### Example 68

A mixture of 3-[4-methyl-6-(trifluoromethyl)pyrimidin-2-yl]-4-[4-(trifluoromethyl) piperidine-1-carbonyl]benzonitrile (35.0 mg, 0.080 mmol), dicesium carbonate (77.34 mg, 0.240 mmol), ethanimidamide hydrochloride (11.22 mg, 0.120 mmol) and CuBr (0.57 mg, 0 mmol) in DMSO (0.791 mL) was stirred at 120° C. for 2 h. After this time the reaction was cooled down to RT and H₂O was added. The mixture was then extracted with AcOEt 3 times, washed with brine, dried over Na₂SO₄, filtered and concentrated under vacuum. The residue was purified by FC on NH column (eluting from 100% of DCM to DCM/MeOH 95:5) affording the product of formula [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone.

### Yield: 3 mg

1H NMR (400 MHZ, MeOD) δ 9.18-8.88 (m, 1H), 8.24 (d, 1H), 7.72 (s, 1H), 7.53 (d, 1H), 4.75 (d, 1H), 3.68 (d, 1H), 3.29-2.77 (m, 3H), 2.72 (s, 3H), 2.52 (s, 3H), 2.06 (t, 1H), 1.83 (d, 1H), 1.64 (p, 2H); LC-MS: m/z 499.39 (MH+).

### Preparation: [2-(4-cyclopropylthiazol-2-yl)-4-(5-methyl-4H-1,2,4-triazol-3-yl)phenyl]-[4-(trifluoromethyl)-1-piperidyl]methanone

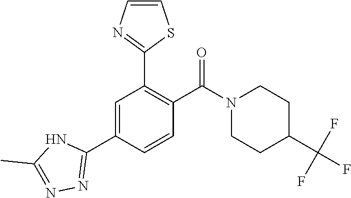 [SMILES: Cc1nnc(-c2ccc(C(=O)N3CCC(C(F)(F)F)CC3)c(-c3nccs3)c2)[nH]1]

### Example 69

The synthesis of the title compound was effected analogously to the synthesis of the compound of Example 68 [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone wherein 3-(4-cyclopropylthiazol-2-yl)-4-[4-(trifluoromethyl) piperidine-1-carbonyl]benzonitrile was used instead of 3-[4-methyl-6-(trifluoromethyl)pyrimidin-2-yl]-4-[4-(trifluoromethyl) piperidine-1-carbonyl]benzonitrile. The title compound was obtained in a yield of 48% (18 mg).

1H NMR (400 MHZ, DMSO) δ 13.87 (s, 1H), 8.34 (dd, 1), 8.12-8.03 (m, 1H), 7.56-7.29 (m, 2H), 4.62 (s, 1H), 3.41 (d, 1H), 3.07-2.57 (m, 3H), 2.42 (s, 3H), 2.11 (tt, 1H), 2.02-1.84 (m, 1H), 1.74-1.53 (m, 1H), 1.52-1.01 (m, 2H), 0.99-0.66 (m, 4H); LC-MS: m/z 462.22 (MH+).

### Preparation: [2-(6-isopropyl-2-pyridyl)-4-(5-methyl-4H-1,2,4-triazol-3-yl)phenyl]-[4-(trifluoromethyl)-1-piperidyl]methanone

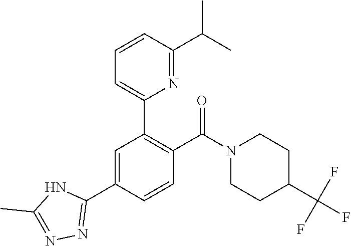 [SMILES: Cc1nnc(-c2ccc(C(=O)N3CCC(C(F)(F)F)CC3)c(-c3cccc(C(C)C)n3)c2)[nH]1]

### Example 70

The synthesis of the title compound was effected analogously to the synthesis of the compound of Example 68 [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone wherein 3-(6-propan-2-ylpyridin-2-yl)-4-[4-(trifluoromethyl) piperidine-1-carbonyl]benzonitrile was used instead of 3-[4-methyl-6-(trifluoromethyl)pyrimidin-2-yl]-4-[4-(trifluoromethyl) piperidine-1-carbonyl]benzonitrile. The title compound was obtained in a yield of 47% (25 mg).

1H NMR (400 MHZ, DMSO) δ 13.83 (s, 1H), 8.31-8.25 (m, 1H), 8.10-8.02 (m, 1H), 7.82 (t, 1H), 7.58-7.39 (m, 2H), 7.34-7.23 (m, 1H), 4.55 (d, 1H), 3.61-2.55 (m, 4H), 2.41 (s, 4H), 1.97-1.33 (m, 3H), 1.30-1.20 (m, 6H), 0.98-0.13 (m, 1H); LC-MS: m/z 458.48 (MH+).

### Preparation: [2-(6-cyclopropylpyridin-2-yl)-4-(5-methyl-4H-1,2,4-triazol-3-yl)phenyl]-[4-(trifluoromethyl)piperidin-1-yl]methanone

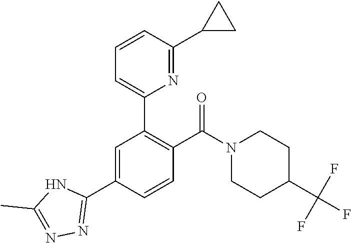 [SMILES: Cc1nnc(-c2ccc(C(=O)N3CCC(C(F)(F)F)CC3)c(-c3cccc(C4CC4)n3)c2)[nH]1]

### Example 71

The synthesis of the title compound was effected analogously to the synthesis of the compound of Example 68 [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone wherein 3-(6-cyclopropylpyridin-2-yl)-4-[4-(trifluoromethyl) piperidine-1-carbonyl]benzonitrile was used instead of 3-[4-methyl-6-(trifluoromethyl)pyrimidin-2-yl]-4-[4-(trifluoromethyl) piperidine-1-carbonyl]benzonitrile. The title compound was obtained in a yield of 17% (17 mg).

¹H NMR (400 MHZ, DMSO-d₆) δ 13.83 (s, 1H), 8.30-8.15 (m, 1H), 8.06 (dt, 1H), 7.76 (td, 1H), 7.51-7.35 (m, 2H), 7.28 (dd, 1H), 4.57 (d, 1H), 4.24-3.43 (m, 1H), 3.22-2.58 (m, 3H), 2.42 (s, 5H), 2.00-1.63 (m, 1H), 1.55-0.19 (m, 6H); LC-MS: m/z 456.22 (MH+).

### Preparation: [4-(5-methyl-4H-1,2,4-triazol-3-yl)-2-[4-(trifluoromethyl)-1,3-thiazol-2-yl]phenyl]-[4-(trifluoromethyl)piperidin-1-yl]methanone

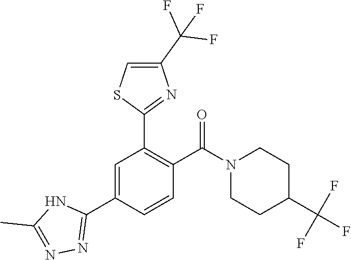 [SMILES: Cc1nnc(-c2ccc(C(=O)N3CCC(C(F)(F)F)CC3)c(-c3nc(C(F)(F)F)cs3)c2)[nH]1]

### Example 72

The synthesis of the title compound was effected analogously to the synthesis of the compound of Example 68 [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone wherein 4-[4-(trifluoromethyl) piperidine-1-carbonyl]-3-[4-(trifluoromethyl)-1,3-thiazol-2-yl]benzonitrile was used instead of 3-[4-methyl-6-(trifluoromethyl)pyrimidin-2-yl]-4-[4-(trifluoromethyl) piperidine-1-carbonyl]benzonitrile. The title compound was obtained in a yield of 48% (37 mg).

1H NMR (400 MHZ, DMSO) δ 13.91 (s, 1H), 8.65 (d, 1H), 8.44 (d, 1H), 8.18 (dd, 1H), 7.53 (dd, 1H), 4.62 (d, 1H), 3.49 (d, 1H), 3.12 (t, 1H), 2.80 (t, 1H), 2.60 (s, 1H), 2.44 (s, 3H), 2.05-1.29 (m, 3H), 1.03 (d, 1H); LC-MS: m/z 490.13 (MH+).

### Preparation: [4-(5-methyl-4H-1,2,4-triazol-3-yl)-2-[6-(trifluoromethyl)pyridin-2-yl]phenyl]-[4-(trifluoromethyl)piperidin-1-yl]methanone

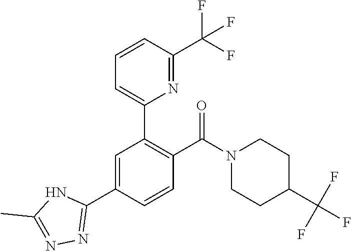 [SMILES: Cc1nnc(-c2ccc(C(=O)N3CCC(C(F)(F)F)CC3)c(-c3cccc(C(F)(F)F)n3)c2)[nH]1]

### Example 73

The synthesis of the title compound was effected analogously to the synthesis of the compound of Example 68 [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone wherein 4-[4-(trifluoromethyl) piperidine-1-carbonyl]-3-[6-(trifluoromethyl)pyridin-2-yl]benzonitrile was used instead of 3-[4-methyl-6-(trifluoromethyl)pyrimidin-2-yl]-4-[4-(trifluoromethyl) piperidine-1-carbonyl]benzonitrile. The title compound was obtained in a yield of 36% (31 mg).

1H NMR (400 MHZ, DMSO) δ 13.86 (s, 1H), 8.32 (d, 1H), 8.23 (t, 1H), 8.13 (dd, 1H), 8.06 (d, 1H), 7.92 (s, 1H), 7.52 (d, 1H), 4.51 (d, 1H), 3.50 (d, 1H), 3.10-2.70 (m, 2H), 2.59 (s, 1H), 2.43 (s, 3H), 2.00-1.37 (m, 3H), 0.77 (d, 1H); LC-MS: m/z 484.13 (MH+).

### Preparation: 4,4-difluoro-1-piperidyl)-[2-(6-isopropyl-2-pyridyl)-4-(5-methyl-4H-1,2,4-triazol-3-yl)phenyl]methanone

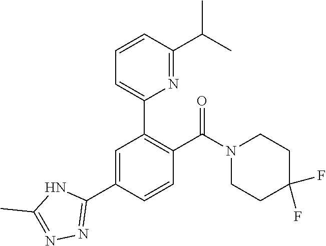 [SMILES: Cc1nnc(-c2ccc(C(=O)N3CCC(F)(F)CC3)c(-c3cccc(C(C)C)n3)c2)[nH]1]

### Example 74

The synthesis of the title compound was effected analogously to the synthesis of the compound of Example 68 [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone wherein 4-(4,4-difluoropiperidine-1-carbonyl)-3-(6-isopropyl-2-pyridyl)benzonitrile was used instead of 3-[4-methyl-6-(trifluoromethyl)pyrimidin-2-yl]-4-[4-(trifluoromethyl) piperidine-1-carbonyl]benzonitrile. The title compound was obtained in a yield of 50% (80 mg).

¹H NMR (400 MHZ, DMSO-d₆) δ 13.75 (bs, 1H), 8.30 (d, 1H), 8.07 (dd, 1H), 7.85 (t, 1H), 7.54 (dd, 2H), 7.30 (d, 1H), 3.93-3.83 (m, 1H), 3.45-3.37 (m, 2H), 3.15-3.07 (m, 1H), 3.01 (p, 1H), 2.42 (s, 3H), 2.16-1.74 (m, 3H), 1.56-1.44 (m, 1H), 1.25 (dd, 6H); LC-MS: m/z 426.35 (MH+).

### Preparation: (4,4-difluoropiperidin-1-yl)-[4-(5-methyl-4H-1,2,4-triazol-3-yl)-2-[6-(trifluoromethyl)pyridin-2-yl]phenyl]methanone

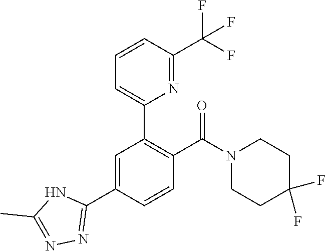 [SMILES: Cc1nnc(-c2ccc(C(=O)N3CCC(F)(F)CC3)c(-c3cccc(C(F)(F)F)n3)c2)[nH]1]

### Example 75

The synthesis of the title compound was effected analogously to the synthesis of the compound of Example 68 [4-(5-methyl-4H-1,2,4-triazol-3-yl)-2-[4-methyl-6-(trifluoromethyl)pyrimidin-2-yl]phenyl]-[4-(trifluoromethyl)-1-piperidyl]methanone wherein 4-(4,4-difluoropiperidine-1-carbonyl)-3-[6-(trifluoromethyl)pyridin-2-yl]benzonitrile was used instead of 3-[4-methyl-6-(trifluoromethyl)pyrimidin-2-yl]-4-[4-(trifluoromethyl) piperidine-1-carbonyl]benzonitrile. The title compound was obtained in a yield of 26% (25 mg).

1H NMR (400 MHZ, DMSO-d6) δ ppm 8.33 (d, 1H), 8.28-8.20 (m, 1H), 8.17-8.08 (m, 2H), 7.92 (d, 1H), 7.59 (d, 1H), 3.96-3.83 (m, 2H), 3.58-3.34 (m, 3H), 2.42 (s, 3H), 2.18-1.71 (m, 4H); LC-MS: m/z 452.20 (MH+).

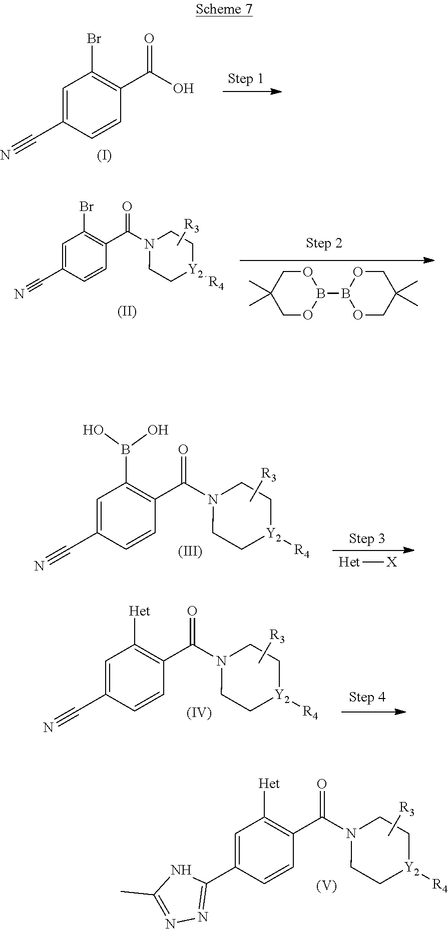

### Step 1

Compound of formula II may be obtained from compound of formula I (commercially available from Astatech) by coupling with a proper amine in the presence of coupling agent, e.g. HATU, and an organic base, e.g. DIPEA. The reaction is carried out in a suitable solvent such as DMF, typically at room temperature. The reaction takes from about 3 hours to about 12 hours to complete.

### Step 2

Compound of formula III may be obtained via Miyaura Borylation between compound of formula II (commercially available from Astatech) and 2-(5,5-dimethyl-1,3,2-dioxaborinan-2-yl)-5,5-dimethyl-1,3,2-dioxaborinane, in the presence of a suitable transition metal catalyst, e.g. Pd(dppf)Cl₂, a suitable base, e.g. potassium acetate, in a suitable solvents, e.g. 1,4-Dioxane, typically at 95° C. The reaction takes about 12 hours to complete.

### Step 3

Compound of formula IV may be obtained via Suzuki coupling between compound of formula III and commercially available heteroaryl halide in the presence of a suitable palladium catalyst, e.g. palladium tetrakis triphenylphosphine, suitable inorganic base Na₂CO₃, in a suitable mixture of solvents, 1,2-dimethoxyethane/water, typically at 95° C. The reaction takes from about 3 hours to about 12 hours to complete.

### Step 4

Compound of formula V may be obtained by nitrile cyclization of compound of formula IV with ethanimidamide hydrochloride in presence of a suitable catalyst, e.g. CuBr, a suitable base, e.g. Cs₂CO₃, in a suitable solvent, such as DMSO, typically at 120° C. The reaction takes about from 3 hours to about 12 hours to complete.

### Scheme 7, Cpd (II)*a Preparation: 3-bromo-4-(4,4-difluoropiperidine-1-carbonyl)benzonitrile

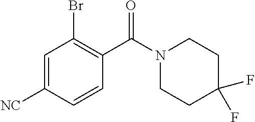 [SMILES: N#Cc1ccc(C(=O)N2CCC(F)(F)CC2)c(Br)c1]

To a solution of [dimethylamino (3-triazolo[4,5-b]pyridinyloxy)methylidene]-dimethylammonium hexafluorophosphate HATU (1.14 g, 2.99 mmol), 2-bromo-4-cyanobenzoic acid (450.0 mg, 1.99 mmol) and N,N-Diisopropylethylamine (2.03 mL, 11.95 mmol) in DMF (13.27 mL), 4,4-difluoropiperidine hydrochloride (376.49 mg, 2.39 mmol) was added. The reaction was stirred at RT for 3 h. After this time a saturated solution of NaHCO₃ was added to the reaction mixture and the aqueous phase was extracted with EtOAc (×3). The organic portions were collected, washed with brine, residual water was removed by adding Na₂SO₄ and the solvent was evaporated under reduced pressure. The residue was purified by FC on silica gel (eluting from 100% of cHex to cHexAcOEt/65:35) affording the product of formula 3-bromo-4-(4,4-difluoropiperidine-1-carbonyl)benzonitrile.

### Yield: 500 mg

1H NMR (400 MHZ, DMSO-d6) δ ppm 8.31 (d, 1H) 7.98 (dd, 1H) 7.66 (d, 1H) 3.82-3.93 (m, 1H) 3.60-3.73 (m, 1H) 3.22 (t, 2H) 1.88-2.22 (m, 4H); LC-MS: m/z 329.00, 331.00 (MH+).

### Scheme 7, Cpd (II)*b Preparation: 3-bromo-4-[4-(trifluoromethoxy) piperidine-1-carbonyl]benzonitrile

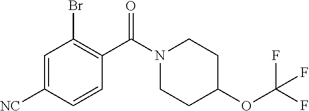 [SMILES: N#Cc1ccc(C(=O)N2CCC(OC(F)(F)F)CC2)c(Br)c1]

To a solution of [dimethylamino (3-triazolo[4,5-b]pyridinyloxy)methylidene]-dimethylammonium hexafluorophosphate HATU (630.83 mg, 1.66 mmol), 2-bromo-4-cyanobenzoic acid (250.0 mg, 1.11 mmol) and 4-(trifluoromethoxy) piperidine hydrochloride (272.9 mg, 1.33 mmol) in DMF (5.821 mL) N,N-Diisopropylethylamine (1.13 mL, 6.64 mmol) was added. The reaction was stirred at RT for 6 h. After this time the reaction was diluted with H₂O. The mixture was then extracted with AcOEt 3 times. The combined organic fractions were washed with brine, dried over a Na₂SO₄ filtered and concentrated under vacuum. The residue was purified by FC on silica gel (eluting from AcOEt:cHex from 5:95 to 60:40) affording the product of formula 3-bromo-4-[4-(trifluoromethoxy) piperidine-1-carbonyl]benzonitrile.

### Yield: 260 mg

1H NMR (400 MHZ, DMSO) δ 8.29 (dd, 1H), 7.96 (ddd, 1H), 7.72-7.49 (m, 1H), 4.74 (dt, 1H), 4.02 (td, 1H), 3.42 (dddd, 1H), 3.30-3.06 (m, 2H), 2.09-1.97 (m, 1H), 1.90 (s, 1H), 1.84-1.56 (m, 2H); LC-MS: m/z 377.36, 379.26 (MH+).

### Scheme 7, Cpd (III)*a Preparation: [5-cyano-2-(4,4-difluoropiperidine-1-carbonyl)phenyl]boronic acid

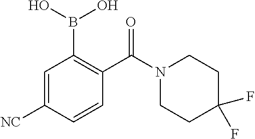 [SMILES: N#Cc1ccc(C(=O)N2CCC(F)(F)CC2)c(B(O)O)c1]

Potassium acetate (40.67 mg, 0.410 mmol), [1,1′-Bis(diphenylphosphino)ferrocene]dichloropalladium (II), complex with dichloromethane (5.6 mg, 0.010 mmol), 3-bromo-4-(4,4-difluoropiperidine-1-carbonyl)benzonitrile (45.0 mg, 0.140 mmol) and 2-(5,5-dimethyl-1,3,2-dioxaborinan-2-yl)-5,5-dimethyl-1,3,2-dioxaborinane (46.33 mg, 0.210 mmol) were suspended in 1,4-Dioxane (1 mL). The mixture was then degassed through N₂-vacuum cycles 3 times. The vial was sealed and the mixture was stirred at 90° C. for 3 h. After this time the reaction was cooled down, filtered through a pad of celite washing with EtOAc. The organic phase was washed with a s.s. of NaHCO₃, brine, dried over Na₂SO₄, filtered and concentrated under vacuum affording the product of formula [5-cyano-2-(4,4-difluoropiperidine-1-carbonyl)phenyl]boronic acid. The product was used in the next step without further purification.

### Yield: 40 mg

### LC-MS: m/z 295.24 (MH+).

### Scheme 7, Cpd (III)*b Preparation: [5-cyano-2-[4-(trifluoromethoxy) piperidine-1-carbonyl]phenyl]boronic acid

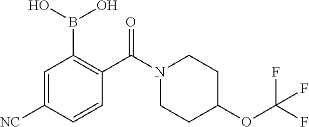 [SMILES: N#Cc1ccc(C(=O)N2CCC(OC(F)(F)F)CC2)c(B(O)O)c1]

The synthesis of the title compound was effected analogously to the synthesis of the compound [5-cyano-2-(4,4-difluoropiperidine-1-carbonyl)phenyl]boronic acid wherein 3-bromo-4-[4-(trifluoromethoxy) piperidine-1-carbonyl]benzonitrile was used instead of 3-bromo-4-(4,4-difluoropiperidine-1-carbonyl)benzonitrile. The title compound was obtained in a yield of 61% (145 mg).

### LC-MS: m/z 343.20 (MH+).

### Scheme 7, Cpd (IV)*a Preparation: 4-(4,4-difluoropiperidine-1-carbonyl)-3-[4-(trifluoromethyl) thiazol-2-yl]benzonitrile

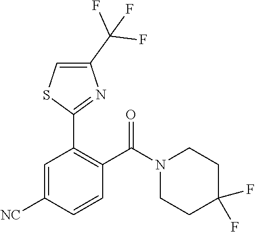 [SMILES: N#Cc1ccc(C(=O)N2CCC(F)(F)CC2)c(-c2nc(C(F)(F)F)cs2)c1]

Palladium tetrakis triphenylphosphine (15.72 mg, 0.010 mmol) and [5-cyano-2-(4,4-difluoropiperidine-1-carbonyl)phenyl]boronic acid (80.0 mg, 0.270 mmol) were suspended in 1,2-dimethoxyethane (2 mL) and then a 2 M aqueous solution of Na₂CO₃ (86.5 mg, 0.820 mmol) was added. The mixture was degassed by 5 cycles of nitrogen/vacuum and then 2-bromo-4-(trifluoromethyl) thiazole (69.43 mg, 0.300 mmol). The reaction mixture was degassed again by 5 cycles of nitrogen/vacuum and heated to 100° C. overnight. After this time the reaction was cooled down to room temperature, diluted with water and extracted with EtOAc (3×). The organic layers were collected together and washed with brine, dried over Na₂SO₄, filtered and the solvent removed under vacuum. The mixture was purified by FC on silica gel (eluting from 100% of cHex to cHex:EtOAc 50:50) to give the product of formula 4-(4,4-difluoropiperidine-1-carbonyl)-3-[4-(trifluoromethyl) thiazol-2-yl]benzonitrile.

### Yield: 35 mg

1H NMR (400 MHZ, DMSO-d6) δ 8.69 (d, 1H), 8.44 (d, 1H), 8.11 (dd, 1H), 7.70 (d, 1H), 3.87-3.78 (m, 1H), 3.63-3.53 (m, 1H), 2.20-1.85 (m, 6H); LC-MS: m/z 402.15 (MH+).

### Scheme 7, Cpd (IV)*b Preparation: 4-(4,4-difluoropiperidine-1-carbonyl)-3-[4-(trifluoromethyl)pyrimidin-2-yl]benzonitrile

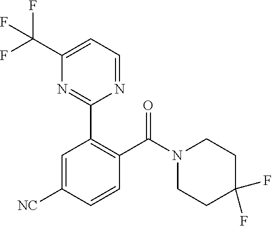 [SMILES: N#Cc1ccc(C(=O)N2CCC(F)(F)CC2)c(-c2nccc(C(F)(F)F)n2)c1]

The synthesis of the title compound was effected analogously to the synthesis of the compound 4-(4,4-difluoropiperidine-1-carbonyl)-3-[4-(trifluoromethyl) thiazol-2-yl]benzonitrile wherein 2-chloro-4-(trifluoromethyl)pyrimidine was used instead of 2-bromo-4-(trifluoromethyl) thiazole. The title compound was obtained in a yield of 40% (44 mg).

1H NMR (400 MHZ, DMSO-d6) δ 9.33 (d, 1H), 8.58 (d, 1H), 8.15 (dd, 1H), 8.09-8.04 (m, 1H), 7.76 (dd, 1H), 4.17-3.86 (m, 1H), 2.21-1.92 (m, 6H); LC-MS: m/z 397.21 (MH+).

### Scheme 7, Cpd (IV)*c Preparation: 3-(6-cyclopropyl-2-pyridyl)-4-(4,4-difluoropiperidine-1-carbonyl)benzonitrile

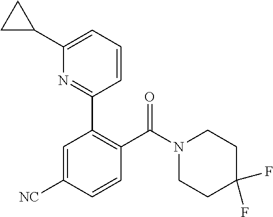 [SMILES: N#Cc1ccc(C(=O)N2CCC(F)(F)CC2)c(-c2cccc(C3CC3)n2)c1]

The synthesis of the title compound was effected analogously to the synthesis of the compound 4-(4,4-difluoropiperidine-1-carbonyl)-3-[4-(trifluoromethyl) thiazol-2-yl]benzonitrile wherein 2-bromo-6-cyclopropylpyridine was used instead of 2-bromo-4-(trifluoromethyl) thiazole. The title compound was obtained in a yield of 47% (28 mg). 1H NMR (400 MHZ, DMSO) δ 8.22 (d, 1H), 7.96 (td, 2H), 7.79 (t, 1H), 7.67 (dd, 2H), 3.93 (d, 1H), 3.74 (s, 1H), 3.41 (t, 1H), 2.98 (d, 1H), 2.20-1.81 (m, 6H), 1.41 (s, 1H), 1.00-0.91 (m, 2H); LC-MS: m/z 368.22 (MH+).

### Scheme 7, Cpd (IV)*d Preparation: 3-(6-tert-butyl-2-pyridyl)-4-(4,4-difluoropiperidine-1-carbonyl)benzonitrile

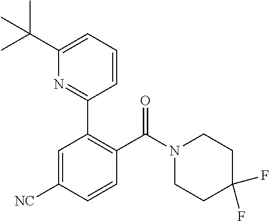 [SMILES: CC(C)(C)c1cccc(-c2cc(C#N)ccc2C(=O)N2CCC(F)(F)CC2)n1]

The synthesis of the title compound was effected analogously to the synthesis of the compound 4-(4,4-difluoropiperidine-1-carbonyl)-3-[4-(trifluoromethyl) thiazol-2-yl]benzonitrile wherein 2-bromo-6-tert-butyl-pyridine was used instead of 2-bromo-4-(trifluoromethyl) thiazole. The title compound was obtained in a yield of 82% (94 mg).

H NMR (400 MHZ, DMSO) δ 8.22 (d, 1H), 7.99 (dd, 1H), 7.87 (t, 1H), 7.73 (d, 1H), 7.61 (dd, 1H), 7.47 (dd, 1H), 4.11-3.96 (m, 1H), 3.25-3.14 (m, 2H), 3.06-2.90 (m, 1H), 2.19-1.77 (m, 3H), 1.56-1.38 (m, 1H), 1.30 (s, 9H); LC-MS: m/z 384.47 (MH+).

### Scheme 7, Cpd (IV)*e Preparation: 3-(1-tert-butylpyrazol-3-yl)-4-[4-(trifluoromethoxy) piperidine-1-carbonyl]benzonitrile

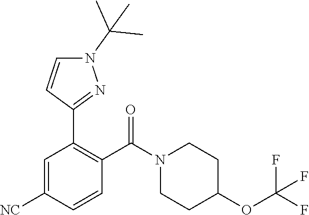 [SMILES: CC(C)(C)n1ccc(-c2cc(C#N)ccc2C(=O)N2CCC(OC(F)(F)F)CC2)n1]

The synthesis of the title compound was effected analogously to the synthesis of the compound 4-(4,4-difluoropiperidine-1-carbonyl)-3-[4-(trifluoromethyl) thiazol-2-yl]benzonitrile wherein [5-cyano-2-[4-(trifluoromethoxy) piperidine-1-carbonyl]phenyl]boronic acid and 1-tert-butyl-3-iodopyrazole were used instead of [5-cyano-2-(4,4-difluoropiperidine-1-carbonyl)phenyl]boronic acid and 2-bromo-4-(trifluoromethyl) thiazole respectively. The title compound was obtained in a yield of 33% (96 mg).

1H NMR (400 MHZ, DMSO) δ 8.22-8.12 (m, 1H), 7.96-7.88 (m, 1H), 7.82 (td, 1H), 7.50 (dd, 1H), 6.61 (dd, 1H), 4.65 (dt, 1H), 3.51 (ddd, 1H), 3.28-2.88 (m, 3H), 1.99 (s, 2H), 1.81-1.63 (m, 2H), 1.54 (d, 9H); LC-MS: m/z 421.23 (MH+).

### Scheme 7, Cpd (V)*a Preparation: (4,4-difluoro-1-piperidyl)-[4-(5-methyl-4H-1,2,4-triazol-3-yl)-2-[4-(trifluoromethyl) thiazol-2-yl]phenyl]methanone

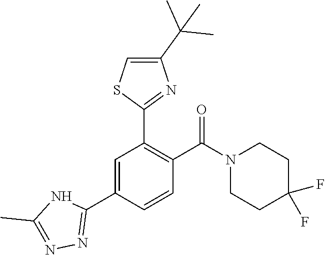 [SMILES: Cc1nnc(-c2ccc(C(=O)N3CCC(F)(F)CC3)c(-c3nc(C(C)(C)C)cs3)c2)[nH]1]

### Example 76

A mixture of 4-(4,4-difluoropiperidine-1-carbonyl)-3-[4-(trifluoromethyl) thiazol-2-yl]benzonitrile (35.0 mg, 0.070 mmol), CuBr (0.52 mg, 0 mmol), dicesium carbonate (94.33 mg, 0.290 mmol) and ethanimidamide hydrochloride (13.69 mg, 0.140 mmol) in DMSO (1.334 mL) was stirred at 120° C. for 2 h. After this time the reaction was cooled down to RT. H₂O was added and the mixture was then extracted with AcOEt 3 times. The combined organic fractions were washed with Brine, dried over Na₂SO₄, filtered and concentrated under vacuum. The residue was purified by FC on NH column (eluting from 100% of DCM to DCM:MeOH 95:5) and again by FC on reverse phase using acid conditions (eluting from 5:95 of CH₃CN/H₂O+0.1% of HCOOH to 55:45 of CH₃CN/H₂O+0.1% of HCOOH) affording the product of formula (4,4-difluoro-1-piperidyl)-[4-(5-methyl-4H-1,2,4-triazol-3-yl)-2-[4-(trifluoromethyl) thiazol-2-yl]phenyl]methanone.

### Yield: 14 mg

¹H NMR (400 MHz, DMSO-d₆) δ 8.65 (d, 1H), 8.43 (d, 1H), 8.19 (dd, 1H), 7.56 (d, 1H), 3.94-3.82 (m, 1H), 3.67-3.52 (m, 1H), 3.38-3.30 (m, 2H), 2.44 (s, 3H), 2.20-1.79 (m, 4H); LC-MS: m/z 458.38 (MH+).

### Preparation: (4,4-difluoro-1-piperidyl)-[4-(5-methyl-4H-1,2,4-triazol-3-yl)-2-[4-(trifluoromethyl)pyrimidin-2-yl]phenyl]methanone

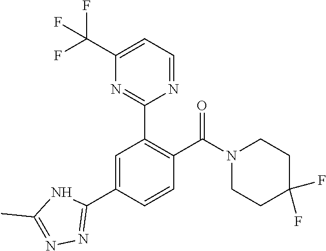 [SMILES: Cc1nnc(-c2ccc(C(=O)N3CCC(F)(F)CC3)c(-c3nccc(C(F)(F)F)n3)c2)[nH]1]

### Example 77

The synthesis of the title compound was effected analogously to the synthesis of the compound of Example 76 (4,4-difluoro-1-piperidyl)-[4-(5-methyl-4H-1,2,4-triazol-3-yl)-2-[4-(trifluoromethyl) thiazol-2-yl]phenyl]methanone wherein 4-(4,4-difluoropiperidine-1-carbonyl)-3-[4-(trifluoromethyl)pyrimidin-2-yl]benzonitrile was used instead of 4-(4,4-difluoropiperidine-1-carbonyl)-3-[4-(trifluoromethyl) thiazol-2-yl]benzonitrile. The title compound was obtained in a yield of 11% (5 mg).

1H NMR (400 MHZ, DMSO-d6) δ 9.30 (d, 1H), 8.86 (d, 1H), 8.19 (dd, 1H), 8.01 (d, 1H), 7.56 (d, 1H), 3.95-3.82 (m, 1H), 3.58-3.40 (m, 2H) 2.55-2.52 (m, 2H), 2.43 (s, 3H), 2.20-1.90 (m, 4H); LC-MS: m/z 453.26 (MH+).

### Preparation: [2-(6-cyclopropyl-2-pyridyl)-4-(5-methyl-4H-1,2,4-triazol-3-yl)phenyl]-(4,4-difluoro-1-piperidyl)methanone

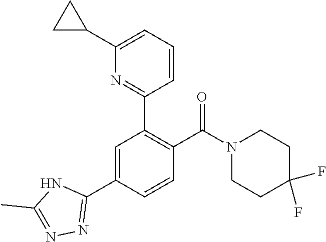 [SMILES: Cc1nnc(-c2ccc(C(=O)N3CCC(F)(F)CC3)c(-c3cccc(C4CC4)n3)c2)[nH]1]

### Example 78

The synthesis of the title compound was effected analogously to the synthesis of the compound of Example 76 (4,4-difluoro-1-piperidyl)-[4-(5-methyl-4H-1,2,4-triazol-3-yl)-2-[4-(trifluoromethyl) thiazol-2-yl]phenyl]methanone wherein 3-(6-cyclopropyl-2-pyridyl)-4-(4,4-difluoropiperidine-1-carbonyl)benzonitrile was used instead of 4-(4,4-difluoropiperidine-1-carbonyl)-3-[4-(trifluoromethyl) thiazol-2-yl]benzonitrile. The title compound was obtained in a yield of 33% (8 mg).

¹H NMR (400 MHZ, DMSO-d₆) δ 13.80 (bs, 1H), 8.26 (d, 1H), 8.06 (dd, 1H), 7.78 (t, 1H), 7.56-7.46 (m, 2H), 7.30 (dd, 1H), 3.92-3.80 (m, 1H), 3.54-3.45 (m, 2H), 3.08-2.93 (m, 1H), 2.42 (s, 3H), 2.19-1.80 (m, 3H), 1.36 (s, 1H), 1.12-0.69 (m, 4H); LC-MS: m/z 424.31 (MH+).

### Preparation: [2-(6-tert-butyl-2-pyridyl)-4-(5-methyl-4H-1,2,4-triazol-3-yl)phenyl]-(4,4-difluoro-1-piperidyl)methanone

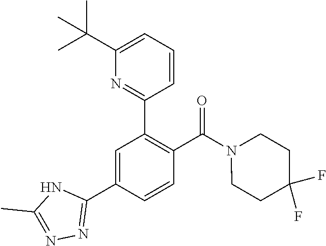 [SMILES: Cc1nnc(-c2ccc(C(=O)N3CCC(F)(F)CC3)c(-c3cccc(C(C)(C)C)n3)c2)[nH]1]

### Example 79

The synthesis of the title compound was effected analogously to the synthesis of the compound of Example 76 (4,4-difluoro-1-piperidyl)-[4-(5-methyl-4H-1,2,4-triazol-3-yl)-2-[4-(trifluoromethyl) thiazol-2-yl]phenyl]methanone wherein 3-(6-tert-butyl-2-pyridyl)-4-(4,4-difluoropiperidine-1-carbonyl)benzonitrile was used instead of 4-(4,4-difluoropiperidine-1-carbonyl)-3-[4-(trifluoromethyl) thiazol-2-yl]benzonitrile. The title compound was obtained in a yield of 35% (38 mg).

1H NMR (400 MHZ, DMSO) δ 13.85 (s, 1H), 8.29 (d, 1H), 8.07 (dd, 1H), 7.86 (t, 1H), 7.62-7.49 (m, 2H), 7.45 (dd, 1H), 3.93 (d, 1H), 3.41 (s, 2H), 3.01 (t, 1H), 2.42 (s, 3H), 1.93 (d, 3H), 1.32 (s, 10H); LC-MS: m/z 440.31 (MH+)

### Preparation [2-(1-tert-butylpyrazol-3-yl)-4-(5-methyl-4H-1,2,4-triazol-3-yl)phenyl]-[4-(trifluoromethoxy)-1-piperidyl]methanone

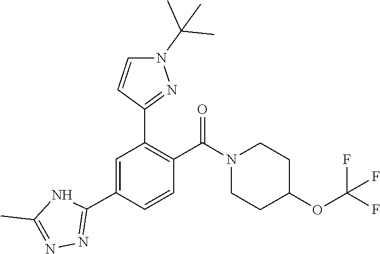 [SMILES: Cc1nnc(-c2ccc(C(=O)N3CCC(OC(F)(F)F)CC3)c(-c3ccn(C(C)(C)C)n3)c2)[nH]1]

### Example 80

The synthesis of the title compound was effected analogously to the synthesis of the compound of Example 76 (4,4-difluoro-1-piperidyl)-[4-(5-methyl-4H-1,2,4-triazol-3-yl)-2-[4-(trifluoromethyl) thiazol-2-yl]phenyl]methanone wherein 3-(1-tert-butylpyrazol-3-yl)-4-[4-(trifluoromethoxy) piperidine-1-carbonyl]benzonitrile was used instead of 4-(4,4-difluoropiperidine-1-carbonyl)-3-[4-(trifluoromethyl) thiazol-2-yl]benzonitrile. The title compound was obtained in a yield of 3.5% (4 mg).

1H NMR (400 MHZ, DMSO) δ 13.73 (s, 1H), 8.30 (dd, 1H), 8.01-7.81 (m, 2H), 7.33 (d, 1H), 6.53-6.43 (m, 1H), 4.67-4.55 (m, 1H), 4.14-3.95 (m, 1H), 3.29-2.85 (m, 3H), 2.42 (s, 3H), 1.99-1.88 (m, 1H), 1.74-1.61 (m, 1H), 1.56 (d, 11H); LC-MS: m/z 477.49 (MH+).

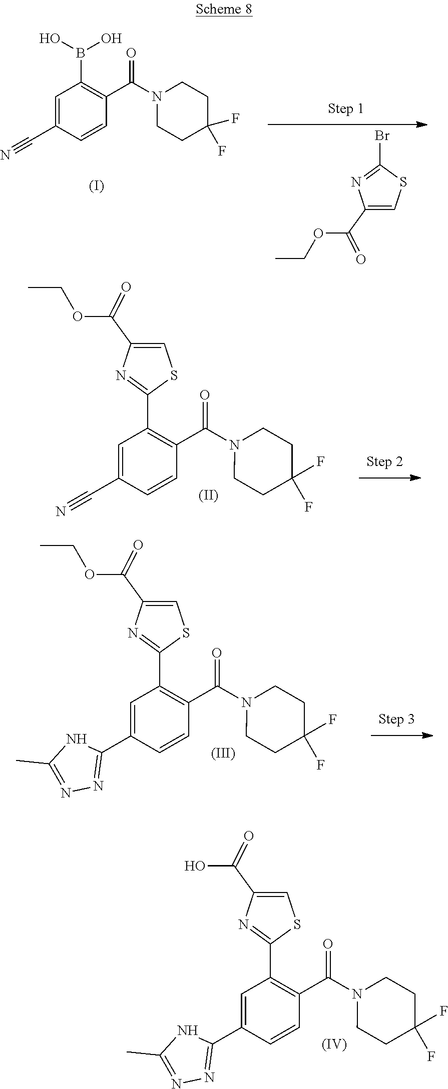

### Step 1

Compound of formula II may be obtained from compound of formula I (synthesized according to the Scheme 7) via Suzuki coupling with 2-bromo-4-thiazolecarboxylic acid ethyl ester, in the presence of a suitable precatalyst, e.g. XPhos Pd G2, a suitable base, e.g. K₃PO₄, in a suitable mixture of solvents, e.g. 1,2-dimethoxyethane/water, typically at 90° C. The reaction takes about 5 hours to complete.

### Step 2

Compound of formula III may be obtained by nitrile cyclization of compound of formula II with ethanimidamide hydrochloride in presence of a suitable catalyst, e.g. CuBr, a suitable base, e.g. Cs₂CO₃, in a suitable solvent, such as DMSO, typically at 120° C. The reaction takes about from 3 hours to about 12 hours to complete.

### Step 3

Compound of formula IV may be obtained from compound of formula III by hydrolysis, e.g. using LiOH, in a suitable mixture of solvents such as MeOH/THF/H₂O, typically at 50° C. The reaction takes about 12 hours to complete.

### Scheme 8, Cpd (II) Preparation: ethyl 2-[5-cyano-2-(4,4-difluoropiperidine-1-carbonyl)phenyl]thiazole-4-carboxylate

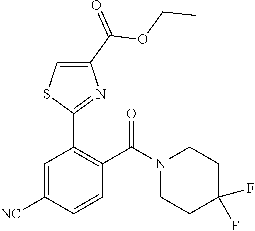 [SMILES: CCOC(=O)c1csc(-c2cc(C#N)ccc2C(=O)N2CCC(F)(F)CC2)n1]

### Example 81

In a proper vial 2-bromo-4-thiazolecarboxylic acid ethyl ester (134.88 mg, 0.570 mmol), [5-cyano-2-(4,4-difluoropiperidine-1-carbonyl)phenyl]boronic acid (560.0 mg, 0.380 mmol), X-phos aminobiphenyl palladium chloride precatalyst (29.93 mg, 0.040 mmol), tripotassium phosphate (242.54 mg, 1.14 mmol) were suspended in a mixture of Water (0.6 mL), 1,2-dimethoxyethane (5.7 mL). The resulting suspension was degassed with nitrogen and heated to 90° C. for 5 h. After this time the reaction mixture was cooled down to room temperature, diluted with water and extracted with EtOAc (3×). The organic layers were collected together and washed with brine, dried over Na₂SO₄, filtered and the solvent removed under vacuum. Crude was purified by FC on silica gel (eluting from cHex/EtOAc 95:5 to cHex/EtOAc 50:50) affording the product of formula ethyl 2-[5-cyano-2-(4,4-difluoropiperidine-1-carbonyl)phenyl]thiazole-4-carboxylate.

### Yield: 115 mg

1H NMR (400 MHZ, DMSO) δ 8.70 (s, 1H), 8.42 (d, 1H), 8.09 (dd, 1H), 7.68 (d, 1H), 4.40-4.26 (m, 2H), 3.97-3.83 (m, 1H), 3.68-3.52 (m, 1H), 3.43-3.33 (m, 2H), 2.29-1.86 (m, 4H), 1.32 (t, 3H); LC-MS: m/z 406.37 (MH+).

### Scheme 8, Cpd (III) Preparation: ethyl 2-[2-(4,4-difluoropiperidine-1-carbonyl)-5-(5-methyl-4H-1,2,4-triazol-3-yl)phenyl]thiazole-4-carboxylate

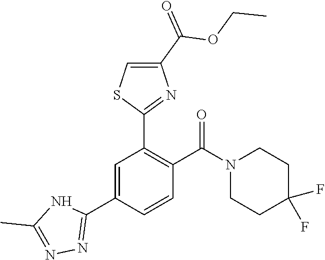 [SMILES: CCOC(=O)c1csc(-c2cc(-c3nnc(C)[nH]3)ccc2C(=O)N2CCC(F)(F)CC2)n1]

### Example 82

A mixture of ethyl 2-[5-cyano-2-(4,4-difluoropiperidine-1-carbonyl)phenyl]thiazole-4-carboxylate (115.0 mg, 0.280 mmol), CuBr (2.03 mg, 0.010 mmol), dicesium carbonate (277.26 mg, 0.850 mmol) and ethanimidamide hydrochloride (40.23 mg, 0.430 mmol) in DMSO (3.618 mL) was stirred at 120° C. for 90 min. After this time the reaction was cooled down to RT and H₂O was added. The mixture was then extracted with AcOEt 3 times. The combined organic fractions were washed with Brine, dried over Na₂SO₄, filtered and concentrated under vacuum. The residue was purified by FC on silica gel (eluting from 100% of DCM to DCM:EtOH 90:10) affording the product of formula ethyl 2-[2-(4,4-difluoropiperidine-1-carbonyl)-5-(5-methyl-4H-1,2,4-triazol-3-yl)phenyl]thiazole-4-carboxylate.

### Yield: 30 mg

¹H NMR (400 MHZ, DMSO-d₆) δ 13.90 (bs, 1H), 8.66 (s, 1H), 8.42 (d, 1H), 8.16 (dd, 1H), 7.54 (d, 1H), 4.34 (q, 2H), 4.01-3.92 (m, 1H), 3.65-3.55 (m, 1H), 3.41-3.36 (m, 2H), 2.44 (s, 3H), 2.29-1.83 (m, 4H), 1.33 (t, 3H); LC-MS: m/z 462.38 (MH+).

### Scheme 8, Cpd (IV) Preparation: 2-[2-(4,4-difluoropiperidine-1-carbonyl)-5-(5-methyl-4H-1,2,4-triazol-3-yl)phenyl]thiazole-4-carboxylic acid

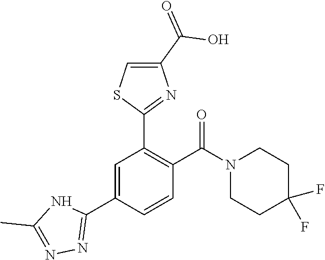 [SMILES: Cc1nnc(-c2ccc(C(=O)N3CCC(F)(F)CC3)c(-c3nc(C(=O)O)cs3)c2)[nH]1]

### Example 83

To a solution of ethyl 2-[2-(4,4-difluoropiperidine-1-carbonyl)-5-(5-methyl-4H-1,2,4-triazol-3-yl)phenyl]thiazole-4-carboxylate (23.0 mg, 0.040 mmol) in Methanol (0.142 mL)/THF (0.712 mL) a solution of lithium hydroxide (1.61 mg, 0.070 mmol) in Water (0.142 mL) was added. The reaction was stirred at 50° C. ON. The day after the reaction was cooled down to RT, acidified with HCl 1 N until pH=1 and directly purified by FC on reverse phase using acid conditions (eluting from CH₃CN/H₂O+0.1% of FA 5:95+0.1% of FA to CH₃CN/H₂O+0.1% of FA 35:65) affording the product of formula 2-[2-(4,4-difluoropiperidine-1-carbonyl)-5-(5-methyl-4H-1,2,4-triazol-3-yl)phenyl]thiazole-4-carboxylic acid.

### Yield: 6 mg

1H NMR (400 MHZ, DMSO) δ 13.86 (s, 1H), 8.71-8.30 (m, 2H), 8.14 (dd, 1H), 7.50 (d, 1H), 4.17-4.03 (m, 1H), 3.52-3.29 (m, 3H), 2.45 (s, 3H), 2.31-1.78 (m, 4H).

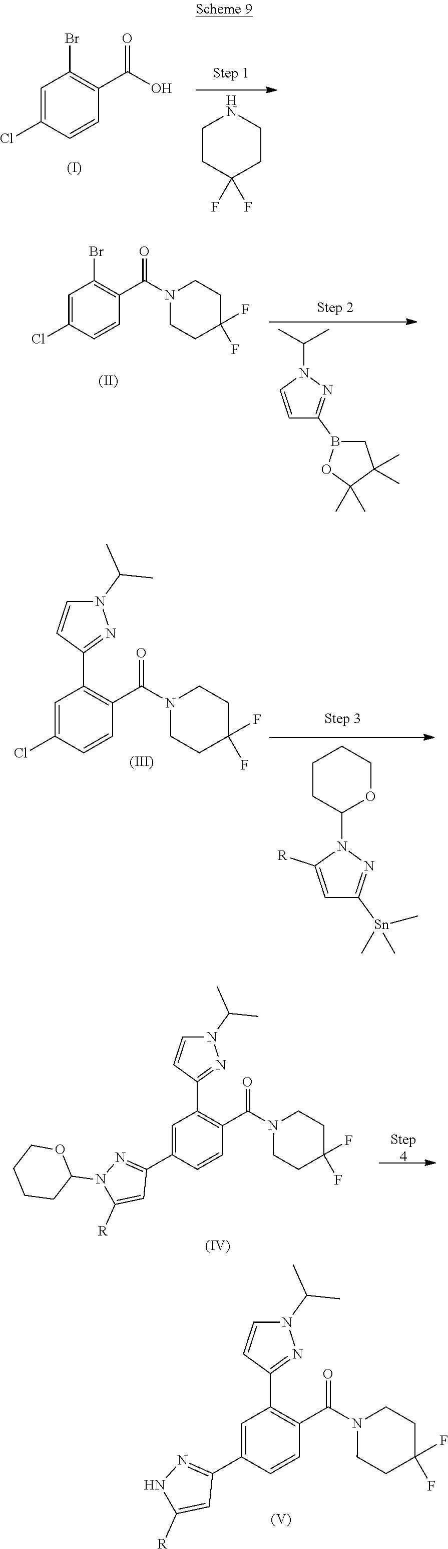

### Step 1

Compound of formula II may be obtained from compound of formula I (commercially available from Enamine) by coupling with 4,4-difluoropiperidine hydrochloride in the presence of a suitable coupling agent, e.g. HATU, and an organic base, e.g. DIPEA. The reaction is carried out in a suitable solvent such as DMF, typically at room temperature. The reaction takes about 12 hours to complete.

### Step 2

Compound of formula III may be obtained via Suzuki coupling between compound of formula II and 1-(1-Methylethyl)-3-(4,4,5,5-tetramethyl-1,3,2-dioxaborolan-2-yl)-1H-pyrazole (commercially available from Enamine), in the presence of a suitable precatalyst, e.g. XPhos Pd G2, a suitable base, e.g. K₃PO₄, in a suitable mixture of solvents, e.g. 1,2-dimethoxyethane/water, typically at 95° C. The reaction takes about 3 hours to complete.

### Step 3

Compound of formula IV may be obtained via Stille coupling reaction between compound of formula III and a proper THP protected stannane, in the presence of a suitable palladium pre-catalyst, e.g. palladium tritert-butylphosphine, a suitable base, e.g. CsF, in a suitable solvent, e.g. 1,4-dioxane, typically at 100° C. The reaction takes from about 3 hours to about 5 hours to complete.

### Step 4

Compound V may be obtained from compound of formula IV by removing the THP protecting group under acidic conditions, e.g. HCl 6N solution, typically at 50° C. The reaction takes about 3 hours to complete.

### Scheme 9, Cpd (II) Preparation: (2-bromo-4-chlorophenyl)-(4,4-difluoropiperidin-1-yl)methanone

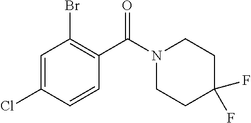 [SMILES: O=C(c1ccc(Cl)cc1Br)N1CCC(F)(F)CC1]

To a solution of HATU (1.45 g, 3.82 mmol), 2-bromo-4-chlorobenzoic acid (600.0 mg, 2.55 mmol) and N,N-Diisopropylethylamine (2.66 mL, 15.29 mmol) in DMF (16.99 mL), 4,4-difluoropiperidine; hydrochloride (481.89 mg, 3.06 mmol) was added. The reaction was stirred at RT overnight. The day after a saturated solution of NaHCO₃ was added to the reaction mixture and the aqueous phase was extracted with EtOAc (×3). The organic portions were collected, washed with brine, residual water was removed by adding Na₂SO₄ and the solvent was evaporated under reduced pressure. The residue was purified by FC on silica gel (eluting from 100% of cHex to AcOEt/cHex 30:70) affording the product of formula (2-bromo-4-chlorophenyl)-(4,4-difluoropiperidin-1-yl)methanone.

### Yield: 715 mg

1H NMR (400 MHZ, DMSO) δ 7.87 (d, 1H), 7.58 (dd, 1H), 7.48 (d, 1H), 3.86 (dt, 1H), 3.66 (ddd, 1H), 3.24 (t, 2H), 2.31-1.85 (m, 4H); LC-MS: m/z 338.00, 340.03 (MH+).

### Scheme 9, Cpd (III) Preparation: [4-chloro-2-(1-propan-2-ylpyrazol-3-yl)phenyl]-(4,4-difluoropiperidin-1-yl)methanone

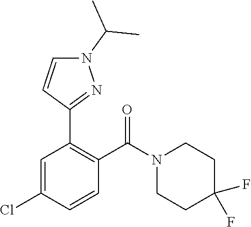 [SMILES: CC(C)n1ccc(-c2cc(Cl)ccc2C(=O)N2CCC(F)(F)CC2)n1]

In a proper vial, a mixture of X-phos aminobiphenyl palladium chloride precatalyst (162.46 mg, 0.210 mmol), tripotassium phosphate (1316.58 mg, 6.2 mmol), (2-bromo-4-chlorophenyl)-(4,4-difluoropiperidin-1-yl)methanone (700.0 mg, 2.07 mmol) and 1-(1-Methylethyl)-3-(4,4,5,5-tetramethyl-1,3,2-dioxaborolan-2-yl)-1H-pyrazole (390.53 mg, 1.65 mmol) in Water (0.952 mL)/1,2-dimethoxyethane (14.95 mL) was degassed bubbling N₂ for 10 min. The mixture was then subjected to N₂-vacuum cycles and then stirred at 95° C. for 3 h. After this time the reaction was cooled down to RT. Water was added to the mixture and aqueous phase was extracted with AcOEt 3 times. Organic phases were combined, washed with brine, dried over Na₂SO₄ and concentrated under vacuum. The residue was purified by FC on silica gel (eluting from 100% of cHex to cHex/EtOAc 40:60) affording the product of formula [4-chloro-2-(1-propan-2-ylpyrazol-3-yl)phenyl]-(4,4-difluoropiperidin-1-yl)methanone.

### Yield: 450 mg,

¹H NMR (400 MHZ, DMSO-d₆) δ 7.84 (d, 1H), 7.76 (d, 1H), 7.45 (dd, 1H), 7.34 (d, 1H), 6.55 (d, 1H), 4.51 (hept, 1H), 3.82-3.61 (m, 2H), 3.24-3.02 (m, 2H), 2.16-1.72 (m, 4H), 1.43 (dd, 6H); LC-MS: m/z 368.36 (MH+).

### Scheme 9, Cpd (IV)*a Preparation: (4,4-difluoro-1-piperidyl)-[2-(1-isopropylpyrazol-3-yl)-4-[1-tetrahydropyran-2-yl-5-(trifluoromethyl) pyrazol-3-yl]phenyl]methanone

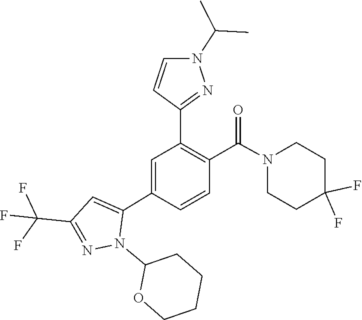 [SMILES: CC(C)n1ccc(-c2cc(-c3cc(C(F)(F)F)nn3C3CCCCO3)ccc2C(=O)N2CCC(F)(F)CC2)n1]

In a proper vial palladium tritert-butylphosphine (4.42 mg, 0.010 mmol) and cesium fluoride (72.21 mg, 0.480 mmol) were placed and N₂-vacuum cycles were performed 3 times. Then, a solution of trimethyl-[1-tetrahydropyran-2-yl-5-(trifluoromethyl) pyrazol-3-yl]stannane (283.73 mg, 0.260 mmol) and [4-chloro-2-(1-propan-2-ylpyrazol-3-yl)phenyl]-(4,4-difluoropiperidin-1-yl)methanone (85.0 mg, 0.220 mmol) in 1,4-Dioxane (3 mL) was added. The resulting suspension was degassed bubbling N₂ for 5 min and then 5 cycles of N₂-vacuum were performed. The mixture was stirred at 100° C. for 4 h. After this time the mixture was cooled down to RT, diluted with EtOAc, filtered over a pad of celite washing with EtOAc and concentrated under high vacuum. The crude was purified by FC on reverse phase using acid conditions (eluting from 5:95 of CH₃CN/H₂O+0.1% of HCOOH to 65:35 of CH₃CN/H₂O+0.1% of HCOOH) affording the product of formula (4,4-difluoro-1-piperidyl)-[2-(1-isopropylpyrazol-3-yl)-4-[1-tetrahydropyran-2-yl-5-(trifluoromethyl) pyrazol-3-yl]phenyl]methanone.

### Yield: 51 mg

1H NMR (400 MHZ, DMSO) δ 7.98 (dd, 1H), 7.90-7.81 (m, 1H), 7.63-7.53 (m, 1H), 7.49 (dd, 1H), 7.12 (d, 1H), 6.54 (t, 1H), 5.46-5.37 (m, 1H), 4.52 (p, 1H), 4.11-3.95 (m, 1H), 3.89-3.76 (m, 1H), 3.75-3.49 (m, 2H), 3.26-3.21 (m, 1H), 3.21-3.11 (m, 1H), 2.43-2.35 (m, 1H), 2.18-1.80 (m, 5H), 1.74-1.47 (m, 5H), 1.47-1.33 (m, 6H); LC-MS: m/z 552.29 (MH+)

### Scheme 9, Cpd (IV)*b Preparation: (4,4-difluoro-1-piperidyl)-[2-(1-isopropylpyrazol-3-yl)-4-(5-isopropyl-1-tetrahydropyran-2-yl-pyrazol-3-yl)phenyl]methanone

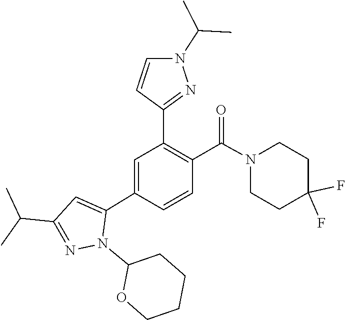 [SMILES: CC(C)c1cc(-c2ccc(C(=O)N3CCC(F)(F)CC3)c(-c3ccn(C(C)C)n3)c2)n(C2CCCCO2)n1]

The synthesis of the title compound was effected analogously to the synthesis of the compound (4,4-difluoro-1-piperidyl)-[2-(1-isopropylpyrazol-3-yl)-4-[1-tetrahydropyran-2-yl-5-(trifluoromethyl) pyrazol-3-yl]phenyl]methanone wherein (5-isopropyl-1-tetrahydropyran-2-yl-pyrazol-3-yl)-trimethyl-stannane was used instead of trimethyl-[1-tetrahydropyran-2-yl-5-(trifluoromethyl) pyrazol-3-yl]stannane. The title compound was obtained in a yield of 59% (55 mg).

1H NMR (400 MHZ, DMSO) δ 8.07 (d, 1H), 7.85 (d, 1H), 7.80 (d, 1H), 7.32 (d, 1H), 6.70 (s, 1H), 6.54 (d, 1H), 5.45 (d, 1H), 4.54 (p, 1H), 3.97-3.80 (m, 2H), 3.76-3.62 (m, 1H), 3.62-3.51 (m, 1H), 3.15-3.04 (m, 2H), 2.45-2.36 (m, 1H), 2.15-2.01 (m, 3H), 1.98-1.67 (m, 6H), 1.64-1.51 (m, 5H), 1.48-1.39 (m, 8H); LC-MS: m/z 526.33 (MH+).

### Scheme 9, Cpd (IV)*c Preparation: (4,4-difluoro-1-piperidyl)-[2-(1-isopropylpyrazol-3-yl)-4-(5-methyl-1-tetrahydropyran-2-yl-pyrazol-3-yl)phenyl]methanone

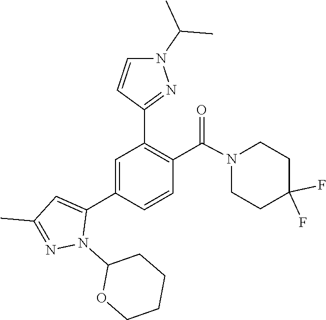 [SMILES: Cc1cc(-c2ccc(C(=O)N3CCC(F)(F)CC3)c(-c3ccn(C(C)C)n3)c2)n(C2CCCCO2)n1]

The synthesis of the title compound was effected analogously to the synthesis of the compound (4,4-difluoro-1-piperidyl)-[2-(1-isopropylpyrazol-3-yl)-4-[1-tetrahydropyran-2-yl-5-(trifluoromethyl) pyrazol-3-yl]phenyl]methanone wherein trimethyl-(5-methyl-1-tetrahydropyran-2-yl-pyrazol-3-yl) stannane was used instead of trimethyl-[1-tetrahydropyran-2-yl-5-(trifluoromethyl) pyrazol-3-yl]stannane. The title compound was obtained in a yield of 40% (24 mg).

1H NMR (400 MHZ, DMSO) δ 8.05 (d, 1H), 7.84 (d, 1H), 7.77 (d, 1H), 7.32 (d, 1H), 6.65 (s, 1H), 6.51 (d, 1H), 5.43 (d, 1H), 4.53 (p, 1H), 3.92 (d, 2H), 3.69 (d, 1H), 3.57 (s, 1H), 3.23-3.04 (m, 2H), 2.40-2.31 (m, 3H), 2.12-2.01 (m, 2H), 1.97-1.51 (m, 7H), 1.46-1.42 (m, 7H); LC-MS: m/z 498.49 (MH+).

### Scheme 9, Cpd (V)*a Preparation: (4,4-difluoro-1-piperidyl)-[2-(1-isopropylpyrazol-3-yl)-4-[5-(trifluoromethyl)-1H-pyrazol-3-yl]phenyl]methanone

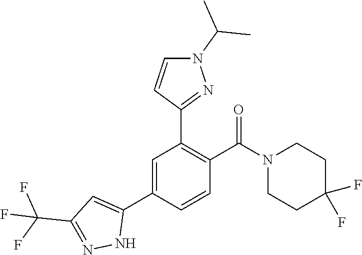 [SMILES: CC(C)n1ccc(-c2cc(-c3cc(C(F)(F)F)n[nH]3)ccc2C(=O)N2CCC(F)(F)CC2)n1]

### Example 84

To a solution of (4,4-difluoro-1-piperidyl)-[2-(1-isopropylpyrazol-3-yl)-4-[1-tetrahydropyran-2-yl-5-(trifluoromethyl) pyrazol-3-yl]phenyl]methanone (50.0 mg, 0.090 mmol) in Ethanol (2 mL) a 6 N aqueous solution of HCl (45.33 uL, 0.270 mmol) was added. The mixture was stirred at 60° C. for 2 h. After this time the reaction mixture was cooled down to RT, a s.s. of NaHCO₃ was added and the mixture was extracted with EtOAc (3 times). The organic portions were combined, washed with brine, dried over Na₂SO₄ and concentrated under vacuum. The crude was purified by FC on reverse phase using acid conditions (eluting from 5:95 of CH₃CN/H₂O+0.1% of HCOOH to 55:45 of CH₃CN/H₂O+0.1% of HCOOH) affording the product of formula (4,4-difluoro-1-piperidyl)-[2-(1-isopropylpyrazol-3-yl)-4-[5-(trifluoromethyl)-1H-pyrazol-3-yl]phenyl]methanone.

### Yield: 20 mg

1H NMR (400 MHZ, DMSO) δ 14.19 (s, 1H), 8.15 (d, 1H), 7.87 (d, 1H), 7.82 (dd, 1H), 7.42 (d, 1H), 7.33 (s, 1H), 6.62 (d, 1H), 4.52 (hept, 1H), 3.86-3.58 (m, 2H), 3.23-3.07 (m, 2H), 2.17-1.75 (m, 3H), 1.44 (dd, 7H); LC-MS: m/z 468.22 (MH+).

### Preparation: (4,4-difluoro-1-piperidyl)-[2-(1-isopropylpyrazol-3-yl)-4-(5-isopropyl-1H-pyrazol-3-yl)phenyl]methanone

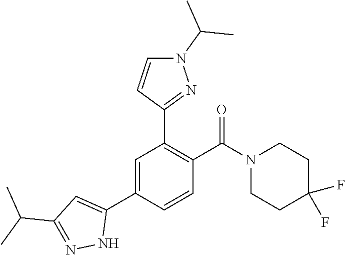 [SMILES: CC(C)c1cc(-c2ccc(C(=O)N3CCC(F)(F)CC3)c(-c3ccn(C(C)C)n3)c2)[nH]n1]

### Example 85

The synthesis of the title compound was effected analogously to the synthesis of the compound of Example 84 (4,4-difluoro-1-piperidyl)-[2-(1-isopropylpyrazol-3-yl)-4-[5-(trifluoromethyl)-1H-pyrazol-3-yl]phenyl]methanone wherein (4,4-difluoro-1-piperidyl)-[2-(1-isopropylpyrazol-3-yl)-4-(5-isopropyl-1-tetrahydropyran-2-yl-pyrazol-3-yl)phenyl]methanone was used instead of (4,4-difluoro-1-piperidyl)-[2-(1-isopropylpyrazol-3-yl)-4-[1-tetrahydropyran-2-yl-5-(trifluoromethyl) pyrazol-3-yl]phenyl]methanone. The title compound was obtained in a yield of 40% (15 mg).

1H NMR (400 MHZ, DMSO) δ 12.67 (s, 1H), 8.07 (s, 1H), 7.84 (d, 1H), 7.76 (d, 1H), 7.30 (d, 1H), 6.56 (d, 2H), 4.52 (hept, 1H), 3.95-3.79 (m, 1H), 3.65-3.57 (m, 1H), 3.23-2.92 (m, 3H), 2.18-1.72 (m, 3H), 1.44 (dd, 7H), 1.28 (d, 6H); LC-MS: m/z 442.48 (MH+).

### Preparation: (4,4-difluoro-1-piperidyl)-[2-(1-isopropylpyrazol-3-yl)-4-(5-methyl-1H-pyrazol-3-yl)phenyl]methanone

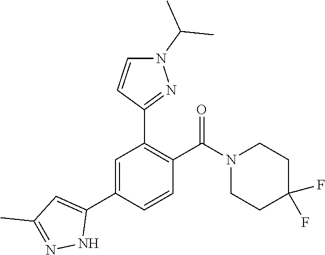 [SMILES: Cc1cc(-c2ccc(C(=O)N3CCC(F)(F)CC3)c(-c3ccn(C(C)C)n3)c2)[nH]n1]

### Example 86

The synthesis of the title compound was effected analogously to the synthesis of the compound of Example 84 (4,4-difluoro-1-piperidyl)-[2-(1-isopropylpyrazol-3-yl)-4-[5-(trifluoromethyl)-1H-pyrazol-3-yl]phenyl]methanone wherein (4,4-difluoro-1-piperidyl)-[2-(1-isopropylpyrazol-3-yl)-4-(5-methyl-1-tetrahydropyran-2-yl-pyrazol-3-yl)phenyl]methanone was used instead of (4,4-difluoro-1-piperidyl)-[2-(1-isopropylpyrazol-3-yl)-4-[1-tetrahydropyran-2-yl-5-(trifluoromethyl) pyrazol-3-yl]phenyl]methanone. The title compound was obtained in a yield of 66% (14 mg).

1H NMR (400 MHZ, DMSO) δ 12.63 (s, 1H), 8.07 (s, 1H), 7.84 (d, 1H), 7.74 (d, 1H), 7.30 (d, 1H), 6.53 (d, 2H), 4.52 (hept, 1H), 3.92-3.75 (m, 1H), 3.73-3.52 (m, 1H), 3.24-3.06 (m, 2H), 2.29 (s, 3H), 2.15-1.71 (m, 3H), 1.44 (dd, 7H); LC-MS: m/z 414.47 (MH+).

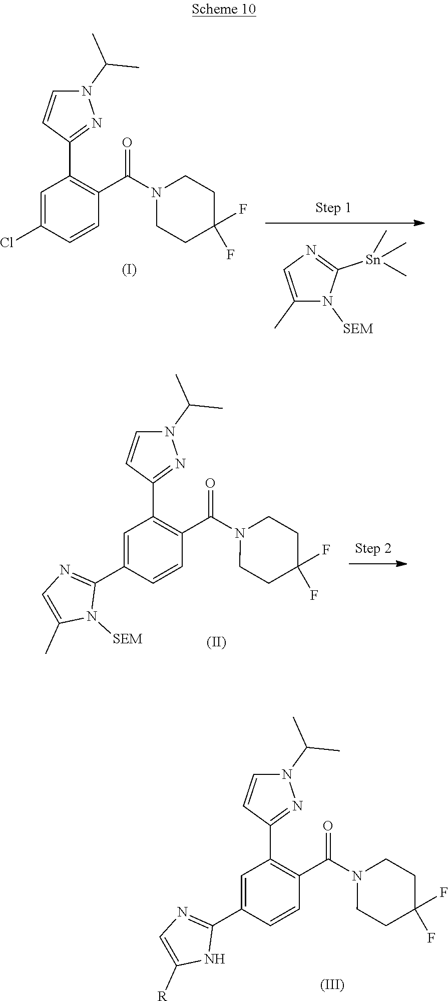

### Step 1

Compound of formula II may be obtained via Stille coupling reaction between compound of formula I (synthesized according to the Scheme 9) and trimethyl-[2-[(5-methyl-2-trimethylstannyl-imidazol-1-yl)methoxy]ethyl]silane, in the presence of a suitable catalyst, e.g. palladium tritert-butylphosphine, a suitable base, e.g. CsF, in a suitable solvent, e.g. 1,4-dioxane, typically at 100° C. The reaction takes about 5 hours to complete.

### Step 2

Compound of formula III may be obtained from compound of formula II by removing the SEM protecting group under acidic conditions, e.g. HCl 6N solution, typically at 75° C. The reaction takes about 16 hours to complete.

### Scheme 10, Cpd (II)*a Preparation: (4,4-difluoro-1-piperidyl)-[2-(1-isopropylpyrazol-3-yl)-4-[5-methyl-1-(2-trimethylsilylethoxymethyl) imidazol-2-yl]phenyl]methanone and (4,4-difluoro-1-piperidyl)-[2-(1-isopropylpyrazol-3-yl)-4-[4-methyl-1-(2-trimethylsilylethoxymethyl) imidazol-2-yl]phenyl]methanone

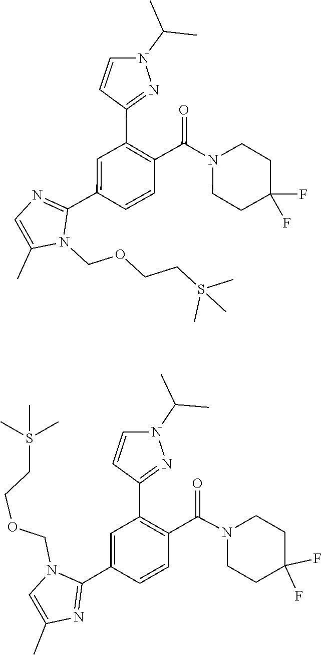 [SMILES: Cc1cnc(-c2ccc(C(=O)N3CCC(F)(F)CC3)c(-c3ccn(C(C)C)n3)c2)n1COCCS(C)(C)C ; Cc1cn(COCCS(C)(C)C)c(-c2ccc(C(=O)N3CCC(F)(F)CC3)c(-c3ccn(C(C)C)n3)c2)n1]

In a proper vial palladium tritert-butylphosphine (3.21 mg, 0.010 mmol) and cesium fluoride (69.96 mg, 0.460 mmol) were placed and 3 cycles of N₂-vacuum were performed. Then, a solution of trimethyl-[2-[(5-methyl-2-trimethylstannyl-imidazol-1-yl)methoxy]ethyl]silane/trimethyl-[2-[(4-methyl-2-trimethylstannyl-imidazol-1-yl)methoxy]ethyl]silane (78.54 mg, 0.210 mmol) and [4-chloro-2-(1-propan-2-ylpyrazol-3-yl)phenyl]-(4,4-difluoropiperidin-1-yl)methanone (77.0 mg, 0.210 mmol) in 1,4-Dioxane (2.093 mL) was added. The resulting suspension was degassed bubbling N₂ for 5 min and, then 5 cycles of N₂-vacuum were performed. The mixture was stirred at 100° C. for 5 h. After this time the mixture was cooled down to RT, diluted with EtOAc, filtered over a pad of celite washing with EtOAc and concentrated under high vacuum. The crude was purified by FC on reverse phase using acid conditions (eluting from 5:95 of CH₃CN/H₂O+0.1% of HCOOH to 45:55 of CH₃CN/H₂O+0.1% of HCOOH) affording the products of formula (4,4-difluoro-1-piperidyl)-[2-(1-isopropylpyrazol-3-yl)-4-[5-methyl-1-(2-trimethylsilylethoxymethyl) imidazol-2-yl]phenyl]methanone and (4,4-difluoro-1-piperidyl)-[2-(1-isopropylpyrazol-3-yl)-4-[4-methyl-1-(2-trimethylsilylethoxymethyl) imidazol-2-yl]phenyl]methanone as mixture of regioisomers. The mixture of regioisomers was used in the next step without further purification.

### Yield: 18 mg

### LC-MS: m/z 544.57 (MH+).

### Scheme 10, Cpd (III)*a Preparation: (4,4-difluoro-1-piperidyl)-[2-(1-isopropylpyrazol-3-yl)-4-(5-methyl-1H-imidazol-2-yl)phenyl]methanone

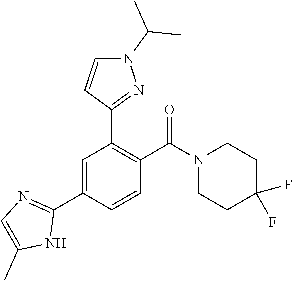 [SMILES: Cc1cnc(-c2ccc(C(=O)N3CCC(F)(F)CC3)c(-c3ccn(C(C)C)n3)c2)[nH]1]

### Example 87

To a solution of (4,4-difluoro-1-piperidyl)-[2-(1-isopropylpyrazol-3-yl)-4-[5-methyl-1-(2-trimethylsilylethoxymethyl) imidazol-2-yl]phenyl]methanone/(4,4-difluoro-1-piperidyl)-[2-(1-isopropylpyrazol-3-yl)-4-[4-methyl-1-(2-trimethylsilylethoxymethyl) imidazol-2-yl]phenyl]methanone (mixture of regioisomers) (17.0 mg, 0.030 mmol) in Ethanol (1 mL) a 6 N aqueous solution of HCl (15.63 uL, 0.090 mmol) was added. The mixture was stirred for 16 h at 75° C. After this time the mixture was cooled down, a s.s. of NaHCO₃ was added to the solution and the mixture was extracted with EtOAc (3×). The organic portions were combined, washed with brine, dried over Na₂SO₄ and concentrated under vacuum. The crude was purified by FC on reverse phase using acid conditions (eluting from 5:95 of CH₃CN/H₂O+0.1% of HCOOH to 30:70 of CH₃CN/H₂O+0.1% of HCOOH) affording the product of formula (4,4-difluoro-1-piperidyl)-[2-(1-isopropylpyrazol-3-yl)-4-(5-methyl-1H-imidazol-2-yl)phenyl]methanone.

### Yield: 8 mg

1H NMR (400 MHZ, DMSO) δ 12.18 (d, 1H), 8.01 (d, 1H), 7.82 (d, 1H), 7.69-7.57 (m, 2H), 7.28 (d, 1H), 6.45 (d, 1H), 4.52 (hept, 1H), 3.98-3.80 (m, 1H), 3.68-3.48 (m, 1H), 3.23-3.07 (m, 2H), 2.45 (s, 3H), 2.16-1.71 (m, 3H), 1.44 (dd, 7H); LC-MS: m/z 414.28 (MH+).

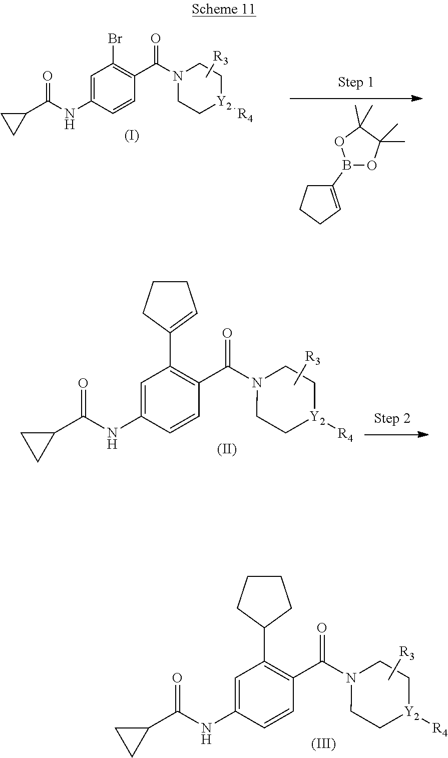

### Step 1

Compound of formula II may be obtained via Suzuki coupling between compound of formula I (synthesized according to the Scheme 3) and the desired boronic ester, such as 2-(cyclopenten-1-yl)-4,4,5,5-tetramethyl-1,3,2-dioxaborolane, in the presence of a suitable catalyst, e.g. palladium tetrakis triphenylphosphine, a suitable base, e.g. Na₂CO₃, in a suitable mixture of solvents, e.g. Toluene/Ethanol/water, typically at 100° C. The reaction takes from about 2 hours to 12 hours to complete.

### Step 2

Compound of formula III may be obtained by reduction of double bond of compound of formula II under catalytic hydrogenation conditions, using for example Pd/C in a suitable solvent such as Ethanol, typically at room temperature. The reaction takes about 2 hours to complete

### Preparation: N-[3-(cyclopenten-1-yl)-4-(1,1-dioxo-1,4-thiazinane-4-carbonyl)phenyl]cyclopropanecarboxamide

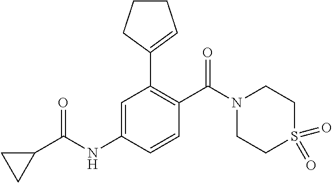 [SMILES: O=C(Nc1ccc(C(=O)N2CCS(=O)(=O)CC2)c(C2=CCCC2)c1)C1CC1]

### Example 88

In a vial were sequentially added palladium tetrakis triphenylphosphine (11.55 mg, 0.010 mmol), sodium carbonate (369.78 mg, 3.49 mmol) and 2-(cyclopenten-1-yl)-4,4,5,5-tetramethyl-1,3,2-dioxaborolane (96.73 mg, 0.500 mmol). Solids were suspended in a mixture of Ethanol (1.7 mL), Toluene (1.7 mL) Water (0.500 mL) and the resulting suspension was degassed using shlenk line technique. N-[3-bromo-4-(1,1-dioxo-1,4-thiazinane-4-carbonyl)phenyl]cyclopropanecarboxamide (200.0 mg, 0.500 mmol) was added under nitrogen flux. The mixture was degassed again and heated to 100° C. for 2 h. After this time the reaction mixture was cooled down to room temperature, diluted with water and extracted with EtOAc. The organic layers were collected together and washed with brine, dried over Na₂SO₄, filtered and the solvent removed under vacuum. The crude was purified by FC on reverse phase using acid conditions (eluting from 5:95 of CH₃CN/H₂O+0.1% of HCOOH to 40:60 of CH₃CN/H₂O+0.1% of HCOOH) affording the product of formula N-[3-(cyclopenten-1-yl)-4-(1,1-dioxo-1,4-thiazinane-4-carbonyl)phenyl]cyclopropanecarboxamide.

### Yield: 23 mg

1H NMR (400 MHZ, DMSO-d6) δ 10.31 (s, 1H), 7.61 (d, 1H), 7.56 (dd, 1H), 7.28 (d, 1H), 5.88 (p, 1H), 4.28 (s, 1H), 3.73 (s, 1H), 3.47 (d, 3H), 3.14 (d, 3H), 2.88 (s, 1H), 2.68-2.52 (m, 1H), 2.46 (s, 2H), 1.97-1.84 (m, 2H), 1.77 (tt, 1H), 0.85-0.75 (m, 4H); LC-MS: m/z 389.2 (MH+).

### Preparation: N-[3-(cyclopenten-1-yl)-4-(4-methyl-2-phenylpiperazine-1-carbonyl)phenyl]cyclopropanecarboxamide

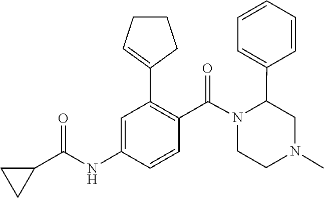 [SMILES: CN1CCN(C(=O)c2ccc(NC(=O)C3CC3)cc2C2=CCCC2)C(c2ccccc2)C1]

### Example 89

The synthesis of the title compound was effected analogously to the synthesis of the compound of Example 88 N-[3-(cyclopenten-1-yl)-4-(1,1-dioxo-1,4-thiazinane-4-carbonyl)phenyl]cyclopropanecarboxamide wherein N-[3-bromo-4-(4-methyl-2-phenylpiperazine-1-carbonyl)phenyl]cyclopropanecarboxamide was used instead of N-[3-bromo-4-(1,1-dioxo-1,4-thiazinane-4-carbonyl)phenyl]cyclopropanecarboxamide. The title compound was obtained in a yield of 51% (30 mg).

¹H NMR (400 MHZ, DMSO-d₆) δ 10.42-10.17 (m, 1H), 7.70-7.53 (m, 3H), 7.50-6.93 (m, 5H), 6.16-4.25 (m, 2H), 3.53-3.33 (m, 1H), 3.09-2.50 (m, 5H), 2.38-1.51 (m, 10H), 0.92-0.70 (m, 4H); LC-MS: m/z 430.27 (MH+).

### Preparation: N-[3-cyclopentyl-4-(1,1-dioxo-1,4-thiazinane-4-carbonyl)phenyl]cyclopropanecarboxamide

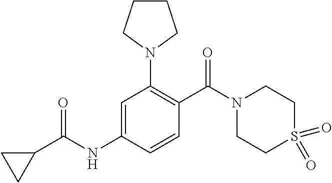 [SMILES: O=C(Nc1ccc(C(=O)N2CCS(=O)(=O)CC2)c(N2CCCC2)c1)C1CC1]

### Example 90

A mixture of N-[3-(cyclopenten-1-yl)-4-(1,1-dioxo-1,4-thiazinane-4-carbonyl)phenyl]cyclopropanecarboxamide (23.0 mg, 0.060 mmol) and Pd—C 10% (6.3 mg, 0.010 mmol) in MeOH (5 mL) was stirred under H₂ atmosphere (1 atm) at RT for 2 h. After this time the reaction was filtered and concentrated under vacuum. The residue was purified by FC on reverse phase using acid conditions (eluting from 5:95 of CH₃CN/H₂O+0.1% of HCOOH to 60:40 of CH₃CN/H₂O+0.1% of HCOOH) affording the product of formula N-[3-cyclopentyl-4-(1,1-dioxo-1,4-thiazinane-4-carbonyl)phenyl]cyclopropanecarboxamide.

### Yield: 7.6 mg

1H NMR (400 MHZ, DMSO-d6) δ 10.24 (s, 1H), 7.63 (d, 1H), 7.48 (dd, 1H), 7.26 (d, 1H), 4.46 (d, 1H), 3.71-3.44 (m, 3H), 3.20 (d, 3H), 2.93 (p, 2H), 1.94 (dt, 2H), 1.75 (td, 3H), 1.61 (q, 2H), 1.47 (dq, 2H), 0.84-0.74 (m, 4H); LC-MS: m/z 391.5 (MH+).

### Preparation: N-[3-cyclopentyl-4-(4-methyl-2-phenylpiperazine-1-carbonyl)phenyl]cyclopropanecarboxamide

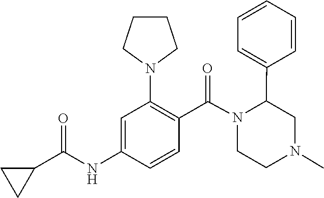 [SMILES: CN1CCN(C(=O)c2ccc(NC(=O)C3CC3)cc2N2CCCC2)C(c2ccccc2)C1]

### Example 91

The synthesis of the title compound was effected analogously to the synthesis of the compound of Example 90 N-[3-cyclopentyl-4-(1,1-dioxo-1,4-thiazinane-4-carbonyl)phenyl]cyclopropanecarboxamide wherein N-[3-(cyclopenten-1-yl)-4-(4-methyl-2-phenylpiperazine-1-carbonyl)phenyl]cyclopropanecarboxamide was used instead of N-[3-(cyclopenten-1-yl)-4-(1,1-dioxo-1,4-thiazinane-4-carbonyl)phenyl]cyclopropanecarboxamide. The title compound was obtained in a yield of 54% (16 mg).

¹H NMR (400 MHZ, DMSO-d₆) δ 10.22 (s, 1H), 7.76-7.53 (m, 3H), 7.50-7.07 (m, 5H), 5.95-4.29 (m, 1H), 3.53-3.40 (m, 1H), 3.22-2.62 (m, 4H), 2.42-2.13 (m, 4H), 2.07-1.10 (m, 10H), 0.86-0.70 (m, 4H); LC-MS: m/z 432.29 (MH+)

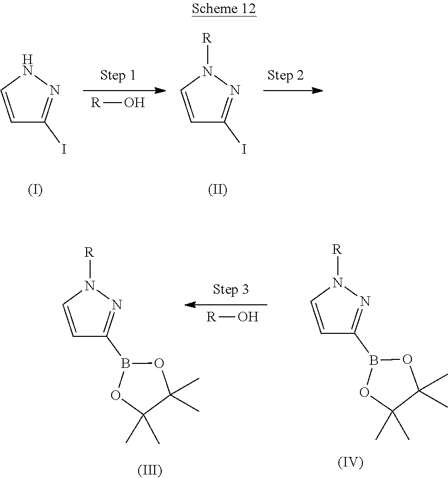

### Step 1

Compound of formula II may be obtained from compound of formula I (commercially available from Combi-Blocks) via Mitsunobu-Tsunoda reaction in presence of desired alcohol. The reaction is carried out in a suitable solvent, e.g. 1,4-Dioxane, under microwave irradiation, typically at 150° C. The reaction takes about 30 minutes to complete. Alternatively compound of formula II may be obtained by N-alkylation of compound of formula I with a suitable electrophile reagent, e.g. a proper tosylate, in the presence of a suitable base, e.g. NaH, in a suitable solvent such as DMF, typically at 50° C. The reaction takes about 12 hours to complete.

### Step 2

Compound of formula III may be obtained via Miyaura Borylation between compound of formula II and B₂Pin₂, in presence of a suitable transition metal catalyst, e.g. Pd(dppf)Cl₂, a suitable base, e.g. potassium acetate, in a suitable solvent, e.g. 1,4-Dioxane, typically at 100° C. The reaction takes about 12 hours to complete.

### Step 3

Compound of formula III may be obtained from compound of formula IV (commercially available from Activate Scientific) via Mitsunobu-Tsunoda reaction in presence of desired alcohol in 1′4-Dioxane as solvent, under microwave irradiation at 150° C. The reaction takes about 30 minutes to complete.

### Scheme 12, Cpd (II)*a Preparation: 1-cyclobutyl-3-iodopyrazole

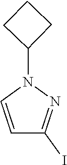

A mixture of 3-iodo-1H-pyrazole (1.0 g, 5.16 mmol), cyclobutanol (371.76 mg, 5.16 mmol), 2-tributylphosphoranylideneacetonitrile (2.7 mL, 10.31 mmol) in 1,4-Dioxane (17.78 mL) was stirred under microwave irradiation at 150° C. for 30 minutes. The mixture was diluted with water (20 mL) and extracted with EtOAc. The organic phase was washed with brine, dried over Na₂SO₄, filtered and then concentrated under high vacuum. The crude was purified FC on silica gel (eluting from 100% of cHex to cHex/EtOAc 90:10) affording the product of formula 1-cyclobutyl-3-iodopyrazole.

### Yield: 295 mg.

1H NMR (400 MHZ, DMSO-d₆) δ ppm 7.27 (d, 1H), 6.40 (d, 1H), 4.82-4.70 (m, 1H), 2.61-2.39 (m, 4H), 1.95-1.79 (m, 2H); LC-MS: m/z 249.06 (MH+).

### Scheme 12, Cpd (II)*b Preparation: 1-(1-cyclopropylethyl)-3-iodopyrazole

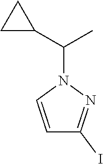

The synthesis of the title compound was effected analogously to the synthesis of the compound 1-cyclobutyl-3-iodopyrazole wherein 1-cyclopropylethanol was used instead of cyclobutanol. The title compound was obtained in in a yield of 44% (877 mg).

1H NMR (400 MHZ, DMSO-d₆) δ ppm 7.56 (dd, 1H), 6.43 (t, 1H), 3.81 (dq, 1H), 1.46 (dd, 3H), 1.40-1.29 (m, 1H), 0.62-0.22 (m, 4H); LC-MS: m/z 263.1 (MH+).

### Scheme 12, Cpd (II)*c Preparation: 1-tert-butyl-3-iodopyrazole

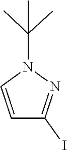

To a solution of 3-iodo-1H-pyrazole (1.0 g, 5.16 mmol) in 2-methyl-2-propanol (5.08 mL, 53.1 mmol), H₂SO₄ 95-97% (0.35 mL, 6.19 mmol) was added. The mixture was stirred at 100° C. overnight. After this time the mixture was diluted with a saturated solution of NaHCO₃ until pH=7 and extracted with EtOAc. Organic phase was washed with brine, dried over Na₂SO₄ and concentrated to give a crude that was purified by FC on silica gel (eluting from 100% of cHex to cHex/EtOAc 80:20) to afford the product of formula 1-tert-butyl-3-iodopyrazole.

### Yield: 713 mg.

1H NMR (400 MHZ, DMSO-d₆) δ ppm 7.73 (d, 1H), 6.43 (d, 1H), 1.49 (s, 9H); LC-MS: m/z 251.00 (MH+).

### Scheme 12, Cpd (II)*d Preparation: 3-iodo-1-(oxetan-3-yl)pyrazole

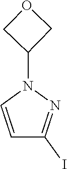

To a solution of 3-iodo-1H-pyrazole (0.51 g, 2.63 mmol) in DMF (10 mL), sodium hydride (0.42 g, 10.52 mmol) was added at 0° C. and the reaction was stirred at the same temperature for 10 min. After this time oxetan-3-yl 4-methylbenzenesulfonate (0.9 g, 3.94 mmol) was added. The reaction was allowed to reach RT and, then, stirred at 50° C. overnight. After this time the mixture was diluted with water and extracted with EtOAt. The organic phase was washed with Brine, dried over Na₂SO₄ and concentrated under reduced pressure. The crude was purified by FC on NH column (eluting from 100% of cHex to cHex/EtOAc 90:10) to afford the product of formula 3-iodo-1-(oxetan-3-yl) pyrazole.

### Yield: 445 mg.

1H NMR (400 MHZ, DMSO-d₆) δ ppm 7.80 (d, 1H), 6.52 (d, 1H), 5.57 (tt, 1H), 4.91-4.81 (m, 4H); LC-MS: m/z 251.12 (MH+).

### Preparation: oxetan-3-yl 4-methylbenzenesulfonate

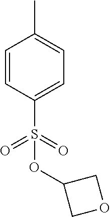

To a solution of 3-oxetanol (1.0 g, 13.5 mmol) in DCM (20 mL), Pyridine (2.17 mL, 27 mmol) was added followed by DMAP (0.33 g, 2.7 mmol) and 4-methylbenzenesulfonyl chloride (2.83 g, 14.85 mmol). The reaction was allowed to stir overnight at room temperature. After this time the mixture was diluted with a 1N aqueous solution of HCl and extracted with DCM. Organic phase was washed with a s.s. of NaHCO₃. The organic phase was dried using a phase separator and concentrated to afford a crude that was purified by FC on silica gel (eluting from 100% of cHex 100% to cHex/EtOAc 80:20) to afford the product of formula oxetan-3-yl 4-methylbenzenesulfonate.

### Yield: 1.96 g.

1H NMR (400 MHZ, DMSO-d₆) δ ppm 7.80 (d, 2H), 7.49 (d, 2H), 5.39-5.31 (m, 1H), 4.71-4.63 (m, 2H), 4.49-4.37 (m, 2H), 2.42 (s, 3H); LC-MS: m/z 229.07 (MH+).

### Scheme 12, Cpd (III)*a Preparation: 1-cyclobutyl-3-(4,4,5,5-tetramethyl-1,3,2-dioxaborolan-2-yl) pyrazole

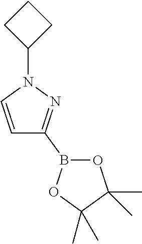 [SMILES: CC1(C)OB(c2ccn(C3CCC3)n2)OC1(C)C]

A mixture of 1-cyclobutyl-3-iodopyrazole (290.0 mg, 1.17 mmol), 4,4,5,5-tetramethyl-2-(4,4,5,5-tetramethyl-1,3,2-dioxaborolan-2-yl)-1,3,2-dioxaborolane (356.25 mg, 1.4 mmol), potassium acetate (401.56 mg, 4.09 mmol) and [1,1′-Bis(diphenylphosphino) ferrocene]dichloropalladium (II), complex with dichloromethane (382.82 mg, 0.470 mmol) in 1,4-Dioxane (5 mL) was degassed with N₂ for 5 min followed by 3 cycles of vacuum/N₂. The reaction was stirred at 100° C. overnight. The day after the mixture was cooled down to room temperature and diluted with water. The mixture was extracted with EtOAc 3 times. The combined organic fractions were washed with brine, dried over Na₂SO₄, filtered and concentrated under vacuum affording the product of formula 1-cyclobutyl-3-(4,4,5,5-tetramethyl-1,3,2-dioxaborolan-2-yl)pyrazole (see Scheme 12, Cpd (III)*a). The product was used as such in the next step.

### Yield: 314.6 mg

LC-MS: m/z 167.06 (MH+) mass of boronic acid.

### Scheme 12, Cpd (III)*b Preparation: 1-(1-cyclopropylethyl)-3-(4,4,5,5-tetramethyl-1,3,2-dioxaborolan-2-yl)pyrazole

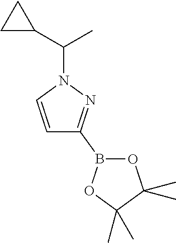 [SMILES: CC(C1CC1)n1ccc(B2OC(C)(C)C(C)(C)O2)n1]

The synthesis of the title compound was effected analogously to the synthesis of the compound of Scheme 12, Cpd (III)*a 1-cyclobutyl-3-(4,4,5,5-tetramethyl-1,3,2-dioxaborolan-2-yl)pyrazole wherein 1-(1-cyclopropylethyl)-3-iodopyrazole was used instead of 1-cyclobutyl-3-iodopyrazole. The title compound was obtained in a yield of 76% (459 mg).

1H NMR (400 MHZ, DMSO-d₆) δ ppm 7.79 (d, 1H), 6.58 (d, 1H), 4.20-4.09 (m, 1H), 1.55-1.43 (m, 3H), 1.25-1.20 (m, 1H), 1.07 (s, 12H), 0.63-0.17 (m, 4H); LC-MS: m/z 181.1 (MH+) mass of boronic acid.

### Scheme 12, Cpd (III)*c Preparation: 1-tert-butyl-3-(4,4,5,5-tetramethyl-1,3,2-dioxaborolan-2-yl) pyrazole

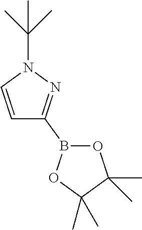 [SMILES: CC(C)(C)n1ccc(B2OC(C)(C)C(C)(C)O2)n1]

The synthesis of the title compound was effected analogously to the synthesis of the compound of Scheme 12, Cpd (III)*a 1-cyclobutyl-3-(4,4,5,5-tetramethyl-1,3,2-dioxaborolan-2-yl)pyrazole wherein 1-tert-butyl-3-iodopyrazole was used instead of 1-cyclobutyl-3-iodopyrazole. The title compound was obtained in a quantitative yield (472 mg).
LC-MS: m/z 167.06 (MH+) mass of boronic acid.

### Scheme 12, Cpd (III)*d Preparation: 1-(oxetan-3-yl)-3-(4,4,5,5-tetramethyl-1,3,2-dioxaborolan-2-yl) pyrazole

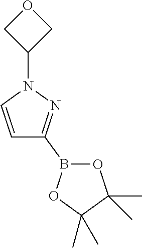 [SMILES: CC1(C)OB(c2ccn(C3COC3)n2)OC1(C)C]

The synthesis of the title compound was effected analogously to the synthesis of the compound of Scheme 12, Cpd (III)*a 1-cyclobutyl-3-(4,4,5,5-tetramethyl-1,3,2-dioxaborolan-2-yl)pyrazole wherein 3-iodo-1-(oxetan-3-yl)pyrazole was used instead of 1-cyclobutyl-3-iodopyrazole. The title compound was obtained in a quantitative yield (228 mg).

### LC-MS: m/z 247.18 (MH+)

### Scheme 12, Cpd (III)*e Preparation: 1-(2-methylpropyl)-3-(4,4,5,5-tetramethyl-1,3,2-dioxaborolan-2-yl)pyrazole

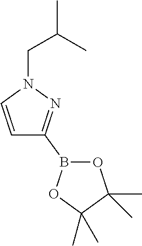 [SMILES: CC(C)Cn1ccc(B2OC(C)(C)C(C)(C)O2)n1]

A mixture of 2-methyl-1-propanol (0.57 mL, 6.18 mmol), 3-(4,4,5,5-tetramethyl-1,3,2-dioxaborolan-2-yl)-1H-pyrazole (1.2 g, 6.18 mmol) and 2-tributylphosphoranylideneacetonitrile (3.24 mL, 12.37 mmol) in 1,4-Dioxane (12 mL) was stirred at 150° C. for 30 min under microwave irradiation. The reaction mixture was cooled down to room temperature and diluted with water and extracted with EtOAc (3×). The organic layers were collected together and washed with brine, dried over Na₂SO₄, filtered and the solvent removed under vacuum affording the product of formula 1-(2-methylpropyl)-3-(4,4,5,5-tetramethyl-1,3,2-dioxaborolan-2-yl)pyrazole (see Scheme 12, Cpd (III)*e), which was used in the next step without further purifications.

### Yield: 1 g.

1H NMR (400 MHZ, MeOD) δ ppm 7.48 (d, 1H), 6.68 (d, 1H), 4.21 (d, 2H), 2.13 (dp, 1H), 1.35 (s, 12H), 0.86 (d, 6H); LC-MS: m/z 169.0 (MH+) mass of boronic acid.

### Scheme 12, Cpd (III)*f Preparation: 1-ethyl-5-(4,4,5,5-tetramethyl-1,3,2-dioxaborolan-2-yl)pyrazole

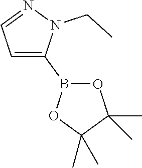 [SMILES: CCn1nccc1B1OC(C)(C)C(C)(C)O1]

The synthesis of the title compound was effected analogously to the synthesis of the compound of Scheme 12, Cpd (III)*e 1-(2-methylpropyl)-3-(4,4,5,5-tetramethyl-1,3,2-dioxaborolan-2-yl)pyrazole wherein ethanol was used instead of 2-methyl-1-propanol. The title compound was obtained in a quantitative yield (2.99 g).
LC-MS: m/z 141.01 (MH+) mass of boronic acid.

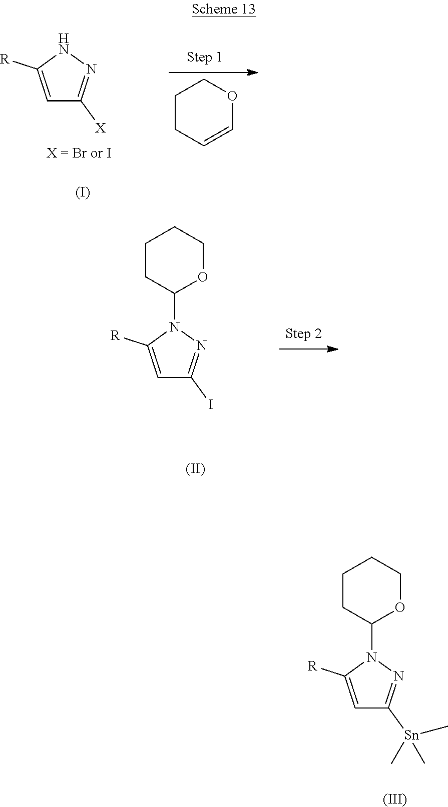

### Step 1

Compound of formula II may be obtained by N-protection of compound of formula I with 3,4-dihydro-2H-pyran in the presence of a catalytic amount of 4-toluensulfonic acid, in a suitable solvent, e.g. DCM, typically at RT. The reaction takes about 12 hours to complete.

### Step 2

Compound of formula III may be obtained from compound of formula II by reaction with trimethyl(trimethylstannyl)stannane in the presence of a suitable catalyst, e.g. palladium tetrakis triphenylphosphine, a suitable solvent, e.g. toluene, typically at 100° C. The reaction takes from about 1 hour to 20 hours to complete.

### Scheme 13, Cpd (II)*a Preparation: 3-bromo-1-tetrahydropyran-2-yl-5-(trifluoromethyl) pyrazole

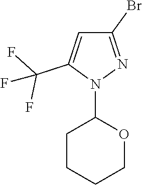 [SMILES: FC(F)(F)c1cc(Br)nn1C1CCCCO1]

To a stirred solution of 3-bromo-5-(trifluoromethyl)-1H-pyrazole (200.0 mg, 0.930 mmol) in DCM (9.304 mL), 3,4-dihydro-2H-pyran (126.5 uL, 1.4 mmol) and 4-toluenesulfonic acid (16.02 mg, 0.090 mmol) were added. The mixture was stirred overnight at RT. After this time, the mixture was washed with a s.s. of NaHCO₃ and the aqueous phase was counterextracted with DCM. The organic phases were combined, washed with brine, dried over a phase separator and concentrated under vacuum. The crude was purified by FC on silica gel (eluting from 100% of cHex to cHex/EtOAc 85:15) affording the product of formula 3-bromo-1-tetrahydropyran-2-yl-5-(trifluoromethyl) pyrazole (see Scheme 13, Cpd (II)*a).

### Yield: 218 mg

¹H NMR (400 MHz, DMSO-d₆) δ 7.14 (d, 1H), 5.59 (dd, 1H), 3.98-3.87 (m, 1H), 3.72-3.58 (m, 1H), 2.30-2.15 (m, 1H), 2.04-1.86 (m, 2H), 1.81-1.64 (m, 1H), 1.60-1.50 (m, 2H).

### Scheme 13, Cpd (II)*b Preparation: 3-iodo-1-(oxan-2-yl)-5-propan-2-ylpyrazole

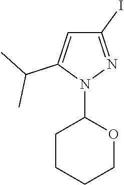

The synthesis of the title compound was effected analogously to the synthesis of the compound of Scheme 13, Cpd (II)*a 3-bromo-1-tetrahydropyran-2-yl-5-(trifluoromethyl) pyrazole wherein 3-iodo-5-propan-2-yl-1H-pyrazole was used instead of 3-bromo-5-(trifluoromethyl)-1H-pyrazole. The title compound was obtained in yield of 94% (320 mg).

### LC-MS: m/z 321.07

### Scheme 13, Cpd (II)*c Preparation: 3-iodo-5-methyl-1-(oxan-2-yl)pyrazole

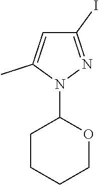

The synthesis of the title compound was effected analogously to the synthesis of the compound of Scheme 13, Cpd (II)*a 3-bromo-1-tetrahydropyran-2-yl-5-(trifluoromethyl) pyrazole wherein 3-iodo-5-propan-2-yl-1H-pyrazole was used instead of 3-bromo-5-(trifluoromethyl)-1H-pyrazole. The title compound was obtained in yield of 79% (560 mg).

### LC-MS: m/z 293.14 (MH+).

### Scheme 13, Cpd (III)*a Preparation: trimethyl-[1-tetrahydropyran-2-yl-5-(trifluoromethyl) pyrazol-3-yl]stannane

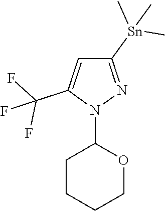 [SMILES: [CH3][Sn]([CH3])([CH3])[c]1cc(C(F)(F)F)n(C2CCCCO2)n1]

In a proper vial trimethyl(trimethylstannyl)stannane (178.87 uL, 0.860 mmol), 3-bromo-1-tetrahydropyran-2-yl-5-(trifluoromethyl) pyrazole (215.0 mg, 0.720 mmol, see Scheme 13, Cpd (II)*a), palladium tetrakis triphenylphosphine (16.61 mg, 0.010 mmol) were placed and suspended in Toluene (5 mL). The mixture was degassed bubbling N₂ for 5 min and then 5 cycles of N₂-vacuum were performed. The mixture was stirred at 100° C. for 20 h. After this time the reaction was cooled down to RT, filtered and concentrated under vacuum. The residue was dissolved in EtOAc and washed with water. The aqueous phase was counterextracted with EtOAc (×2). The combined organic portions were washed with brine, dried over Na₂SO₄, filtered and concentrated under vacuum affording the product of formula trimethyl-[1-tetrahydropyran-2-yl-5-(trifluoromethyl) pyrazol-3-yl]stannane, which was used in the next step without further purification.

### Yield: 270 mg

### LC-MS: m/z 385.11 (MH+).

### Scheme 13, Cpd (III)*b Preparation: (5-isopropyl-1-tetrahydropyran-2-yl-pyrazol-3-yl)-trimethyl-stannane

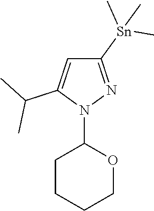 [SMILES: CC(C)c1c[c]([Sn]([CH3])([CH3])[CH3])nn1C1CCCCO1]

The synthesis of the title compound was effected analogously to the synthesis of the compound of Scheme 13, Cpd (II)*a 3-bromo-1-tetrahydropyran-2-yl-5-(trifluoromethyl) pyrazole wherein 3-iodo-1-(oxan-2-yl)-5-propan-2-ylpyrazole was used instead of the 3-bromo-1-tetrahydropyran-2-yl-5-(trifluoromethyl) pyrazole. The title compound was obtained in yield of 83% (250 mg).

### LC-MS: m/z 359.18 (MH+).

### Scheme 13, Cpd (III)*c Preparation: trimethyl-(5-methyl-1-tetrahydropyran-2-yl-pyrazol-3-yl) stannane

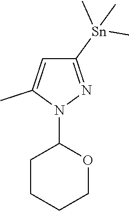

The synthesis of the title compound was effected analogously to the synthesis of the compound of Scheme 13, Cpd (II)*a 3-bromo-1-tetrahydropyran-2-yl-5-(trifluoromethyl) pyrazole wherein 3-iodo-5-methyl-1-(oxan-2-yl)pyrazole was used instead of the 3-bromo-1-tetrahydropyran-2-yl-5-(trifluoromethyl) pyrazole. The title compound was obtained in yield of 88% (150 mg).

### LC-MS: m/z 331.20 (MH+).

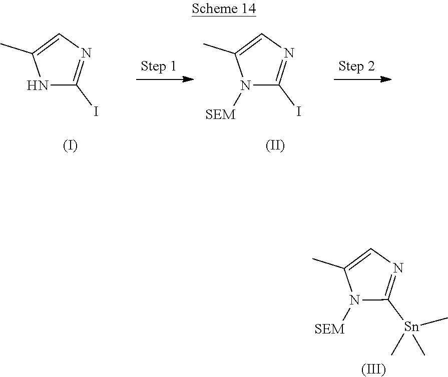

### Step 1

Compound of formula II may be obtained via N-protection of compound of formula I with SEM-Cl, in the presence of a suitable base, e.g. K₂CO₃, in a suitable solvent, e.g. acetone, typically at RT. The reaction takes about 2 hours to complete.

### Step 2

Compound of formula III may be obtained from compound of formula II by reaction with trimethyl(trimethylstannyl)stannane in the presence of a suitable catalyst, e.g. palladium tetrakis triphenylphosphine, a suitable solvent, e.g. toluene, typically at 100° C. The reaction takes from about 3 hours to complete.

### Scheme 14, Cpd (II) Preparation: 2-[(2-iodo-5-methyl-imidazol-1-yl) methoxy]ethyl-trimethyl-silane and 2-[(2-iodo-4-methyl-imidazol-1-yl) methoxy]ethyl-trimethyl-silane

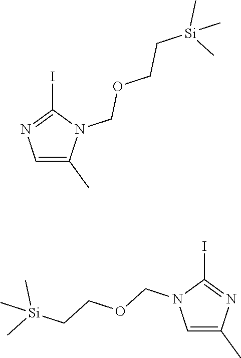

A mixture of 2-iodo-5-methyl-1H-imidazole (200.0 mg, 0.960 mmol) and potassium carbonate (398.68 mg, 2.88 mmol) in Acetone (8.013 mL) was treated by dropwise addition of 2-(chloromethoxy)ethyl-trimethylsilane (204.21 uL, 1.15 mmol) and the resulting mixture was stirred for 2 h. After this time, the mixture was filtered and concentrated under vacuum. The crude was purified by FC on silica gel (eluting from cHex/EtOAc 90:10 to cHex/EtOAc 30:70) affording the products of formula 2-[(2-iodo-5-methyl-imidazol-1-yl) methoxy]ethyl-trimethyl-silane and 2-[(2-iodo-4-methyl-imidazol-1-yl) methoxy]ethyl-trimethyl-silane as mixture as regioisomers. The mixture was used in the next step without further purification.

### Yield: 100 mg

### LC-MS: m/z 339.25 (MH+).

### Scheme 14, Cpd (III) Preparation: trimethyl-[2-[(5-methyl-2-trimethylstannyl-imidazol-1-yl)methoxy]ethyl]silane and trimethyl-[2-[(4-methyl-2-trimethylstannyl-imidazol-1-yl)methoxy]ethyl]silane

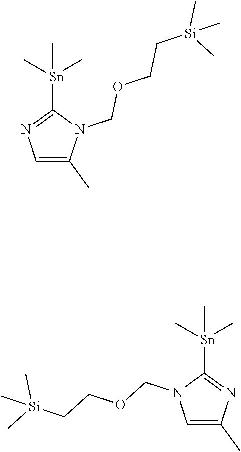

A mixture of trimethyl(trimethylstannyl)stannane (40.46 uL, 0.200 mmol), 2-[(2-iodo-5-methyl-imidazol-1-yl) methoxy]ethyl-trimethyl-silane (55.0 mg, 0.160 mmol) and palladium tetrakis triphenylphosphine (3.76 mg, 0 mmol) in Toluene (1.7 mL) was degassed bubbling N₂ for 15 min. Then 5 cycles of N₂-vacuum were performed and the mixture was stirred at 100° C. for 3 h. The mixture was concentrated under vacuum, the residue was dissolved in EtOAc and washed with water. The aqueous phase was extracted with EtOAc (×2). The combined organic portions were washed with brine, dried over Na₂SO₄, filtered and concentrated under vacuum affording the products of formula trimethyl-[2-[(5-methyl-2-trimethylstannyl-imidazol-1-yl)methoxy]ethyl]silane and trimethyl-[2-[(4-methyl-2-trimethylstannyl-imidazol-1-yl)methoxy]ethyl]silane as mixture of regioisomers, The mixture was used in the next step without further purification.

### Yield: 65 mg

### LC-MS: m/z 377.2 (MH+)

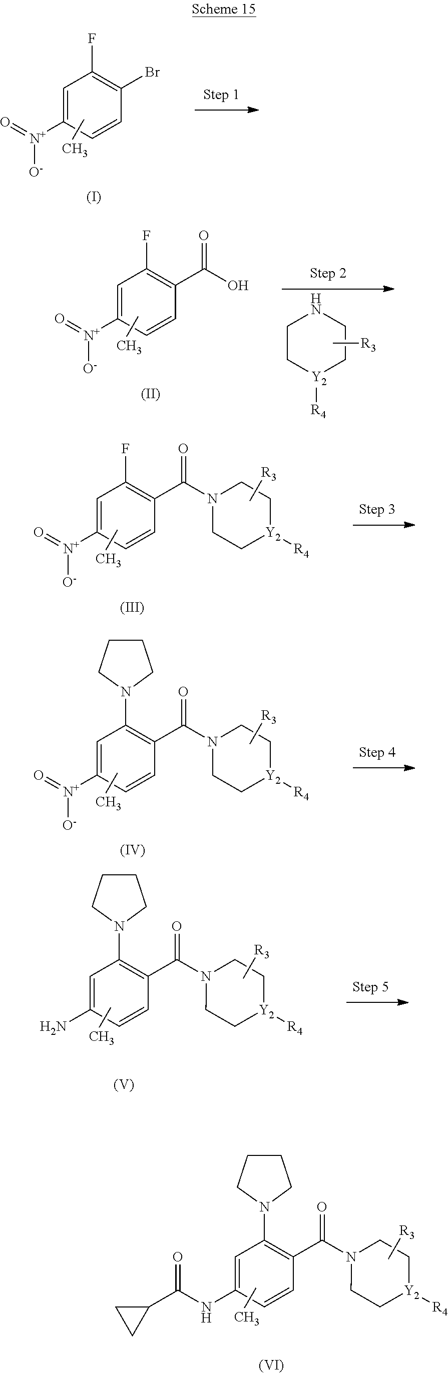

### Step 1

Compound of formula II may be obtained from compound of formula I (commercially available from Combi-Blocks) by hydroxycarbonylation using oxalic acid and acetic anhydride as the in situ carbon monoxide generator. The reaction is carried out in the presence of a suitable organic base, e.g. DIPEA, a suitable catalyst, e.g. Palladium diacetate, with a suitable phosphine ligand, e.g. Xantphos; in a suitable solvent such as DMF, typically at 100° C. The reaction takes about 12 hours to complete.

### Step 2

Compound of formula III may be obtained via acyl chloride formation of compound of formula II by treatment with a suitable chlorinating agent, such as SOCl₂, typically at 90° C., followed by coupling with the proper amine in the presence of a suitable organic base, e.g. TEA. The reaction is carried out in a suitable solvent such as DCM, typically at room temperature. The reaction takes from about 1 hour to about 12 hours to complete.

### Step 3

Compound of formula IV may be obtained from compound of formula III by SNAr substitution with pyrrolidine in a suitable aprotic solvent, e.g. DMSO, typically at 120° C. The reaction takes from about 12 hours to about 24 hours to complete.

### Step 4

Compound of formula V may be obtained by nitro reduction of compound of formula IV under catalytic hydrogenation conditions, using a suitable catalyst, such as Pd/C, in a suitable solvent such as methanol, typically at room temperature. The reaction takes from about 4 hours to about 12 hours to complete.

### Step 5

Compound of formula VI may be obtained by coupling reaction between compound of formula V and a proper commercially available acyl chloride, e.g. cyclopropanecarbonyl chloride, in presence of a suitable organic base, e.g. DIPEA. The reaction is carried out in a suitable solvent such as DCM, typically at room temperature. The reaction takes from about 1 hour to about 12 hours to complete.

### Scheme 15, Cpd (II)*a Preparation: 2-fluoro-5-methyl-4-nitrobenzoic acid

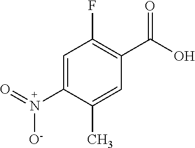

In a 40 mL vial equipped with magnetic bar 1-bromo-2-fluoro-5-methyl-4-nitrobenzene (500.0 mg, 2.14 mmol), oxalic acid (288.53 mg, 3.2 mmol), [Pd(AcO)₂]₃ (4.8 mg, 0.020 mmol) and (5-diphenylphosphino-9,9-dimethyl-4-xanthenyl)-diphenylphosphine (12.36 mg, 0.020 mmol) were introduced. A solution of Acetic acid acetyl ester (0.3 mL, 3.2 mmol) and N,N-Diisopropylethylamine (0.56 mL, 3.2 mmol) in DMF (11 mL) was added and the mixture was bubbled with N₂ for 10 min and then 3 cycles of N₂-vacuum were performed. The mixture was stirred at 100° C. overnight. After this time a s.s of NaHCO₃ was added and the aqueous phase was washed with DCM. The aqueous phase was then acidified with concentrated HCl until pH=1 and extracted with EtOAc. The organic fractions were combined, washed with brine, dried over Na₂SO₄ and then concentrated under vacuum to afford the product of formula 2-fluoro-5-methyl-4-nitrobenzoic acid (see Scheme 15, Cpd (II)*a), which was used in the next step without further purification.

### Yield: 410 mg

1H NMR (400 MHZ, DMSO-d6) δ ppm 8.32-7.83 (m, 2H), 2.51 (s, 3H); LC-MS: m/z 198.18 (MH+).

### Scheme 15, Cpd (II)*b Preparation: 2-fluoro-3-methyl-4-nitrobenzoic acid

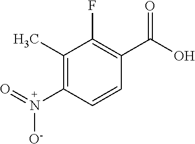

The synthesis of the title compound was effected analogously to the synthesis of the compound of Scheme 15, Cpd (II)*a 2-fluoro-5-methyl-4-nitrobenzoic acid wherein 1-bromo-2-fluoro-3-methyl-4-nitrobenzene was used instead of 1-bromo-2-fluoro-5-methyl-4-nitrobenzene. The title compound was obtained in a yield of 68% (289 mg).

1H NMR (400 MHZ, DMSO-d6) δ ppm 13.81 (s, 1H), 7.91-7.83 (m, 2H), 2.39 (s, 3H); LC-MS: m/z 198.17 (MH+).

### Scheme 15, Cpd (III)*a Preparation: (2-fluoro-5-methyl-4-nitrophenyl)-(4-methyl-2-phenylpiperazin-1-yl)methanone

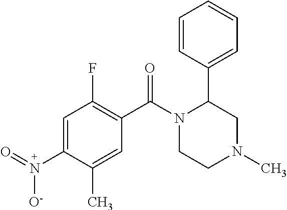

A mixture of 2-fluoro-5-methyl-4-nitrobenzoic acid (205.0 mg, 1.03 mmol, Scheme 15, Cpd (II)*a) and thionyl dichloride (1.5 mL, 20.59 mmol) was heated to reflux for 2 hours. After this time the reaction was cooled down to RT and then concentrated under vacuum. The mixture was stripped three times with cyclohexane in order to remove the excess of thionyl dichloride. The residue was suspended in dry DCM (4.1 mL) and triethylamine (0.43 mL, 3.09 mmol) was added followed by 1-methyl-3-phenylpiperazine (181.45 mg, 1.03 mmol). The reaction mixture was stirred overnight at RT. The day after the reaction was diluted with DCM and washed with a s.s of NaHCO₃3 times. The aqueous phases were combined and counterextracted with DCM. The combined organic phases were washed with brine, dried over a phase separator and concentrated under vacuum affording the product of formula (2-fluoro-5-methyl-4-nitrophenyl)-(4-methyl-2-phenylpiperazin-1-yl)methanone. The product was used in the next step without further purification.

### Yield: 297 mg

1H NMR (400 MHZ, DMSO-d6) δ ppm 8.07 (d, 1H), 7.68 (s, 1H), 7.55-7.03 (m, 5H), 5.90-5.33 (m, 1H), 4.92-4.30 (m, 1H), 4.00-3.43 (m, 2H), 3.26-2.71 (m, 3H), 2.53 (s, 3H), 2.29-2.17 (m, 3H); LC-MS: m/z 358.36 (MH+).

### Scheme 15, Cpd (III)*b Preparation: (1,1-dioxo-1,4-thiazinan-4-yl)-(2-fluoro-5-methyl-4-nitrophenyl)methanone

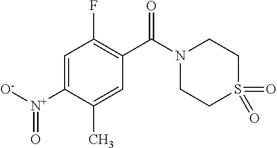 [SMILES: Cc1cc(C(=O)N2CCS(=O)(=O)CC2)c(F)cc1[N+](=O)[O-]]

The synthesis of the title compound was effected analogously to the synthesis of the compound (2-fluoro-5-methyl-4-nitrophenyl)-(4-methyl-2-phenylpiperazin-1-yl)methanone wherein 1,4-thiazinane 1,1-dioxide hydrochloride was used instead of 1-methyl-3-phenylpiperazine. The title compound was obtained in a yield of 81% (297 mg).

¹H NMR (400 MHZ, DMSO-d₆) δ 8.08 (d, 1H), 7.78 (d, 1H), 4.58-4.16 (m, 1H), 3.85-3.58 (m, 3H), 3.19-2.92 (m, 4H), 2.51 (s, 3H); LC-MS: m/z 317.10 (MH+).

### Scheme 15, Cpd (III)*c Preparation: (2-fluoro-3-methyl-4-nitrophenyl)-(4-methyl-2-phenylpiperazin-1-yl)methanone

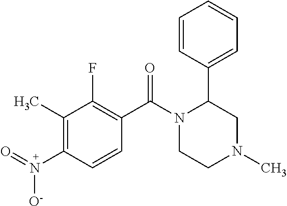

The synthesis of the title compound was effected analogously to the synthesis of the compound (2-fluoro-5-methyl-4-nitrophenyl)-(4-methyl-2-phenylpiperazin-1-yl)methanone wherein 2-fluoro-3-methyl-4-nitrobenzoic acid was used instead of 2-fluoro-5-methyl-4-nitrobenzoic acid (see Scheme 15, Cpd (II)*a). The title compound was obtained in a quantitative yield (272 mg).

1H NMR (400 MHZ, DMSO-d6) δ ppm 7.99-7.78 (m, 1H), 7.66-7.15 (m, 6H), 5.79 (s, 1H), 4.73-4.34 (m, 1H), 3.62 (dd, 1H), 3.26-2.69 (m, 4H), 2.42 (s, 2H), 2.25-2.13 (m, 3H), 2.01-1.88 (m, 1H); LC-MS: m/z 358.36 (MH+).

### Scheme 15, Cpd (III)*d Preparation: (1,1-dioxo-1,4-thiazinan-4-yl)-(2-fluoro-3-methyl-4-nitrophenyl)methanone

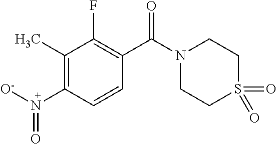

The synthesis of the title compound was effected analogously to the synthesis of the compound (2-fluoro-5-methyl-4-nitrophenyl)-(4-methyl-2-phenylpiperazin-1-yl)methanone wherein 1,4-thiazinane 1,1-dioxide hydrochloride was used instead of 1-methyl-3-phenylpiperazine and 2-fluoro-3-methyl-4-nitrobenzoic acid was used instead of 2-fluoro-5-methyl-4-nitrobenzoic acid (see Scheme 15, Cpd (II)*a). The title compound was obtained in a yield of 84% (193 mg).

1H NMR (400 MHZ, DMSO-d6) δ ppm 7.95 (dd, 1H), 7.80-7.54 (m, 1H), 4.34 (d, 2H), 3.65 (s, 4H), 2.98 (d, 2H), 2.41 (d, 3H); LC-MS: m/z 317.09 (MH+).

### Scheme 15, Cpd (IV)*a Preparation: (5-methyl-4-nitro-2-pyrrolidin-1-ylphenyl)-(4-methyl-2-phenylpiperazin-1-yl)methanone

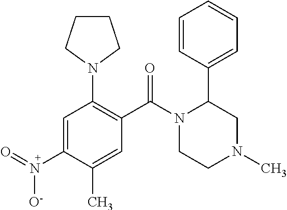

To a solution of (2-fluoro-5-methyl-4-nitrophenyl)-(4-methyl-2-phenylpiperazin-1-yl)methanone (290.0 mg, 0.810 mmol) in DMSO (4.0 mL), pyrrolidine (0.2 mL, 2.43 mmol) was added and the reaction mixture was stirred overnight at 120° C. After this time the reaction mixture was cooled down to RT, diluted with H₂O and extracted three times with EtOAc. The organic portions were combined, washed with brine, dried over Na₂SO₄, filtered and concentrated under vacuum affording the product of formula (5-methyl-4-nitro-2-pyrrolidin-1-ylphenyl)-(4-methyl-2-phenylpiperazin-1-yl)methanone. The crude was used in the next step without further purification.

### Yield: 351 mg

### LC-MS: m/z 409.27 (MH+).

### Scheme 15, Cpd (IV)*b Preparation: (1,1-dioxo-1,4-thiazinan-4-yl)-(5-methyl-4-nitro-2-pyrrolidin-1-ylphenyl)methanone

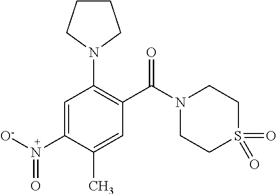

The synthesis of the title compound was effected analogously to the synthesis of the compound (5-methyl-4-nitro-2-pyrrolidin-1-ylphenyl)-(4-methyl-2-phenylpiperazin-1-yl)methanone wherein (1,1-dioxo-1,4-thiazinan-4-yl)-(2-fluoro-5-methyl-4-nitrophenyl)methanone was used instead of (2-fluoro-5-methyl-4-nitrophenyl)-(4-methyl-2-phenylpiperazin-1-yl)methanone. The title compound was obtained in a yield of 92% (247 mg).

1H NMR (400 MHZ, DMSO-d6) δ ppm 7.38 (d, 1H), 7.18 (s, 1H), 4.52 (d, 1H), 3.77 (d, 1H), 3.69-3.49 (m, 2H), 3.40-3.35 (m, 2H), 3.27-3.01 (m, 6H), 2.37 (s, 3H), 1.90 (d, 4H); LC-MS: m/z 368.13 (MH+).

### Scheme 15, Cpd (IV)*c Preparation: (1,1-dioxo-1,4-thiazinan-4-yl)-(3-methyl-4-nitro-2-pyrrolidin-1-ylphenyl)methanone

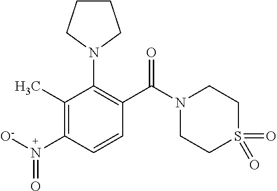

The synthesis of the title compound was effected analogously to the synthesis of the compound (5-methyl-4-nitro-2-pyrrolidin-1-ylphenyl)-(4-methyl-2-phenylpiperazin-1-yl)methanone wherein (1,1-dioxo-1,4-thiazinan-4-yl)-(2-fluoro-3-methyl-4-nitrophenyl)methanone was used instead of (2-fluoro-5-methyl-4-nitrophenyl)-(4-methyl-2-phenylpiperazin-1-yl)methanone. The title compound was obtained in a yield of 37% (82 mg).

1H NMR (400 MHZ, DMSO-d6) δ ppm 7.63 (d, 1H), 7.47 (d, 1H), 4.58 (d, 1H), 3.73 (d, 1H), 3.63-3.33 (m, 4H), 3.26-2.95 (m, 6H), 2.28 (s, 3H), 1.93-1.77 (m, 4H); LC-MS: m/z 368.2 (MH+).

### Scheme 15, Cpd (V)*a Preparation: (4-amino-5-methyl-2-pyrrolidin-1-ylphenyl)-(4-methyl-2-phenylpiperazin-1-yl)methanone

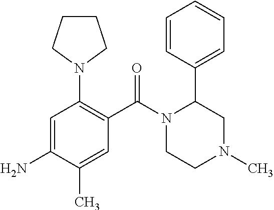 [SMILES: Cc1cc(C(=O)N2CCN(C)CC2c2ccccc2)c(N2CCCC2)cc1N]

A mixture of (5-methyl-4-nitro-2-pyrrolidin-1-ylphenyl)-(4-methyl-2-phenylpiperazin-1-yl)methanone (350.0 mg, 0.860 mmol) and palladium (182.36 mg, 0.090 mmol) was stirred under H₂ atmosphere (1 atm) at RT for 4 h. After this time the reaction was filtered and concentrated under vacuum affording the product of formula (4-amino-5-methyl-2-pyrrolidin-1-ylphenyl)-(4-methyl-2-phenylpiperazin-1-yl)methanone. The product was used in the next step without further purification.

### Yield: 292 mg

### LC-MS: m/z 379.3 (MH+).

### Scheme 15, Cpd (V)*b Preparation: (4-amino-5-methyl-2-pyrrolidin-1-ylphenyl)-(1,1-dioxo-1,4-thiazinan-4-yl)methanone

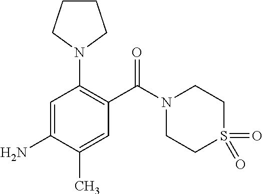 [SMILES: Cc1cc(C(=O)N2CCS(=O)(=O)CC2)c(N2CCCC2)cc1N]

The synthesis of the title compound was effected analogously to the synthesis of the compound (4-amino-5-methyl-2-pyrrolidin-1-ylphenyl)-(4-methyl-2-phenylpiperazin-1-yl)methanone wherein (1,1-dioxo-1,4-thiazinan-4-yl)-(5-methyl-4-nitro-2-pyrrolidin-1-ylphenyl)methanone was used instead of (5-methyl-4-nitro-2-pyrrolidin-1-ylphenyl)-(4-methyl-2-phenylpiperazin-1-yl)methanone. The title compound was obtained in a yield of 96% (215 mg).

1H NMR (400 MHZ, DMSO-d6) δ ppm 6.76 (d, 1H), 6.04 (s, 1H), 4.95 (s, 2H), 4.22 (s, 1H), 3.96-3.45 (m, 3H), 3.27-2.92 (m, 8H), 1.96 (s, 3H), 1.89-1.77 (m, 4H); LC-MS: m/z 338.17 (MH+).

### Scheme 15, Cpd (V)*c Preparation: (4-amino-3-methyl-2-pyrrolidin-1-ylphenyl)-(1,1-dioxo-1,4-thiazinan-4-yl)methanone

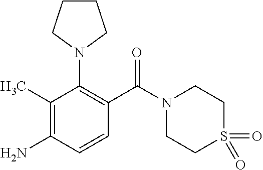 [SMILES: Cc1c(N)ccc(C(=O)N2CCS(=O)(=O)CC2)c1N1CCCC1]

The synthesis of the title compound was effected analogously to the synthesis of the compound (4-amino-5-methyl-2-pyrrolidin-1-ylphenyl)-(4-methyl-2-phenylpiperazin-1-yl)methanone wherein (1,1-dioxo-1,4-thiazinan-4-yl)-(3-methyl-4-nitro-2-pyrrolidin-1-ylphenyl)methanone was used instead of (5-methyl-4-nitro-2-pyrrolidin-1-ylphenyl)-(4-methyl-2-phenylpiperazin-1-yl)methanone. The title compound was obtained in a quantitative yield (95 mg).

### LC-MS: m/z 338.2 (MH+).

### Scheme 15, Cpd (VI)*a Preparation: N-[2-methyl-4-(4-methyl-2-phenylpiperazine-1-carbonyl)-5-pyrrolidin-1-ylphenyl]cyclopropanecarboxamide

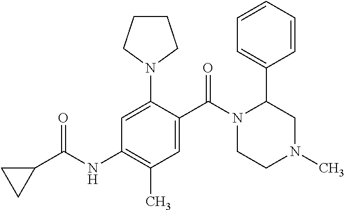 [SMILES: Cc1cc(C(=O)N2CCN(C)CC2c2ccccc2)c(N2CCCC2)cc1NC(=O)C1CC1]

### Example 92

To a mixture of N, N-Diisopropylethylamine (0.27 mL, 1.54 mmol) and (4-amino-5-methyl-2-pyrrolidin-1-ylphenyl)-(4-methyl-2-phenylpiperazin-1-yl)methanone (292.0 mg, 0.770 mmol) in DCM (20 mL) at 0° C., cyclopropanecarbonyl chloride (84.0 uL, 0.930 mmol) was added dropwise. The mixture was stirred overnight at RT. Then MeOH was added and concentrated under vacuum. The residue was purified by FC on reverse phase using acid conditions (eluting from 5:95 of CH₃CN/H₂O+0.1% of HCOOH to 50:50 of CH₃CN/H₂O+0.1% of HCOOH). Pure fractions were collected and concentrated under vacuum. The residue was taken up with a s.s. of NaHCO₃ and extracted three times with AcOEt. The combined organic fractions were washed with brine, dried over Na₂SO₄, filtered and concentrated under vacuum affording the product of formula N-[2-methyl-4-(4-methyl-2-phenylpiperazine-1-carbonyl)-5-pyrrolidin-1-ylphenyl]cyclopropanecarboxamide.

### Yield: 138 mg

1H NMR (400 MHZ, DMSO-d6) δ ppm 9.50-9.23 (m, 1H), 7.82-7.16 (m, 5H), 7.09-6.68 (m, 2H), 5.86-4.34 (m, 1H), 3.63-3.35 (m, 1H), 3.28-2.57 (m, 6H), 2.42-2.05 (m, 7H), 1.93 (d, 5H), 1.63 (dt, 2H), 0.85-0.67 (m, 4H); LC-MS: m/z 447.3 (MH+).

### Preparation: N-[4-(1,1-dioxo-1,4-thiazinane-4-carbonyl)-2-methyl-5-pyrrolidin-1-ylphenyl]cyclopropanecarboxamide

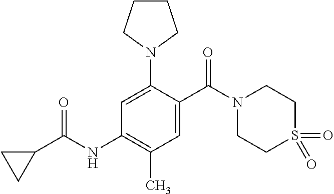 [SMILES: Cc1cc(C(=O)N2CCS(=O)(=O)CC2)c(N2CCCC2)cc1NC(=O)C1CC1]

### Example 93

The synthesis of the title compound was effected analogously to the synthesis of the compound of Example 92 N-[2-methyl-4-(4-methyl-2-phenylpiperazine-1-carbonyl)-5-pyrrolidin-1-ylphenyl]cyclopropanecarboxamide wherein (4-amino-5-methyl-2-pyrrolidin-1-ylphenyl)-(1,1-dioxo-1,4-thiazinan-4-yl)methanone was used instead of (4-amino-5-methyl-2-pyrrolidin-1-ylphenyl)-(4-methyl-2-phenylpiperazin-1-yl)methanone. The title compound was obtained in a yield of 86% (224 mg).

1H NMR (400 MHZ, DMSO-d6) δ ppm 9.37 (s, 1H), 7.01 (d, 2H), 4.38 (d, 1H), 3.89-3.45 (m, 3H), 3.25-2.98 (m, 8H), 2.12 (s, 3H), 1.97-1.77 (m, 5H), 0.84-0.71 (m, 4H); LC-MS: m/z 406.2 (MH+).

### Preparation: N-[4-(1,1-dioxo-1,4-thiazinane-4-carbonyl)-2-methyl-3-pyrrolidin-1-ylphenyl]cyclopropanecarboxamide

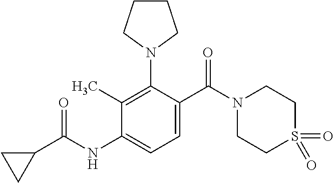 [SMILES: Cc1c(NC(=O)C2CC2)ccc(C(=O)N2CCS(=O)(=O)CC2)c1N1CCCC1]

### Example 94

The synthesis of the title compound was effected analogously to the synthesis of the compound of Example 92 N-[2-methyl-4-(4-methyl-2-phenylpiperazine-1-carbonyl)-5-pyrrolidin-1-ylphenyl]cyclopropanecarboxamide wherein (4-amino-3-methyl-2-pyrrolidin-1-ylphenyl)-(1,1-dioxo-1,4-thiazinan-4-yl)methanone was used instead of (4-amino-5-methyl-2-pyrrolidin-1-ylphenyl)-(4-methyl-2-phenylpiperazin-1-yl)methanone. The title compound was obtained in a yield of 19% (22 mg).

1H NMR (400 MHZ, DMSO-d6) δ ppm 9.55 (s, 1H), 7.21 (d, 1H), 7.13 (d, 1H), 4.53 (d, 1H), 3.76 (d, 1H), 3.60-3.46 (m, 1H), 3.42-3.33 (m, 2H), 3.28-3.13 (m, 2H), 3.11-2.91 (m, 5H), 2.10 (s, 3H), 1.94-1.71 (m, 5H), 0.79 (d, 4H); LC-MS: m/z 406.2 (MH+).

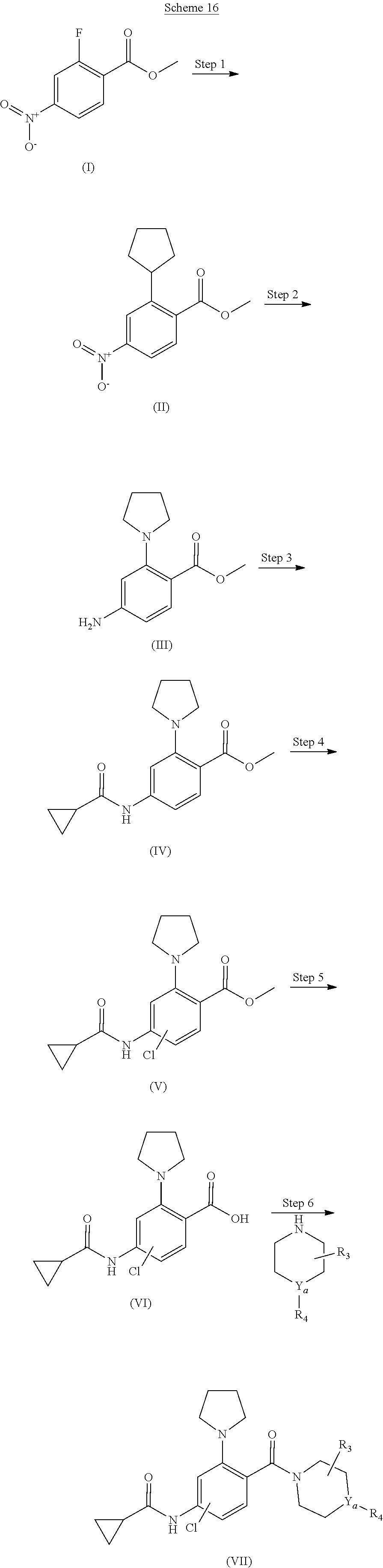

### Step 1

Compound of formula II may be obtained from compound of formula I (commercially available from Fluorochem) by SNAr substitution with pyrrolidine in a suitable aprotic solvent, e.g. CH₃CN, typically at 80° C. The reaction takes about 1 hour to complete.

### Step 2

Compound of formula III may be obtained by nitro reduction of compound of formula II using Zn dust in a suitable solvent, e.g. AcOH, typically at room temperature. The reaction takes about 4 hours to complete.

### Step 3

Compound of formula IV may be obtained by coupling reaction between compound of formula III and a proper commercially available acyl chloride, e.g. cyclopropanecarbonyl chloride, in presence of a suitable organic base, e.g. DIPEA. The reaction is carried out in a suitable solvent such as DCM, typically at room temperature. The reaction takes about 12 hours to complete.

### Step 4

Compound of formula V may be obtained by chlorination of compound of formula IV using N-Chlorosuccinimide in a suitable solvent such as 2-propanol, typically at 60° C. The reaction takes about 2 hours to complete.

### Step 5

Compound of formula VI may be obtained from compound of formula V by hydrolysis, e.g. using LiOH, in a suitable mixture of solvents such as MeOH/THF/H₂O, typically at 50° C. The reaction takes from about 3 hours to about 24 hours to complete.

### Step 6

Compound of formula VII may be obtained from compound of formula VI by coupling with a proper amine in the presence of coupling agent, e.g. HATU, and an organic base, e.g. DIPEA. The reaction is carried out in a suitable solvent such as DMF, typically at room temperature. The reaction takes from about 1 hour to about 30 hours to complete.

### Scheme 16, Cpd (II) Preparation: methyl 4-nitro-2-pyrrolidin-1-ylbenzoate

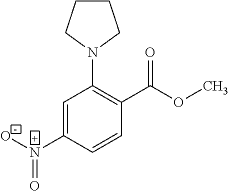

To a solution of 2-fluoro-4-nitrobenzoate (5.0 g, 25.11 mmol) in CH₃CN (50 mL), pyrrolidine (5.36 g, 75.32 mmol) was added and the reaction mixture was stirred at 80° C. for 1 h. After this time the mixture was cooled down to RT and then concentrated under vacuum. The residue was suspended in water and filtered. The solid was dried under vacuum affording the product of formula methyl 4-nitro-2-pyrrolidin-1-ylbenzoate, which was used in the next step without further purification.

### Yield: 6.0 g

1H NMR (400 MHZ, DMSO-d6) δ ppm 7.64 (d, 1H), 7.50 (d, 1H), 7.44 (dd, 1H), 3.86 (s, 3), 3.19-3.27 (m, 4H), 1.88-1.99 (m, 4H); LC-MS: m/z 251.10 (MH+).

### Scheme 16, Cpd (III) Preparation: methyl 4-amino-2-pyrrolidin-1-ylbenzoate

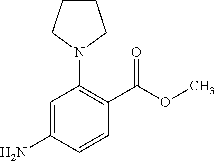 [SMILES: COC(=O)c1ccc(N)cc1N1CCCC1]

To a solution of methyl 4-nitro-2-pyrrolidin-1-ylbenzoate (6.0 g, 22.06 mmol) in Acetic acid (150 mL), zinc (14.4 g, 220.58 mmol) was added. The reaction was stirred at RT for 4 h. After this time the reaction was filtered washing with EtOAc and concentrated under vacuum. The residue was taken up with a s.s. of NaHCO₃ and extracted three times with EtOAc. The organic portions were combined, washed with brine, dried over Na₂SO₄, filtered and concentrated under vacuum affording the product of formula methyl 4-amino-2-pyrrolidin-1-ylbenzoate, which was used in the next step without further purifications.

### Yield: 5.75 g

1H NMR (400 MHZ, DMSO-d6) δ ppm 7.28 (d, 1H), 5.98 (d, 1H), 5.94 (dd, 1H), 5.46 (s, 2H), 3.67 (s, 3H), 3.18-2.94 (m, 4H), 1.91-1.71 (m, 4H); LC-MS: m/z 221.99 (MH+).

### Scheme 16, Cpd (IV) Preparation: methyl 4-(cyclopropanecarbonylamino)-2-pyrrolidin-1-ylbenzoate

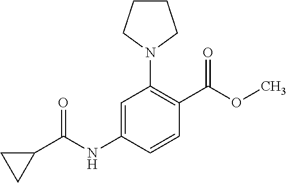 [SMILES: COC(=O)c1ccc(NC(=O)C2CC2)cc1N1CCCC1]

To a stirred solution of methyl 4-amino-2-pyrrolidin-1-ylbenzoate (5.75 g, 26.1 mmol) and N,N-Diisopropylethylamine (9.09 mL, 52.21 mmol) in DCM (145.57 mL) at 0° C., cyclopropanecarbonyl chloride (3.08 mL, 33.94 mmol) was added dropwise and the reaction mixture was stirred at RT overnight. Reaction was diluted with DCM and washed twice with a s.s. of NaHCO₃. The organic portion was washed with water and then with brine, dried over a phase separator and the solvent was evaporated under vacuum. The crude was purified by FC on silica gel (eluting from cHex/EtOAc 9:1 to cHex/EtOAc 50:50) to afford the product of formula methyl 4-(cyclopropanecarbonylamino)-2-pyrrolidin-1-ylbenzoate.

### Yield: 5.57 g

1H NMR (400 MHZ, DMSO-d6) δ ppm 10.21 (s, 1H), 7.41 (d, 1H), 7.22 (d, 1H), 6.92 (dd, 1H), 3.75 (s, 3H), 3.16-3.02 (m, 4H), 1.93-1.73 (m, 5H), 0.84-0.76 (m, 4H); LC-MS: m/z 288.74 (MH+).

### Scheme 16, Cpd (V) Preparation: methyl 5-chloro-4-(cyclopropanecarbonylamino)-2-pyrrolidin-1-ylbenzoate and methyl 3-chloro-4-(cyclopropanecarbonylamino)-2-pyrrolidin-1-ylbenzoate

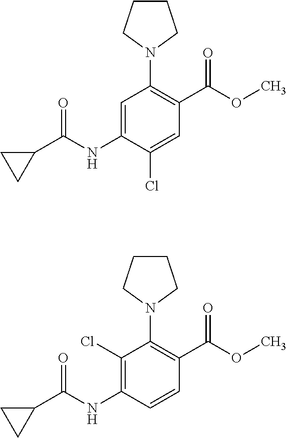 [SMILES: COC(=O)c1cc(Cl)c(NC(=O)C2CC2)cc1N1CCCC1 ; COC(=O)c1ccc(NC(=O)C2CC2)c(Cl)c1N1CCCC1]

1-Chloropyrrolidine-2,5-dione (272.3 mg, 2.04 mmol) was added one-pot to a stirred solution of methyl 4-(cyclopropanecarbonylamino)-2-pyrrolidin-1-ylbenzoate (490.0 mg, 1.7 mmol) in 2-Propanol (15 mL). The reaction was stirred at 60° C. for 2 hours. After this time, the mixture was cooled down to RT, water was added and the mixture was concentrated under high vacuum. The residue was dissolved in EtOAc and washed with a s.s of NaHCO₃. The aqueous phase was extracted twice with EtoAc. Organic portions were combined, washed with brine, dried over Na₂SO₄ and concentrated under vacuum. The crude was purified by FC on silica gel (eluting from Chex 100% to Chex/EtOAc 80:20) affording the products of formula methyl 5-chloro-4-(cyclopropanecarbonylamino)-2-pyrrolidin-1-ylbenzoate and methyl 3-chloro-4-(cyclopropanecarbonylamino)-2-pyrrolidin-1-ylbenzoate.

Yield of methyl 5-chloro-4-(cyclopropanecarbonylamino)-2-pyrrolidin-1-ylbenzoate: 175 mg

1H NMR (400 MHZ, DMSO-d6) δ ppm 9.60 (s, 1H), 7.51 (s, 1H), 7.46 (s, 1H), 3.79 (s, 3H), 3.18-2.95 (m, 4H), 2.08 (dd, 1H), 1.87 (q, 4H), 0.94-0.66 (m, 4H); LC-MS: m/z 323.14 (MH+).

Yield of methyl 3-chloro-4-(cyclopropanecarbonylamino)-2-pyrrolidin-1-ylbenzoate: 80 mg

1H NMR (400 MHZ, DMSO-d6) δ ppm 9.75 (s, 1H), 7.64 (d, 1H), 7.44 (d, 1H), 3.82 (s, 3H), 3.18 (q, 4H), 2.07 (p, 1H), 1.96-1.85 (m, 4H), 0.83 (d, 4H); LC-MS: m/z 323.12 (MH+).

### Scheme 16, Cpd (VI)*a Preparation: 5-chloro-4-(cyclopropanecarbonylamino)-2-pyrrolidin-1-ylbenzoic acid

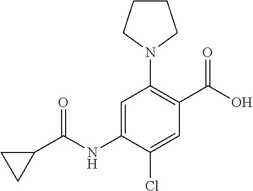 [SMILES: O=C(O)c1cc(Cl)c(NC(=O)C2CC2)cc1N1CCCC1]

To a solution of methyl 5-chloro-4-(cyclopropanecarbonylamino)-2-pyrrolidin-1-ylbenzoate (175.0 mg, 0.540 mmol) in Methanol (6 mL) and THF (6 mL) a solution of lithium hydroxide hydrate (52.32 mg, 1.25 mmol) in 0.5 mL of water was added and the reaction mixture was stirred at 50° C. for 24 hours. After this time the reaction was cooled down, diluted with Water and washed with DCM 3 times. The aqueous phase was acidified with HCl 6 N until pH=2 and then extracted three times with EtOAc. The organic portions were combined, washed with brine, dried over Na₂SO₄ and concentrated under high vacuum to afford the product of formula methyl 3-chloro-4-(cyclopropanecarbonylamino)-2-pyrrolidin-1-ylbenzoate, which was used in the next step without further purification.

### Yield: 76 mg

1H NMR (400 MHZ, DMSO-d6) δ ppm 9.58 (s, 1H), 7.50 (s, 1H), 7.43 (s, 1H), 3.20-3.00 (m, 4H), 1.96-1.70 (m, 4H), 1.50 (tt, 1H), 0.95-0.43 (m, 4H); LC-MS: m/z 309.24 (MH+).

### Scheme 16, Cpd (VI)*b Preparation: 3-chloro-4-(cyclopropanecarbonylamino)-2-pyrrolidin-1-ylbenzoic acid

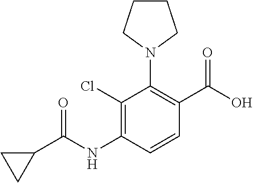 [SMILES: O=C(O)c1ccc(NC(=O)C2CC2)c(Cl)c1N1CCCC1]

The synthesis of the title compound was effected analogously to the synthesis of the compound 5-chloro-4-(cyclopropanecarbonylamino)-2-pyrrolidin-1-ylbenzoic acid wherein methyl 3-chloro-4-(cyclopropanecarbonylamino)-2-pyrrolidin-1-ylbenzoate was used instead of methyl 5-chloro-4-(cyclopropanecarbonylamino)-2-pyrrolidin-1-ylbenzoate. The title compound was obtained in a yield of 80% (58 mg).

1H NMR (400 MHZ, DMSO-d6) δ ppm 9.94 (s, 1H), 7.96-7.85 (m, 2H), 3.57-3.33 (m, 4H), 2.20-1.97 (m, 5H), 1.00-0.64 (m, 4H); LC-MS: m/z 309.24 (MH+).

### Scheme 16, Cpd (VII)*a Preparation: N-[2-chloro-4-(4-methyl-2-phenylpiperazine-1-carbonyl)-5-pyrrolidin-1-ylphenyl]cyclopropanecarboxamide

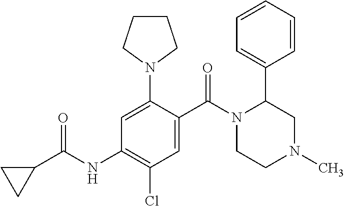 [SMILES: CN1CCN(C(=O)c2cc(Cl)c(NC(=O)C3CC3)cc2N2CCCC2)C(c2ccccc2)C1]

### Example 95

To a solution of HATU (70.19 mg, 0.180 mmol), 5-chloro-4-(cyclopropanecarbonylamino)-2-pyrrolidin-1-ylbenzoic acid (38.0 mg, 0.120 mmol) and N,N-Diisopropylethylamine (0.74 mL, 0.740 mmol) in DMF (1.5 mL), 1-methyl-3-phenylpiperazine (26.03 mg, 0.150 mmol) was added. The reaction was stirred at RT for 30 hours. After this time a saturated solution of NaHCO₃ was added to the reaction mixture and the aqueous phase was extracted three times with EtOAc. The organic portions were collected, washed with brine, residual water was removed by adding Na₂SO₄ and the solvent was evaporated under reduced pressure. The residue was purified by FC on reverse phase using acid conditions (eluting from 5:95 of CH₃CN/H₂O+0.1% of HCOOH to 35:65 of CH₃CN/H₂O+0.1% of HCOOH). Pure fractions were collected and concentrated under vacuum. The residue was taken up with a s.s. of NaHCO₃ and extracted three times with AcOEt. The combined organic fractions were washed with brine, dried over Na₂SO₄, filtered and concentrated under vacuum affording the product of formula N-[2-chloro-4-(4-methyl-2-phenylpiperazine-1-carbonyl)-5-pyrrolidin-1-ylphenyl]cyclopropanecarboxamide as yellow powder.

### Yield: 13 mg

1H NMR (400 MHZ, DMSO-d6) δ ppm 9.57 (d, 1H), 7.73 (d, 1H), 7.47 (d, 1H), 7.42-7.06 (m, 5H), 6.91 (s, 1H), 5.89-4.32 (m, 1H), 3.52-3.38 (m, 1H), 3.14-2.58 (m, 5H), 2.45-1.82 (m, 9H), 1.77-1.51 (m, 1H), 1.40-1.05 (m, 1H), 0.80 (d, 4H); LC-MS: m/z 467.48 (MH+).

### Preparation: N-[2-chloro-4-(1,1-dioxo-1,4-thiazinane-4-carbonyl)-5-pyrrolidin-1-ylphenyl]cyclopropanecarboxamide

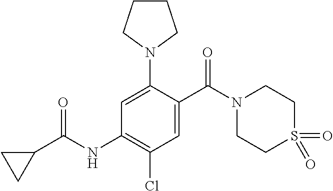 [SMILES: O=C(Nc1cc(N2CCCC2)c(C(=O)N2CCS(=O)(=O)CC2)cc1Cl)C1CC1]

### Example 96

The synthesis of the title compound was effected analogously to the synthesis of the compound of Example 95 N-[2-chloro-4-(4-methyl-2-phenylpiperazine-1-carbonyl)-5-pyrrolidin-1-ylphenyl]cyclopropanecarboxamide wherein 1,4-thiazinane 1,1-dioxide hydrochloride was used instead of 1-methyl-3-phenylpiperazine. The title compound was obtained in a yield of 62% (32 mg).

1H NMR (400 MHZ, DMSO-d6) δ ppm 9.54 (s, 1H), 7.38 (s, 1H), 7.24 (s, 1H), 4.46 (d, 1H), 3.80 (d, 1H), 3.64-3.51 (m, 2H), 3.39 (t, 1H), 3.17 (p, 3H), 3.05 (d, 3H), 2.70 (s, 1H), 2.10-1.96 (m, 1H), 1.87 (s, 4H), 0.80 (d, 4H); LC-MS: m/z 426.13 (MH+).

### Preparation: N-[2-chloro-4-(4-methyl-2-phenylpiperazine-1-carbonyl)-3-pyrrolidin-1-ylphenyl]cyclopropanecarboxide

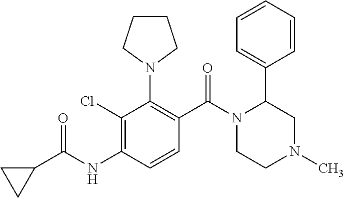 [SMILES: CN1CCN(C(=O)c2ccc(NC(=O)C3CC3)c(Cl)c2N2CCCC2)C(c2ccccc2)C1]

### Example 97

The synthesis of the title compound was effected analogously to the synthesis of the compound of Example 95 N-[2-chloro-4-(4-methyl-2-phenylpiperazine-1-carbonyl)-5-pyrrolidin-1-ylphenyl]cyclopropanecarboxamide wherein 3-chloro-4-(cyclopropanecarbonylamino)-2-pyrrolidin-1-ylbenzoic acid was used instead of 5-chloro-4-(cyclopropanecarbonylamino)-2-pyrrolidin-1-ylbenzoic acid. The title compound was obtained in a yield of 68% (29 mg).

1H NMR (400 MHz, DMSO-d6) δ ppm 9.73 (d, 1H), 7.60 (dd, 2H), 7.54-7.10 (m, 5H), 7.02-5.75 (m, 1H), 5.75-4.65 (m, 1H), 3.07-2.95 (m, 3H), 2.82 (d, 1H), 2.63 (d, 1H), 2.36-2.10 (m, 4H), 2.05-1.74 (m, 8H), 0.82 (d, 4H); LC-MS: m/z 467.48 (MH+).

### Preparation: N-[2-chloro-4-(1,1-dioxo-1,4-thiazinane-4-carbonyl)-3-pyrrolidin-1-ylphenyl]cyclopropanecarboxamide

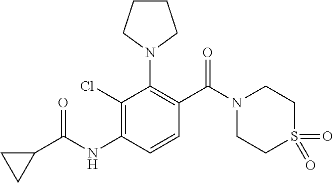 [SMILES: O=C(Nc1ccc(C(=O)N2CCS(=O)(=O)CC2)c(N2CCCC2)c1Cl)C1CC1]

### Example 98

The synthesis of the title compound was effected analogously to the synthesis of the compound of Example 95 N-[2-chloro-4-(4-methyl-2-phenylpiperazine-1-carbonyl)-5-pyrrolidin-1-ylphenyl]cyclopropanecarboxamide wherein 3-chloro-4-(cyclopropanecarbonylamino)-2-pyrrolidin-1-ylbenzoic acid was used instead of 5-chloro-4-(cyclopropanecarbonylamino)-2-pyrrolidin-1-ylbenzoic acid and 1,4-thiazinane 1,1-dioxide hydrochloride was used instead of 1-methyl-3-phenylpiperazine. The title compound was obtained in a yield of 31% (12 mg).

1H NMR (400 MHZ, DMSO-d6) δ ppm 9.71 (s, 1H), 7.54 (d, 1H), 7.30 (d, 1H), 4.52 (d, 1H), 3.74 (d, 1H), 3.55 (t, 2H), 3.36 (t, 1H), 3.24-3.08 (m, 6H), 3.01 (t, 1H), 2.08-1.99 (m, 1H), 1.85 (s, 4H), 0.82 (d, 4H); LC-MS: m/z 426.13 (MH+).

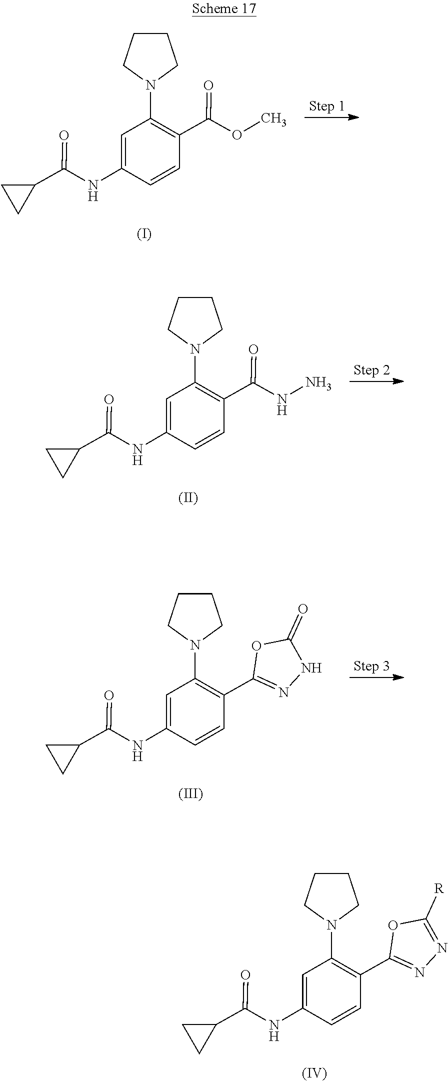

### Step 1

Compound of formula II may be obtained from compound of formula I (synthesized according to the Scheme 1 7) by reaction with hydrazine hydrate in a suitable solvent such as EtOH, typically at 80° C. The reaction takes about 3 days to complete.

### Step 2

Compound of formula III may be obtained by cyclization of compound of formula II with triphosgene in presence of a suitable organic base, e.g. DIPEA. The reaction is carried out in a suitable solvent such as DCM, typically at room temperature. The reaction takes about 1 hour to complete.

### Step 3

Compound of formula IV may be obtained by activation of compound of formula III with a suitable phosphonium reagent, e.g. pyBOP, in presence of an organic base, e.g. DIPEA, followed by SnAr substitution, with a proper amine. The reaction is carried out in a suitable solvent, such as DMF, typically at room temperature. The reaction takes about 12 hours to complete.

### Scheme 17, Cpd (II) Preparation: N-[4-(hydrazinecarbonyl)-3-pyrrolidin-1-ylphenyl]cyclopropanecarboxamide

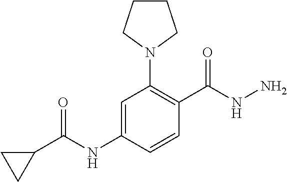 [SMILES: NNC(=O)c1ccc(NC(=O)C2CC2)cc1N1CCCC1]

To a suspension of methyl 4-(cyclopropanecarbonylamino)-2-pyrrolidin-1-ylbenzoate (2.0 g, 6.45 mmol) in Ethanol (10 mL), hydrazine hydrate (2.48 g, 32.25 mmol) was added and the reaction was stirred at 80° C. for 3 days. After this time, the reaction mixture was cooled down to RT and concentrated under vacuum. The residue was taken up with H₂O and filtered. The solid was dried under vacuum affording the product of formula N-[4-(hydrazinecarbonyl)-3-pyrrolidin-1-ylphenyl]cyclopropanecarboxamide. The product was used in the next step without further purification.

### Yield: 1.2 g

1H NMR (400 MHZ, DMSO-d6) δ ppm 10.07 (s, 1H), 9.20 (s, 1H), 7.07-6.99 (m, 2H), 6.88 (dd, 1H), 4.32 (d, 2H), 3.19-3.09 (m, 4H), 1.88-1.82 (m, 4H), 1.78-1.69 (m, 1H), 0.77 (dq, 4H); LC-MS: m/z 289.25 (MH+).

### Scheme 17, Cpd (III) Preparation: N-[4-(2-oxo-3H-1,3,4-oxadiazol-5-yl)-3-pyrrolidin-1-ylphenyl]cyclopropanecarboxamide

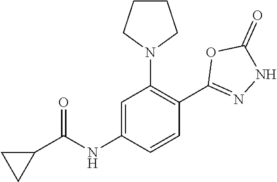 [SMILES: O=C(Nc1ccc(-c2n[nH]c(=O)o2)c(N2CCCC2)c1)C1CC1]

To a suspension of N-[4-(hydrazinecarbonyl)-3-pyrrolidin-1-ylphenyl]cyclopropanecarboxamide (0.5 g, 1.73 mmol) and N,N-Diisopropylethylamine (0.6 mL, 3.47 mmol) in DCM (35 mL), a solution of carbonic acid bis(trichloromethyl) ester (205.83 mg, 0.690 mmol) in DCM (1 mL) was added. The reaction mixture was stirred at RT for 1 hour. After this time, MeOH was added and the mixture was concentrated under vacuum. The residue was taken up with DCM and filtered. The solid was washed with DCM and dried under vacuum affording the product of formula N-[4-(2-oxo-3H-1,3,4-oxadiazol-5-yl)-3-pyrrolidin-1-ylphenyl]cyclopropanecarboxamide.

### Yield: 300 mg

1H NMR (400 MHZ, DMSO-d6) δ ppm 12.33 (s, 1H), 10.25 (s, 1H), 7.36-7.12 (m, 2H), 7.01 (dd, 1H), 3.16-3.03 (m, 4H), 1.92-1.82 (m, 4H), 1.78 (dt, 1H), 0.87-0.71 (m, 4H); LC-MS: m/z 315.2 (MH+).

### Scheme 17, Cpd (V)*a Preparation: N-[3-pyrrolidin-1-yl-4-(5-pyrrolidin-1-yl-1,3,4-oxadiazol-2-yl)phenyl]cyclopropanecarboxamide

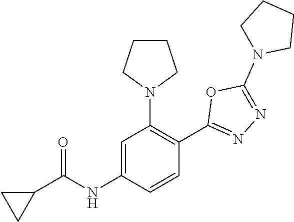 [SMILES: O=C(Nc1ccc(-c2nnc(N3CCCC3)o2)c(N2CCCC2)c1)C1CC1]

### Example 99

To a solution of N-[4-(2-oxo-3H-1,3,4-oxadiazol-5-yl)-3-pyrrolidin-1-ylphenyl]cyclopropanecarboxamide (50.0 mg, 0.160 mmol) (1.5 mL), N,N-Diisopropylethylamine (0.06 mL, 0.320 mmol) was added followed by 1-benzotriazolyloxy-tris(1-pyrrolidinyl)phosphonium hexafluorophosphate (91.05 mg, 0.170 mmol) and pyrrolidine (0.03 mL, 0.320 mmol). The reaction mixture was stirred overnight at RT. The day after, the reaction mixture was directly purified by FC on reverse phase using basic conditions (from 5:95 of CH₃CN/H₂O+10 mM ammonium bicarbonate aqueous solution adjusted to pH 10 with ammonia to 95:5 of CH₃CN/H₂O+10 mM ammonium bicarbonate aqueous solution adjusted to pH 10 with ammonia). Fractions containing the desired product were collected and concentrated under vacuum. The residue was again purified by FC on silica gel (eluting from DCM/EtOAc 8:2 to DCM/EtOAc 2:8) affording the product of formula N-[3-pyrrolidin-1-yl-4-(5-pyrrolidin-1-yl-1,3,4-oxadiazol-2-yl)phenyl]cyclopropanecarboxamide.

### Yield: 8.1 mg

1H NMR (400 MHZ, DMSO-d6) δ ppm 10.22 (s, 1H), 7.34-7.17 (m, 2H), 7.00 (dd, 1H), 3.43 (d, 4H), 3.07-2.96 (m, 4H), 2.02-1.89 (m, 4H), 1.86-1.72 (m, 5H), 0.79 (dt, 4H); LC-MS: m/z 368.26 (MH+).

### Preparation: N-[4-[5-(4,4-difluoropiperidin-1-yl)-1,3,4-oxadiazol-2-yl]-3-pyrrolidin-1-ylphenyl]cyclopropanecarboxamide

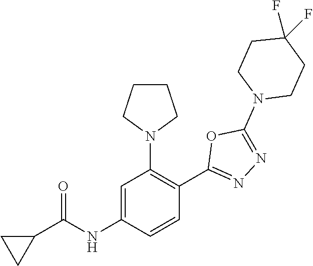 [SMILES: O=C(Nc1ccc(-c2nnc(N3CCC(F)(F)CC3)o2)c(N2CCCC2)c1)C1CC1]

### Example 100

The synthesis of the title compound was effected analogously to the synthesis of the compound of Example 99 N-[3-pyrrolidin-1-yl-4-(5-pyrrolidin-1-yl-1,3,4-oxadiazol-2-yl)phenyl]cyclopropanecarboxamide wherein 4,4-difluoropiperidine was used instead of pyrrolidine. The title compound was obtained in a yield of 19% (13 mg).

1H NMR (400 MHZ, DMSO-d6) δ ppm 10.24 (s, 1H), 7.32-7.23 (m, 2H), 7.01 (dd, 1H), 3.58 (t, 4H), 3.10-2.96 (m, 4H), 2.11 (dt, 4H), 1.81 (d, 5H), 0.80 (d, 4H); LC-MS: m/z 418.24 (MH+).

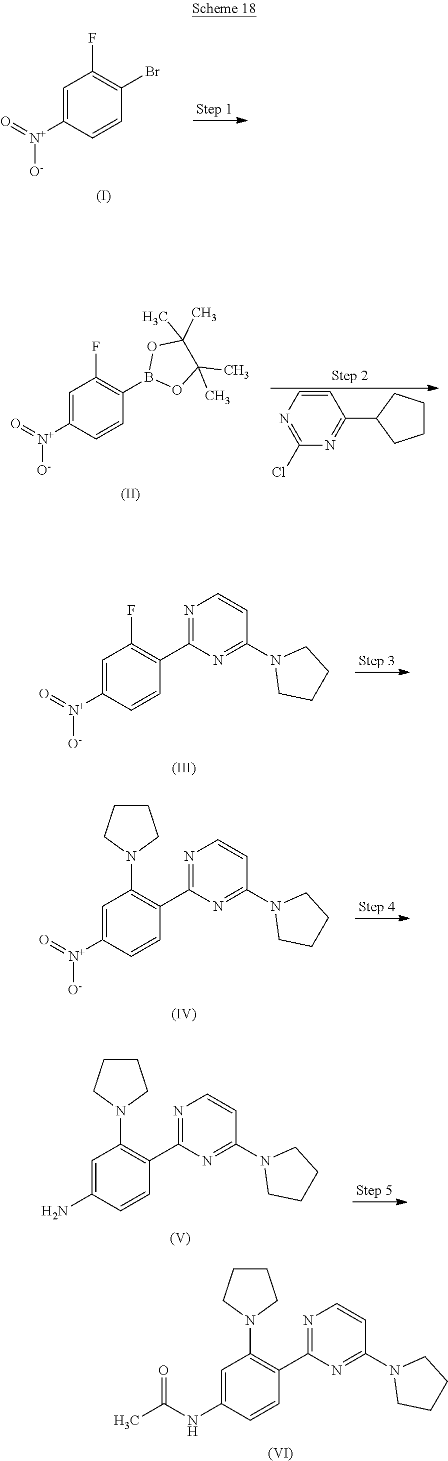

### Step 1

Compound of formula II may be obtained via Miyaura coupling between compound of formula I (commercially available from Fluorochem) and Bis(pinacolato)diboron, in presence of a suitable catalyst, such as PdCl₂(dppf)·CH₂Cl₂, a suitable base, e.g. AcOK, in a suitable solvent, e.g. 1,4 dioxane, typically at 100° C. The reaction takes about 20 hours to complete.

### Step 2

Compound of formula III may be obtained via Suzuki coupling between compound of formula II and the desired commercially available heteroaryl halide, in presence of a suitable palladium precatalyst, e.g. XPhos Pd G2, a suitable base, e.g. K₃PO₄, in a suitable mixture of solvents, e.g. 1,2-dimethoxyethane/water, typically at 95° C. The reaction takes about 12 hours to complete.

### Step 3

Compound of formula IV may be obtained from compound of formula III by SNAr substitution with pyrrolidine in a suitable aprotic solvent, e.g. DMSO, typically at 120° C. The reaction takes about 12 hours to complete.

### Step 4

Compound of formula V may be obtained by nitro reduction of compound of formula IV under catalytic hydrogenation conditions, using for example Pd/C in a suitable solvent such as MeOH, typically at room temperature. The reaction takes about 2 hours to complete.

### Step 5

Compound of formula VI may be obtained by coupling reaction between compound of formula V and a proper commercially available acyl chloride, e.g. acetyl chloride, in presence of an organic base, e.g. TEA. The reaction is carried out in a suitable solvent such as DCM, typically at room temperature. The reaction takes about 2 hours to complete.

### Scheme 18, Cpd (II) Preparation: 2-(2-fluoro-4-nitrophenyl)-4,4,5,5-tetramethyl-1,3,2-dioxaborolane

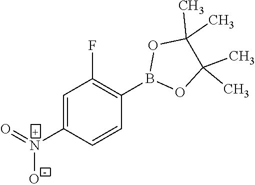

4-bromo-3-fluoronitrobenzene (1.6 g, 7.27 mmol), 4,4,5,5-tetramethyl-2-(4,4,5,5-tetramethyl-1,3,2-dioxaborolan-2-yl)-1,3,2-dioxaborolane (1.94 g, 7.64 mmol), potassium acetate (2.16 g, 21.82 mmol) and [1,1′-Bis(diphenylphosphino) ferrocene]dichloropalladium (II), complex with dichloromethane (119.08 mg, 0.150 mmol) were suspended in 1,4-Dioxane (70 mL). The mixture was degassed through N₂-vacuum cycles and then stirred under N₂ atmosphere at 100° C. for 20 h. After this time, the mixture was filtered through a celite pad, washed with EtOAc and concentrated under vacuum affording the product of formula 2-(2-fluoro-4-nitrophenyl)-4,4,5,5-tetramethyl-1,3,2-dioxaborolane, which was used in the next step without further purification.

### Yield: 2.76 g

1H NMR (400 MHZ, CDCl₃) δ ppm 8.02 (dd, 1H), 7.97-7.84 (m, 2H), 1.40 (s, 12H).

### Scheme 18, Cpd (III) Preparation: 2-(2-fluoro-4-nitrophenyl)-4-pyrrolidin-1-ylpyrimidine

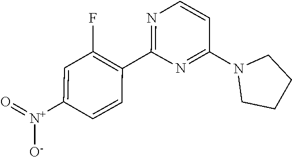 [SMILES: O=[N+]([O-])c1ccc(-c2nccc(N3CCCC3)n2)c(F)c1]

In a proper vial 2-chloro-4-pyrrolidin-1-ylpyrimidine (250.0 mg, 1.36 mmol), 2-(2-fluoro-4-nitrophenyl)-4,4,5,5-tetramethyl-1,3,2-dioxaborolane (0.62 g, 1.63 mmol), X-phos aminobiphenyl palladium chloride precatalyst (106.98 mg, 0.140 mmol) and tripotassium phosphate (879.26 mg, 4.08 mmol) were suspended in Water (2 mL), and 1,2-dimethoxyethane (15 mL). The vial was sealed and the resulting suspension was degassed under nitrogen flux for 5 min and then heated to 95° C. for 12 h. After this time, the reaction mixture was cooled down to room temperature and diluted with water and extracted three times with EtOAc. The organic portions were combined, washed with brine, dried over Na₂SO₄, filtered and the solvent removed under vacuum affording the product of formula 2-(2-fluoro-4-nitrophenyl)-4-pyrrolidin-1-ylpyrimidine, which was used in the next step without further purification.

### Yield: 570 mg

1H NMR (400 MHZ, DMSO-d6) δ ppm 8.32 (d, 1H), 8.27-8.05 (m, 3H), 6.54 (d, 1H), 3.71-3.38 (m, 4H), 1.99 (d, 4H); LC-MS: m/z 289.18 (MH+).

### Scheme 18, Cpd (IV) Preparation: 2-(4-nitro-2-pyrrolidin-1-ylphenyl)-4-pyrrolidin-1-ylpyrimidine

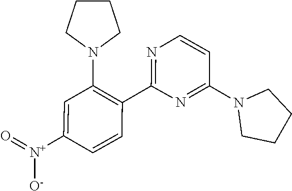 [SMILES: O=[N+]([O-])c1ccc(-c2nccc(N3CCCC3)n2)c(N2CCCC2)c1]

To a stirred solution of 2-(2-fluoro-4-nitrophenyl)-4-pyrrolidin-1-ylpyrimidine (275.0 mg, 0.670 mmol) in DMSO (4 mL), pyrrolidine (0.17 mL, 2 mmol) was added and the mixture was stirred overnight at 120° C. The day after, the reaction was cooled down to RT, diluted with water and the aqueous phase extracted three times with EtOAc. The combined organic portions were washed with brine, dried over Na₂SO₄, filtered and concentrated under vacuum. The residue was purified by FC on NH column (eluting from 100% of cHex to AcOEt/cHex 70:30) affording the product of formula 2-(4-nitro-2-pyrrolidin-1-ylphenyl)-4-pyrrolidin-1-ylpyrimidine.

### Yield: 140 mg

1H NMR (400 MHZ, DMSO-d6) δ ppm 8.22 (d, 1H), 7.47 (d, 3H), 6.42 (d, 1H), 3.31 (s, 4H), 3.03 (d, 4H), 1.94 (d, 4H), 1.78 (d, 4H); LC-MS: m/z 340.2 (MH+).

### Scheme 18, Cpd (V) Preparation: 3-pyrrolidin-1-yl-4-(4-pyrrolidin-1-ylpyrimidin-2-yl) aniline

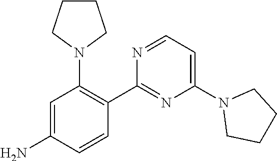 [SMILES: Nc1ccc(-c2nccc(N3CCCC3)n2)c(N2CCCC2)c1]

To a solution of 2-(4-nitro-2-pyrrolidin-1-ylphenyl)-4-pyrrolidin-1-ylpyrimidine (140.0 mg, 0.410 mmol) in MeOH, Pd/C 10% (43.9 mg, 0.040 mmol) was added. The reaction mixture was stirred for 2 hours under H₂ atmosphere at room temperature. After this time, the mixture was filtered through a celite pad, washed with MeOH and concentrated under vacuum affording the product of formula 3-pyrrolidin-1-yl-4-(4-pyrrolidin-1-ylpyrimidin-2-yl) aniline, which was used in the next step without further purification.

### Yield: 142 mg

1H NMR (400 MHz, DMSO-d6) δ ppm 8.11 (d, 1H), 7.12 (d, 1H), 6.20 (d, 1H), 6.06-5.85 (m, 2H), 4.99 (s, 2H), 3.49-3.10 (m, 4H), 3.17-2.75 (m, 4H), 1.93 (t, 4H), 1.80-1.67 (m, 4H); LC-MS: m/z 310.26 (MH+).

### Scheme 18, Cpd (VI) Preparation: N-[3-pyrrolidin-1-yl-4-(4-pyrrolidin-1-ylpyrimidin-2-yl)phenyl]acetamide

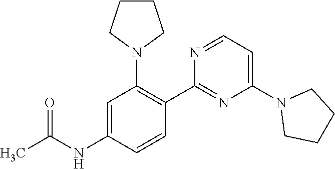 [SMILES: CC(=O)Nc1ccc(-c2nccc(N3CCCC3)n2)c(N2CCCC2)c1]

### Example 101

To a mixture of N,N-Diisopropylethylamine (119.91 uL, 0.690 mmol) and 3-pyrrolidin-1-yl-4-(4-pyrrolidin-1-ylpyrimidin-2-yl) aniline (142.0 mg, 0.460 mmol) in DCM (4.494 mL) at 0° C., acetyl chloride (30 uL, 0.410 mmol) was added dropwise and the mixture was stirred at RT for 2 hours. After this time, MeOH was added and the reaction mixture was concentrated under vacuum. The residue was purified by FC on reverse phase using acid conditions (eluting from 5:95 of CH₃CN/H₂O+0.1% of HCOOH to 40:60 of CH₃CN/H₂O+0.1% of HCOOH). Pure fractions were collected and concentrated under vacuum. The residue was taken up with a s.s. of NaHCO₃ and extracted three times with EtOAc. The combined organic portions were washed with brine, dried over Na₂SO₄, filtered and concentrated under vacuum affording the product of formula N-[3-pyrrolidin-1-yl-4-(4-pyrrolidin-1-ylpyrimidin-2-yl)phenyl]acetamide.

### Yield: 22 mg

1H NMR (400 MHZ, DMSO-d6) δ ppm 9.80 (s, 1H), 8.16 (d, 1H), 7.23 (d, 1H), 7.05 (d, 1H), 6.95 (dd, 1H), 6.30 (d, 1H), 3.42 (s, 4H), 3.00-2.89 (m, 4H), 2.04 (s, 3H), 1.94 (s, 4H), 1.80-1.68 (m, 4H); LC-MS: m/z 352.46 (MH+).

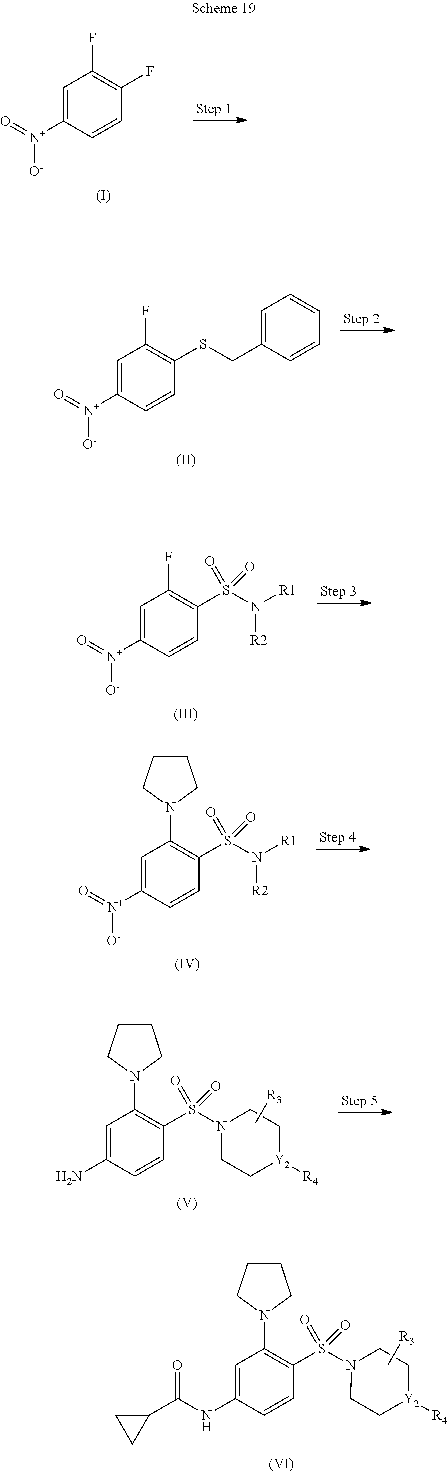

### Step 1

Compound of formula II may be obtained by SNAr substitution between compound of formula I and benzyl mercaptan, in presence of a suitable base, e.g. K₂CO₃, in a suitable aprotic solvent, e.g. DMF, typically at RT. The reaction takes about 20 hours to complete.

### Step 2

Compound of formula III may be obtained by oxidative chlorination of compound of formula II to the corresponding sulphonyl chloride in the presence of a suitable oxidizing agent, e.g. dichloro-5,5-dimethylimidazolidine-2,4-dione, followed by treatment with a proper amine. The reaction is carried out in a suitable solvent, e.g. DCM, at a temperature between 0° C. and room temperature. The reaction takes about 16 hours to complete

### Step 3

Compound of formula IV may be obtained from compound of formula III by SNAr substitution with pyrrolidine in a suitable aprotic solvent, e.g. DMSO, typically at 120° C. The reaction takes about 12 hours to complete.

### Step 4

Compound of formula V may be obtained by nitro reduction of compound of formula IV in the presence of Tin (III) chloride dehydrate in a suitable solvent, e.g. EtOH, typically at 80° C. The reaction takes about 1 hour to complete.

### Step 5

Compound of formula VI may be obtained by coupling reaction between compound of formula V and a proper commercially available acyl chloride, e.g. cyclopropanecarbonyl chloride, in presence of an organic base, e.g. TEA. The reaction is carried out in a suitable solvent such as DCM, typically at room temperature. The reaction takes about 12 hours to complete.

### Scheme 19, Cpd (II) Preparation: 1-benzylsulfanyl-2-fluoro-4-nitrobenzene

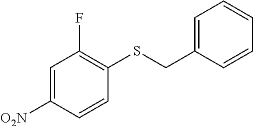 [SMILES: O=[N+]([O-])c1ccc(SCc2ccccc2)c(F)c1]

To a solution of 1,2-difluoro-4-nitrobenzene (2.0 g, 12.57 mmol) in DMF (13.5 mL), phenylmethanethiol (1.48 mL, 12.57 mmol) was added followed by potassium carbonate (1.91 g, 13.83 mmol). The reaction was stirred at room temperature overnight. After this time the reaction was diluted with H₂O and filtered. The solid was washed with H₂O and dried under vacuum affording the product of formula 1-benzylsulfanyl-2-fluoro-4-nitrobenzene (N0806-30-1:3.3 g, 12.53 mmol, 99.7% yield) which was used in the next step without further purification.

1H NMR (400 MHZ, DMSO-d6) δ ppm 8.13-8.03 (m, 2H), 7.72 (t, 1H), 7.46 (d, 2H), 7.39-7.24 (m, 3H) 4.47 (s, 2H); LC-MS: m/z 264.01 (MH+).

### Scheme 19, Cpd (III)*a Preparation: 1-(2-fluoro-4-nitrophenyl) sulfonyl-4-methylpiperazine

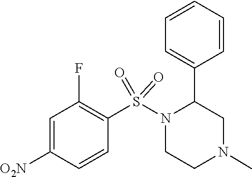 [SMILES: CN1CCN(S(=O)(=O)c2ccc([N+](=O)[O-])cc2F)C(c2ccccc2)C1]

To a suspension of 1-benzylsulfanyl-2-fluoro-4-nitrobenzene (100.0 mg, 0.380 mmol) in a mixture of MeCN (3.8 mL), Water (0.100 mL) and acetic acid (49.0 uL, 0.860 mmol) was added 1,3-dichloro-5,5-dimethylimidazolidine-2,4-dione (149.66 mg, 0.760 mmol) at 0° C. After stirring for 2 h at 0° C., the reaction mixture was concentrated under reduced pressure, the residue was dissolved in DCM (1.5 mL) and again cooled to 0° C. Then a saturated solution of NaHCO₃ was added and the mixture was stirred for 15 minutes at 0° C. Afterwards the mixture was extracted with DCM (×2) and the combined organic layers were washed with brine, dried over Na₂SO₄, filtered and evaporated under reduced pressure. The residue was dissolved in DCM (1.5 mL) and 1-methyl-3-phenylpiperazine (66.95 mg, 0.380 mmol) was added at 0° C. followed by 1,4-diazabicyclo[2.2.2]octane (51.12 mg, 0.460 mmol) and the mixture was allowed to warm to room temperature. After stirring 16 h, the reaction mixture was quenched with water and extracted with DCM (×3). The organic layers were washed with brine, dried using a phase separator and concentrated to afford a crude that was purified by FC on silica gel (eluting from 100% of cHex to cHex/EtOAc 80:20) affording the product of formula 1-(2-fluoro-4-nitrophenyl) sulfonyl-4-methyl-2-phenylpiperazine.

### Yield: 130 mg

¹H NMR (400 MHZ, DMSO-d₆) δ 8.37 (dd, 1H), 8.24-8.17 (m, 1H), 8.15-8.05 (m, 1H), 7.47-7.43 (m, 2H), 7.36-7.18 (m, 3H), 5.09-5.02 (m, 1H), 3.75-3.66 (m, 1H), 3.31-3.18 (m, 2H), 2.77-2.62 (m, 1H), 2.20-2.07 (m, 4H), 1.79 (td, 1H); LC-MS: m/z 380.12 (MH+).

### Scheme 19, Cpd (III)*b Preparation: 1-(2-fluoro-4-nitrophenyl) sulfonyl-4-methylpiperazine

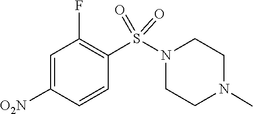 [SMILES: CN1CCN(S(=O)(=O)c2ccc([N+](=O)[O-])cc2F)CC1]

The synthesis of the title compound was effected analogously to the synthesis of the compound 1-(2-fluoro-4-nitrophenyl) sulfonyl-4-methylpiperazine wherein 1-methylpiperazine was used instead of 1-methyl-3-phenylpiperazine. The title compound was obtained in a quantitative yield (700 mg).

¹H NMR (400 MHZ, DMSO-d₆) δ 8.41 (dd, 1H), 8.28-8.22 (m, 1H), 8.08-8.02 (m, 1H), 3.15 (t, 4H), 2.37 (t, 4H), 2.17 (s, 3H); LC-MS: m/z 304.05 (MH+).

### Scheme 19, Cpd (IV)*a Preparation: 4-methyl-1-(4-nitro-2-pyrrolidin-1-ylphenyl) sulfonyl-2-phenylpiperazine

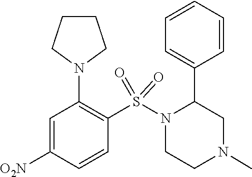 [SMILES: CN1CCN(S(=O)(=O)c2ccc([N+](=O)[O-])cc2N2CCCC2)C(c2ccccc2)C1]

A mixture of pyrrolidine (0.56 g, 7.91 mmol) and 1-(2-fluoro-4-nitrophenyl) sulfonyl-4-methyl-2-phenylpiperazine (1.0 g, 2.64 mmol) in DMSO (15 mL) was heated to 120° C. for 12 h. After this time the reaction was diluted with water and then extracted with AcOEt ×3. Organic phase was dried over Na₂SO₄, filtered and concentrated under vacuum. The crude was purified by FC on silica gel (eluting from 100% of cHex 100% to cHex/EtOAc 80:20) to afford the product of formula 4-methyl-1-(4-nitro-2-pyrrolidin-1-ylphenyl) sulfonyl-2-phenylpiperazine.

### Yield: 397 mg

### LC-MS: m/z 431.19 (MH+).

### Scheme 19, Cpd (IV)*b Preparation: 1-methyl-4-(4-nitro-2-pyrrolidin-1-ylphenyl) sulfonylpiperazine

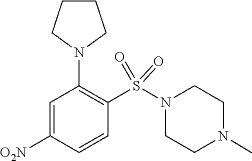 [SMILES: CN1CCN(S(=O)(=O)c2ccc([N+](=O)[O-])cc2N2CCCC2)CC1]

The synthesis of the title compound was effected analogously to the synthesis of the compound 4-methyl-1-(4-nitro-2-pyrrolidin-1-ylphenyl) sulfonyl-2-phenylpiperazine wherein 1-(2-fluoro-4-nitrophenyl) sulfonyl-4-methylpiperazine was used instead of 1-(2-fluoro-4-nitrophenyl) sulfonyl-4-methyl-2-phenylpiperazine. The title compound was obtained in yield of 70% (490 mg).

### LC-MS: m/z 355.15 (MH+).

### Scheme 19, Cpd (V)*a Preparation: 4-(4-methyl-2-phenylpiperazin-1-yl) sulfonyl-3-pyrrolidin-1-ylaniline

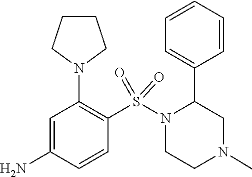 [SMILES: CN1CCN(S(=O)(=O)c2ccc(N)cc2N2CCCC2)C(c2ccccc2)C1]

To a solution of 4-methyl-1-(4-nitro-2-pyrrolidin-1-ylphenyl) sulfonyl-2-phenylpiperazine (397.0 mg, 0.920 mmol) in Ethanol (10 mL), tin (III) chloride dihydrate (1.3 g, 5.53 mmol) was added and the reaction was stirred at 80° C. for 1 h. After this time EtOH was evaporated under reduced pressure and the residue dissolved in EtOAc. A precipitate was formed and it was filtered on Hirsch funnel under reduced pressure. The filtrate was evaporated under reduced pressure and then purified by SCX first washing with MeOH and then eluting with NH₃ 1N in MeOH. Basic fractions were collected and concentrated under vacuum affording the product of formula 4-(4-methyl-2-phenylpiperazin-1-yl) sulfonyl-3-pyrrolidin-1-ylaniline. The product was used in the next step without further purification.

### Yield: 200 mg

### LC-MS: m/z 401.27 (MH+).

### Scheme 19, Cpd (V)*b Preparation: 4-(4-methylpiperazin-1-yl) sulfonyl-3-pyrrolidin-1-ylaniline

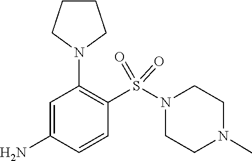 [SMILES: CN1CCN(S(=O)(=O)c2ccc(N)cc2N2CCCC2)CC1]

The synthesis of the title compound was effected analogously to the synthesis of the compound 4-(4-methyl-2-phenylpiperazin-1-yl) sulfonyl-3-pyrrolidin-1-ylaniline wherein 1-methyl-4-(4-nitro-2-pyrrolidin-1-ylphenyl) sulfonylpiperazine was used instead of 4-methyl-1-(4-nitro-2-pyrrolidin-1-ylphenyl) sulfonyl-2-phenylpiperazine. The title compound was obtained in yield of 9% (40 mg).

### LC-MS: m/z 323.18 (MH+).

### Scheme 19, Cpd (VI)*a Preparation: N-[4-(4-methyl-2-phenylpiperazin-1-yl) sulfonyl-3-pyrrolidin-1-ylphenyl]cyclopropanecarboxamide

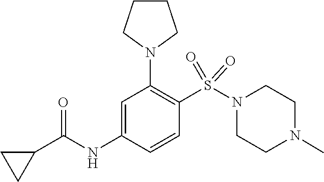 [SMILES: CN1CCN(S(=O)(=O)c2ccc(NC(=O)C3CC3)cc2N2CCCC2)CC1]

### Example 102

To a solution of 4-(4-methyl-2-phenylpiperazin-1-yl) sulfonyl-3-pyrrolidin-1-ylaniline (199.0 mg, 0.500 mmol) in DCM (6.387 mL), N,N-Diisopropylethylamine (0.13 mL, 0.750 mmol) was added followed by cyclopropanecarbonyl chloride (0.06 mL, 0.650 mmol). The reaction was stirred at RT/ON. The day after the reaction was diluted with DCM, washed with a s.s. of NaHCO₃, brine, dried over Na₂SO₄, filtered and concentrated under vacuum. The residue was purified by FC on RP using basic conditions (eluting from 100% of water+0.1% of NH₄OH to 20:80 of CH₃CN/water+0.1% of NH₄OH) to afford the product of formula N-[4-(4-methyl-2-phenylpiperazin-1-yl) sulfonyl-3-pyrrolidin-1-ylphenyl]cyclopropanecarboxamide.

### Yield: 72 mg

¹H NMR (400 MHZ, DMSO-d₆) δ 10.46 (s, 1H), 7.75 (d, 1H), 7.66 (d, 1H), 7.43-7.35 (m, 2H), 7.32-7.12 (m, 4H), 5.01-4.93 (m, 1H), 3.57-3.47 (m, 1H), 3.26-2.91 (m, 8H), 2.63-2.57 (m, 1H), 2.19 (dd, 1H), 1.90-1.65 (m, 6H), 0.91-0.67 (m, 5H); LC-MS: m/z 467.21 (MH+).

### Preparation: N-[4-(4-methylpiperazin-1-yl) sulfonyl-3-pyrrolidin-1-ylphenyl]cyclopropanecarboxamide

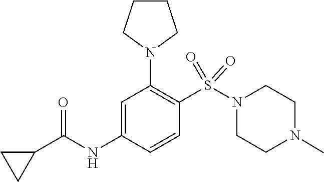 [SMILES: CN1CCN(S(=O)(=O)c2ccc(NC(=O)C3CC3)cc2N2CCCC2)CC1]

### Example 103

The synthesis of the title compound was effected analogously to the synthesis of the compound of Example 102 N-[4-(4-methyl-2-phenylpiperazin-1-yl) sulfonyl-3-pyrrolidin-1-ylphenyl]cyclopropanecarboxamide wherein 4-(4-methylpiperazin-1-yl) sulfonyl-3-pyrrolidin-1-ylaniline was used instead of 4-(4-methyl-2-phenylpiperazin-1-yl) sulfonyl-3-pyrrolidin-1-ylaniline. The title compound was obtained in yield of 73% (33 mg).

1H NMR (400 MHZ, DMSO-d6) δ ppm 10.52-10.43 (m, 1H), 7.72-7.61 (m, 2H), 7.26 (d, 1H), 3.24-3.13 (m, 4H), 3.06-2.95 (m, 3H), 2.37-2.21 (m, 5H), 2.18-2.10 (m, 3H), 1.93-1.73 (m, 5H), 0.83 (d, 4H); LC-MS: m/z 393.18 (MH+).

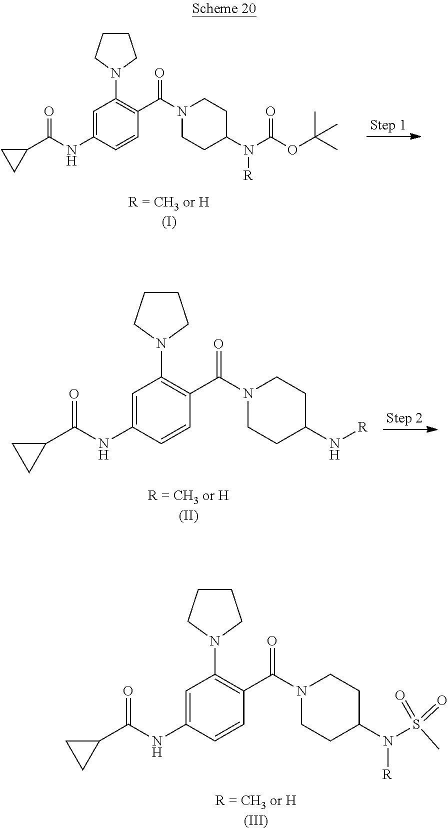

### Step 1

Compound of formula II may be obtained from compound of formula I (synthesized according to the Scheme 1) by removing the Boc protecting group under acidic conditions, e.g. TFA, typically at RT. The reaction takes from about 3 hours to 12 hours to complete.

### Step 2

Compound of formula III may be obtained via sulphonamide formation between compound of formula II and methanesulfonyl chloride in presence of a suitable organic base, e.g. TEA, in a suitable solvent, e.g. DCM, typically at RT. The reaction takes about 5 minutes to complete

### Scheme 20, Cpd (II)*a Preparation: N-[4-[4-(methylamino)piperidine-1-carbonyl]-3-pyrrolidin-1-ylphenyl]cyclopropanecarboxamide

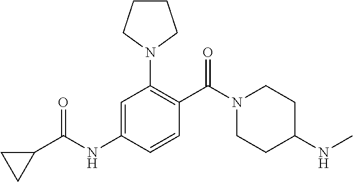 [SMILES: CNC1CCN(C(=O)c2ccc(NC(=O)C3CC3)cc2N2CCCC2)CC1]

To a solution of tert-butyl N-[1-[4-(cyclopropanecarbonylamino)-2-pyrrolidin-1-ylbenzoyl]piperidin-4-yl]-N-methylcarbamate (160.0 mg, 0.340 mmol) in DCM (5.128 mL), at RT, Trifluoroacetic acid (0.26 mL, 3.4 mmol) was added and the resulting reaction mixture was stirred at RT overnight. The mixture was concentrated under vacuum, dissolved in 1 mL of MeOH and purified by SCX first washing with MeOH and then eluting with NH₃ 1N in MeOH. Basic fractions were collected and concentrated under vacuum affording the product of formula N-[4-[4-(methylamino)piperidine-1-carbonyl]-3-pyrrolidin-1-ylphenyl]cyclopropanecarboxamide, which was used in the next step without further purification.

### Yield: 86 mg

### LC-MS: m/z 371.28 (MH+).

### Scheme 20, Cpd (II)*b Preparation: N-[4-(4-aminopiperidine-1-carbonyl)-3-pyrrolidin-1-ylphenyl]cyclopropanecarboxamide

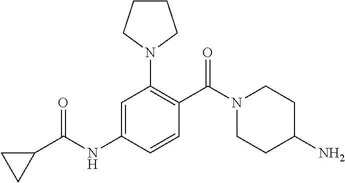 [SMILES: NC1CCN(C(=O)c2ccc(NC(=O)C3CC3)cc2N2CCCC2)CC1]

The synthesis of the title compound was effected analogously to the synthesis of the compound N-[4-[4-(methylamino)piperidine-1-carbonyl]-3-pyrrolidin-1-ylphenyl]cyclopropanecarboxamide wherein tert-butyl N-[1-[4-(cyclopropanecarbonylamino)-2-pyrrolidin-1-ylbenzoyl]piperidin-4-yl]carbamate was used instead of tert-butyl N-[1-[4-(cyclopropanecarbonylamino)-2-pyrrolidin-1-ylbenzoyl]piperidin-4-yl]-N-methylcarbamate. The title compound was obtained in a yield 70% (86 mg).

### LC-MS: m/z 457.48 (MH+)

### Scheme 20, Cpd (III)*a Preparation: N-[4-[4-[methyl(methylsulfonyl)amino]piperidine-1-carbonyl]-3-pyrrolidin-1-ylphenyl]cyclopropanecarboxamide

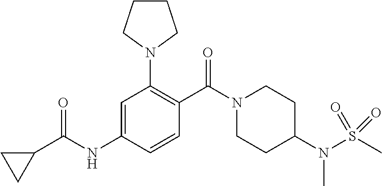 [SMILES: CN(C1CCN(C(=O)c2ccc(NC(=O)C3CC3)cc2N2CCCC2)CC1)S(C)(=O)=O]

### Example 104

To a 0° C. cooled solution of N-[4-[4-(methylamino)piperidine-1-carbonyl]-3-pyrrolidin-1-ylphenyl]cyclopropanecarboxamide (86.0 mg, 0.230 mmol) and triethylamine (0.05 mL, 0.350 mmol) in DCM (3.479 mL), methanesulfonyl chloride (0.02 mL, 0.280 mmol) was added. The mixture was warmed at room temperature and stirred for 5 minutes. The mixture was diluted with DCM and washed with brine. The organic layer was filtered through a phase separator and concentrated under reduced pressure. The residue was purified by FC on RP using acid conditions (eluting from 5:95 of CH₃CN/H₂O+0.1% of HCOOH to 35:65 of CH₃CN/H₂O+0.1% of HCOOH) to give the product of formula N-[4-[4-[methyl(methylsulfonyl)amino]piperidine-1-carbonyl]-3-pyrrolidin-1-ylphenyl]cyclopropanecarboxamide.

### Yield: 33 mg

1H NMR (400 MHZ, DMSO-d6) δ 10.09 (d, 1H), 7.17-6.79 (m, 3H), 4.70-4.50 (m, 1H), 3.85-3.77 (m, 1H), 3.62-3.49 (m, 1H), 3.23-2.82 (m, 8H), 2.68 (d, 4H), 1.94-1.48 (m, 9H), 0.78 (dd, 4H); LC-MS: m/z 449.26 (MH+).

### Preparation: N-[4-[4-(methanesulfonamido) piperidine-1-carbonyl]-3-pyrrolidin-1-ylphenyl]cyclopropanecarboxamide

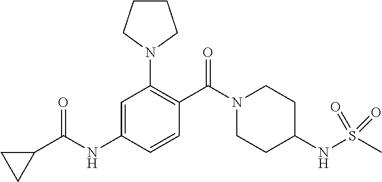 [SMILES: CS(=O)(=O)NC1CCN(C(=O)c2ccc(NC(=O)C3CC3)cc2N2CCCC2)CC1]

### Example 105

The synthesis of the title compound was effected analogously to the synthesis of the compound N-[4-[4-(methylamino)piperidine-1-carbonyl]-3-pyrrolidin-1-ylphenyl]cyclopropanecarboxamide wherein N-[4-(4-aminopiperidine-1-carbonyl)-3-pyrrolidin-1-ylphenyl]cyclopropanecarboxamide was used instead of N-[4-[4-(methylamino)piperidine-1-carbonyl]-3-pyrrolidin-1-ylphenyl]cyclopropanecarboxamide. The title compound was obtained in a yield 33% (35 mg).

1H NMR (400 MHZ, DMSO-d6) δ 10.09 (s, 1H), 7.16 (dd, 1H), 7.09-7.02 (m, 1H), 6.96-6.83 (m, 2H), 4.31 (dd, 1H), 3.53-3.35 (m, 2H), 3.21-2.78 (m, 9H), 1.87 (dd, 5H), 1.76 (h, 2H), 1.33 (dd, 2H), 0.77 (dt, 4H); LC-MS: m/z 435.23 (MH+).

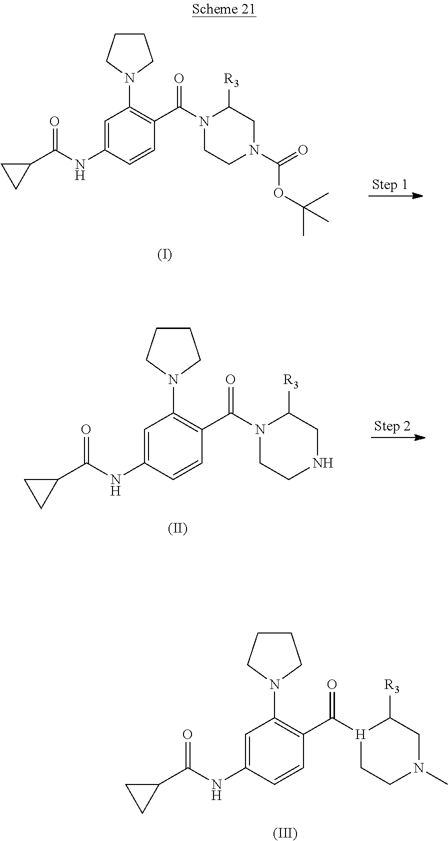

### Step 1

Compound of formula II may be obtained from compound of formula I (synthesized according to the Scheme 1) by removing the Boc protecting group under acidic conditions, e.g. TFA, typically at RT. The reaction takes about 2 hours to complete.

### Step 2

Compound of formula III may be obtained via reductive amination between compound of formula II and a proper aldehyde, e.g. formaldehyde, in the presence of a suitable reducing agent, e.g. sodium triacetoxyborohydride. The reaction is carried out in a suitable solvent, e.g. MeOH, typically at RT. The reaction takes about 12 hours to complete.

### Scheme 21, Cpd (II)*a Preparation: N-[3-pyrrolidin-1-yl-4-[2-[2-(trifluoromethyl)phenyl]piperazine-1-carbonyl]phenyl]cyclopropanecarboxamide

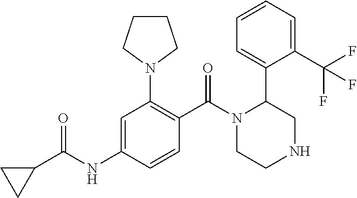 [SMILES: O=C(Nc1ccc(C(=O)N2CCNCC2c2ccccc2C(F)(F)F)c(N2CCCC2)c1)C1CC1]

To a solution of tert-butyl 4-[4-(cyclopropanecarbonylamino)-2-pyrrolidin-1-ylbenzoyl]-3-[2-(trifluoromethyl)phenyl]piperazine-1-carboxylate (24.3 mg, 0.041 (2 mmol) in DCM mL), trifluoroacetic acid (0.095 mL, 1.243 mmol) was added. The reaction mixture was stirred at RT for 2 hour. After this time the mixture was concentrated to dryness under vacuum. The residue was purified by SCX first washing with MeOH and then eluting with NH₃ 1N in MeOH. Basic fractions were collected and concentrated under vacuum affording the product of formula N-[3-pyrrolidin-1-yl-4-[2-[2-(trifluoromethyl)phenyl]piperazine-1-carbonyl]phenyl]cyclopropanecarboxamide as a racemic mixture. The product was used in the next step without further purification.

### Yield: 19 mg

### LC-MS: m/z 487.18 (MH+).

### Scheme 21, Cpd (II)*b Preparation: N-[4-[2-(3-cyanophenyl) piperazine-1-carbonyl]-3-pyrrolidin-1-ylphenyl]cyclopropanecarboxamide

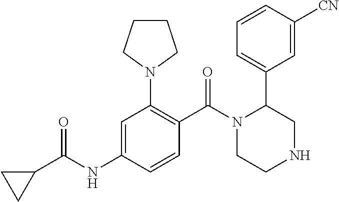 [SMILES: N#Cc1cccc(C2CNCCN2C(=O)c2ccc(NC(=O)C3CC3)cc2N2CCCC2)c1]

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein tert-butyl 3-(3-cyanophenyl)-4-[4-(cyclopropanecarbonylamino)-2-pyrrolidin-1-ylbenzoyl]piperazine-1-carboxylate was used instead of tert-butyl 4-[4-(cyclopropanecarbonylamino)-2-pyrrolidin-1-ylbenzoyl]-3-[2-(trifluoromethyl)phenyl]piperazine-1-carboxylate. The title compound was obtained as a racemic mixture in a yield 97% (44 mg).

### Scheme 21, Cpd (II)*c Preparation: N-[4-[2-(4,5-dimethyl-1,3-thiazol-2-yl) piperazine-1-carbonyl]-3-pyrrolidin-1-ylphenyl]cyclopropanecarboxamide

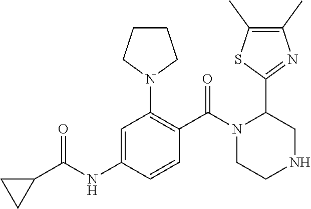 [SMILES: Cc1nc(C2CNCCN2C(=O)c2ccc(NC(=O)C3CC3)cc2N2CCCC2)sc1C]

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein tert-butyl 4-[4-(cyclopropanecarbonylamino)-2-pyrrolidin-1-ylbenzoyl]-3-(4,5-dimethyl-1,3-thiazol-2-yl) piperazine-1-carboxylate was used instead of tert-butyl 4-[4-(cyclopropanecarbonylamino)-2-pyrrolidin-1-ylbenzoyl]-3-[2-(trifluoromethyl)phenyl]piperazine-1-carboxylate. The title compound was obtained as a racemic mixture in a quantitative yield (59 mg).

### LC-MS: m/z 454.24 (MH+).

### Scheme 21, Cpd (II)*d Preparation: N-[4-[2-(4-methylphenyl) piperazine-1-carbonyl]-3-pyrrolidin-1-ylphenyl]cyclopropanecarboxamide

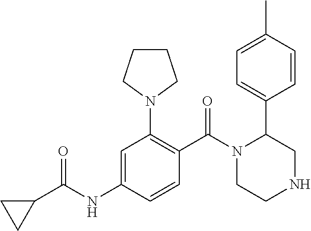 [SMILES: Cc1ccc(C2CNCCN2C(=O)c2ccc(NC(=O)C3CC3)cc2N2CCCC2)cc1]

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein tert-butyl 4-[4-(cyclopropanecarbonylamino)-2-pyrrolidin-1-ylbenzoyl]-3-(4-methylphenyl) piperazine-1-carboxylate was used instead of tert-butyl 4-[4-(cyclopropanecarbonylamino)-2-pyrrolidin-1-ylbenzoyl]-3-[2-(trifluoromethyl)phenyl]piperazine-1-carboxylate. The title compound was obtained as a racemic mixture in a yield of 46% (36 mg).

### LC-MS: m/z 433.38 (MH+).

### Scheme 21, Cpd (II)*e Preparation: N-[4-[2-(3-chloro-5-methylphenyl) piperazine-1-carbonyl]-3-pyrrolidin-1-ylphenyl]cyclopropanecarboxamide

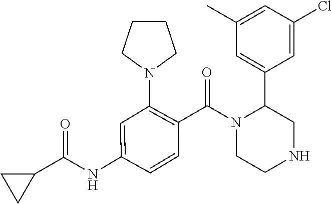

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein tert-butyl 3-(3-chloro-5-methylphenyl)-4-[4-(cyclopropanecarbonylamino)-2-pyrrolidin-1-ylbenzoyl]piperazine-1-carboxylate was used instead of tert-butyl 4-[4-(cyclopropanecarbonylamino)-2-pyrrolidin-1-ylbenzoyl]-3-[2-(trifluoromethyl)phenyl]piperazine-1-carboxylate. The title compound was obtained as a racemic mixture in a yield of 63% (68 mg).

### LC-MS: m/z 467.25 (MH+).

### Scheme 21, Cpd (II)*f Preparation: N-[4-[2-(3-methylphenyl) piperazine-1-carbonyl]-3-pyrrolidin-1-ylphenyl]cyclopropanecarboxamide

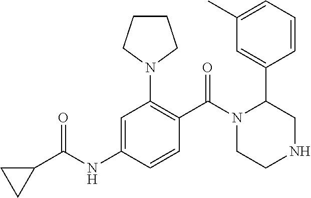 [SMILES: Cc1cccc(C2CNCCN2C(=O)c2ccc(NC(=O)C3CC3)cc2N2CCCC2)c1]

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein tert-butyl 4-[4-(cyclopropanecarbonylamino)-2-pyrrolidin-1-ylbenzoyl]-3-(3-methylphenyl) piperazine-1-carboxylate was used instead of tert-butyl 4-[4-(cyclopropanecarbonylamino)-2-pyrrolidin-1-ylbenzoyl]-3-[2-(trifluoromethyl)phenyl]piperazine-1-carboxylate. The title compound was obtained as a racemic mixture in a yield of 90% (118 mg).

### LC-MS: m/z 433.31 (MH+).

### Scheme 21, Cpd (III)*a Preparation: N-[4-[4-methyl-2-[2-(trifluoromethyl)phenyl]piperazine-1-carbonyl]-3-pyrrolidin-1-ylphenyl]cyclopropanecarboxamide

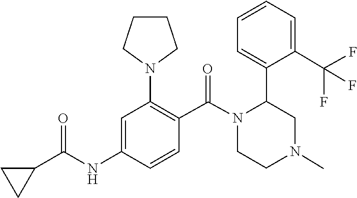 [SMILES: CN1CCN(C(=O)c2ccc(NC(=O)C3CC3)cc2N2CCCC2)C(c2ccccc2C(F)(F)F)C1]

### Example 106

To a solution of N-[3-pyrrolidin-1-yl-4-[2-[2-(trifluoromethyl)phenyl]piperazine-1-carbonyl]phenyl]cyclopropanecarboxamide (19.0 mg, 0.040 mmol) in Methanol (1.000 mL) formaldehyde (0.03 mL, 0.390 mmol) was added followed by sodium triacetoxyborohydride (99.66 mg, 0.234 mmol). The reaction was stirred at room temperature ON. The day after the reaction was diluted with EtOAc and washed with a saturated solution of NaHCO₃. The organic phase was dried using a phase separator and concentrated. The crude was purified by FC on silica gel (eluting from cHex/EtOAc 50:50 to cHex/EtOAc 20:80), obtaining the product of formula N-[4-[4-methyl-2-[2-(trifluoromethyl)phenyl]piperazine-1-carbonyl]-3-pyrrolidin-1-ylphenyl]cyclopropanecarboxamide as a racemic mixture.

### Yield: 6.5 mg

¹H NMR (400 MHZ, DMSO-d₆) δ 10.15-10.02 (m, 1H), 7.99-7.77 (m, 1H), 7.77-7.58 (m, 2H), 7.56-7.44 (m, 1H), 7.13-7.06 (m, 1H), 7.00-6.97 (m, 1H), 6.91-6.61 (m, 1H), 5.84-5.32 (m, 1H), 3.88-3.60 (m, 2H), 3.26-3.05 (m, 3H), 2.86-2.72 (m, 3H), 2.47-2.38 (m, 1H), 2.24-2.05 (m, 4H), 2.05-1.84 (m, 3H), 1.82-1.57 (m, 2H), 0.81-0.67 (m, 4H); LC-MS: m/z 501.31 (MH+).

### Preparation: N-[4-[2-(3-cyanophenyl)-4-methylpiperazine-1-carbonyl]-3-pyrrolidin-1-ylphenyl]cyclopropanecarboxamide

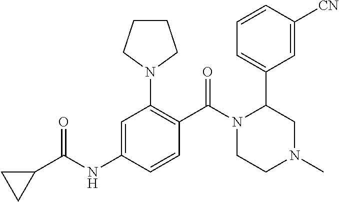 [SMILES: CN1CCN(C(=O)c2ccc(NC(=O)C3CC3)cc2N2CCCC2)C(c2cccc(C#N)c2)C1]

### Example 107

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein N-[4-[2-(3-cyanophenyl) piperazine-1-carbonyl]-3-pyrrolidin-1-ylphenyl]cyclopropanecarboxamide was used instead of N-[3-pyrrolidin-1-yl-4-[2-[2-(trifluoromethyl)phenyl]piperazine-1-carbonyl]phenyl]cyclopropanecarboxamide. The title compound was obtained as a racemic mixture in a yield 35% (16 mg).

¹H NMR (400 MHZ, DMSO-d₆) δ 10.11 (s, 1H), 8.16-7.86 (m, 1H), 7.82-7.37 (m, 3H), 7.21-6.67 (m, 3H), 5.92-5.54 (m, 1H), 3.44 (dd, 2H), 3.24-2.83 (m, 4H), 2.78-2.59 (m, 2H), 2.40-2.10 (m, 4H), 2.09-1.86 (m, 3H), 1.83-1.57 (m, 3H), 0.82-0.62 (m, 4H); LC-MS: m/z 458.29 (MH+).

### Preparation: N-[4-[2-(4,5-dimethyl-1,3-thiazol-2-yl)-4-methylpiperazine-1-carbonyl]-3-pyrrolidin-1-ylphenyl]cyclopropanecarboxamide

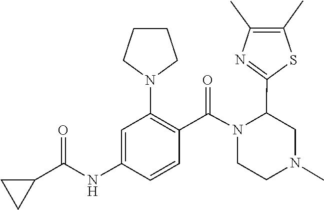 [SMILES: Cc1nc(C2CN(C)CCN2C(=O)c2ccc(NC(=O)C3CC3)cc2N2CCCC2)sc1C]

### Example 108

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein N-[4-[2-(4,5-dimethyl-1,3-thiazol-2-yl) piperazine-1-carbonyl]-3-pyrrolidin-1-ylphenyl]cyclopropanecarboxamide was used instead of N-[3-pyrrolidin-1-yl-4-[2-[2-(trifluoromethyl)phenyl]piperazine-1-carbonyl]phenyl]cyclopropanecarboxamide. The title compound was obtained as a racemic mixture in a yield 89% (54 mg).

¹H NMR (400 MHZ, DMSO-d₆) δ 10.15-10.06 (m, 1H), 7.21-6.77 (m, 3H), 5.90-4.26 (m, 1H), 3.61-3.35 (m, 2H), 3.30-2.56 (m, 6H), 2.39-2.12 (m, 10H), 2.08-1.61 (m, 6H), 0.89-0.70 (d, 4H); LC-MS m/z 468.49 (MH+).

### Preparation: N-[4-[4-methyl-2-(4-methylphenyl) piperazine-1-carbonyl]-3-pyrrolidin-1-ylphenyl]cyclopropanecarboxamide

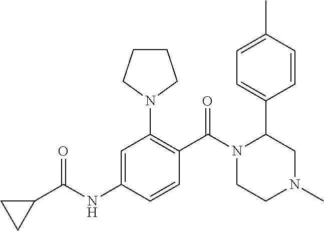 [SMILES: Cc1ccc(C2CN(C)CCN2C(=O)c2ccc(NC(=O)C3CC3)cc2N2CCCC2)cc1]

### Example 109

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein N-[4-[2-(4-methylphenyl) piperazine-1-carbonyl]-3-pyrrolidin-1-ylphenyl]cyclopropanecarboxamide was used instead of N-[3-pyrrolidin-1-yl-4-[2-[2-(trifluoromethyl)phenyl]piperazine-1-carbonyl]phenyl]cyclopropanecarboxamide. The title compound was obtained as a racemic mixture in a yield 32% (11 mg).

¹H NMR (400 MHZ, DMSO-d₆) δ 10.21-9.95 (m, 1H), 7.66-7.23 (m, 2H), 7.22-6.69 (m, 5H), 5.84-4.25 (m, 1H), 3.46-3.38 (m, 1H), 3.27-2.55 (m, 6H), 2.46-2.08 (m, 7H), 2.06-1.85 (m, 4H), 1.81-1.53 (m, 3H), 0.93-0.65 (m, 4H); LC-MS: m/z 447.30 (MH+).

### Preparation: N-[4-[2-(3-chloro-5-methylphenyl)-4-methylpiperazine-1-carbonyl]-3-pyrrolidin-1-ylphenyl]cyclopropanecarboxamide

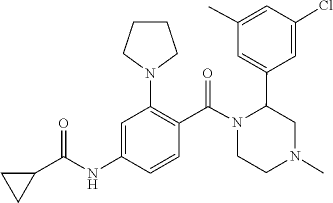 [SMILES: Cc1cc(Cl)cc(C2CN(C)CCN2C(=O)c2ccc(NC(=O)C3CC3)cc2N2CCCC2)c1]

### Example 110

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein N-[4-[2-(3-chloro-5-methylphenyl) piperazine-1-carbonyl]-3-pyrrolidin-1-ylphenyl]cyclopropanecarboxamide was used instead of N-[3-pyrrolidin-1-yl-4-[2-[2-(trifluoromethyl)phenyl]piperazine-1-carbonyl]phenyl]cyclopropanecarboxamide. The title compound was obtained as a racemic mixture in a yield 60% (41 mg).

1H NMR (400 MHZ, DMSO-d6) δ ppm 10.10 (s, 1H), 7.59-6.76 (m, 6H), 5.79-5.64 (m, 1H), 4.72-4.35 (m, 1H), 3.48-2.60 (m, 7H), 2.40-2.13 (m, 7H), 2.08-1.63 (m, 6H), 0.85-0.69 (m, 4H); LC-MS: m/z 481.28 (MH+)

The racemic mixture (Example 110) was then separated into single enantiomers by chiral semi-preparative HPLC

| | | |
|---|---|---|
| Preparative chiral | Column | Chiralpak IC (25 x 3.0 cm), |
| chromatography | | 5 μ |
| protocol: | Mobile phase | n-Hexane/(Ethanol + |
| | 0.1% IPA) | 80/20% v/v |
| | Flow rate (ml/min) | 37 ml/min |
| | DAD detection | 220 nm |
| | Loop | 1000 μL |
| | Total amount | 39 mg |
| | Injection | 11.1 mg (each injection) |

| | |
|---|---|
| 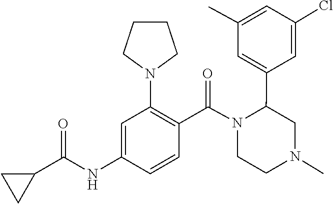 [SMILES: Cc1cc(Cl)cc(C2CN(C)CCN2C(=O)c2ccc(NC(=O)C3CC3)cc2N2CCCC2)c1] | Enantiomer 1 Yield: 16 Rt 10.84 min 100% ee LC-MS: m/z 481.28 (MH+) N-[4-[2S or 2R-(3-chloro-5-methylphenyl)-4- methylpiperazine-1-carbonyl]-3-pyrrolidin-1- ylphenyl]cyclopropanecarboxamide Example 110a |
| 2R or 2S enantiomer | |
| 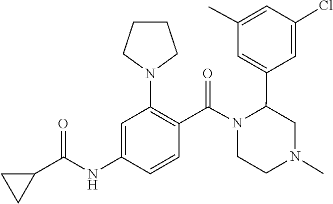 [SMILES: Cc1cc(Cl)cc(C2CN(C)CCN2C(=O)c2ccc(NC(=O)C3CC3)cc2N2CCCC2)c1] | Enantiomer 2 Yield: Rt 14.49 min 100% ee LC-MS: m/z 481.28 (MH+) N-[4-[2S or 2R-(3-chloro-5-methylphenyl)-4- methylpiperazine-1-carbonyl]-3-pyrrolidin-1- ylphenyl]cyclopropanecarboxamide Example 110b |
| 2R or 2S enantiomer | |

### Preparation of N-[4-[2S or 2R-(3-chloro-5-methylphenyl)-4-methylpiperazine-1-carbonyl]-3-pyrrolidin-1-ylphenyl]cyclopropanecarboxamide hydrochloride

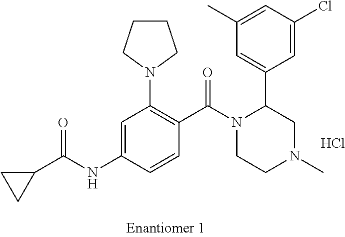 [SMILES: Cc1cc(Cl)cc(C2CN(C)CCN2C(=O)c2ccc(NC(=O)C3CC3)cc2N2CCCC2)c1 ; Cl]

### Example 110a

N-[4-[2S or 2R-(3-chloro-5-methylphenyl)-4-methylpiperazine-1-carbonyl]-3-pyrrolidin-1-ylphenyl]cyclopropanecarboxamide (Enantiomer 1) (15.7 mg, 0.033 mmol) was dissolved in Methanol dry (1 mL) and treated with 1 eq of HCl in dioxane (0.033 mL, 0.033 mmol) to afford, after evaporation, the product of formula N-[4-[2R or 2S-(3-chloro-5-methylphenyl)-4-methylpiperazine-1-carbonyl]-3-pyrrolidin-1-ylphenyl]cyclopropanecarboxamide hydrochloride.

### Yield: 16.1 mg

### LC-MS: m/z 481.25 (MH+).

### Preparation of N-[4-[2S or 2R-(3-chloro-5-methylphenyl)-4-methylpiperazine-1-carbonyl]-3-pyrrolidin-1-ylphenyl]cyclopropanecarboxamide hydrochloride

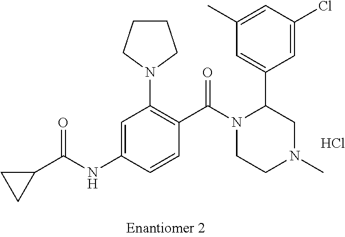 [SMILES: Cc1cc(Cl)cc(C2CN(C)CCN2C(=O)c2ccc(NC(=O)C3CC3)cc2N2CCCC2)c1 ; Cl]

### Example 110b

N-[4-[2S or 2R-(3-chloro-5-methylphenyl)-4-methylpiperazine-1-carbonyl]-3-pyrrolidin-1-ylphenyl]cyclopropanecarboxamide (Enantiomer 2) (14.9 mg, 0.031 mmol) was dissolved in Methanol dry (1 mL) and treated with 1 eq of HCl in dioxane (0.031 mL, 0.031 mmol) to afford, after evaporation, the product of formula N-[4-[2R or 2S-(3-chloro-5-methylphenyl)-4-methylpiperazine-1-carbonyl]-3-pyrrolidin-1-ylphenyl]cyclopropanecarboxamide hydrochloride.

### Yield: 13.7

### LC-MS: m/z 481.25 (MH+).

### Preparation: N-[4-[4-methyl-2-(3-methylphenyl) piperazine-1-carbonyl]-3-pyrrolidin-1-ylphenyl]cyclopropanecarboxamide

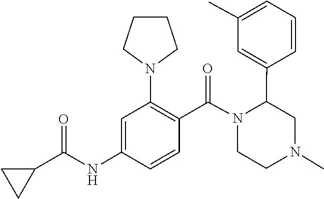 [SMILES: Cc1cccc(C2CN(C)CCN2C(=O)c2ccc(NC(=O)C3CC3)cc2N2CCCC2)c1]

### Example 111

The synthesis of the title compound was effected analogously to the synthesis of the compound N-[3-pyrrolidin-1-yl-4-[2-[2-(trifluoromethyl)phenyl]piperazine-1-carbonyl]phenyl]cyclopropanecarboxamide wherein N-[4-[2-(3-methylphenyl) piperazine-1-carbonyl]-3-pyrrolidin-1-ylphenyl]cyclopropanecarboxamide was used instead of N-[3-pyrrolidin-1-yl-4-[2-[2-(trifluoromethyl)phenyl]piperazine-1-carbonyl]phenyl]cyclopropanecarboxamide. The title compound was obtained as a racemic mixture in a yield 65% (79 mg).

¹H NMR (400 MHZ, DMSO-d₆) δ 10.17-10.03 (m, 1H), 7.54-7.42 (m, 1H), 7.30-7.12 (m, 2H), 7.11-6.92 (m, 3H), 6.87-6.81 (m, 1H), 5.84-4.30 (m, 1H), 3.50-3.34 (m, 1H), 3.26-2.59 (m, 6H), 2.40-2.11 (m, 7H), 2.05-1.84 (m, 4H), 1.80-1.58 (m, 3H), 0.95-0.66 (m, 4H); LC-MS: m/z 447.29 (MH+)

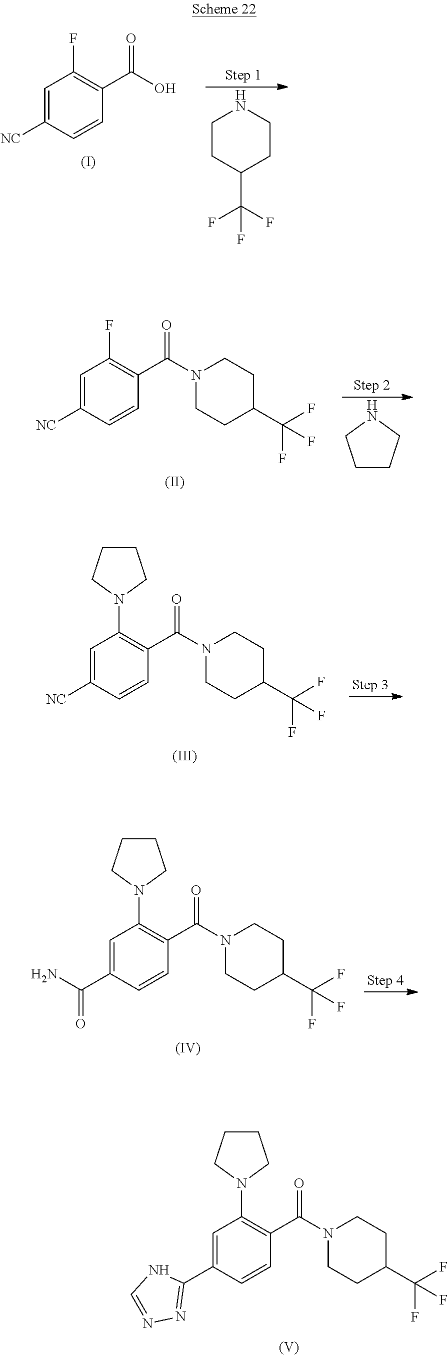

### Step 1

Compound of formula II may be obtained from compound of formula I (commercially available from Enamine) by coupling with 4-(trifluoromethyl) piperidine hydrochloride in presence of a suitable coupling agent, e.g. HATU, and a suitable organic base, e.g. DIPEA. The reaction is carried out in a suitable solvent such as DMF, typically at room temperature. The reaction takes about 5 hours to complete.

### Step 2

Compound of formula III may be obtained from compound of formula II by SNAr substitution with pyrrolidine in a suitable aprotic solvent, e.g. CH₃CN, typically at 80° C. The reaction takes about 12 hours to complete.

### Step 3

Compound of formula IV may be obtained by nitrile hydrolysis of compound of formula III under standard literature conditions such as by reaction with NaOH in the presence of H₂O2. The reaction is carried out in a suitable solvent, e.g. MeOH, typically at RT. The reaction takes about 12 hours to complete

### Step 4

Compound of formula V may be obtained from compound of formula IV via N-formyl imide formation, by reaction with a suitable formulating agent such as 1,1-dimethoxy-N,N-dimethylmethanamine, followed by cyclization with NH₂NH₂. The reaction is carried out in a suitable solvents, e.g. DMF, typically at a temperature between RT and 120° C.

### Scheme 22, Cpd (II) Preparation: 3-fluoro-4-[4-(trifluoromethyl) piperidine-1-carbonyl]benzonitrile

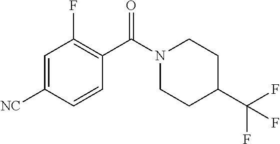 [SMILES: N#Cc1ccc(C(=O)N2CCC(C(F)(F)F)CC2)c(F)c1]

To a solution of HATU (3454.12 mg, 9.08 mmol), 4-cyano-2-fluorobenzoic acid (1.5 g, 9.08 mmol) and 4-(trifluoromethyl) piperidine hydrochloride (1722.47 mg, 9.08 mmol) in DMF (30 mL) DIPEA (4.85 mL, 27.25 mmol) was added. The reaction was stirred at RT for 5 h. After this time the reaction was diluted with H₂O and extracted with AcOEt 3 times. The combined organic fractions were washed with brine, dried over Na₂SO₄, filtered and concentrated under vacuum. The residue was purified by FC on silica gel (eluting from 100% of cHex to AcOEt/cHex 1:1) affording the product of formula 3-fluoro-4-[4-(trifluoromethyl) piperidine-1-carbonyl]benzonitrile.

### Yield: 2.5 g

¹H NMR (400 MHZ, DMSO-d₆) δ 8.00 (dd, 1H), 7.81 (dd, 1H), 7.75-7.62 (m, 1H), 4.60 (d, 1H), 3.41 (d, 1H), 3.14 (t, 1H), 2.85 (td, 1H), 2.75-2.60 (m, 2H), 2.00-1.90 (m, 1H), 1.85-1.76 (m, 1H), 1.53-1.28 (m, 1H); LC-MS: m/z 301.19 (MH+).

### Scheme 22, Cpd (III) Preparation: 3-pyrrolidin-1-yl-4-[4-(trifluoromethyl) piperidine-1-carbonyl]benzonitrile

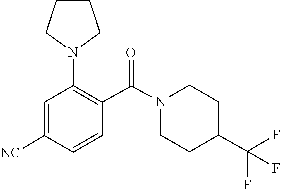 [SMILES: N#Cc1ccc(C(=O)N2CCC(C(F)(F)F)CC2)c(N2CCCC2)c1]

To a solution of 3-fluoro-4-[4-(trifluoromethyl) piperidine-1-carbonyl]benzonitrile (1.5 g, 5 mmol) in MeCN (20 mL) pyrrolidine (1.67 mL, 19.98 mmol) was added and the reaction was stirred at 80° C. ON. After this time the reaction was cooled down to RT and H₂O was added. The mixture was then extracted with AcOEt 3 times. The combined organic fractions were washed with brine, dried over Na₂SO₄ filtered and concentrated under vacuum. The residue was purified by FC on silica gel (eluting from 100% of cHex to cHex/AcOEt 3:7) affording the product of formula 3-pyrrolidin-1-yl-4-[4-(trifluoromethyl) piperidine-1-carbonyl]benzonitrile.

### Yield: 1.6 g

1H NMR (400 MHZ, DMSO-d6) δ 7.19 (dd, 1H), 7.10-7.00 (m, 2H), 4.60 (t, 1H), 3.51 (dd, 1H), 3.21-2.56 (m, 7H), 1.97-1.68 (m, 6H), 1.55-1.24 (m, 2H); LC-MS: m/z 352.20 (MH+).

### Scheme 22, Cpd (IV) Preparation: 3-pyrrolidin-1-yl-4-[4-(trifluoromethyl) piperidine-1-carbonyl]benzamide

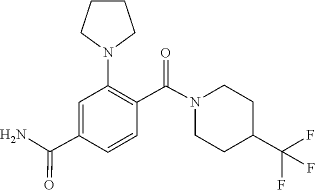 [SMILES: NC(=O)c1ccc(C(=O)N2CCC(C(F)(F)F)CC2)c(N2CCCC2)c1]

To a solution of 3-pyrrolidin-1-yl-4-[4-(trifluoromethyl) piperidine-1-carbonyl]benzonitrile (285.37 mg, 0.810 mmol) in Methanol (3 mL) sodium hydroxide (0.27 mL, 0.810 mmol) was added and the reaction was stirred ON at RT. The day after hydrogen peroxide (0.2 mL, 1.97 mmol) was added followed by further sodium hydroxide (0.27 mL, 0.810 mmol) and the reaction was stirred at RT for 4 h. After this time the reaction was diluted with H₂O and extracted with AcOEt 3 times. The organic phase was washed with brine, dried over Na₂SO₄, filtered and concentrated under vacuum affording the product of formula 3-pyrrolidin-1-yl-4-[4-(trifluoromethyl) piperidine-1-carbonyl]benzamide. The product was used in the next step without further purification.

### Yield: 120 mg

1H NMR (400 MHZ, DMSO-d6) δ 7.24-6.97 (m, 3H), 4.62 (t, 1H), 3.53 (t, 1H), 3.26-2.52 (m, 7H), 1.96-1.67 (m, 6H), 1.49-1.27 (m, 2H); LC-MS: m/z 370.18 (MH+).

### Scheme 22, Cpd (V) Preparation: [2-pyrrolidin-1-yl-4-(4H-1,2,4-triazol-3-yl)phenyl]-[4-(trifluoromethyl)piperidin-1-yl]methanone

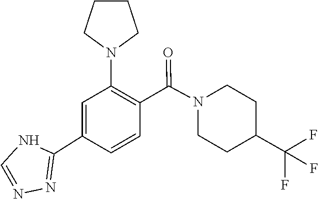 [SMILES: O=C(c1ccc(-c2nnc[nH]2)cc1N1CCCC1)N1CCC(C(F)(F)F)CC1]

### Example 112

To a solution of 3-pyrrolidin-1-yl-4-[4-(trifluoromethyl) piperidine-1-carbonyl]benzamide (120.0 mg, 0.320 mmol) in DMF (1.793 mL), 1,1-dimethoxy-N,N-dimethylmethanamine (0.09 mL, 0.650 mmol) was added and the reaction was stirred for 1.5 h at 120° C. After this time the reaction was cooled down to RT and H₂O was added. The reaction was with EtOAc (×3), washed with brine, dried on Na₂SO₄ and filtered and concentrated under vacuum. The residue was dissolved with Ethanol (5 mL) and hydrazine (0.5 mL, 5.52 mmol) was added. The reaction was stirred at RT for 2 h. After this time the reaction was diluted with H₂O and extracted with AcOEt 3 times. The combined organic fractions were washed with brine, filtered and concentrated under vacuum. The residue was purified by FC on NH column (eluting from 100% of DCM to DCM/MeOH 9:1) to give the product of formula [2-pyrrolidin-1-yl-4-(4H-1,2,4-triazol-3-yl)phenyl]-[4-(trifluoromethyl)piperidin-1-yl]methanone.

### Yield: 40 mg

1H NMR (400 MHZ, DMSO-d6) δ 14.17 (s, 1H), 8.36 (s, 1H), 7.46-7.29 (m, 2H), 7.12 (dd, 1H), 4.64 (t, 1H), 3.61 (t, 1H), 3.28 (s, 2H), 3.17-2.94 (m, 2H), 2.90-2.59 (m, 3H), 2.05-1.72 (m, 6H), 1.53-1.27 (m, 2H); LC-MS: m/z 394.22 (MH+).

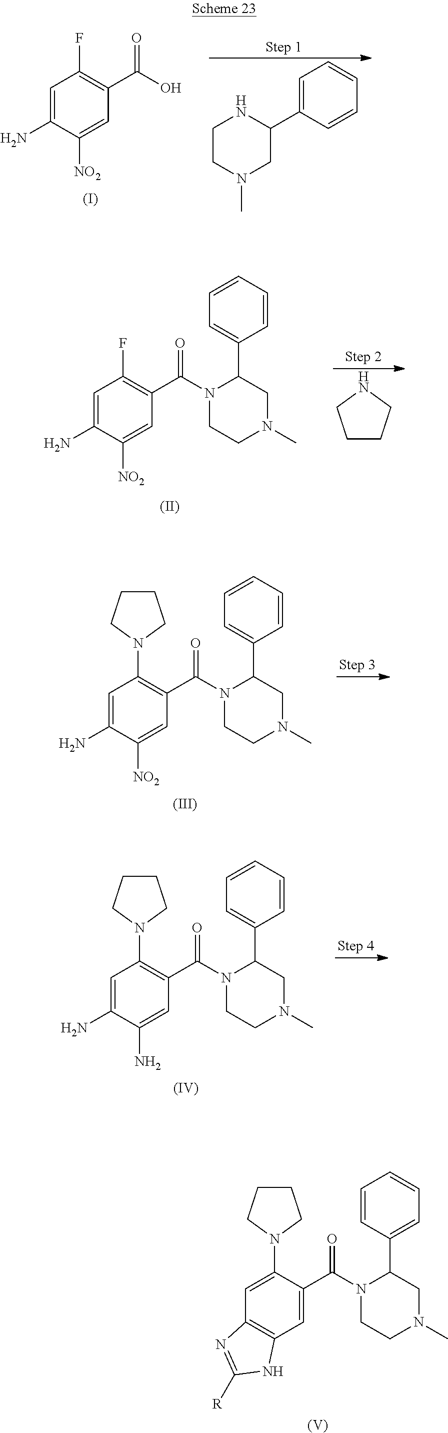

### Step 1

Compound of formula II may be obtained via acyl chloride formation of compound of formula I (commercially available from Enamine) by treatment with a suitable chlorinating agent, such as SOCl₂, typically at 90° C., followed by coupling with 1-Methyl-3-phenylpiperazine (commercially available from Enamine) in the presence of a suitable organic base, e.g. TEA. The reaction is carried out in a suitable solvent such as DCM, typically at room temperature. The reaction takes about 16 hours to complete.

### Step 2

Compound of formula III may be obtained from compound of formula II by SNAr substitution with pyrrolidine in a suitable aprotic solvent, e.g. DMSO, typically at 120° C. The reaction takes about 3 hours to complete.

### Step 3

Compound of formula IV may be obtained by nitro reduction of compound of formula III using Zn dust in a suitable solvent, e.g. AcOH, typically at room temperature. The reaction takes about 2 hours to complete.

### Step 4

Compound of formula V may be obtained by cyclization of compound of formula IV with a proper aldehyde, e.g. cyclopropanecarboxaldehyde, in the presence of a suitable oxidating agent, e.g. oxone. The reaction is carried out in a suitable mixture of solvents, e.g. DMF/H₂O typically at RT. The reaction takes about 4 hours to complete.

### Scheme 23, Cpd (II) Preparation: (4-amino-2-fluoro-5-nitrophenyl)-(4-methyl-2-phenylpiperazin-1-yl)methanone

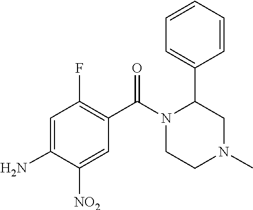 [SMILES: CN1CCN(C(=O)c2cc([N+](=O)[O-])c(N)cc2F)C(c2ccccc2)C1]

A mixture of 4-amino-2-fluoro-5-nitrobenzoic acid (200.0 mg, 1 mmol) and thionyl dichloride (1.46 mL, 19.99 mmol) was heated to reflux for 4 hours. After this time the reaction was cooled down to RT and then concentrated under vacuum. The mixture was stripped three times with cyclohexane in order to remove thionyl dichloride in excess. The residue was suspended in DCM (4 mL) and 1-methyl-3-phenylpiperazine (176.15 mg, 1 mmol) was added followed by TEA (0.42 mL, 3 mmol) and the reaction was stirred overnight at RT. After this time the mixture was diluted with DCM and washed with a s.s. of NaHCO₃3 times. The aqueous phases were collected together and counterextracted with DCM. The combined organic phases were dried over a phase separator and concentrated under vacuum, affording the product of formula (4-amino-2-fluoro-5-nitrophenyl)-(4-methyl-2-phenylpiperazin-1-yl)methanone. The product was used in the next step without further purification.

### Yield: 380 mg

### LC-MS: m/z 359.36 (MH+).

### Scheme 23, Cpd (III) Preparation: (4-amino-5-nitro-2-pyrrolidin-1-ylphenyl)-(4-methyl-2-phenylpiperazin-1-yl)methanone

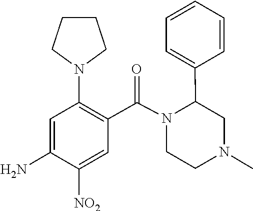 [SMILES: CN1CCN(C(=O)c2cc([N+](=O)[O-])c(N)cc2N2CCCC2)C(c2ccccc2)C1]

To a solution of (4-amino-2-fluoro-5-nitrophenyl)-(4-methyl-2-phenylpiperazin-1-yl)methanone (386.0 mg, 1.08 mmol) in DMSO (8 mL) pyrrolidine (0.27 mL, 3.23 mmol) was added. The mixture was stirred at 120° C. for 2 h. After this time the mixture was cooled down to RT and diluted with H₂O. The mixture was extracted with AcOEt 3 times. The combined organic fractions were washed with brine, dried over Na₂SO₄, filtered and concentrated under vacuum. The residue was purified by FC on silica gel (eluting from cHex/AcOEt 8:2 to cHex/AcOEt 3:7) affording the product of formula (4-amino-5-nitro-2-pyrrolidin-1-ylphenyl)-(4-methyl-2-phenylpiperazin-1-yl)methanone.

### Yield: 230 mg

¹H NMR (400 MHZ, DMSO-d₆) δ 7.77-7.70 (m, 1H), 7.59-7.44 (m, 1H), 7.44-7.15 (m, 5H), 6.11-4.31 (m, 1H), 3.63-3.31 (m, 3H), 3.20-2.98 (m, 2H), 2.88-2.76 (m, 1H), 2.74-2.66 (m, 1H), 2.45-2.11 (m, 5H), 2.10-1.85 (m, 3H), 1.82-1.59 (m, 2H); LC-MS: m/z 410.22 (MH+).

### Scheme 23, Cpd (IV) Preparation: (4,5-diamino-2-pyrrolidin-1-ylphenyl)-(4-methyl-2-phenylpiperazin-1-yl)methanone

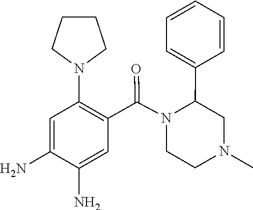 [SMILES: CN1CCN(C(=O)c2cc(N)c(N)cc2N2CCCC2)C(c2ccccc2)C1]

To a solution of (4-amino-5-nitro-2-pyrrolidin-1-ylphenyl)-(4-methyl-2-phenylpiperazin-1-yl)methanone (232.3 mg, 0.570 mmol) in AcOH (8 mL) zinc (370.9 mg, 5.67 mmol) was added and the reaction was stirred at RT for 2 hours. After this time the mixture was filtered and concentrated to residue by rotavapor. The crude was then purified by SCX first washing with MeOH and then eluting with NH₃ 1N in MeOH. Basic fractions were collected and concentrated under vacuum affording the product of formula (4,5-diamino-2-pyrrolidin-1-ylphenyl)-(4-methyl-2-phenylpiperazin-1-yl)methanone. The product was used in the next step without further purification.

### Yield: 190 mg

¹H NMR (400 MHZ, DMSO-d₆) δ 7.63 (d, 1H), 7.53-7.10 (m, 5H), 6.49-5.58 (m, 2H), 4.92-3.97 (m, 2H), 3.23-2.60 (m, 7H), 2.29-2.13 (m, 5H), 1.88 (d, 5H), 1.62 (d, 2H); LC-MS: m/z m/z 381.06 (M+2H).

### Scheme 23, Cpd (V)*a Preparation: (2-cyclopropyl-6-pyrrolidin-1-yl-3H-benzimidazol-5-yl)-(4-methyl-2-phenylpiperazin-1-yl)methanone

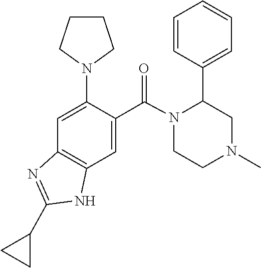 [SMILES: CN1CCN(C(=O)c2cc3[nH]c(C4CC4)nc3cc2N2CCCC2)C(c2ccccc2)C1]

### Example 113

To a mixture of (4,5-diamino-2-pyrrolidin-1-ylphenyl)-(4-methyl-2-phenylpiperazin-1-yl)methanone (90.0 mg, 0.240 mmol) and cyclopropanecarboxaldehyde (24.93 mg, 0.360 mmol) in DMF (1.5 mL)/Water (0.250 mL) Oxone (87.48 mg, 0.140 mmol) was added. The reaction was stirred at RT for 4 h. After this time a s.s. of NaHCO₃ was added and the reaction was extracted with AcOEt 3 times. The combined organic fractions were washed with brine, dried over Na₂SO₄, filtered and concentrated by rotavapor. The residue was purified by FC on RP using basic conditions (eluting from 5:95 of CH₃CN/H₂O+0.1% of NH₄OH to 40:60 of CH₃CN/H₂O+0.1% of NH₄OH) affording the product of formula (2-cyclopropyl-6-pyrrolidin-1-yl-3H-benzimidazol-5-yl)-(4-methyl-2-phenylpiperazin-1-yl)methanone.

### Yield: 45 mg

¹H NMR (400 MHZ, DMSO-d₆) δ 11.95 (s, 1H), 7.76-7.45 (m, 2H), 7.37 (dt, 2H), 7.28 (t, 1H), 7.21-7.14 (m, 1H), 7.12-6.69 (m, 1H), 5.92-3.44 (m, 2H), 3.17-2.53 (m, 6H), 2.46-2.12 (m, 5H), 2.05-1.78 (m, 5H), 1.73-1.60 (m, 1H), 1.10-0.83 (m, 4H); LC-MS: m/z 430.52 (MH+).

### Preparation: (4-methyl-2-phenylpiperazin-1-yl)-(2-methyl-6-pyrrolidin-1-yl-3H-benzimidazol-5-yl)methanone

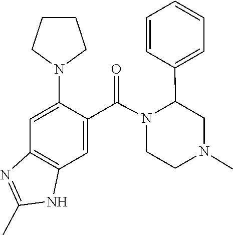

### Example 114

The synthesis of the title compound was effected analogously to the synthesis of the compound of Example 113 (2-cyclopropyl-6-pyrrolidin-1-yl-3H-benzimidazol-5-yl)-(4-methyl-2-phenylpiperazin-1-yl)methanone wherein acetaldehyde was used instead of cyclopropanecarboxaldehyde. The title compound was obtained in a yield of 49% (47 mg).

¹H NMR (400 MHZ, DMSO-d₆) δ 12.05-11.81 (m, 1H), 7.78-7.44 (m, 1H), 7.44-7.21 (m, 3H), 7.20-6.93 (m, 2H), 6.85-6.67 (m, 1H), 5.88-3.44 (m, 2H), 3.24-2.74 (m, 6H), 2.64-2.56 (m, 1H), 2.45-2.11 (m, 5H), 2.07-1.87 (m, 5H), 1.80-1.59 (m, 2H); LC-MS: m/z 403.68 (MH+).

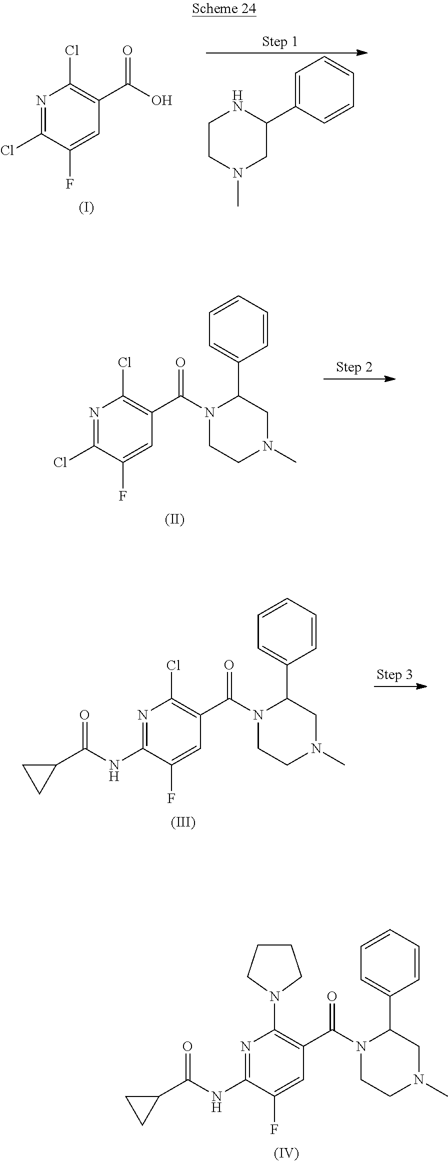

### Step 1

Compound of formula II may be obtained via acyl chloride formation from compound of formula I (commercially available from Enamine) by treatment with a suitable chlorinating agent, such as SOCl₂, typically at 90° C., followed by coupling with 1-Methyl-3-phenylpiperazine (commercially available from Enamine) in the presence of a suitable organic base, e.g. TEA. The reaction is carried out in a suitable solvent such as DCM, typically at room temperature. The reaction takes about 16 hours to complete.

### Step 2

Compound of formula III may be obtained from compound of formula II by Buchwald reaction with the proper amide, e.g. cyclopropanecarboxamide, in the presence of a suitable palladium precatalyst, e.g. XPhos Pd G3, and a suitable base, such as Cs₂CO₃. The reaction is carried out in a suitable solvent, e.g. 1, 4-dioxane, typically at 80° C. The reaction takes about 12 hours to complete.

### Step 3

Compound of formula IV may be obtained from compound of formula III by Buchwald reaction with the proper amine, e.g. pyrrolidine, in the presence of a suitable palladium precatalyst, e.g. XPhos Pd G3, and a suitable base, such as K₂CO₃. The reaction is carried out in a suitable solvent, e.g. 1, 4-dioxane, typically at 100° C. The reaction takes about 12 hours to complete.

### Scheme 24, Cpd (II) Preparation: (2,6-dichloro-5-fluoropyridin-3-yl)-(4-methyl-2-phenylpiperazin-1-yl)methanone

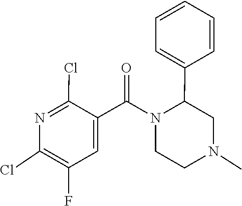 [SMILES: CN1CCN(C(=O)c2cc(F)c(Cl)nc2Cl)C(c2ccccc2)C1]

A mixture of 2,6-dichloro-5-fluoropyridine-3-carboxylic acid (1.0 g, 4.76 mmol) and thionyl dichloride (3.47 mL, 47.62 mmol) was heated to reflux for 3 h. After this time the reaction was cooled down to RT and concentrated under vacuum. The residue was dissolved in DCM (1.667 mL) and cooled to 0° C. After 10 min a solution of DIPEA (1.66 mL, 9.52 mmol) and 1-methyl-3-phenylpiperazine (0.84 g, 4.76 mmol) in DCM (10 mL) was added and the reaction was stirred at RT overnight. After this time the reaction was diluted with DCM and washed with a s.s. of NaHCO₃3 times. The organic phase was washed with brine, dried over a phase separator and concentrated under vacuum affording the product of formula (2,6-dichloro-5-fluoropyridin-3-yl)-(4-methyl-2-phenylpiperazin-1-yl)methanone. The product was used in the next step without further purification.

### Yield: 1.6 g

¹H NMR (400 MHZ, DMSO-d₆) δ 8.42-8.10 (m, 1H), 7.53 (dd, 1H), 7.44-7.19 (m, 4H), 5.83-3.45 (m, 1H), 3.16-2.56 (m, 2H), 2.47-2.29 (m, 1H), 2.25-2.12 (m, 3H), 2.12-1.89 (m, 1H), 0.95 (d, 2H); LC-MS: m/z 368.04 (MH+)

### Scheme 24, Cpd (III) Preparation: N-[6-chloro-3-fluoro-5-(4-methyl-2-phenylpiperazine-1-carbonyl)pyridin-2-yl]cyclopropanecarboxamide

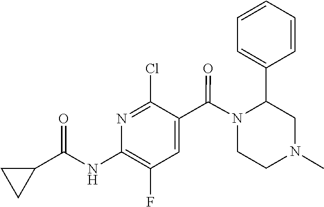 [SMILES: CN1CCN(C(=O)c2cc(F)c(NC(=O)C3CC3)nc2Cl)C(c2ccccc2)C1]

A solution of (2,6-dichloro-5-fluoropyridin-3-yl)-(4-methyl-2-phenylpiperazin-1-yl)methanone (200.0 mg, 0.540 mmol), cyclopropanecarboxamide (50.84 mg, 0.600 mmol), dicesium carbonate (356.13 mg, 1.09 mmol) and XPHOS PD G3 (23.01 mg, 0.030 mmol) in dry 1,4-Dioxane (4.964 mL) was degassed by Schlenk technique. The resulting mixture was heated to 80° C. and stirred ON. The day after the reaction mixture was diluted with EtOAc and filtered through a pad of celite. The filtrate was concentrated under reduced pressure and purified by FC on RP using basic conditions (eluting from 5:95 of CH₃CN/H₂O+0.1% of HCOOH to 60:40 of CH₃CN/H₂O+0.1% of HCOOH). Pure fraction were collected and evaporated under vacuum affording the product of formula (N-[6-chloro-3-fluoro-5-(4-methyl-2-phenylpiperazine-1-carbonyl)pyridin-2-yl]cyclopropanecarboxamide.

### Yield: 65 mg

1H NMR (500 MHZ, DMSO-d6) δ ppm 11.28-10.70 (m, 1H), 8.33-7.83 (m, 1H), 7.60-7.31 (m, 4H), 7.31-7.24 (m, 1H), 5.85-4.58 (m, 1H), 3.57-3.24 (m, 1H), 4.46-3.19 (m, 1H), 3.17-2.84 (m, 1H), 2.83-2.54 (m, 1H), 2.50 (dt, 1H), 2.26-2.14 (m, 3H), 2.13-1.97 (m, 1H), 1.97-1.83 (m, 1H), 0.96-0.70 (m, 4H); LC-MS: m/z 417.17 (MH+).

### Scheme 24, Cpd (IV) Preparation: N-[3-fluoro-5-(4-methyl-2-phenylpiperazine-1-carbonyl)-6-pyrrolidin-1-ylpyridin-2-yl]cyclopropanecarboxamide

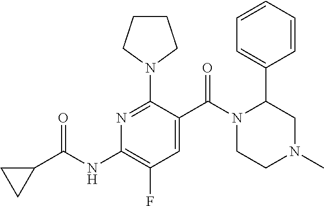 [SMILES: CN1CCN(C(=O)c2cc(F)c(NC(=O)C3CC3)nc2N2CCCC2)C(c2ccccc2)C1]

### Example 115

A solution of N-[6-chloro-3-fluoro-5-(4-methyl-2-phenylpiperazine-1-carbonyl)pyridin-2-yl]cyclopropanecarboxamide (65.0 mg, 0.160 mmol), potassium carbonate (0.04 mL, 0.310 mmol), XPHOS PD G3 (65.99 mg, 0.080 mmol) and pyrrolidine (11.09 mg, 0.160 mmol) in 1,4-Dioxane (1.5 mL) was by Schlenk technique and stirred ON at 100° C. The day after the reaction was cooled down to RT, diluted with water and extracted with EtOAc 3 times. The organic phase was washed with brine, dried over Na₂SO₄, filtered and concentrated under vacuum. The residue was purified by FC on silica gel (eluting from 100% of cHex to cHex/AcOEt 50:50) and again by FC on RP using acid conditions (eluting from 5:95 of CH₃CN/H₂O+0.1% of HCOOH to 50:50 of CH₃CN/H₂O+0.1% of HCOOH). Pure fractions were collected and concentrated under vacuum. The residue was taken up with a s.s. of NaHCO₃ and extracted 3 times with AcOEt. The combined organic fractions were washed with brine, dried over Na₂SO₄, filtered and concentrated under vacuum affording the product of formula N-[3-fluoro-5-(4-methyl-2-phenylpiperazine-1-carbonyl)-6-pyrrolidin-1-ylpyridin-2-yl]cyclopropanecarboxamide.

### Yield: 6 mg

1H NMR (500 MHZ, DMSO-d6) δ ppm 10.39-10.07 (m, 1H), 7.83-7.00 (m, 6H), 5.84-4.74 (m, 1H), 4.56-2.60 (m, 8H), 2.47-1.80 (m, 5H), 2.03-1.47 (m, 5H), 0.91-0.63 (m, 4H); LC-MS: m/z 452.23 (MH+).

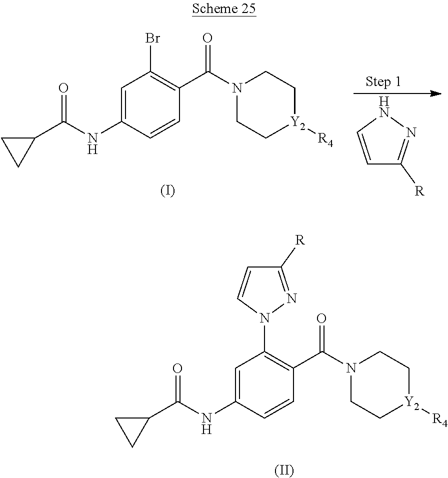

### Step 1

Compound of formula II may be obtained by Ullmann reaction between compound of formula I (synthesized according to the Scheme 3) and the appropriate commercially available pyrazole in the presence of a suitable catalyst, e.g. CuI, a suitable ligand, e.g. trans-N,N′-Dimethylcyclohexane-1,2-diamine, typically at 120° C. The reaction takes about 12 hours to complete.

### Preparation: N-[3-[3-(difluoromethyl) pyrazol-1-yl]-4-(1,1-dioxo-1,4-thiazinane-4-carbonyl)phenyl]cyclopropanecarboxamide

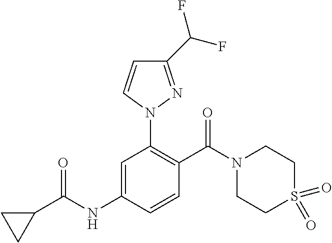 [SMILES: O=C(Nc1ccc(C(=O)N2CCS(=O)(=O)CC2)c(-n2ccc(C(F)F)n2)c1)C1CC1]

### Example 116

To a mixture of N-[3-bromo-4-(1,1-dioxo-1,4-thiazinane-4-carbonyl)phenyl]cyclopropanecarboxamide (100.0 mg, 0.240 mmol), tripotassium phosphate (107.03 mg, 0.500 mmol) and 3-(difluoromethyl)-1H-pyrazole (33.55 mg, 0.280 mmol) copper (I) iodide (2.27 mg, 0.010 mmol) and N,N′-Dimethylcyclohexane-1,2-diamine (3.37 mg, 0.020 mmol) were added. The reaction was degassed with 5 cycles of N₂/vacuum and stirred ON at 120° C. The day after the reaction mixture was cooled down to RT, diluted with EtOAc and filtered through a pad of celite. The organic layer was evaporated under reduced pressure and purified by FC on RP using acid conditions (eluting from 5:95 of CH₃CN/H₂O+0.1% of HCOOH to 40:60 of CH₃CN/H₂O+0.1% of HCOOH) affording the product of formula N-[3-[3-(difluoromethyl) pyrazol-1-yl]-4-(1,1-dioxo-1,4-thiazinane-4-carbonyl)phenyl]cyclopropanecarboxamide.

### Yield: 3.4 mg

1H NMR (400 MHZ, MeOD) δ 8.11 (d, 1H), 8.05 (d, 1H), 7.61 (dd, 1H), 7.45 (d, 1H), 7.01-6.67 (m, 2H), 4.49 (d, 1H), 3.97 (d, 1H), 3.78 (dd, 2H), 3.45 (d, 2H), 3.25-3.14 (m, 2H), 3.00 (d, 1H), 1.79 (tt, 1H), 1.10-0.86 (m, 4H); LC-MS: 439.11 (MH+).

### Preparation: N-[3-(3-cyanopyrazol-1-yl)-4-(1,1-dioxo-1,4-thiazinane-4-carbonyl)phenyl]cyclopropanecarboxamide

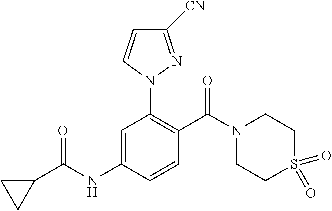 [SMILES: N#Cc1ccn(-c2cc(NC(=O)C3CC3)ccc2C(=O)N2CCS(=O)(=O)CC2)n1]

### Example 117

The synthesis of the title compound was effected analogously to the synthesis of the compound of Example 116 N-[3-[3-(difluoromethyl) pyrazol-1-yl]-4-(1,1-dioxo-1,4-thiazinane-4-carbonyl)phenyl]cyclopropanecarboxamide wherein 1H-pyrazole-3-carbonitrile was used instead of 3-(difluoromethyl)-1H-pyrazole. The title compound was obtained in a yield of 4% (6 mg).

1H NMR (400 MHZ, DMSO-d6) δ ppm 10.68 (s, 1H), 8.41 (d, 1H), 8.05 (d, 1H), 7.67 (dd, 1H), 7.55 (d, 1H), 7.24 (d, 1H), 4.05-3.51 (m, 4H), 3.43-2.90 (m, 4H), 1.88-1.70 (m, 1H), 0.92-0.72 (m, 4H); LC-MS: m/z 414.18 (MH+).

### Preparation: N-[3-(3-tert-butylpyrazol-1-yl)-4-(1,1-dioxo-1,4-thiazinane-4-carbonyl)phenyl]cyclopropanecarboxamide

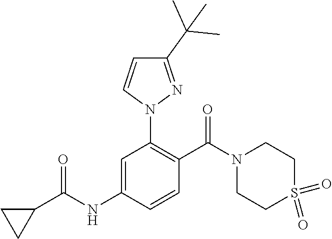 [SMILES: CC(C)(C)c1ccn(-c2cc(NC(=O)C3CC3)ccc2C(=O)N2CCS(=O)(=O)CC2)n1]

### Example 118

The synthesis of the title compound was effected analogously to the synthesis of the compound of Example 116 N-[3-[3-(difluoromethyl) pyrazol-1-yl]-4-(1,1-dioxo-1,4-thiazinane-4-carbonyl)phenyl]cyclopropanecarboxamide wherein 3-tert-butyl-1H-pyrazole was used instead of 3-(difluoromethyl)-1H-pyrazole. The title compound was obtained in a yield of 15% (9 mg).

1H NMR (400 MHZ, DMSO) δ 10.53 (s, 1H), 7.88 (dd, 2H), 7.59 (dd, 1H), 7.44 (d, 1H), 6.42 (d, 1H), 4.13 (s, 1H), 3.75 (t, 1H), 3.61 (dd, 1H), 3.36 (s, 1H), 3.19-3.09 (m, 1H), 2.99 (dd, 1H), 2.52 (d, 2H), 1.80 (p, 1H), 1.26 (s, 9H), 0.83 (d, 4H); LC-MS: m/z 445.16 (MH+).

### Preparation: N-[4-(4,4-difluoropiperidine-1-carbonyl)-3-[3-(trifluoromethyl) pyrazol-1-yl]phenyl]cyclopropanecarboxamide

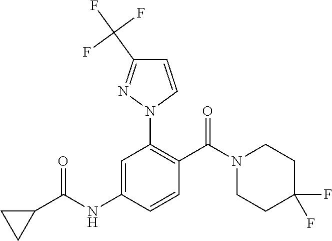 [SMILES: O=C(Nc1ccc(C(=O)N2CCC(F)(F)CC2)c(-n2ccc(C(F)(F)F)n2)c1)C1CC1]

### Example 119

The synthesis of the title compound was effected analogously to the synthesis of the compound of Example 116 N-[3-[3-(difluoromethyl) pyrazol-1-yl]-4-(1,1-dioxo-1,4-thiazinane-4-carbonyl)phenyl]cyclopropanecarboxamide wherein N-[3-bromo-4-(4,4-difluoropiperidine-1-carbonyl)phenyl]cyclopropanecarboxamide and 3-(trifluoromethyl) pyrazole were used instead of N-[3-bromo-4-(1,1-dioxo-1,4-thiazinane-4-carbonyl)phenyl]cyclopropanecarboxamide and 3-(difluoromethyl)-1H-pyrazole respectively. The title compound was obtained in a yield of 44% (60 mg).

1H NMR (400 MHZ, DMSO) δ 10.63 (s, 1H), 8.33 (dd, 1H), 8.01 (d, 1H), 7.66 (dd, 1H), 7.44 (d, 1H), 7.00 (d, 1H), 3.98-3.82 (m, 1H), 3.38-3.31 (m, 3H), 2.17-1.63 (m, 5H), 0.89-0.80 (d, 4H); LC-MS: m/z 443.14 (MH+).

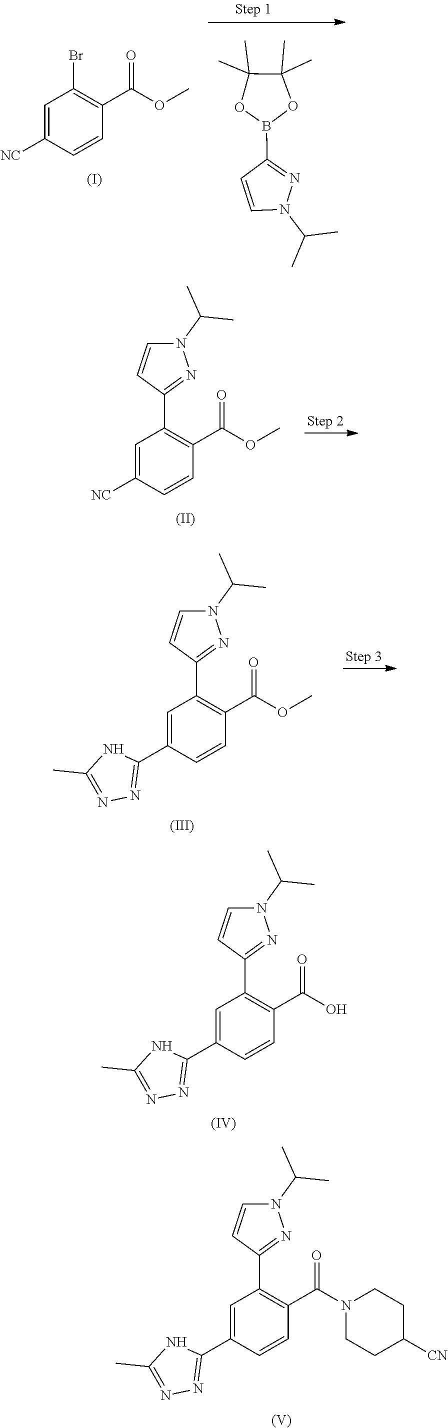

### Step 1

Compound of formula II may be obtained via Suzuki coupling between compound of formula I and the appropriate heterocycle boronic ester, e.g. 1-(propan-2-yl)-3-(tetramethyl-1,3,2-dioxaborolan-2-yl)-1H-pyrazole (commercially available from Enamine), in the presence of a suitable precatalyst, e.g. XPhos Pd G2, a suitable base, e.g. K₃PO₄, in a suitable mixture of solvents, e.g. 1,2-dimethoxyethane/water, typically at 90° C. The reaction takes about 3 hours to complete

### Step 2

Compound of formula III may be obtained by nitrile cyclization of compound of formula II with ethanimidamide hydrochloride in presence of a suitable catalyst, e.g. CuBr, a suitable base, e.g. Cs₂CO₃, in a suitable solvent, such as DMSO, typically at 120° C. The reaction takes about 6 hours to complete.

### Step 3

Compound of formula IV may be obtained from compound of formula III by hydrolysis with LiOH, in a suitable mixture of solvents such as MeOH/THF/H₂O, typically at 50° C. The reaction takes about 12 hours to complete.

### Step 4

Compound of formula V may be obtained from compound of formula IV by coupling with the appropriate amine, e.g. 4-piperidinecarbonitrile, in the presence of a suitable coupling agent, e.g. HATU, and an organic base, e.g. DIPEA. The reaction is carried out in a suitable solvent such as DMF, typically at room temperature. The reaction takes about 12 hours to complete.

### Scheme 26, Cpd (II) Preparation: methyl 4-cyano-2-(1-isopropylpyrazol-3-yl)benzoate

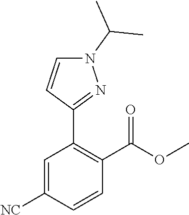 [SMILES: COC(=O)c1ccc(C#N)cc1-c1ccn(C(C)C)n1]

A mixture of X-phos aminobiphenyl palladium chloride precatalyst (98.2 mg, 0.120 mmol), tripotassium phosphate (795.85 mg, 3.75 mmol), 1-(propan-2-yl)-3-(tetramethyl-1,3,2-dioxaborolan-2-yl)-1H-pyrazole (236.07 mg, 1 mmol) and methyl 2-bromo-4-cyanobenzoate (300.0 mg, 1.25 mmol) in Water (0.397 mL)/1,2-dimethoxyethane (7.935 mL) was degassed with Shlenk line technique and then stirred at 90° C. for 3 h. After this time the reaction was cooled down. Water was added to the mixture and aqueous phase was extracted with AcOEt 3 times. Organic phases were combined, washed with brine, dried over Na₂SO₄, filtered and concentrated under vacuum. The residue was purified by FC on silica gel (eluting from 100% of cHex to 40:60 of cHex/AcOEt) affording the product of formula methyl 4-cyano-2-(1-isopropylpyrazol-3-yl)benzoate.

### Yield: 250 mg

1H NMR (400 MHZ, DMSO-d6) δ 8.00-7.96 (m, 2H), 7.93-7.89 (m, 1H), 7.86 (d, 1H), 6.67 (d, 1H), 4.52 (p, 1H), 3.76 (s, 3H), 1.42 (d, 6H); LC-MS: m/z 270.24 (MH+).

### Scheme 26, Cpd (III) Preparation: methyl 2-(1-isopropylpyrazol-3-yl)-4-(5-methyl-4H-1,2,4-triazol-3-yl)benzoate

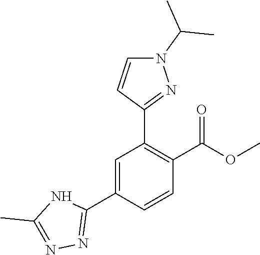

A mixture of methyl 4-cyano-2-(1-isopropylpyrazol-3-yl)benzoate (250.0 mg, 0.930 mmol), dicesium carbonate (907.41 mg, 2.78 mmol), ethanimidamide hydrochloride (131.65 mg, 1.39 mmol) and CuBr (6.66 mg, 0.050 mmol) in DMSO (10 mL) was stirred at 120° C. for 6 h. After this time the reaction was cooled down to RT and H₂O was added. The mixture was then extracted with AcOEt 3 times. The combined organic fractions were washed with brine, dried over Na₂SO₄, filtered and concentrated under vacuum. The residue was purified by FC on NH column (eluting from 100% of DCM to 95:5 of DCM/MeOH) affording the product of formula methyl 2-(1-isopropylpyrazol-3-yl)-4-(5-methyl-4H-1,2,4-triazol-3-yl)benzoate.

### Yield: 118 mg

1H NMR (400 MHZ, DMSO-d6) δ 13.81 (s, 1H), 8.16-7.99 (m, 2H), 7.88-7.63 (m, 2H), 6.52 (d, 1H), 4.51 (p, 1H), 3.74 (s, 3H), 2.40 (s, 3H), 1.43 (d, 6H); LC-MS: m/z 326.4 (MH+).

### Scheme 26, Cpd (IV) Preparation: 2-(1-isopropylpyrazol-3-yl)-4-(5-methyl-4H-1,2,4-triazol-3-yl)benzoic acid

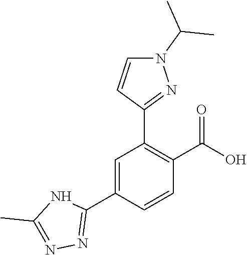 [SMILES: Cc1nnc(-c2ccc(C(=O)O)c(-c3ccn(C(C)C)n3)c2)[nH]1]

To a solution of methyl 2-(1-isopropylpyrazol-3-yl)-4-(5-methyl-4H-1,2,4-triazol-3-yl)benzoate (118.0 mg, 0.360 mmol) in Methanol (0.400 mL)/THF (2 mL) a solution of lithium hydroxide (13.03 mg, 0.540 mmol) in Water (0.400 mL) was added. The reaction was stirred at 50° C. ON. The day after the reaction was cooled down to RT, acidified with HCl 3 N and concentrated under vacuum. The residue was purified by FC on reverse phase using acid conditions (eluting from 5:95 of CH₃CN/H₂O+0.1% of HCOOH to 50:50 of CH₃CN/H₂O+0.1% of HCOOH) affording the product of formula 2-(1-isopropylpyrazol-3-yl)-4-(5-methyl-4H-1,2,4-triazol-3-yl)benzoic acid.

### Yield: 80 mg

1H NMR (400 MHZ, DMSO-d6) δ 13.74 (bs, 1H), 12.89 (bs, 1H), 8.15-7.99 (m, 2H), 7.85-7.63 (m, 2H), 6.48 (s, 1H), 4.52 (p, 1H), 2.43 (s, 3H), 1.46 (d, 6H); LC-MS: m/z 312.35 (MH+).

### Scheme 26, Cpd (V) Preparation: 1-[2-(1-isopropylpyrazol-3-yl)-4-(5-methyl-4H-1,2,4-triazol-3-yl)benzoyl]piperidine-4-carbonitrile

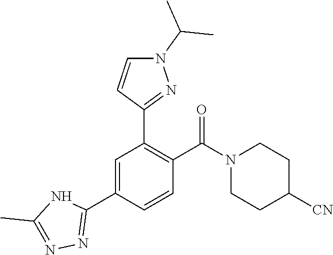 [SMILES: Cc1nnc(-c2ccc(C(=O)N3CCC(C#N)CC3)c(-c3ccn(C(C)C)n3)c2)[nH]1]

### Example 120

To a solution of, 2-(1-isopropylpyrazol-3-yl)-4-(5-methyl-4H-1,2,4-triazol-3-yl)benzoic acid (33.3 mg, 0.110 mmol) in DMF (0.491 mL), N,N-Diisopropylethylamine (0.06 mL, 0.320 mmol) and HATU (52.87 mg, 0.140 mmol) were added. The reaction was stirred at RT for 15 min. After this time 4-piperidinecarbonitrile (11.78 mg, 0.110 mmol) was added and the reaction was stirred ON at RT. After this time the reaction was diluted with a saturated aqueous solution of NaHCO₃ and extracted with AcOEt 3 times. The combined organic fractions were dried over Na₂SO₄, filtered and concentrated under vacuum. The residue was purified by FC on silica gel (eluting from 100% of DCM to EtOAc 100%) and again by FC on RP using acid conditions (eluting from 5:95 of CH₃CN/H₂O+0.1% of HCOOH to 20:80 of CH₃CN/H₂O+0.1% of HCOOH). Pure fractions were collected and evaporated. The residue was taken up with a s.s. of NaHCO₃ and extracted with AcOEt 3 times. The combined organic fractions were washed with brine, dried over Na₂SO₄, filtered and concentrated under vacuum affording the product of formula 1-[2-(1-isopropylpyrazol-3-yl)-4-(5-methyl-4H-1,2,4-triazol-3-yl)benzoyl]piperidine-4-carbonitrile.

### Yield: 19 mg.

1H NMR (400 MHZ, DMSO-d6) δ ppm 8.02 (dt, 1H), 7.77-7.87 (m, 3H), 6.49 (dd, 1H), 4.53 (e, 1H), 3.88-4.10 (m, 1H), 3.35-3.44 (m, 1H), 3.00-3.11 (m, 2H), 2.89 (dt, 1H), 2.37-2.43 (m, 3H), 1.87-1.98 (m, 1H), 1.51-1.82 (m, 3H), 1.41-1.50 (m, 6H), 0.98-1.10 (m, 1H); LC-MS: m/z 404.37 (MH+).

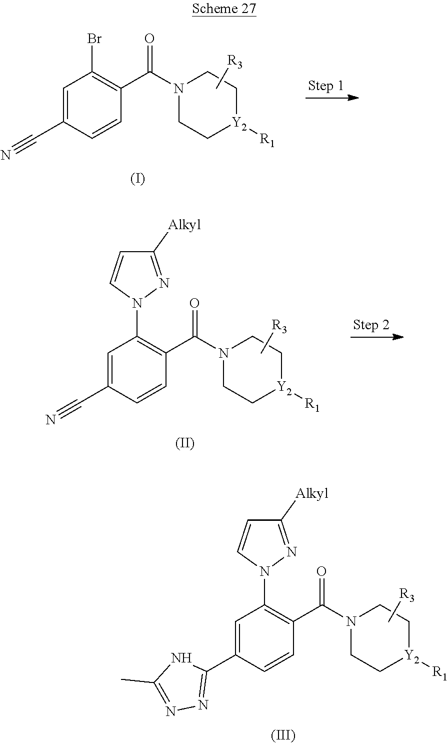

### Step 1

Compound of formula II may be obtained by Ullmann reaction between compound of formula I (synthesized according to the Scheme 4) and the appropriate commercially available pyrazole in the presence of a suitable catalyst, e.g. CuI, a suitable ligand, e.g. trans-N,N′-Dimethylcyclohexane-1,2-diamine, typically at 120° C. The reaction takes about 2 days to complete.

### Step 2

Compound of formula III may be obtained by nitrile cyclization of compound of formula II with ethanimidamide hydrochloride in presence of a suitable catalyst, e.g. CuBr, a suitable base, e.g. Cs₂CO₃, in a suitable solvent, such as DMSO, typically at 120° C. The reaction takes about 2 hours to complete.

### Scheme 27, Cpd (II)*a Preparation: 3-(3-cyclopropylpyrazol-1-yl)-4-[4-(trifluoromethyl) piperidine-1-carbonyl]benzonitrile

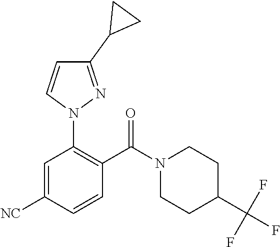 [SMILES: N#Cc1ccc(C(=O)N2CCC(C(F)(F)F)CC2)c(-n2ccc(C3CC3)n2)c1]

In a via 3-bromo-4-[4-(trifluoromethyl) piperidine-1-carbonyl]benzonitrile (200.0 mg, 0.550 mmol), tripotassium phosphate (250.36 mg, 1.16 mmol), 3-cyclopropyl-1H-pyrazole (71.86 mg, 0.660 mmol) were suspended in dry 1,4-Dioxane (3.038 mL). The resulting suspension was degassed bubbling N₂ for 15 minutes, then copper (I) iodide (5.3 mg, 0.030 mmol) and N,N′-Dimethylcyclohexane-1,2-diamine (7.88 mg, 0.060 mmol) were added under nitrogen flux and degassed again with Schlenk line technique. The vial was sealed and stirred at 120° C. for 2 days. The day after the reaction mixture was diluted with EtOAc and filtered through a pad of celite. The organic layer was washed with brine, dried over Na₂SO₄, filtered and evaporated under reduced pressure affording the product of formula 3-(3-cyclopropylpyrazol-1-yl)-4-[4-(trifluoromethyl) piperidine-1-carbonyl]benzonitrile, which was used in the next step without further purification.

### Yield: 180 mg

### LC-MS: m/z 389.20 (MH+).

### Scheme 27, Cpd (II)*b Preparation: 3-(3-tert-butylpyrazol-1-yl)-4-(4,4-difluoropiperidine-1-carbonyl)benzonitrile

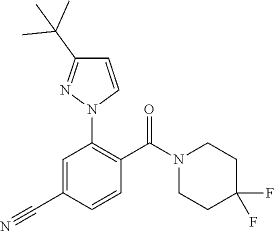 [SMILES: CC(C)(C)c1ccn(-c2cc(C#N)ccc2C(=O)N2CCC(F)(F)CC2)n1]

The synthesis of the title compound was effected analogously to the synthesis of the compound 3-(3-cyclopropylpyrazol-1-yl)-4-[4-(trifluoromethyl) piperidine-1-carbonyl]benzonitrile wherein 3-bromo-4-(4,4-difluoropiperidine-1-carbonyl)benzonitrile and 3-tert-butyl-1H-pyrazole were used instead of 3-bromo-4-[4-(trifluoromethyl) piperidine-1-carbonyl]benzonitrile and 3-cyclopropyl-1H-pyrazole respectively. The title compound was obtained in a quantitative yield (150 mg).

1H NMR (400 MHZ, DMSO-d6) δ ppm 8.20 (s, 2H), 7.90-7.83 (m, 1H), 7.70-7.63 (m, 1H), 6.51-6.45 (m, 1H), 4.02-3.94 (m, 1H), 3.42-3.32 (m, 1H), 3.08-2.95 (m, 1H), 2.20-1.85 (m, 3H), 1.72-1.55 (m, H), 1.25 (s, 9H); LC-MS: m/z 373.36 (MH+).

### Scheme 27, Cpd (III)*a Preparation: [2-(3-cyclopropylpyrazol-1-yl)-4-(5-methyl-4H-1,2,4-triazol-3-yl)phenyl]-[4-(trifluoromethyl)piperidin-1-yl]methanone

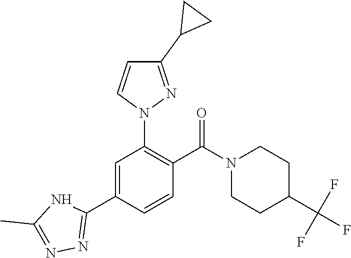 [SMILES: Cc1nnc(-c2ccc(C(=O)N3CCC(C(F)(F)F)CC3)c(-n3ccc(C4CC4)n3)c2)[nH]1]

### Example 121

A mixture of 3-(3-cyclopropylpyrazol-1-yl)-4-[4-(trifluoromethyl) piperidine-1-carbonyl]benzonitrile (180.0 mg, 0.460 mmol), dicesium carbonate (453.01 mg, 1.39 mmol), ethanimidamide hydrochloride (65.72 mg, 0.700 mmol) and CuBr (3.32 mg, 0.020 mmol) in DMSO (5.143 mL) was stirred at 120° C. for 2 h. After this time the reaction was cooled down to RT and H₂O was added. The mixture was then extracted with AcOEt 3 times. The combined organic fractions were washed with brine, dried over Na₂SO₄, filtered and concentrated under vacuum. The residue was purified by FC on NH column (eluting from 100% of DCM to DCM/MeOH 9:1) affording the product of formula [2-(3-cyclopropylpyrazol-1-yl)-4-(5-methyl-4H-1,2,4-triazol-3-yl)phenyl]-[4-(trifluoromethyl)piperidin-1-yl]methanone.

### Yield: 16 mg

¹H NMR (400 MHZ, DMSO-d₆) δ 13.86 (bs, 1H), 8.12-8.09 (m, 1H), 8.06-7.87 (m, 2H), 7.51-7.39 (m, 1H), 6.30-6.15 (m, 1H), 4.66-4.44 (m, 1H), 3.47-3.36 (m, 1H), 3.04-2.91 (m, 1H), 2.78-2.57 (m, 2H), 2.41 (s, 3H), 2.00-1.62 (m, 3H), 1.54-1.06 (m, 2H), 0.97-0.84 (m, 2H), 0.79-0.56 (m, 2H); LC-MS: m/z 445.2 (MH+).

### Preparation: [2-(3-tert-butylpyrazol-1-yl)-4-(5-methyl-4H-1,2,4-triazol-3-yl)phenyl]-(4,4-difluoropiperidin-1-yl)methanone

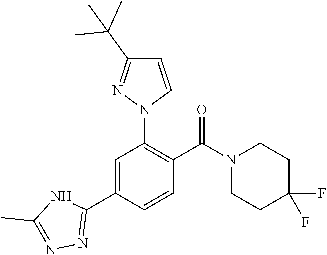 [SMILES: Cc1nnc(-c2ccc(C(=O)N3CCC(F)(F)CC3)c(-n3ccc(C(C)(C)C)n3)c2)[nH]1]

### Example 122

The synthesis of the title compound was effected analogously to the synthesis of the compound of Example 121 [2-(3-cyclopropylpyrazol-1-yl)-4-(5-methyl-4H-1,2,4-triazol-3-yl)phenyl]-[4-(trifluoromethyl)piperidin-1-yl]methanone wherein 3-(3-tert-butylpyrazol-1-yl)-4-(4,4-difluoropiperidine-1-carbonyl)benzonitrile was used instead of 3-(3-cyclopropylpyrazol-1-yl)-4-[4-(trifluoromethyl) piperidine-1-carbonyl]benzonitrile. The title compound was obtained in a yield of 35% (50 mg).

¹H NMR (400 MHZ, DMSO-d₆) δ 8.08 (d, 1H), 7.99-7.89 (m, 2H), 7.41 (d, 1H), 6.43 (d, 1H), 3.66 (q, 2H), 3.14-3.02 (m, 1H), 2.30 (s, 3H), 2.12-1.99 (m, 1H), 1.94-1.77 (m, 2H), 1.43-1.21 (m, 11H); LC-MS: m/z 429.26 (MH+).

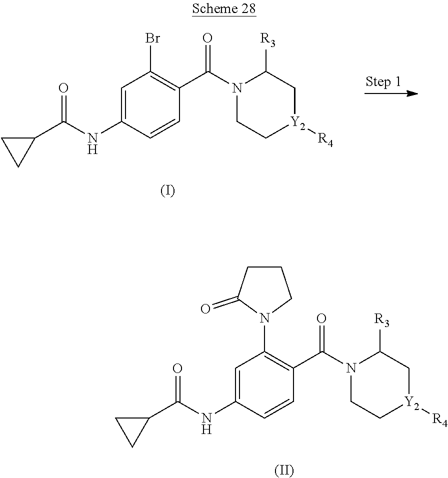

### Step 1

Compound of formula II may be obtained by reaction between compound I (synthesized according to the Scheme 3) and 2-pyrrolidinone in the presence of a suitable catalyst, e.g. CuI, a suitable ligand, e.g. trans-N,N′-Dimethylcyclohexane-1,2-diamine, and a suitable base, e.g. K₂CO₃. The reaction is carried out in a suitable solvent, such as 1, 4-dioxane, typically at 120° C. The reaction takes about 12 hours to complete. Alternatively compound of formula II may be obtained by Buchwald reaction between compound I (synthesized according to the Scheme 3) and 2-pyrrolidinone in the presence of a suitable catalyst, e.g. Pd₂(dba)₃, a suitable ligand, such as XPhos, and a suitable base, e.g. Cs₂CO₃. The reaction is carried out in a suitable solvent, such as 1, 4-dioxane, typically at 110° C. The reaction takes about 12 hours to complete.

### Preparation: N-[4-(1,1-dioxo-1,4-thiazinane-4-carbonyl)-3-(2-oxopyrrolidin-1-yl)phenyl]cyclopropanecarboxamide

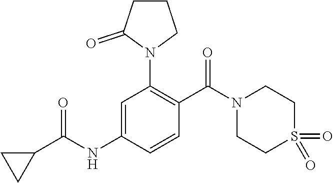 [SMILES: O=C(Nc1ccc(C(=O)N2CCS(=O)(=O)CC2)c(N2CCCC2=O)c1)C1CC1]

### Example 123

To a suspension of N-[3-bromo-4-(1,1-dioxo-1,4-thiazinane-4-carbonyl)phenyl]cyclopropanecarboxamide (200.0 mg, 0.500 mmol), potassium carbonate (137.77 mg, 1 mmol) and 2-pyrrolidinone (0.05 mL, 0.600 mmol) in 1,4-Dioxane (3 mL) N,N′-Dimethylcyclohexane-1,2-diamine (0.02 mL, 0.100 mmol) and copper (I) iodide (9.49 mg, 0.050 mmol) were added. The mixture was degassed with N₂ for 10 min and then heated to 120° C. ON. The day after the reaction was diluted with AcOEt and washed with brine. The organic phase was dried over Na₂SO₄, filtered and concentrated under vacuum. The residue was purified by FC on silica gel (eluting from 100% of DCM to DCM/MeOH 9:1) affording the product of formula N-[4-(1,1-dioxo-1,4-thiazinane-4-carbonyl)-3-(2-oxopyrrolidin-1-yl)phenyl]cyclopropanecarboxamide.

### Yield: 54 mg

1H NMR (400 MHZ, DMSO-d6) δ 10.45 (s, 1H), 7.67 (d, 1H), 7.47 (dd, 1H), 7.38 (d, 1H), 4.21-3.56 (m, 6H), 3.39 (s, 1H), 3.21 (t, 3H), 2.45-2.31 (m, 1H), 2.19-1.63 (m, 4H), 0.83 (d, 4H); LC-MS: m/z 406.2 (MH+)

### Preparation: N-[4-(4-methyl-2-phenylpiperazine-1-carbonyl)-3-(2-oxopyrrolidin-1-yl)phenyl]cyclopropanecarboxamide

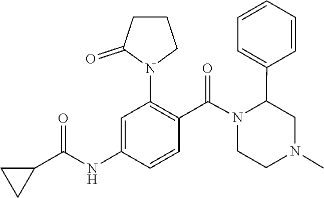 [SMILES: CN1CCN(C(=O)c2ccc(NC(=O)C3CC3)cc2N2CCCC2=O)C(c2ccccc2)C1]

### Example 124

To a suspension of N-[3-bromo-4-(4-methyl-2-phenylpiperazine-1-carbonyl)phenyl]cyclopropanecarboxamide (200.0 mg, 0.450 mmol), 2-pyrrolidinone (57.71 mg, 0.680 mmol) and (5-diphenylphosphino-9,9-dimethyl-4-xanthenyl)-diphenylphosphine (130.81 mg, 0.230 mmol) in 1,4-Dioxane (1.5 mL) dicesium carbonate (294.63 mg, 0.900 mmol) and (1E,4E)-1,5-diphenyl-3-penta-1,4-dienone palladium (62.1 mg, 0.070 mmol) were added. The mixture was degassed with N₂ for 10 min and then heated to 110° C. ON. The day after the reaction was diluted with AcOEt and washed with brine. The organic phase was dried over Na₂SO₄, filtered and concentrated under vacuum. The residue was purified by FC on NH column (eluting from cHex/AcOEt 80:20 to cHex/AcOEt 40:60) and again by FC on RP using basic conditions (eluting from 5:95 of CH₃CN/H₂O+0.1% of NH₄OH to 30:70 of CH₃CN/H₂O+0.1% of NH₄OH) affording the product of formula (N-[4-(4-methyl-2-phenylpiperazine-1-carbonyl)-3-(2-oxopyrrolidin-1-yl)phenyl]cyclopropanecarboxamide.

### Yield: 66 mg

¹H NMR (400 MHZ, DMSO-d₆) δ 10.42 (s, 1H), 7.73-7.62 (m, 1H), 7.59-7.40 (m, 2H), 7.41-7.21 (m, 5H), 5.86-4.34 (m, 1H), 3.82-3.35 (m, 4H), 2.89 (d, 2H), 2.39-1.67 (m, 10H), 0.86-0.79 (m, 4H); LC-MS: m/z 447.25 (MH+).

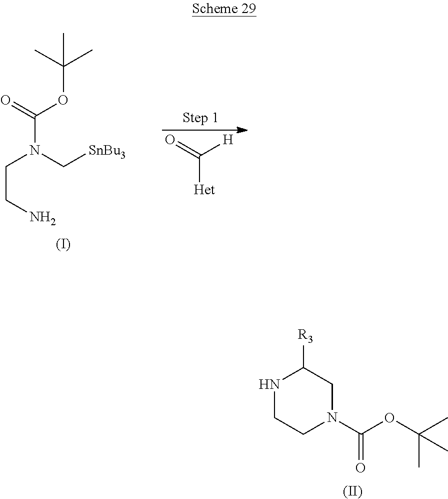

### Step 1

Compound of formula II may be obtained from the commercially available SnAP reagent (Compound I) by reaction with the appropriate aromatic aldehyde, followed by Cu mediated cyclization. The reaction is carried out in a proper solvent, e.g. DCM, typically at RT. The reaction takes about 12 hours to complete.

### Scheme 29, Cpd (II)*a Preparation: tert-butyl 3-(3-methylphenyl) piperazine-1-carboxylate

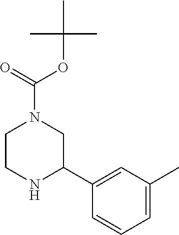 [SMILES: Cc1cccc(C2CN(C(=O)OC(C)(C)C)CCN2)c1]

To a solution of SnAP Pip reagent (771.19 mg, 1.66 mmol) in dry DCM (8.3 mL) at room temperature 3-methylbenzaldehyde (200.0 mg, 1.66 mmol) and previously activated MS 3A were added. The reaction was stirred at rt overnight and then filtered through a short layer of celite, washing with DCM. The filtrate was concentrated under reduced pressure to afford the imine. Separately, 2, 6-Lutidine (178.36 mg, 1.66 mmol) was added in one portion to a suspension of dry Copper (II) trifluoromethanesulfonate (602.05 mg, 1.66 mmol) in anhydrous HFIP (6.6 mL, 1.66 mmol) and stirred at RT for 1 h, during which time a homogeneous suspension was formed. A solution of imine in dry DCM (26 mL) was added in one portion and the resulting mixture was stirred at rt overnight. After 18 h, the reaction was quenched at rt with 15 mL of a 10% aqueous solution of NH40H and stirred vigorously for 15 min. The layers were separated and the aqueous layer was extracted with DCM ×3. The combined organic layers were washed with H₂O, brine, dried using a phase separator and concentrated. The crude was purified by FC on RP using basic conditions (eluting from 5:95 of CH₃CN/H₂O+0.1% of NH₄OH to 30:70 of CH₃CN/H₂O+0.1% of NH₄OH) affording the product of formula tert-butyl 3-(3-methylphenyl) piperazine-1-carboxylate.

### Yield: 336 mg

1H NMR (400 MHZ, DMSO-d6) δ ppm 7.25-7.15 (m, 3H), 7.08 (d, 1H), 3.92-3.74 (m, 2H), 3.51 (dd, 1H), 2.99-2.57 (m, 5H), 2.29 (s, 3H), 1.40 (s, 9H); LC-MS: m/z 277.46 (MH+).

### Scheme 29, Cpd (II)*b Preparation: tert-butyl 3-(3-chloro-5-methylphenyl) piperazine-1-carboxylate

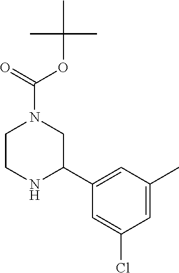 [SMILES: Cc1cc(Cl)cc(C2CN(C(=O)OC(C)(C)C)CCN2)c1]

The synthesis of the title compound was effected analogously to the synthesis of the compound tert-butyl 3-(3-methylphenyl) piperazine-1-carboxylate wherein 3-chloro-5-methylbenzaldehyde was used instead of 3-methylbenzaldehyde. The title compound was obtained in a yield of 69% (276 mg).

1H NMR (400 MHZ, DMSO-d6) δ ppm 7.25 (s, 1H), 7.22-7.14 (m, 2H), 3.88-3.74 (m, 2H), 3.54 (dt, 1H), 2.97-2.88 (m, 1H), 2.87-2.76 (m, 2H), 2.69-2.55 (m, 2H), 2.30 (s, 3H), 1.40 (s, 9H); LC-MS: m/z 311.14 (MH+).

### Scheme 29, Cpd (II)*c Preparation: tert-butyl 3-(4-methylphenyl) piperazine-1-carboxylate

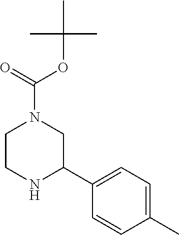 [SMILES: Cc1ccc(C2CN(C(=O)OC(C)(C)C)CCN2)cc1]

The synthesis of the title compound was effected analogously to the synthesis of the compound tert-butyl 3-(3-methylphenyl) piperazine-1-carboxylate wherein 4-methylbenzaldehyde was used instead of 3-methylbenzaldehyde. The title compound was obtained in a yield of 66% (306 mg).

1H NMR (400 MHZ, DMSO-d6) δ ppm 7.32-7.10 (m, 4H), 3.92-3.74 (m, 2H), 3.56-3.47 (m, 1H), 2.99-2.58 (m, 5H), 2.28 (s, 3H), 1.40 (s, 9H); LC-MS: m/z 277.71 (MH+).

Scheme 29, Cpd (II)*d Preparation: tert-butyl 3-[2-(trifluoromethyl)phenyl]piperazine-1-carboxylate

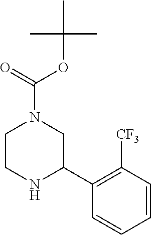 [SMILES: CC(C)(C)OC(=O)N1CCNC(c2ccccc2C(F)(F)F)C1]

The synthesis of the title compound was effected analogously to the synthesis of the compound tert-butyl 3-(3-methylphenyl) piperazine-1-carboxylate wherein 2-(trifluoromethyl)benzaldehyde was used instead of 3-methylbenzaldehyde. The title compound was obtained in a yield of 70% (200 mg).

1H NMR (400 MHZ, DMSO-d6) δ ppm 7.96 (d, 1H), 7.76-7.64 (m, 2H), 7.49 (t, 1H), 3.96-3.81 (m, 3H), 2.96 (d, 2H), 2.89-2.74 (m, 1H), 2.74-2.59 (m, 2H), 1.38 (s, 9H); LC-MS: m/z 275.12 (MH+).

### Scheme 29, Cpd (II)*e Preparation: tert-butyl 3-(3-cyanophenyl) piperazine-1-carboxylate

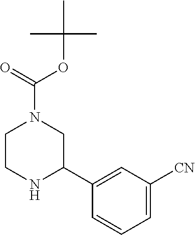 [SMILES: CC(C)(C)OC(=O)N1CCNC(c2cccc(C#N)c2)C1]

The synthesis of the title compound was effected analogously to the synthesis of the compound tert-butyl 3-(3-methylphenyl) piperazine-1-carboxylate wherein 3-formylbenzonitrile was used instead of 3-methylbenzaldehyde. The title compound was obtained in a yield of 51% (168 mg).

1H NMR (400 MHZ, DMSO-d6) δ ppm 7.85 (s, 1H), 7.81-7.71 (m, 2H), 7.59-7.53 (m, 1H), 3.92-3.75 (m, 2H), 3.70-3.61 (m, 1H), 2.98-2.78 (m, 3H), 2.70-2.57 (m, 2H), 1.40 (s, 9H); LC-MS: m/z 288.23 (MH+).

### Scheme 29, Cpd (II)*f Preparation: tert-butyl 3-(4,5-dimethyl-1,3-thiazol-2-yl) piperazine-1-carboxylate

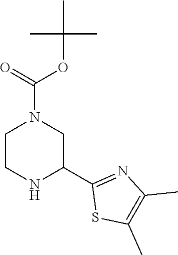 [SMILES: Cc1nc(C2CN(C(=O)OC(C)(C)C)CCN2)sc1C]

The synthesis of the title compound was effected analogously to the synthesis of the compound tert-butyl 3-(3-methylphenyl) piperazine-1-carboxylate wherein 4,5-dimethylthiazole-2-carboxaldehyde was used instead of 3-methylbenzaldehyde. The title compound was obtained in a yield of 64% (203 mg).

¹H NMR (400 MHZ, DMSO-d₆) δ 3.99 (s, 1H), 3.86-3.75 (m, 1H), 3.71 (d, 1H), 3.10-3.05 (m, 1H), 2.98-2.80 (m, 2H), 2.71-2.62 (m, 2H), 2.30 (s, 3H), 2.23 (s, 3H), 1.41 (s, 9H); LC-MS: m/z 297.61 (MH+).

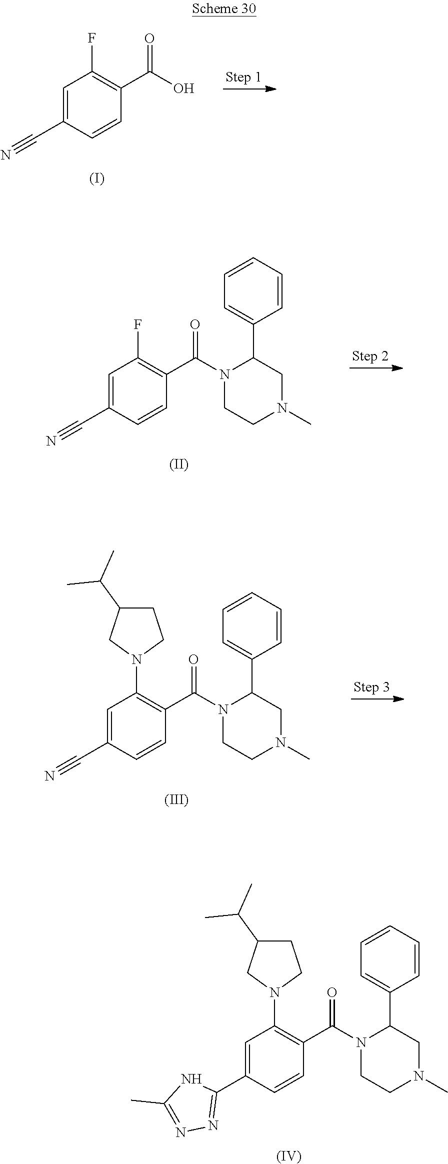

### Step 1

Compound of formula II may be obtained by coupling reaction between compound of formula I (commercially available from Zantec) and the appropriate amine, e.g. 1-methyl-3-phenylpiperazine, in presence of a suitable coupling agent, e.g. HATU, a suitable organic base, e.g. DIPEA. The reaction is carried out in a suitable solvent, e.g. DMF, typically at RT. The reaction takes about 2 hours to complete.

### Step 2

Compound of formula III may be obtained from compound of formula II by SNAr substitution with 3-(propan-2-yl) pyrrolidine in a suitable aprotic solvent, e.g. DMSO, typically at 120° C. The reaction takes about 2 days to complete.

### Scheme 30, Cpd (II) Preparation: 3-fluoro-4-(4-methyl-2-phenylpiperazine-1-carbonyl)benzonitrile

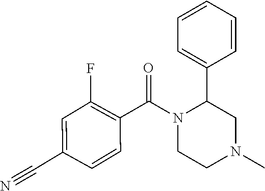 [SMILES: CN1CCN(C(=O)c2ccc(C#N)cc2F)C(c2ccccc2)C1]

A solution of 4-cyano-2-fluorobenzoic acid (500.0 mg, 3.03 mmol), N,N-Diisopropylethylamine (2.64 mL, 15.14 mmol) and HATU (1727.06 mg, 4.54 mmol) in DMF (15.14 mL) was stirred for 15 minutes at RT. Then 1-methyl-3-phenylpiperazine (640.48 mg, 3.63 mmol) was added and the reaction was stirred at RT for 2 hours. A saturated solution of NaHCO₃ was added to the reaction mixture and the aqueous phase was extracted with EtOAc (×3). The organic portions were collected, washed with brine, residual water was removed by Na₂SO₄, the solvent was filtered and evaporated under reduced pressure. The crude was purified by FC on silica gel (eluting from 100% of cHex to cHex/AcOEt 1:1) affording the product of formula 3-fluoro-4-(4-methyl-2-phenylpiperazine-1-carbonyl)benzonitrile.

### Yield: 880 mg

¹H NMR (400 MHZ, DMSO-d₆) δ 8.03 (d, 1H), 7.89-7.63 (m, 2H), 7.54-7.22 (m, 5H), 5.91-4.26 (m, 1H), 3.59-3.43 (m, 1H), 3.24-2.60 (m, 3H), 2.41-2.28 (m, 1H), 2.20 (s, 3H), 2.07-1.82 (m, 1H); LC-MS: m/z 324.20 (MH+).

### Scheme 30, Cpd (III) Preparation: 4-(4-methyl-2-phenylpiperazine-1-carbonyl)-3-(3-propan-2-ylpyrrolidin-1-yl)benzonitrile

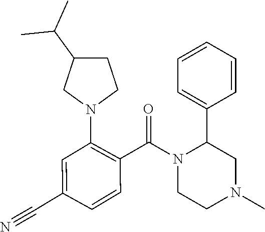 [SMILES: CC(C)C1CCN(c2cc(C#N)ccc2C(=O)N2CCN(C)CC2c2ccccc2)C1]

To a solution of 3-fluoro-4-(4-methyl-2-phenylpiperazine-1-carbonyl)benzonitrile (300.0 mg, 0.930 mmol) and 3-(propan-2-yl) pyrrolidine hydrochloride (166.62 mg, 1.11 mmol) in DMSO (8.737 mL) N,N-Diisopropylethylamine (0.4 mL, 2.32 mmol) was added. The mixture was stirred at 120° C. for 2 days. After this time the reaction was cooled down to RT and diluted with H₂O and was extracted with Acoet. The combined organic fractions were washed with brine, dried over Na₂SO₄ and concentrated under vacuum. The crude was purified by FC on NH column (eluting from 100% of cHex to cHex/AcOEt 75:25) affording the product of formula 4-(4-methyl-2-phenylpiperazine-1-carbonyl)-3-(3-propan-2-ylpyrrolidin-1-yl)benzonitrile as mixture of diastereoisomers.

### Yield: 95 mg

### LC-MS: m/z: m/z 471.34 (MH+).

### Scheme 30, Cpd (IV) Preparation: (4-methyl-2-phenylpiperazin-1-yl)-[4-(5-methyl-4H-1,2,4-triazol-3-yl)-2-(3-propan-2-ylpyrrolidin-1-yl)phenyl]methanone

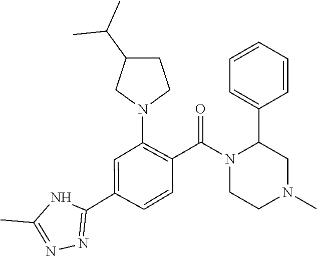

### Example 125a, Example 125b, Example 125c, and Example 125d

A mixture of 4-(4-methyl-2-phenylpiperazine-1-carbonyl)-3-(3-propan-2-ylpyrrolidin-1-yl)benzonitrile (95.0 mg, 0.230 mmol), dicesium carbonate (222.92 mg, 0.680 mmol), ethanimidamide hydrochloride (32.34 mg, 0.340 mmol) and bromocopper (1.64 mg, 0.010 mmol) in DMSO (3.5 mL) was stirred at 120° C. for 6 h. After this time the reaction was cooled down to RT and H₂O was added. The mixture was then extracted with AcOEt 3 times. The combined organic fractions were washed with brine, dried over Na₂SO₄, filtered and concentrated under vacuum. The residue was purified by FC on NH column (eluting from 100% of DCM to DCM/MeOH 95:5) affording the product of formula (4-methyl-2-phenylpiperazin-1-yl)-[4-(5-methyl-4H-1,2,4-triazol-3-yl)-2-(3-propan-2-ylpyrrolidin-1-yl)phenyl]methanone as mixture of diastereoisomers Yield: 58 mg

### LC-MS: m/z 473.59 (MH+)

The mixture of diastereoisomers was then separated into single enantiomers by chiral semi-preparative HPLC

| | | |
|---|---|---|
| Preparative chiral | Column | Whelk 01 (R,R) (25 x |
| chromatography | | 0.46 cm), 10 u |
| protocol: | Mobile phase | n-Hexane/Ethanol |
| | | 65/35% v/v |
| | Flow rate (ml/min) | 37 ml/min |
| | DAD detection | 220 nm |
| | Loop | 1000 μL |
| | Total amount | 58 mg |
| | Injection | 14 mg (each injection) |

| | |
|---|---|
| 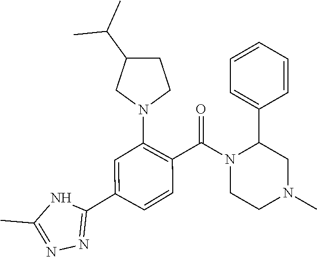 | Diasteroisomer 1 Enantiomer 1, Yield: 11 mg Rt 11.66 min 100% ee LC-MS: m/z 473.29 (MH+) (2S, 3R) or (2R, 3S) or (2R, 3R) or (25, 3S) (4-methyl-2- phenylpiperazin-1-yl)-[4-(5-methyl-4H-1,2,4-triazol-3-yl)- 2-(3-propan-2-ylpyrrolidin-1-yl)phenyl]methanone Example 125a |
| (2S, 3R) or (2R, 3S) or (2R, 3R) or | |
| (2S, 3S) | |
| 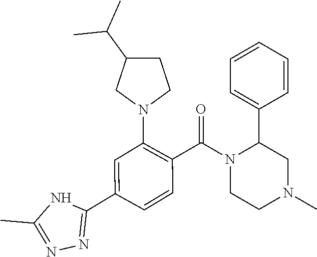 | Enantiomer 2, Yield: 12 mg Rt 16.82 min 100% ee LC-MS: m/z 473.29 (MH+) (2S, 3R) or (2R, 3S) or (2R, 3R) or (2S, 3S) (4-methyl-2- phenylpiperazin-1-yl)-[4-(5-methyl-4H-1,2,4-triazol-3-yl)- 2-(3-propan-2-ylpyrrolidin-1-yl)phenyl]methanone Example 125b |
| (2S, 3R) or (2R, 3S) or (2R, 3R) or | |
| (2S, 3S) | |

¹H NMR (400 MHz, DMSO-d₆) δ 13.72 (s, 1H), 7.78-7.47 (m, 1H), 7.44-7.39 (m, 1H), 7.37-7.23
(m, 4H), 7.23-7.12 (m, 1H), 7.06-6.91 (m, 1H), 5.94-4.37 (m, 1H), 3.78-3.38 (m, 4H), 3.23-
2.57 (m, 5H), 2.41-2.31 (m, 3H), 2.28-2.07 (m, 4H), 2.05-1.81 (m, 2H), 1.77-1.22 (m, 2H), 1.03-
0.94 (m, 4H), 0.87-0.62 (m, 2H).

| | |
|---|---|
| 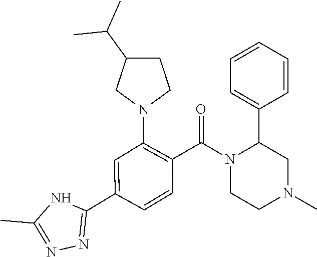 | Diasteroisomer 2 Enantiomer 1, Yield: 7 mg Rt 17.85 min 100% ee LC-MS: m/z 473.29 (MH+) (2S, 3R) or (2R, 3S) or (2R, 3R) or (2S, 3S) (4-methyl-2- phenylpiperazin-1-yl)-[4-(5-methyl-4H-1,2,4-triazol-3-yl)- 2-(3-propan-2-ylpyrrolidin-1-yl)phenyl]methanone Example 125c |
| (2S, 3R) or (2R, 3S) or (2R, 3R) or | |
| (2S, 3S) | |
| 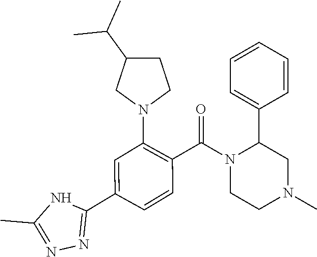 | Enantiomer 2, Yield: 5 mg Rt 25.47 min 100% ee LC-MS: m/z 473.29 (MH+) (2S, 3R) or (2R, 3S) or (2R, 3R) or (2S, 3S) (4-methyl-2- phenylpiperazin-1-yl)-[4-(5-methyl-4H-1,2,4-triazol-3-yl)- 2-(3-propan-2-ylpyrrolidin-1-yl)phenyl]methanone Example 125d |
| (2S, 3R) or (2R, 3S) or (2R, 3R) or | |
| (2S, 3S) | |

¹H NMR (400 MHz, DMSO-d₆) δ 13.73 (bs, 1H), 7.86-7.45 (m, 2H), 7.42-7.21 (m, 5H), 7.19-6.82
(m, 1H), 5.98-4.31 (m, 1H), 3.55-3.38 (m, 3H), 3.24-2.58 (m, 5H), 2.46-2.26 (m, 5H), 2.22-
2.07 (m, 3H), 2.05-1.74 (m, 2H), 1.67-1.53 (m, 1H), 1.45-1.32 (m, 1H), 1.07-0.95 (m, 3H), 0.91-
0.65 (m, 3H).

1. A compound of Formula (I-A), or a pharmaceutically acceptable salt thereof: 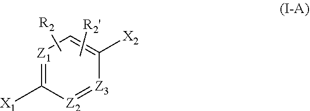
wherein:
X₁ is selected from the group of: 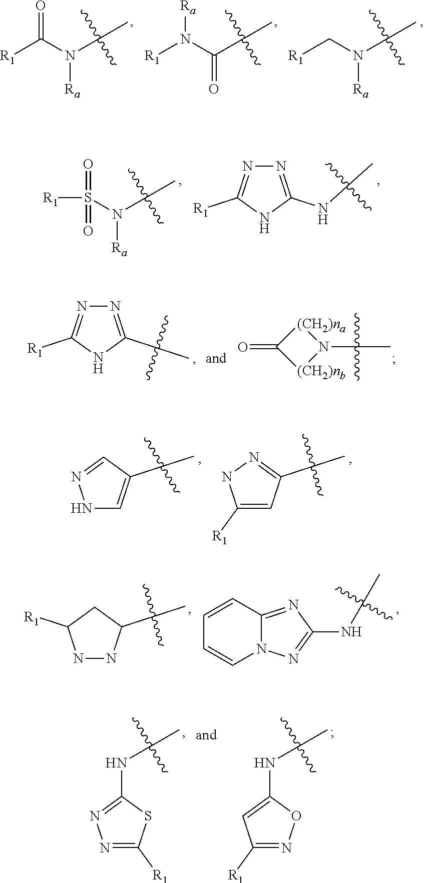
nₐ is an integer selected from the group of 0, 1, and 2;
n_{b} is an integer selected from the group of 0, 1, 2, 3, and 4;
with the proviso that the sum of nₐ+n_{b} is not less than 2 and not greater than 4;
or X₁ and Z₁ together form a fused ring system of the formula (Ia): 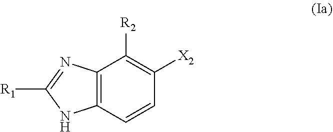
Rₐ is selected from the group of hydrogen and C₁-C₃ alkyl;
X₂ is selected from the group of: 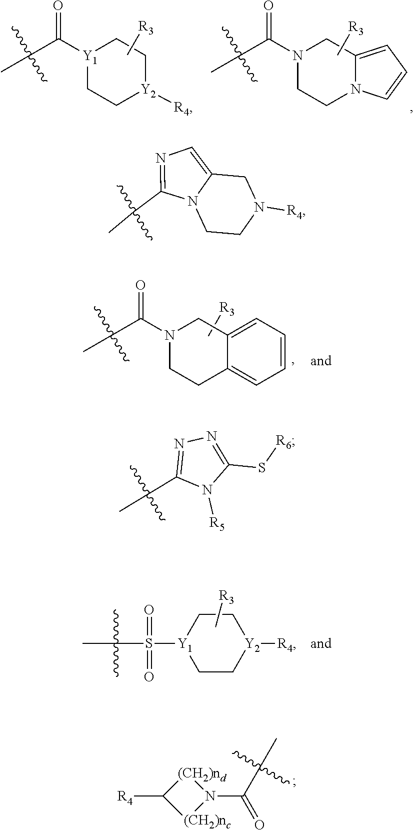
the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} is not less than 2 and not greater than 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of carbocycle and a monocyclic heterocyclic ring or a bicyclic or spirocyclic heterocyclic ring system bound through a carbon atom and containing 3, 4, 5, 6, 7, or 8 ring carbon atoms and 1, 2, 3, or 4 ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂—C₁-C₆ alkyl, —C(═O)—NR^{x}R^{y}, —N(R^{x})(S(═O)₂—C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl, with the proviso that R₂ is not pyridinyl unless substituted at least once by —(CH₂)ₙ₁—C₃-C₆ cycloalkyl or phenyl;
R₂′ is selected from the group of hydrogen, OH, halogen, C₁-C₆ alkyl, and —CF₃;
R₃ is present one or more times and is independently selected from the group of:
a) hydrogen, halogen, cyano, or OH;
b) —CO₂H or —CO₂—(C₁-C₆ alkyl);
c) C₁-C₆ alkyl or —O—C₁-C₆ alkyl substituted by 0, 1, 2, 3, 4 or 5 substituents selected from the group of halogen, CF₃, and OH;
d) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
e) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
f) two R₃ form a bridge between carbon atoms, wherein the atoms of the bridge comprise 1, 2, or 3 atoms independently selected from the group of carbon, N, O, and S;
R₄ is present one or two times and is independently selected from the group of H, halogen, oxo, C₁-C₆ alkyl, —(CH₂)ₙ₂—C₃-C₆ cycloalkyl, —O—(CH₂)ₙ₂—C₃-C₆ cycloalkyl, —C(═O)—O—C₁-C₆ alkyl, —S(═O)₂—C₁-C₆ alkyl, —C(═O)—NR^{x}R^{y}, —N(R^{x})(S(═O)₂—C₁-C₆ alkyl), phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a spirocyclic carbocycle or spirocyclic heterocycle;
wherein the R₄ C₁-C₆ alkyl, —C(═O)—O—C₁-C₆ alkyl, and —O—C₁-C₆ alkyl groups and the rings of the —(CH₂)ₙ₂—C₃-C₆ cycloalkyl and —O—(CH₂)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n₂ in each instance is an integer selected from the group of 0, 1, 2, and 3.

2. A compound of Formula (I-B), or a pharmaceutically acceptable salt thereof: 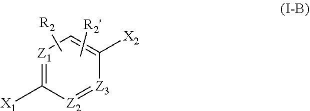
wherein:
X₁ is selected from the group of: 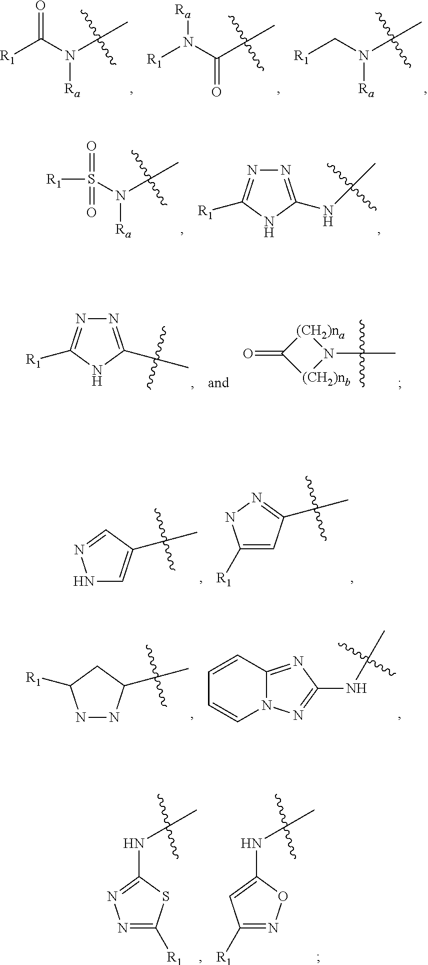
nₐ is an integer selected from the group of 0, 1, and 2;
n_{b} is an integer selected from the group of 0, 1, 2, 3, and 4;
with the proviso that the sum of nₐ+n_{b} is not less than 2 and not greater than 4;
or X₁ and Z₁ together form a fused ring system of the formula (Ia): 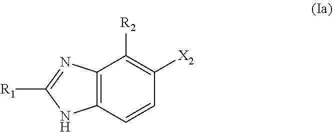
Rₐ is selected from the group of hydrogen and C₁-C₃ alkyl;
X₂ is selected from the group of: 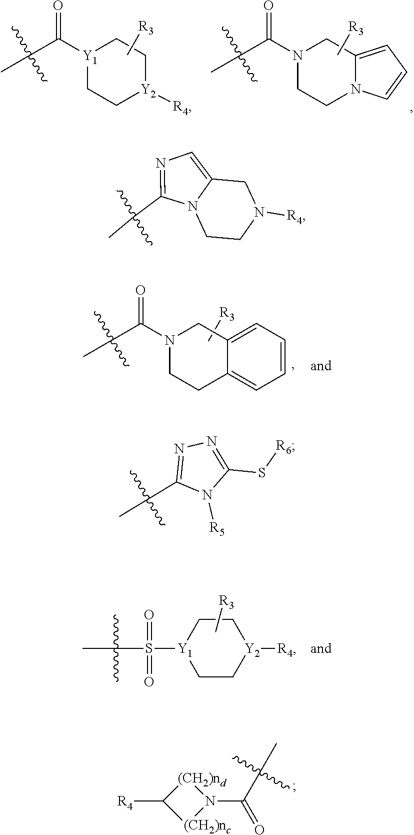
the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} is not less than 2 and not greater than 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl, pyridinyl bound through a carbon atom, and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂—C₁-C₆ alkyl, —C(═O)—NR^{x}R^{y}, —N(R^{x})(S(═O)₂—C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl, with the proviso that R₂ is not pyridinyl unless substituted at least once by —(CH₂)ₙ₁—C₃-C₆ cycloalkyl or phenyl;
R₂′ is selected from the group of OH, halogen, C₁-C₆ alkyl, and —CF₃;
R₃ is present one or more times and is independently selected from the group of:
g) hydrogen, halogen, cyano, or OH;
h) —CO₂H or —CO₂—(C₁-C₆ alkyl);
i) C₁-C₆ alkyl or —O—C₁-C₆ alkyl substituted by 0, 1, 2, 3, 4 or 5 substituents selected from the group of halogen, CF₃, and OH;
j) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
k) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
l) two R₃ form a bridge between carbon atoms, wherein the atoms of the bridge comprise 1, 2, or 3 atoms independently selected from the group of carbon, N, O, and S;
R₄ is present one or two times and is independently selected from the group of H, halogen, oxo, C₁-C₆ alkyl, —(CH₂)ₙ₂—C₃-C₆ cycloalkyl, —O—(CH₂)ₙ₂—C₃-C₆ cycloalkyl, —C(═O)—O—C₁-C₆ alkyl, —S(═O)₂—C₁-C₆ alkyl, —C(═O)—NR^{x}R^{y}, —N(R^{x})(S(═O)₂—C₁-C₆ alkyl), phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a spirocyclic carbocycle or spirocyclic heterocycle;
wherein the R₄ C₁-C₆ alkyl, —C(═O)—O—C₁-C₆ alkyl, and —O—C₁-C₆ alkyl groups and the rings of the —(CH₂)ₙ₂—C₃-C₆ cycloalkyl and —O—(CH₂)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n₂ in each instance is an integer selected from the group of 0, 1, 2, and 3.

3. A compound of Formula (I-C), or a pharmaceutically acceptable salt thereof: 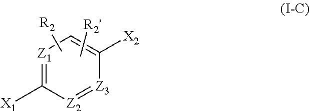
wherein:
X₁ is selected from the group of: 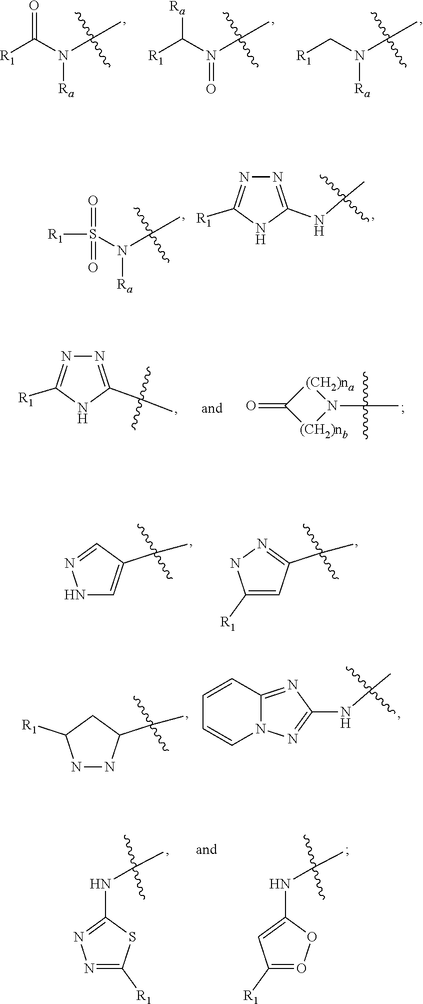
nₐ is an integer selected from the group of 0, 1, and 2;
n_{b} is an integer selected from the group of 0, 1, 2, 3, and 4;
with the proviso that the sum of nₐ+n_{b} is not less than 2 and not greater than 4;
or X₁ and Z₁ together form a fused ring system of the formula (Ia): 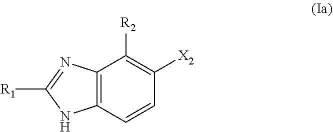
Rₐ is selected from the group of hydrogen and C₁-C₃ alkyl;
X₂ is selected from the group of: 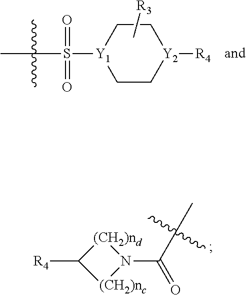
the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} 2, 3, 5, or 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl, pyridinyl bound through a carbon atom, and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂—C₁-C₆ alkyl, —C(═O)—NR^{x}R^{y}, —N(R^{x})(S(═O)₂—C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl, with the proviso that R₂ is not pyridinyl unless substituted at least once by —(CH₂)ₙ₁—C₃-C₆ cycloalkyl or phenyl;
R₂′ is selected from the group of hydrogen, OH, halogen, C₁-C₆ alkyl, and —CF₃;
R₃ is present one or more times and is independently selected from the group of:
m) hydrogen, halogen, cyano, or OH;
n) —CO₂H or —CO₂—(C₁-C₆ alkyl);
o) C₁-C₆ alkyl or —O—C₁-C₆ alkyl substituted by 0, 1, 2, 3, 4 or 5 substituents selected from the group of halogen, CF₃, and OH;
p) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
q) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
r) two R₃ form a bridge between carbon atoms, wherein the atoms of the bridge comprise 1, 2, or 3 atoms independently selected from the group of carbon, N, O, and S;
R₄ is present one or two times and is independently selected from the group of H, halogen, oxo, C₁-C₆ alkyl, —(CH₂)ₙ₂—C₃-C₆ cycloalkyl, —O—(CH₂)ₙ₂—C₃-C₆ cycloalkyl, —C(—O)—O—C₁-C₆ alkyl, —S(═O)₂—C₁-C₆ alkyl, —C(═O)—NR^{x}R^{y}, —N(R^{x})(S(—O)₂—C₁-C₆ alkyl), phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a spirocyclic carbocycle or spirocyclic heterocycle;
wherein the R₄ C₁-C₆ alkyl, —C(═O)—O—C₁-C₆ alkyl, and —O—C₁-C₆ alkyl groups and the rings of the —(CH₂)ₙ₂—C₃-C₆ cycloalkyl and —O—(CH₂)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n₂ in each instance is an integer selected from the group of 0, 1, 2, and 3.

4. A compound of Formula (I-D), or a pharmaceutically acceptable salt thereof: 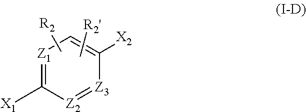
wherein:
X₁ is selected from the group of: 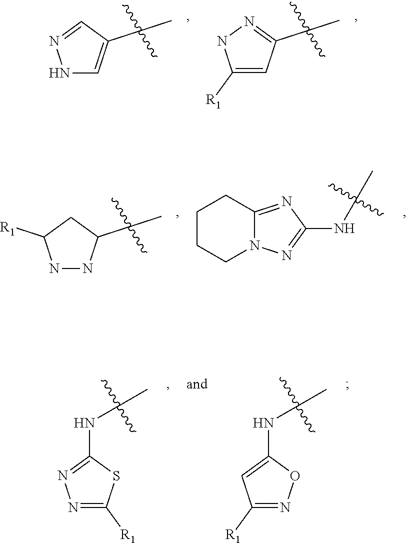
nₐ is an integer selected from the group of 0, 1, and 2;
n_{b} is an integer selected from the group of 0, 1, 2, 3, and 4;
with the proviso that the sum of nₐ+n_{b} is not less than 2 and not greater than 4;
or X₁ and Z₁ together form a fused ring system of the formula (Ia): 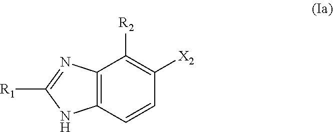
Rₐ is selected from the group of hydrogen and C₁-C₃ alkyl;
X₂ is selected from the group of: 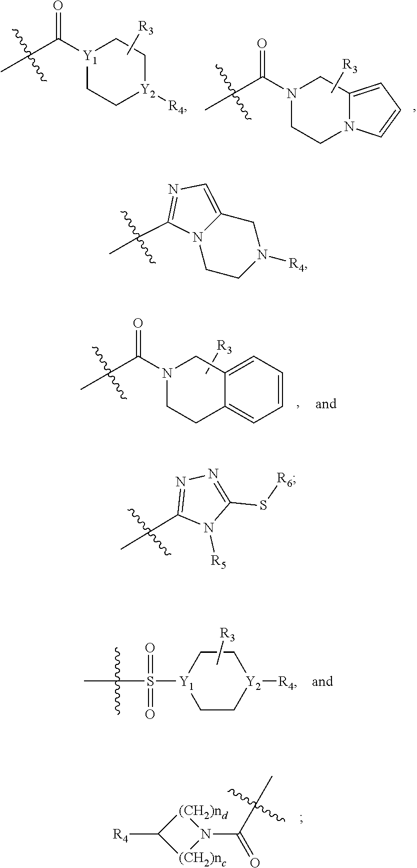
the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} is not less than 2 and not greater than 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl, pyridinyl bound through a carbon atom, and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂—C₁-C₆ alkyl, —C(═O)—NR^{x}R^{y}, —N(R^{x})(S(═O)₂—C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl, with the proviso that R₂ is not pyridinyl unless substituted at least once by —(CH₂)ₙ₁—C₃-C₆ cycloalkyl or phenyl;
R₂′ is selected from the group of hydrogen, OH, halogen, C₁-C₆ alkyl, and —CF₃;
R₃ is present one or more times and is independently selected from the group of:
s) hydrogen, halogen, cyano, or OH;
t) —CO₂H or —CO₂—(C₁-C₆ alkyl);
u) C₁-C₆ alkyl or —O—C₁-C₆ alkyl substituted by 0, 1, 2, 3, 4 or 5 substituents selected from the group of halogen, CF₃, and OH;
v) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
w) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
x) two R₃ form a bridge between carbon atoms, wherein the atoms of the bridge comprise 1, 2, or 3 atoms independently selected from the group of carbon, N, O, and S;
R₄ is present one or two times and is independently selected from the group of H, halogen, oxo, C₁-C₆ alkyl, —(CH₂)ₙ₂—C₃-C₆ cycloalkyl, —O—(CH₂)ₙ₂—C₃-C₆ cycloalkyl, —C(═O)—O—C₁-C₆ alkyl, —S(═O)₂—C₁-C₆ alkyl, —C(═O)—NR^{x}R^{y}, —N(R^{x})(S(═O)₂—C₁-C₆ alkyl), phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a spirocyclic carbocycle or spirocyclic heterocycle;
wherein the R₄ C₁-C₆ alkyl, —C(═O)—O—C₁-C₆ alkyl, and —O—C₁-C₆ alkyl groups and the rings of the —(CH₂)ₙ₂—C₃-C₆ cycloalkyl and —O—(CH₂)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n₂ in each instance is an integer selected from the group of 0, 1, 2, and 3.

5. A compound of Formula (I-E), or a pharmaceutically acceptable salt thereof: 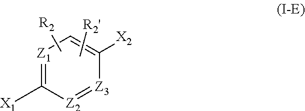
wherein:
X₁ is selected from the group of: 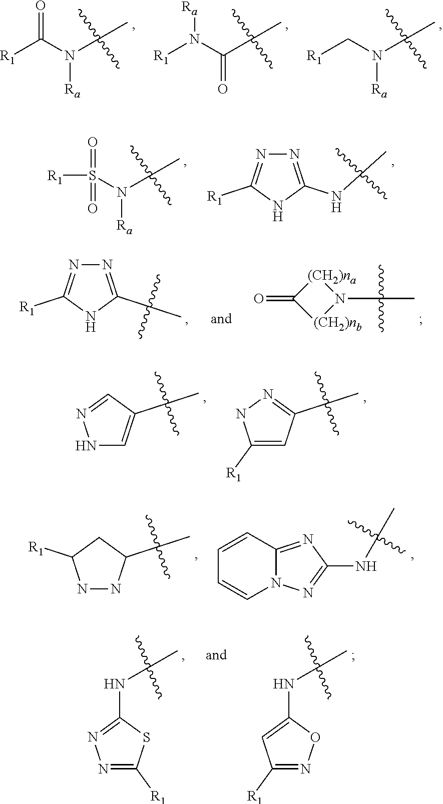
nₐ is an integer selected from the group of 0, 1, and 2;
n_{b} is an integer selected from the group of 0, 1, 2, 3, and 4;
with the proviso that the sum of nₐ+n_{b} is not less than 2 and not greater than 4;
or X₁ and Z₁ together form a fused ring system of the formula (Ia): 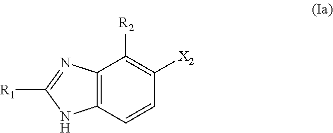
Rₐ is selected from the group of hydrogen and C₁-C₃ alkyl;
X₂ is selected from the group of: 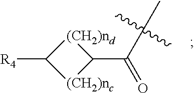
the wavy line [symbol] in each instance represents a bond through which each X₁ and X₂ moiety is bound;
Y₁ is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁ and Y₂ are C;
n_{c} is an integer selected from the group of 1, 2, and 3;
n_{d} is an integer selected from the group of 1, 2, and 3;
with the proviso that the sum of n_{c}+n_{d} is not less than 2 and not greater than 6;
Z₁, Z₂, and Z₃ are each independently selected from the group of C and N, with the proviso that Z₁, Z₂, and Z₃, when bound to R₂ or non-hydrogen R₂′ are C;
R₁ is selected from the group of C₁-C₆ alkyl, —(CH₂)ₙ₁—C₃-C₆ cycloalkyl, —NR^{x}R^{y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl, pyridinyl bound through a carbon atom, and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatoms selected from the group of N, S, and O, with any of the foregoing substituted by 0, 1, 2, or 3 substituents selected from the group of C₁-C₆ alkyl, —O—C₁-C₆ alkyl, —(CH₂)ₙ₁—C₃-C₆ cycloalkyl, —CF₃, halogen, oxo, cyano, —O—(CH₂)ₙ₂—C₃-C₆ cycloalkyl, —C(═O) OH, —C(═O)—O—C₁-C₆ alkyl, —S(═O)₂—C₁-C₆ alkyl, —C(═O)—NR^{x}R^{y}, —N(R^{x})(S(═O)₂—C₁-C₆ alkyl), —(CH₂)ₙ₁-heterocyclyl (containing a monocyclic heterocyclic ring or a bicyclic or spirocyclic heterocyclic ring system and containing 3, 4, 5, 6, 7, or 8 ring carbon atoms and 1, 2, 3, or 4 ring heteroatom selected from the group of N, S, and O), and phenyl, with the proviso that R₂ is not pyridinyl unless substituted at least once by —(CH₂)ₙ₁—C₃-C₆ cycloalkyl or phenyl;
R₂′ is selected from the group of hydrogen, OH, halogen, C₁-C₆ alkyl, and —CF₃;
R₃ is present one or more times and is independently selected from the group of:
y) hydrogen, halogen, cyano, or OH;
z) —CO₂H or —CO₂—(C₁-C₆ alkyl);
aa) C₁-C₆ alkyl or —O—C₁-C₆ alkyl substituted by 0, 1, 2, 3, 4 or 5 substituents selected from the group of halogen, CF₃, and OH;
bb) phenyl, benzyl, C₃-C₆ cycloalkyl, and —CH₂—C₃-C₆ cycloalkyl, the rings of each of the phenyl, benzyl, C₃-C₆ cycloalkyl, and —CH₂—C₃-C₆ cycloalkyl groups being substituted by 0, 1, 2, or 3 substituents selected from OH, halogen, cyano, and C₁-C₆ alkyl, wherein the C₁-C₆ alkyl group is further substituted by 0, 1, 2, 3, 4 or 5 substituents selected from the group of halogen, —CF₃, and OH;
cc) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH; and
dd) two R₃ form a bridge between carbon atoms, wherein the atoms of the bridge comprise 1, 2, or 3 atoms independently selected from the group of carbon, N, O, and S;
R₄ is present one or two times and is halogen;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R and R^{y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₁-C₃ alkyl; and
n₂ in each instance is an integer selected from the group of 0, 1, 2, and 3,
with the proviso that the compound is not N-[4-(4,4-difluoropiperidine-1-carbonyl)-3-pyrrolidin-1-ylphenyl]cyclopropanecarboxamide; (4,4-difluoropiperidin-1-yl)-[4-(5-methyl-4H-1,2,4-triazol-3-yl)-2-[3-(trifluoromethyl) pyrazol-1-yl]phenyl]methanone; [2-(3-cyclopropylpyrazol-1-yl)-4-(5-methyl-4H-1,2,4-triazol-3-yl)phenyl]-(4,4-difluoropiperidin-1-yl)methanone; (4,4-difluoropiperidin-1-yl)-[4-(5-methyl-4H-1,2,4-triazol-3-yl)-2-(3-propan-2-ylpyrazol-1-yl)phenyl]methanone; or N-[4-(4-fluoropiperidine-1-carbonyl)-3-pyrrolidin-1-ylphenyl]cyclopropanecarboxamide.

6. The compound of claim 1, wherein EITHER R₄ is not unsubstituted benzyl, wherein R₄ is not unsubstituted pyridinyl, wherein R₄ is not substituted or unsubstituted phenyl, wherein R₃ is not H, OR combinations thereof.

7-12. (canceled)

13. The compound of claim 1, or a pharmaceutically acceptable salt thereof, having the formula: 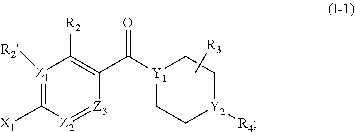
wherein, X₁, Z₁, Z₂, Z₃, Y₁, Y₂, R₂, R₂′, R₃, and R₄ are as defined in claim 0.

14. The compound of claim 1, or a pharmaceutically acceptable salt thereof, having the formula: 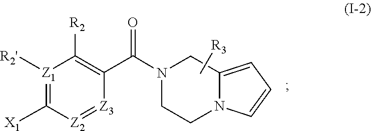
wherein, X₁, Z₁, Z₂, Z₃, R₂, R₂′, and R₃ are as defined in claim 0.

15. The compound of claim 1, or a pharmaceutically acceptable salt thereof, having the formula: 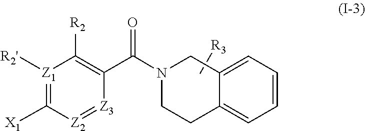
wherein, X₁, Z₁, Z₂, Z₃, R₂, R₂′, and R₃ are as defined in claim 0.

16. The compound of claim 1, or a pharmaceutically acceptable salt thereof, having the formula: 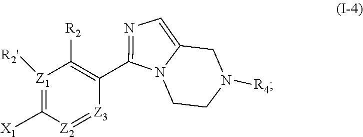
wherein, X₁, Z₁, Z₂, Z₃, R₂, R₂′, and R₄ are as defined in claim 0.

17. The compound of claim 1, or a pharmaceutically acceptable salt thereof, having the formula: 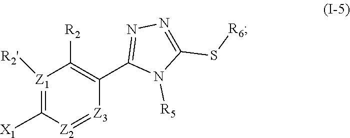
wherein, X₁, Z₁, Z₂, Z₃, R₂, R₂′, R₄, R₅, and R⁶ are as defined in claim 0.

18. The compound of claim 1, or a pharmaceutically acceptable salt thereof, having the formula: 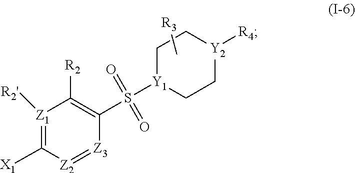
wherein X₁, Z₁, Z₂, Z₃, R₂, R₂′, and R₄ are as defined in claim 1.

19. The compound of claim 1, or a pharmaceutically acceptable salt thereof, having the formula: 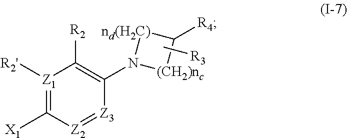
wherein X₁, Z₁, Z₂, Z₃, R₂, R₂′, and R₄ are as defined in claim 1.

20-66. (canceled)

67. The compound of claim 1, or a pharmaceutically acceptable salt thereof, wherein R₂ is selected from the group of: 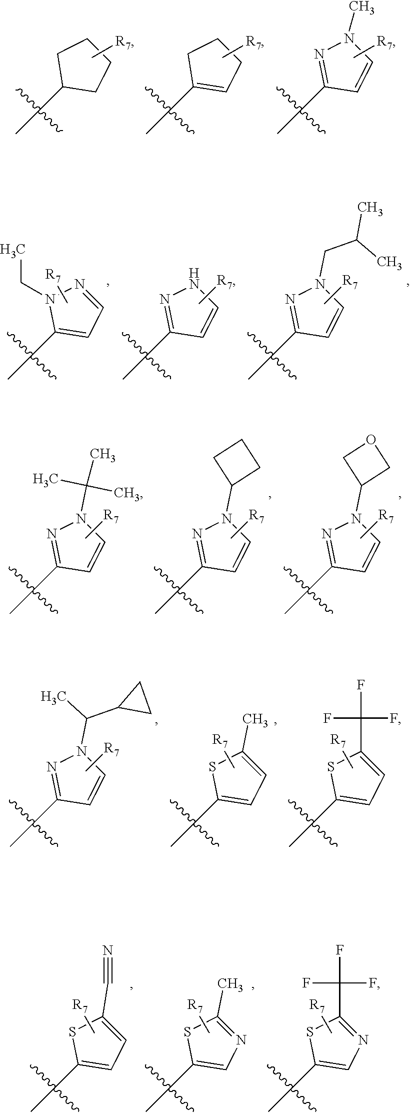 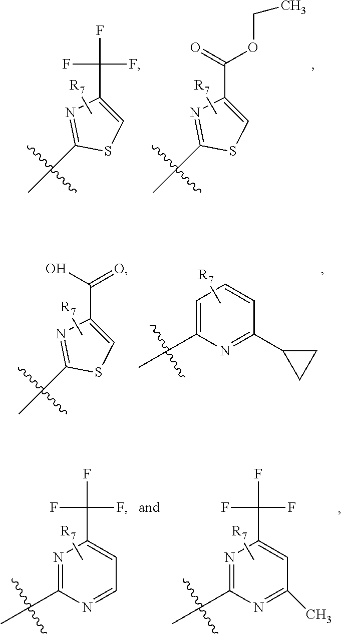
wherein R₇ is present one or two times and is independently selected from the group of hydrogen, —OH; halogen; —C₁-C₆ alkyl; —O—C₁-C₆ alkyl; —(CH₂)ₙ₁—C₃-C₆ cycloalkyl; —(CH₂)ₙ₁—C₃-C₆ heterocycle including at least one heteroatom selected from O, N, and S; cyano; —O—(CH₂)ₙ₂—C₃-C₆ cycloalkyl; —C(═O) OH; —C(═O)—O—C₁-C₆ alkyl; —S(—O)₂—C₁-C₆ alkyl; —C(═O)—NR^{x}R^{y}; —N(R^{x})(S(═O)₂—C₁-C₆ alkyl); and —CF₃, wherein any of the alkyl groups are further substituted with 0, 1, 2, 3, or 4 substituents selected from halogen and —OH, and wherein n1 is an integer selected from the group of 0, 1, 2, and 3 OR wherein R₂ is selected from the group of:

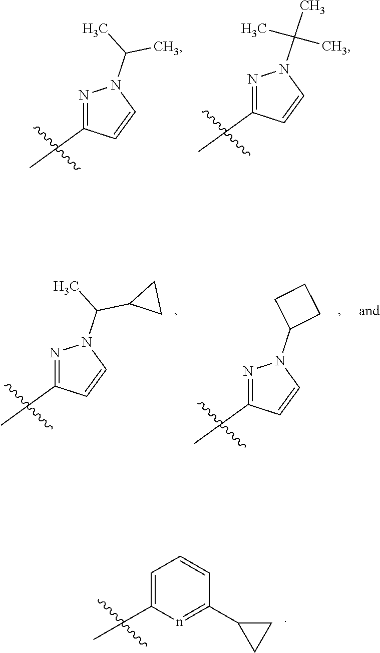

68-73. (canceled)

74. The compound of claim 1, or a pharmaceutically acceptable salt thereof, wherein R₅ and R₆ are each independently selected from the group of hydrogen, C₁-C₄ alkyl, and C₁-C₄ alkoxy.

75. A pharmaceutical composition comprising a pharmaceutically effective amount of a compound of claim 1, a compound selected from Examples 1-125, or a pharmaceutically acceptable salt of any of the foregoing, and a pharmaceutically acceptable carrier or excipient.

76-102. (canceled)

103. A method of inhibiting the activity of a GPR39 protein in a subject, A method of treating hypertension in a human in need thereof, A method of treating heart failure in a human, A method of breast cancer in a human in need thereof, A method of gastric adenocarcinomas in a human in need thereof, A method of promoting or enhancing colon epithelial function and tight junction barrier integrity in a human, A method of treating ulcerative colon disease in a human, A method of treating Inflammatory Bowel Disease in a human, A method of treating diarrhea in a human, A method of enhancing the delivery of an anesthetic to a human experiencing microvascular complications, A method of treating stroke in a human in need thereof, the method comprising administering to the subject in need thereof a pharmaceutically effective amount of a compound according to claim 1, a compound selected from Examples 1-125, a compound of Formula (I′), or a pharmaceutically acceptable salt of any of the foregoing.

104-121. (canceled)

122. A kit comprising:
a) one or more compositions, each composition comprising a pharmaceutically effective amount of a compound selected from claim 1, a compound selected from Examples 1-125, or a pharmaceutically acceptable salt of any of the foregoing, and a pharmaceutically acceptable carrier or excipient; and
b) instructions for administering the one or more compositions to a human in need thereof.

123. A compound of any one of Examples 1-125, or a pharmaceutically acceptable salt thereof.
